(19) United States
(12) Patent Application Publication
(10) Pub. No.: US 2024/0194303 A1
(43) Pub. Date: Jun. 13, 2024

(54) CONTRASTIVE SYSTEMS AND METHODS

(71) Applicant: Cellarity, Inc., Somerville, MA (US)

(72) Inventors: Peter Holderrieth, Cambridge, MA (US); Sergey Kolchenko, Cambridge, MA (US); Diogo Camacho, Sudbury, MA (US); Mahdi Zamanighomi, Belmont, MA (US)

(21) Appl. No.: 18/539,204

(22) Filed: Dec. 13, 2023

Related U.S. Application Data
(60) Provisional application No. 63/387,230, filed on Dec. 13, 2022.

(51) Int. Cl. G16C 20/50

(52) U.S. Cl. CPC G16C 20/50

(57) ABSTRACT

Systems and methods for determining whether first and second compounds are causal for a biological state include inputting a representation of the first compound and a baseline transcriptional representation into a structure encoder, thereby obtaining a first compound embedding. A representation of the second compound and the baseline transcriptional representation is inputted into the structure encoder to obtain a second compound embedding. The first and second compound embeddings are projected into a plurality of overlayed transcriptional embeddings that form clusters, each such cluster representing a corresponding biological state. The transcriptional embeddings are generated from corresponding cellular constituent abundance data set (e.g., exposed to a different perturbation) inputted into a transcriptional encoder. When the first and second compound embeddings fall into a common cluster, the first compound is associated with the biological state of the cluster or of the second compound.

### CROSS REFERENCE TO RELATED APPLICATION

This application claims priority to U.S. Provisional Patent Application No. 63/387,230 entitled “Contrastive Systems and Methods,” filed Dec. 13, 2022, which is hereby incorporated by reference.

### TECHNICAL FIELD

The present invention relates generally to systems and methods for associating compounds with cellular transitions.

### BACKGROUND

Prediction of drug candidates through transcriptomics relies on extensive data sets of transcriptional perturbations. For instance, high-throughput single-cell RNA-sequencing(scRNA-seq) allows profiling of genome-wide expression of thousands of individual cells subjected to such perturbations with single-cell precision. See Chen et al., 2022, “Recent advance in high-throughput single-cell transcriptomics and spatial transcriptomics,” Lab Chip 22, p. 4774. Ideally, these data sets are comprised of varied small molecules with high structural and transcriptional diversity, across a variety of cell systems, to ensure comprehensive coverage of response of biological systems to interventions. However, the generation of such data is expensive and time-consuming, and requires skilled bioinformatics support. Although the cost per cell in high-throughput scRNA-seq is modest, the overall cost for transcriptomic profiling thousands of cells is still high. The high cost is attributed to a plethora of reagents used in scRNA-seq including sample processing, single-cell cDNA synthesis and amplification, library construction, and high-throughput sequencing. Id. at page 4787.

### SUMMARY

Given the above background, what is needed in the art are systems and methods for identification of candidate compounds for drug discovery with reduced cost that are less reliant on wet lab transcriptomics.

The present disclosure addresses the above-identified shortcomings, at least in part, by using machine learning methods that take advantage of both structural information and transcriptional information about candidate compounds to identify potential novel clinical compounds without the need for direct transcriptional measurements of such candidate compounds. The present disclosure makes use of a novel machine learning model, based on metric learning, that learns a joint representation of both data the structural and transcriptional information to predict interventions that impact disease states of interest. The model is trained on a perturbational data set, consisting of plurality of compounds with transcriptional profiles across different cell types, to generate a transcriptional embedding. The transcriptional embedding is combined with the embedding space from a pre-trained chemical model that has been trained on millions of molecular structures. By creating a transcription-structure co-embedding, the search space for hits beyond the compounds for which transcriptional data is available is expanded at least ten fold, thereby reducing costs for screening for compounds that affect biological states of interest. The disclosed model has a number of uses cases including (i) determining whether a first compound and a second compound are causal for a common biological state and (ii) identifying a biological state for which a candidate compound is causal.

A. Determining a Likelihood that a Test Compound Causes a Differential Expression Signature Between a First Cell State and a Second Cell State.

One aspect of the present disclosure provides systems and methods of the present disclosure provides a hit prediction tool in, for example, drug discovery approaches characterized by two steps: (A.1) characterization of a disease by a transition from a healthy cell to a diseased cell and (A.2) identification of compounds that reverse this cell transition. The discloses systems and methods serve a role in step A.2. That is, the disclosed systems and methods allows for the screening of novel organic molecules computationally to find compounds that induce a desired transcriptional response. The above-identified two step drug discovery platform is in contrast to more traditional target-based drug discovery approach that consists of two steps: (B.1) find a protein (“target”) that is believed to be associated with a disease and (B.2) Design a novel molecule that selectively binds to this target. The drug discovery approach involving steps A.1 and B.1 does not reduce the biology of a disease to a single target (compare step A.1. to B.1.) and therefore promises to better capture the complexity of disease biology. However, while in target-based drug discovery design process is guided by the three-dimensional crystal structure of a protein (B.2.), there is no comparable design process to create a molecule inducing a certain cell transition (A.2.). Thus, the discloses systems and method provide a data-driven (or machine learning-based) approach for step A.2.

As outlined above (step A.1.), a disease is characterized by a transition from a healthy cell to a diseased cell (or vice versa). This can be considered a “transcriptional transition”, e.g., a change in the RNA makeup of a cell. The disclosed systems and methods take a differential expression signature (transcriptional perturbations/transitions) as one of its inputs and thereby connects the disease modelling capabilities with chemistry. The disclosed systems and methods are designed such that they can also accept gene modules and other modalities such as chromatin accessibility—allowing flexibility with respect to the characterization of a disease.

Once a chemical hit is found using the disclosed systems and methods, the chemical hit can be optimized for desired properties (e.g. toxicity, induction of a certain phenotypic response, etc.). To further optimize the desired cell transition/transcriptional response, the disclosed systems and methods can also be used.

In some embodiments, a method of determining a likelihood that a test chemical compound causes a differential expression signature between a first cell state and a second cell state. In some such embodiments, the first cell state represents a wild-type disease-free state and the second cell state represents a diseased state. In some embodiments, a fingerprint of a test chemical compound is obtained. In some such embodiments, the test chemical compound is a first organic compound having a molecular weight of less than 2000 Daltons.

In some such embodiments, the test chemical compound satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some such embodiments, the fingerprint of the test chemical compound comprises 100 features. In some such embodiments, the fingerprint of the test chemical compound consists of between 10 features and 100,000 features. In some such embodiments, the fingerprint of the test chemical compound is calculated from a chemical structure of the test chemical compound using a simplified molecular-input line-entry system (SMILES) string representation of the test chemical compound.

In some such embodiments, the fingerprint of the test chemical compound is calculated from a chemical structure of the test chemical compound using Daylight, BCI, ECFP4, EcFC, MDL, APFP, TTFP, UNITY 2D fingerprint, RNNS2S, or GraphConv.

In some such embodiments, the fingerprint of the test chemical compound is calculated as a plurality of features that comprise a plurality of bioactivity descriptors for the test chemical compound. In some embodiments, the plurality of bioactivity descriptors include a numeric representation of the test chemical compound obtained from a two-dimensional fingerprint of the test chemical compound, a mechanism of action of the test chemical compound, a small molecule role possessed by the test chemical compound, a therapeutic area associated with the test chemical compound, a three-dimensional fingerprint of the test chemical compound, an association of the test chemical compound with one or more metabolic genes, an association of the test chemical compound with a small molecule pathway, an association of the test chemical compound with a cancer cell line, a crystal structure of the test chemical compound, a signaling pathway associated with the test chemical compound, a therapeutic side effect association with the test chemical compound, a structural key associated with the test chemical compound, a binding affinity of the test chemical compound against a macromolecular target, a biological process associated with the test chemical compound, a morphology of cells exposed to the test chemical compound, a disease associated with the test chemical compound, a toxicology associated the test chemical compound, a physicochemistry associated with the test chemical compound, a drug-drug interaction associated with the test chemical compound, an inhibitory constant associated with the test chemical compound, a binding interaction of the test chemical compound with one or more residues of a protein, a Gibbs free energy of the binding of the test chemical compound with a protein, or any combination thereof.

In some such embodiments, a model is used that predicts bioactivity descriptors to determine one or more of the plurality of bioactivity descriptors.

In some embodiments, a differential expression signature is obtained. The differential expression signature comprises a plurality of differential values. Each respective differential value in the plurality of differential values corresponds to a respective cellular constituent in a set of cellular constituents. The respective differential value represents a difference between (i) one or more abundance values measured for the respective cellular constituent in a first cell-based assay of a first plurality of cells that represent the first cell state and (ii) one or more abundance values measured for the respective cellular constituent in a second cell-based assay of a second plurality of cells that represent the second cell state.

In some such embodiments, each corresponding differential value in the plurality of differential values is a comparison of: (i) a first measure of central tendency of the one or more abundance values for the respective cellular constituent across the first plurality of cells, and (ii) a second measure of central tendency of the one or more abundance values for the respective cellular constituent across the second plurality of cells.

In some such embodiments, the one or more abundance values measured for the respective cellular constituent in the first cell-based assay are obtained by a first single-cell assay and the one or more abundance values measured for the respective cellular constituent in the second plurality of cell-based assay are obtained by a second single-cell assay.

In some such embodiments, the first single-cell assay and the second single-cell assay is ribonucleic acid (RNA) sequencing (scRNA-seq), CITE-seq, ATAC-seq, or single cell ATAC-seq (scATAC-seq).

In some such embodiments, each cellular constituent in the set of cellular constituents uniquely maps to a different gene.

In some such embodiments, each cellular constituent in the set of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification (e.g., glycosylation, phosphorylation, acetylation, or ubiquitylation) of a protein.

In some such embodiments, the set of cellular constituents comprises 3 cellular constituents, 4 cellular constituents, 5 cellular constituents, 6 cellular constituents, 7 cellular constituents, 8 cellular constituents, 9 cellular constituents, 10 or more cellular constituents, 20 or more cellular constituents, 30 or more cellular constituents, 40 or more cellular constituents, or 50 or more cellular constituents.

In some such embodiments, the set of cellular constituents consists of between 10 and 1000 cellular constituents.

In some such embodiments, the first plurality of cells and the second plurality of cells are cells from an organ, cells from a tissue, a plurality of stem cells, a plurality of primary human cells, cells from umbilical cord blood, cells from peripheral blood, bone marrow cells, cells from a solid tissue, or a plurality of differentiated cells.

In some such embodiments, the one or more abundance values measured for the respective cellular constituent in the first cell-based assay or the one or more abundance values measured for the respective cellular constituent in the second cell-based assay are determined by a colorimetric measurement, a fluorescence measurement, a luminescence measurement, a resonance energy transfer (FRET) measurement, a measurement of a protein-protein interaction, a measurement of a protein-polynucleotide interaction, a measurement of a protein-small molecule interaction. mass spectrometry, nuclear magnetic resonance, or a microarray measurement.

In some embodiments, responsive to inputting the fingerprint of the test chemical compound into a first model, there is retrieved, as output from the first model, a respective chemical embedding.

In some embodiments, a differential expression embedding is retrieved as output from a second model responsive to inputting the differential expression signature into the second model.

In some such embodiments, the first model is a first multilayer perceptron and the second model is a second multilayer perceptron. In some such embodiments, the first model comprises 1000 parameters and the second model comprises 1000 parameters. In some such embodiments, the first model consists of between 10 and 10 million parameters and the second model consists of between 10 and 10 million parameters. In some such embodiments, the first model comprises one million parameters and the second model comprises one million parameters.

In some embodiments, the likelihood that the test chemical compound causes the differential expression signature is determine based on a similarity between the respective chemical embedding and the differential expression embedding. In some such embodiments, the similarity between the respective chemical embedding and the differential expression embedding is determined by a distance between the respective chemical embedding and the differential expression embedding.

In some such embodiments, the differential expression signature is associated with alleviating a condition in a subject, and the method further comprises administering the test chemical compound to the subject as a treatment to alleviate the condition in the subject when the test chemical compound is found to have a threshold likelihood of causing the differential expression signature.

In some such embodiments, the treatment comprises a composition comprising the test chemical compound and one or more excipient and/or one or more pharmaceutically acceptable carrier and/or one or more diluent.

In some such embodiments, the condition is inflammation or pain. In some such embodiments, the condition is a disease. In some such embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

In some such embodiments, the method further comprises training the first model and the second model. In some such embodiments, the training comprises contrastive learning. In some such embodiments, the training comprises training the first and second model jointly against a single loss function.

### B. Methods of Rank Ordering a Plurality of Test Compounds Against a Differential Expression Signature Between a First Cell State and a Second Cell State.

Another aspect of the present disclosure provides methods of rank ordering a plurality of test chemical compounds against a differential expression signature between a first cell state and a second cell state.

In some such embodiments, the first cell state represents a wild-type disease-free state and the second cell state represents a diseased state. In some embodiments, the plurality of test chemical compounds comprises 1000, 10,000, 100,000, or one million chemical compounds.

In some embodiments, a differential expression signature is obtained. The differential expression signature comprises a plurality of differential values. Each respective differential value in the plurality of differential values corresponds to a respective cellular constituent in a set of cellular constituents. The respective differential value represents a difference between (i) one or more abundance values measured for the respective cellular constituent in a first cell-based assay of a first plurality of cells that represent the first cell state and (ii) one or more abundance values measured for the respective cellular constituent in a second cell-based assay of a second plurality of cells that represent the second cell state.

In some such embodiments, each corresponding differential value in the plurality of differential values is a comparison of (i) a first measure of central tendency of the one or more abundance values for the respective cellular constituent across the first plurality of cells, and (ii) a second measure of central tendency of the one or more abundance values for the respective cellular constituent across the second plurality of cells.

In some such embodiments, the one or more abundance values measured for the respective cellular constituent in the first cell-based assay are obtained by a first single-cell assay, and the one or more abundance values measured for the respective cellular constituent in the second cell-based assay are obtained by a second single-cell assay.

In some such embodiments, the first single-cell assay and the second single-cell assay is ribonucleic acid (RNA) sequencing (scRNA-seq), CITE-seq, ATAC-seq, or SCATAC-seq.

In some such embodiments, the one or more abundance values measured for the respective cellular constituent in the first cell-based assay or the one or more abundance values measured for the respective cellular constituent in the second cell-based assay are determined by a colorimetric measurement, a fluorescence measurement, a luminescence measurement, a resonance energy transfer (FRET) measurement, a measurement of a protein-protein interaction, a measurement of a protein-polynucleotide interaction, a measurement of a protein-small molecule interaction. mass spectrometry, nuclear magnetic resonance, or a microarray measurement.

In some such embodiments, each cellular constituent in the set of cellular constituents uniquely maps to a different gene.

In some such embodiments, each cellular constituent in the set of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein.

In some such embodiments, the set of cellular constituents consists of between 100 and 1000 cellular constituents. In some such embodiments, the set of cellular constituents comprises 3 cellular constituents, 4 cellular constituents, 5 cellular constituents, 6 cellular constituents, 7 cellular constituents, 8 cellular constituents, 9 cellular constituents, 10 or more cellular constituents, 20 or more cellular constituents, 30 or more cellular constituents, 40 or more cellular constituents, or 50 or more cellular constituents.

In some such embodiments, the first plurality of cells and the second plurality of cells are cells from an organ, cells from a tissue, a plurality of stem cells, a plurality of primary human cells, cells from umbilical cord blood, cells from peripheral blood, bone marrow cells, cells from a solid tissue, or a plurality of differentiated cells.

In some embodiments, for each respective test chemical compound in the plurality of test chemical compounds, input a respective fingerprint of the respective test chemical compound into a first model, thereby retrieving, as output from the first model, a corresponding chemical embedding, thereby obtaining a plurality of chemical embeddings, each respective chemical embedding corresponding to a respective test chemical compound in the plurality of test chemical compounds.

In some such embodiments, the respective fingerprint of the respective test chemical compound is calculated from a chemical structure of the respective test chemical compound using a simplified molecular-input line-entry system (SMILES) string representation of the test chemical compound.

In some such embodiments, the respective fingerprint of the respective test chemical compound is calculated from a chemical structure of the respective test chemical compound using Daylight, BCI, ECFP4, EcFC, MDL, APFP, TTFP, UNITY 2D fingerprint, RNNS2S, or GraphConv.

In some such embodiments, the fingerprint of the respective test chemical compound is calculated as a plurality of features that comprise a plurality of bioactivity descriptors for the test chemical compound. In some embodiments, the plurality of bioactivity descriptors include a numeric representation of the respective test chemical compound obtained from a two-dimensional fingerprint of the respective test chemical compound, a mechanism of action of the respective test chemical compound, a small molecule role possessed by the respective test chemical compound, a therapeutic area associated with the respective test chemical compound, a three-dimensional fingerprint of the respective test chemical compound, an association of the respective test chemical compound with one or more metabolic genes, an association of the respective test chemical compound with a small molecule pathway, an association of the respective test chemical compound with a cancer cell line, a crystal structure of the respective test chemical compound, a signaling pathway associated with the respective test chemical compound, a therapeutic side effect associated with the respective test chemical compound, a structural key associated with the respective test chemical compound, a binding affinity of the respective test chemical compound against a macromolecular target, a biological process associated with the test respective chemical compound, a morphology of cells that have been exposed to the respective test chemical compound, a disease associated with the respective test chemical compound, a toxicology associated the respective test chemical compound, a physicochemistry associated with the respective test chemical compound, a drug-drug interaction associated with the respective test chemical compound, an inhibitory constant associated with the respective test chemical compound, a binding interaction of the respective test chemical compound with one or more residues of a protein, a Gibbs free energy of the binding of the respective test chemical compound with a protein, or any combination thereof.

In some such embodiments, a model is used that predicts bioactivity descriptors to determine one or more of the plurality of bioactivity descriptors.

In some such embodiments, the respective fingerprint of the respective test chemical compound comprises 100 features. In some such embodiments, the respective fingerprint of the respective test chemical compound consists of between 10 features and 1000 features.

In some such embodiments, the respective test chemical compound is a first organic compound having a molecular weight of less than 2000 Daltons.

In some such embodiments, the respective test chemical compound satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some such embodiments, the method further comprises training the first model and the second model. In some such embodiments, the training comprises contrastive learning. In some such embodiments, the training comprises training the first and second model jointly against a single loss function.

In some such embodiments, the first model comprises 1000 parameters and the second model comprises 1000 parameters. In some embodiments, the first model consists of between 10 and 10 million parameters and the second model consists of between 10 and 10 million parameters.

In some embodiments, responsive to inputting the differential expression signature into a second model, a differential expression embedding is retrieved as output from the second model.

In some such embodiments, the first model is a first multilayer perceptron and the second model is a second multilayer perceptron.

In some embodiments, each respective test chemical compound in the plurality of test chemical compounds is ranked based on a respective similarity between the respective chemical embedding corresponding to the respective test chemical compound and the differential expression embedding.

In some such embodiments, the respective similarity between the respective chemical embedding and the differential expression embedding is determined by a distance between the respective chemical embedding and the differential expression embedding.

In some such embodiments, the differential expression signature is associated with alleviating a condition in a subject, and the method further comprises administering the respective test chemical compound to the subject as a treatment to alleviate the condition in the subject when the respective test chemical compound is found to have a threshold ranking in the plurality of test chemical compounds or a threshold similarity.

In some such embodiments, the treatment comprises a composition comprising the respective test chemical compound and one or more excipient and/or one or more pharmaceutically acceptable carrier and/or one or more diluent.

In some such embodiments, the condition is inflammation or pain. In some such embodiments, the condition is a disease. In some such embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

### C. Determining Whether a First Compound and a Second Compound are Causal for a Common Biological State.

Another aspect of the present disclosure provides systems and methods for determining whether a first compound and a second compound are causal for a common biological state.

A first input data structure is inputted into a structure encoder. The first input data structure comprises a combination of a feature representation of the first compound and a baseline transcriptional representation of the first cell type. The structure encoder comprises a first plurality of parameters. There is retrieved, by operation of the first plurality of parameters on the first input data structure in accordance with an architecture of the structure encoder, as output from the structure encoder, a first compound embedding having a first dimension.

In some embodiments, the feature representation of the first compound is determined from a string representation of a chemical structure of the first compound. In some such embodiments, the string representation is in a SMARTS, DeepSMILES, SELFIES, or SMILES format.

In some embodiments, the determination of the feature representation of the first compound from a string representation of a chemical structure of the first compound comprises inputting the string representation into each featurizer in a set of featurizers to obtain the feature representation. In some such embodiments, the set of featurizers consists of 2, 3, or 4 featurizers in Table 2. In some embodiments, the feature representation of the first compound is a concatenation of an output of each feature in the set of features.

In some such embodiments, the set of featurizers consists of between 2 and 40 featurizers in Table 3. The feature representation of the first compound is a concatenation of an output of each feature in the set of features.

In some embodiments, a featurizer in the set of featurizer is a graph isomorphism network.

In some embodiments, the feature representation of the first compound consists of between 150 and 10,000 features.

In some embodiments, the baseline transcriptional representation of the first cell type comprises pathway activation scores for a plurality of pathways derived from cellular constituent abundance data for a plurality of cellular constituents in a plurality of cells of the first type that are in a baseline state.

In some embodiments, each cellular constituent in the plurality of cellular constituents uniquely maps to a different gene.

In some embodiments, each cellular constituent in the plurality of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein.

In some embodiments, the plurality of cellular constituents comprises 50 or more cellular constituents, 100 or more cellular constituents, 150 or more cellular constituents, 200 or more cellular constituents, 300 or more cellular constituents, 500 or more cellular constituents, 1000 or more cellular constituents, 2000 or more cellular constituents, 4000 or more cellular constituents, or 8000 or more cellular constituents.

In some embodiments, the plurality of pathways comprises 10 or more pathways, 20 or more pathways, 50 or more pathways, 100 or more pathways, or 500 or more pathways.

In some embodiments, the first compound embedding having the first dimension consists of between 40 and 2000 dimensions, or consists of between 50 and 500 dimensions, or consists of between 60 and 250 dimensions, or consists of between 70 dimensions and 100 dimensions.

In some embodiments, the first cell type is a CD34+ cell type in a plurality of CD34+ cell types in which expression of at least 2, 3, 4, 5 or all 6 genes in the group consisting of EPOR, KLF1, TFR2, CSF2RB, APOE, APOC1, and CNRIP1 is enriched relative to other cell types in the plurality of CD34+ cell types.

In some embodiments, the first cell type is a CD34+ cell type in a plurality of CD34+ cell types in which expression of at least 2, 3, 4, or all 5 genes in the group consisting of MPIG6B, PF4, FP9, VWF, and SELP is enriched relative to other cell types in the plurality of CD34+ cell types.

In some embodiments, the first cell type is a CD34+ cell type in a plurality of CD34+ cell types in which expression of at least 2, 3, 4, or all 5 genes in the group consisting of VPREB1, JCHAIN, CD22, IGHD, and LTB is enriched relative to other cell types in the plurality of CD34+ cell types.

In some such embodiments, the first cell type is a CD34+ cell type in a plurality of CD34+ cell types in which expression of at least 2, 3, 4, 5, 6, 7, 8, 9, 10, 11, 12, 13, 14, 15 or all 16 genes in the group consisting of ELANE, AZUI, PRTN3, CFD, MPO, CSFIR, CST7, CTSG, CYBB, FGL2, MARCH1, MRC1, NPL, ACP5, CYP27A1, and PLA2G7 is enriched relative to other cell types in the plurality of CD34+ cell types.

In some embodiments, the first cell type is a CD34+ cell type in a plurality of CD34+ cell types in which expression of at least 2, 3, 4, 5 or all 6 genes in the group consisting of CLC, HDC, PRG2, RNASE2, FCER1A, and CPA3 is enriched relative to other cell types in the plurality of CD34+ cell types.

In some embodiments, the first cell type is a CD34+ cell type in a plurality of CD34+ cell types in which expression of at least 2, 3, 4, 5, 6, 7 or all 8 genes in the group consisting of CRHBP, EMCN, HLF, AVP, RUNX1, HOXA9, MLLT3, PROM1 is enriched relative to other cell types in the plurality of CD34+ cell types.

In some embodiments, the structure encoder is a first multilayer perceptron having a first plurality of hidden layers. In some such embodiments, the first plurality of hidden layers consists of between 2 and 20 hidden layers. In some such embodiments, the first plurality of parameters consists of between 1000 and 1×10⁷ parameters.

In some embodiments, the structure encoder is a convolutional neural network or a graph based neural network.

A second input data structure is inputted into the structure encoder. The second input data structure comprises a combination of a feature representation of the second compound and the baseline transcriptional representation of the first cell type. The is retrieved, by operation of the first plurality of parameters on the second input data structure in accordance with the architecture of the structure encoder, as output from the structure encoder, a second compound embedding having the first dimension.

The first compound embedding and the second compound embedding are projected into a plurality of transcriptional embeddings each having the first dimension. Each respective transcriptional embedding in the plurality of transcriptional embeddings is overlayed onto each other transcriptional embedding in the plurality of transcriptional embeddings. At least a subset of the plurality of transcriptional embeddings collectively populates a plurality of clusters. Each cluster in the plurality of clusters is representative of a corresponding biological state. Each respective transcriptional embedding in the plurality of transcriptional embeddings is generated from inputting a corresponding cellular constituent abundance data set representative of the first cell type into a transcriptional encoder comprising a second plurality of parameters. The structure encoder is trained to minimize a loss against the plurality of transcriptional embeddings.

In some embodiments, the corresponding cellular constituent data set comprises single cell transcriptional data for a plurality of cells of the first type.

In some embodiments, the corresponding cellular constituent data comprises bulk transcriptional data for a plurality of cells of the first type.

In some embodiments, the corresponding cellular constituent data set comprises cellular constituent abundance values for a plurality of cellular constituents.

In some embodiments, each cellular constituent in the plurality of cellular constituents uniquely maps to a different gene.

In some embodiments, each cellular constituent in the plurality of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein.

In some embodiments, the plurality of cellular constituents comprises 50 or more cellular constituents, 100 or more cellular constituents, 150 or more cellular constituents, 200 or more cellular constituents, 300 or more cellular constituents, 500 or more cellular constituents, 1000 or more cellular constituents, 2000 or more cellular constituents, 4000 or more cellular constituents, or 8000 or more cellular constituents.

In some embodiments, the corresponding cellular constituent data set comprises a corresponding differential expression signature for a plurality of cells of the first type.

In some embodiments, the corresponding differential expression signature comprises a plurality of differential values, each respective differential value in the plurality of differential values corresponds to a respective cellular constituent in a set of cellular constituents, and the respective differential value represents a difference between (i) one or more abundance values measured for the respective cellular constituent in a first assay of a first plurality of cells of the first cell type that represent a first cell state and (ii) one or more abundance values measured for the respective cellular constituent in a second assay of a second plurality of cells of the first cell type that represent a second cell state.

In some embodiments, the first cell state is exposure of the first plurality of cells to a perturbation, and the second cell state is exposure of the second plurality of cells to a reference environment.

In some embodiments, the reference environment is exposure to a polar aprotic solvent (e.g., dimethyl sulfoxide)

In some embodiments, the perturbation is exposure of the first plurality of cells to a chemical compound solubilized in a polar aprotic solvent.

In some embodiments, the plurality of transcriptional embeddings collectively represents over 500 different first cell states or over 1000 different first cell states.

In some embodiments, the plurality of transcriptional embeddings collectively represents over 100 different biological pathways.

In some embodiments, the plurality of transcriptional embeddings collectively represents over 200 different biological pathways.

In some embodiments, each different first cell state is exposure of the first plurality of cells with a different chemical compound.

In some embodiments, the transcriptional encoder is a second multilayer perceptron having a second plurality of hidden layers. In some such embodiments, the second plurality of hidden layers consists of between 2 and 20 hidden layers. In some embodiments, the second plurality of parameters consists of between 1000 and 1×10⁷ parameters.

In some embodiments, the transcriptional encoder is a convolutional neural network or a graph based neural network.

In some embodiments, the respective transcriptional embedding consists of between 40 and 2000 dimensions, between 50 and 500 dimensions, between 60 and 250 dimensions, or between 70 dimensions and 100 dimensions.

In some embodiments, the plurality of clusters comprises five or more clusters representing five or more biological states.

In some embodiments, the plurality of clusters comprises 25 or more clusters representing 25 or more biological states.

In some embodiments, the first compound is a first organic compound having a molecular weight of less than 2000 Daltons.

In some embodiments, the first compound is a peptide having a mass of less than 4500 Daltons.

In some embodiments, the first compound is a protein having a mass of more than 4500 Daltons.

In some embodiments, the first compound satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

There is associated, when the first compound embedding and the second compound embedding fall into a first cluster in the plurality of clusters, the first compound with the corresponding biological state of the first cluster or with the corresponding biological state of the second compound.

D. Identifying a Biological State for which a First Compound is Causal.

Another aspect of the present disclosure provides a method of identifying a biological state for which a first compound is causal. A first input data structure is inputted into a structure encoder. The first input data structure comprises a combination of a feature representation of the first compound and a baseline transcriptional representation of a first cell type. The structure encoder comprises a first plurality of parameters. By inputting the first input data structure into the structure encoder there is retrieved, by operation of the first plurality of parameters on the first input data structure in accordance with an architecture of the structure encoder, as output from the structure encoder, a first compound embedding having a first dimension.

The first compound embedding is projected into a plurality of transcriptional embeddings each having the first dimension. Each respective transcriptional embedding in the plurality of transcriptional embeddings is overlayed onto each other transcriptional embedding in the plurality of transcriptional embeddings. At least a subset of the plurality of transcriptional embeddings collectively populates a plurality of clusters. Each cluster in the plurality of clusters is representative of a corresponding biological state. Each respective transcriptional embedding in the plurality of transcriptional embeddings is generated from inputting a corresponding cellular constituent abundance data set representative of the first cell type into a transcriptional encoder comprising a second plurality of parameters. The structure encoder is trained to minimize a loss against the plurality of transcriptional embeddings.

The first compound is associated with the corresponding biological state of a first cluster when the first compound embedding projected into the plurality of transcriptional embeddings falls into the first cluster.

### E. Training a Structure Encoder to Determine a Relationship Between One or More Biological States and a First Compound.

Another aspect of the present disclosure provides methods for training a structure encoder to determine a relationship between one or more biological states and a first compound. A training dataset is obtained that comprises a structure of each compound in a plurality of compounds and, for each respective compound in the plurality of compounds, a corresponding cellular constituent abundance data set representative of a first cell type that has been exposed to the respective compound.

The corresponding cellular constituent abundance data set of each respective compound in the plurality of compounds is used to obtain a separate clustering of the plurality of compounds against each set of pathways in a plurality of sets of pathways thereby obtaining a corresponding plurality of pathway labels for each compound in the plurality of compounds, each pathway label for a respective compound being a cluster assignment for the corresponding compound in a separate clustering of the plurality of compounds.

A transcriptional encoder is trained using the training set by a first procedure comprising (i) inputting, for each respective compound in the plurality of compounds, the corresponding cellular constituent abundance data in the first cell type of the respective compound into a transcriptional encoder comprising a second plurality of parameters thereby obtaining an initial corresponding calculated transcriptional embedding having a first dimension for the respective compound.

The first procedure further comprises shifting the initial corresponding calculated transcriptional embedding for each respective compound in the plurality of compounds toward a corresponding grouping of a corresponding set of compounds in the plurality of compounds based on (a) pathway similarity between the respective compound and the corresponding set of compounds in the plurality of compounds and (b) compound identity between the respective compound and the corresponding set of compounds, thereby obtaining a corresponding calculated transcriptional embedding, for each respective compound in the plurality of compounds.

The first procedure further comprises (iii) updating the second plurality of parameters through application of one or more loss functions to a differential between the corresponding calculated transcriptional embedding and the corresponding cellular constituent abundance data for each respective compound in the plurality of compounds.

The method further comprises training a structure encoder comprising a first plurality of parameters using the training set by a second procedure. The second procedure comprises (i) inputting, for each respective compound in the plurality of compounds, a combination of a feature representation of the respective compound and a baseline transcriptional representation of the first cell type into the structure encoder thereby obtaining a corresponding compound embedding having the first dimension. The second procedure further comprises (ii) updating the first plurality of parameters through minimization of a loss function applied to a differential between (a) the corresponding compound embedding for the respective compound from the structure encoder and (b) the corresponding calculated transcriptional embedding for the respective compound from the transcriptional encoder, thereby enabling the structure encoder to determine a relationship between one or more biological states and the first compound upon inputting a feature representation of the first compound and a baseline transcriptional representation of the first cell type into the structure encoder and comparing the output of the structure encoder to the corresponding calculated transcriptional embeddings of the training dataset.

### F. Non-Transitory Computer Readable Storage Medium Aspects.

Another aspect of the present disclosure provides a non-transitory computer readable storage medium storing one or more programs configured for execution by a computer, the one or more programs comprising instructions for carrying out any of the methods and/or embodiments disclosed herein.

### G. Computer System Aspects.

Another aspect of the present disclosure provides computer system, comprising one or more processors and memory, the memory storing instructions for performing any of the methods and/or embodiments disclosed herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The embodiments disclosed herein are illustrated by way of example, and not by way of limitation, in the figures of the accompanying drawings. Like reference numerals refer to corresponding parts throughout the drawings.

FIGS. 1A-1B illustrate a block diagram of an exemplary system and computing device, in accordance with an embodiment of the present disclosure.

FIGS. 2A, 2B, 2C, 2D, 2E, and 2F collectively provide a flow chart of processes and features of example methods for determining a likelihood that a test chemical compound causes a differential expression signature between a first cell state and a second cell state, in which dashed boxes represent optional elements, in accordance with various embodiments of the present disclosure.

FIGS. 3A, 3B, 3C, 3D, 3E, and 3F collectively provide a flow chart of processes and features of example methods for rank ordering a plurality of test chemical compounds against a differential expression signature between a first cell state and a second cell state is provided, in which dashed boxes represent optional elements, in accordance with various embodiments of the present disclosure.

FIG. 4 illustrates a method for determining a likelihood that a compound causes a differential expression signature between first and second cell states in which a differential expression signature, comprising a plurality of differential values, of the compound. Each differential value corresponds to a cellular constituent in a set of cellular constituents and represents a difference between (i) one or more abundance values for the constituent in a first cell-based representing the first cell state and (ii) one or more abundance values for the constituent in a second cell-based assay representing the second cell state. A chemical embedding is determined responsive to inputting a fingerprint of the compound into a first model. A differential expression embedding is determined responsive to inputting the differential expression signature into a second model. The likelihood that the compound causes the expression signature is determined based on a similarity between the chemical and differential embedding in accordance with an embodiment of the present disclosure.

FIG. 5 illustrates a method of training a structure encoder (first model) and a transcription encoder (second model) through contrastive learning in which a single loss function is used to concurrently train the parameters of the structure encoder and the transcription encoder in accordance with an embodiment of the present disclosure.

FIG. 6 illustrates a method of rank ordering a plurality of test chemical compounds against a differential expression signature between a first cell state and a second cell state in accordance with an embodiment of the present disclosure.

FIG. 7 compares the ability of the disclosed systems and methods to increate hit rate over random compound selection.

FIGS. 8A, 8B, 8C, 8D, 8E, 8F, 8G, 8H, 8I, and 8J collectively provide a flow chart of processes and features of example methods for determining whether a first compound and a second compound are causal for a common biological state, in which dashed boxes represent optional elements, in accordance with various embodiments of the present disclosure.

FIG. 9 provides a flow chart of processes and features of example methods for identifying a biological state for which a first compound is causal, in which dashed boxes represent optional elements, in accordance with various embodiments of the present disclosure.

FIGS. 10A and 10B collectively provide a flow chart of processes and features of example methods for training a structure encoder to determine a relationship between one or more biological states and a first compound, in accordance with various embodiments of the present disclosure.

FIG. 11A illustrates the integration of two distinct encoders, a structure encoder and a transcriptional encoder, into an overall model in accordance with some embodiments of the present disclosure. The structure encoder converts a SMILES string of each respective compound into a numerical representation of the respective compound with added baseline context, while the transcriptional encoder processes cellular constituent abundance data (e.g., in the form of differential expression scores (DES) of biological samples). Both encoders project the molecular information into a cohesive, low-dimensional latent space, with the overall model trained to align these two projections closely.

FIG. 11B provides illustrates a UMAP visualization of the co-embedding of compounds, depicting each compound's transcriptional (black) and structural (grey) embeddings in the latent space in accordance with some embodiments of the present disclosure.

FIG. 12A illustrates how the transcriptional encoder is designed to condense cellular constituent abundance data (e.g., DES) into a lower-dimensional space in accordance with some embodiments of the present disclosure. Through multi-objective metric learning, the transcriptional encoder enhances the similarity of samples from the same compound while differentiating between those from disparate compounds. Pathway enrichment analysis contributes additional grouping information, serving as soft labels to guide the learning process towards biologically relevant embeddings.

FIG. 12B illustrates a resulting UMAP visualization of the transcription-based embeddings outputted by the transcriptional encoder of FIG. 12A in accordance with some embodiments of the present disclosure. Samples are color-marked by their activity in the cholesterol biosynthesis pathway, WP197 from Wiki Pathways database, demonstrating the transcription encoder's effectiveness at grouping compounds with shared pathway influence. Notably, distinct clusters emerge, reflecting significant regulation of this pathway.

FIG. 13A illustrates an example structure encoder that processes diverse molecular representations derived from SMILES strings, along with basal expression data, fusing them into a unified vector, in accordance with some embodiments of the present disclosure. This vector is then mapped into a lower-dimensional space by the structure encoder in the form of a compound embedding. The structure encoder's training objective is to align the structural embeddings with the transcriptional embeddings from the previously trained transcriptional encoder.

FIG. 13B illustrates a UMAP plot that displays structure-based embeddings outputted by the structure encoder of FIG. 13A, grey-scaled by the level of activation of the cholesterol biosynthesis pathway in accordance with some embodiments of the present disclosure.

FIG. 14A illustrates a transcriptional fidelity assessment in accordance with an embodiment of the present disclosure. Using the transcriptional encoder, the embeddings of both training and validation samples are visualized. The embeddings' quality is then evaluated by calculating the average cosine similarity between the DES of each sample and its five nearest neighbors in the latent space. The gradation of grey-scale intensity reflects the degree of similarity, indicating that clusters tend to display higher transcriptional fidelity, while areas closer to the center show a more diverse range of DES, implying lower fidelity.

FIG. 14B is the same as 14A, but instead of using DES, similarity of activation of KEGG pathways is used in accordance with an embodiment of the present disclosure.

FIG. 14C illustrates transcriptional encoder performance on validation set. Two KNN models were evaluated: one leveraging the embedding from the transcriptional encoder of the present disclosure and the other using raw DES. These models were tasked to classify validation samples into their correct compound classes. The bar chart illustrates recall@10 for compounds, segmented by their activity level measured by the number of DEGs. The model, working within the latent space, consistently outperformed the traditional KNN that utilized original DES, demonstrating enhanced signal detection and improved recall across all compound activity levels.

FIG. 15A illustrates compound recall accuracy in accordance with an embodiment of the present disclosure. To assess the quality of the structure encoder the quality of assigning transcription-based projection of a compound to its structure-based counterpart was measured. When the correct structure-based projection was among the top-50 closest neighbors in the embedding it was considered a hit. Compound recall is stratified by the number of DEG in FIG. 15A.

FIG. 15B provides a transcriptional fidelity assessment in accordance with an embodiment of the present disclosure. The ability of the disclosed model to recommend transcriptionally similar compounds (transcriptional mimics) for a query compound was quantified by embedding all test compounds into the latent space, ranking them against all training compounds by cosine similarity in that latent space, and recording how often transcriptional mimics are ranked within top-50 closest compounds. This is called transcriptional hit rate in the figure. Similarity between test and train compounds was also computed using 5 additional models (all were used to convert SMILES string into a numerical vector) and the analysis was repeated using similarities computed using these latent representations.

FIG. 15C illustrates an example of predicting the transcription of an unseen compound using is structure-based projection in accordance with an embodiment of the present disclosure. The x and y axes show the predicted and observed DES, respectively, for the compound.

FIG. 15D illustrates an example of predicting pathway regulation of an unseen compound using its structure-based projection in accordance with an embodiment of the present disclosure. The x and y axes show the predicted pathway regulation score and observed pathway regulation score, respectively.

FIG. 15E illustrates a kernel density estimate (KDE) plot for the R-squared values of transcription reconstruction for those test compounds with more than 1000 DEGs, in accordance with an embodiment of the present disclosure.

FIG. 15F illustrates a KDE plot for the R-squared values of pathway regulation score reconstruction, for those test compounds with more than 1000 DEGs, in accordance with an embodiment of the present disclosure.

FIGS. 16A, 16B, 16C, and 16D illustrate examples of a recommended molecule for a query compound using a model in accordance with the present disclosure. Notably, the model identifies different scaffolds compared to the query, but with similar transcriptional responses, highlighting the ability of the disclosed model to move beyond analogs for novel compound designs.

FIG. 17A illustrates a UMAP of cells from untreated (DMSO) wells, shaded by cell type within a CD34+ population in accordance with an embodiment of the present disclosure.

FIG. 17B illustrates a UMAP of cells from untreated (DMSO) wells, shaded by plate numbers (from earlier to later plates) in accordance with an embodiment of the present disclosure.

FIG. 17C illustrates number of regulated KEGG pathways with respect to the number of compounds (in chronological order of experimentation) in accordance with an embodiment of the present disclosure.

FIG. 17D illustrates number of compound per each target in accordance with an embodiment of the present disclosure.

FIG. 18A illustrates reconstruction loss (RMSE) between transcription-based and structure-based embeddings of train and validation splits in accordance with an embodiment of the present disclosure.

FIG. 18B illustrates a UMAP of CD34+ cell types based on pathway activation scores of baseline gene expression in accordance with an embodiment of the present disclosure.

FIG. 19A illustrates mean cosine similarity of samples' differential expression profile with its five closest neighbors in the latent space in accordance with an embodiment of the present disclosure. Samples from the train split on average have higher similarity with their corresponding neighbors.

FIG. 19B illustrates mean cosine similarity of samples' KEGG 2021 pathways regulation with its five closest neighbors in the latent space in accordance with an embodiment of the present disclosure. On average, neighbors in the latent space have higher pathway regulation similarity than original differential expression profile similarity.

FIG. 19C illustrates ranking of transcription in which structure pairs of test compounds, stratified by test compound Tanimoto similarity, is provided in accordance with an embodiment of the present disclosure. The dashed line illustrates rank cut-off of the correct structure-transcription pairing.

FIG. 19D illustrates an estimation of chemical space applicable for prediction from the ChEMBL 30 database in accordance with an embodiment of the present disclosure.

FIG. 19E illustrates transcriptional similarity threshold for compounds with different activity levels in accordance with an embodiment of the present disclosure.

FIG. 19F illustrates a distribution of a number of transcriptional mimics for test compounds in accordance with an embodiment of the present disclosure.

FIGS. 20A, 20B, and 20C illustrate an example of a hit augmentation task in accordance with an embodiment of the present disclosure. FIG. 20A (left): query compound (one of the test compounds), a JAK2 inhibitor. FIG. 20A (right): scatter plot of embedding similarity between the query compound and all training compounds, shaded by the observed transcriptional similarity between the query compound and ranked train compounds. Note that all top-10 compounds show transcriptional similarity of 0.75 or higher with query. FIGS. 20B and 20C: top twelve predicted compounds, their main targets, and their Tanimoto and transcriptional similarity with the query compound. Notably, from the twelve predicted compounds, eight are from the JAK family.

FIG. 21 illustrates a block diagram of an exemplary system and computing device for training a structure encoder to determine a relationship between one or more biological states and a first compound, in accordance with FIG. 10 of the present disclosure.

FIG. 22 illustrates a method for determining pathway activation scores in accordance with an embodiment of the present disclosure.

FIG. 23 illustrates a method for training a transcriptional encoder in accordance with an embodiment of the present disclosure.

FIG. 24 illustrates a method for training a structure encoder in accordance with an embodiment of the present disclosure.

### DETAILED DESCRIPTION

### Introduction

The present disclosure describes systems and methods for hit prediction in small-molecule drug discovery. Within a drug discovery platform, the disclosed systems and methods allow for the screening of compounds that, for instance, reverse a transcriptional/cellular transition found to be associated with a disease. In some embodiments, the disclosed systems and methods makes use of deep learning models trained by contrastive learning.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

Plural instances may be provided for components, operations or structures described herein as a single instance. Finally, boundaries between various components, operations, and data stores are somewhat arbitrary, and particular operations are illustrated in the context of specific illustrative configurations. Other forms of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first dataset could be termed a second dataset, and, similarly, a second dataset could be termed a first dataset, without departing from the scope of the present invention. The first dataset and the second dataset are both datasets, but they are not the same dataset.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

Furthermore, when a reference number is given an “i^{th}” denotation, the reference number refers to a generic component, set, or embodiment. For instance, a cellular-component termed “cellular-component i” refers to the i^{th} cellular-component in a plurality of cellular-components.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” means within 1 or more than 1 standard deviation, per the practice in the art. In some embodiments, “about” means a range of +20%, +10%, +5%, or +1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. All numerical values within the detailed description herein are modified by “about” the indicated value, and consider experimental error and variations that would be expected by a person having ordinary skill in the art. The term “about” can have the meaning as commonly understood by one of ordinary skill in the art. In some embodiments, the term “about” refers to +10%. In some embodiments, the term “about” refers to +5%.

As used herein, the terms “abundance,” “abundance level,” or “expression level” refers to an amount of a cellular constituent (e.g., a gene product such as an RNA species, e.g., mRNA or miRNA, or a protein molecule) present in one or more cells, or an average amount of a cellular constituent present across multiple cells. When referring to mRNA or protein expression, the term generally refers to the amount of any RNA or protein species corresponding to a particular genomic locus, e.g., a particular gene. However, in some embodiments, an abundance can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used interchangeably herein, a “cell state” or “biological state” refers to a state or phenotype of a cell or a population of cells. For example, a cell state can be healthy or diseased. A cell state can be one of a plurality of diseases. A cell state can be a response to a compound treatment and/or a differentiated cell lineage. A cell state can be characterized by a measure (e.g., an activation, expression, and/or measure of abundance) of one or more cellular constituents, including but not limited to one or more genes, one or more proteins, and/or one or more biological pathways.

As used herein, a “cell state transition” or “cellular transition” refers to a transition in a cell's state from a first cell state to a second cell state. In some embodiments, the second cell state is an altered cell state (e.g., a healthy cell state to a diseased cell state). In some embodiments, one of the respective first cell state and second cell state is an unperturbed state and the other of the respective first cell state and second cell state is a perturbed state caused by an exposure of the cell to a condition. The perturbed state can be caused by exposure of the cell to a compound. A cell state transition can be marked by a change in cellular constituent abundance in the cell, and thus by the identity and quantity of cellular constituents (e.g., mRNA, transcription factors) produced by the cell (e.g., a perturbation signature).

As used herein, the term “dataset” in reference to cellular constituent abundance measurements for a cell or a plurality of cells can refer to a high-dimensional set of data collected from a single cell (e.g., a single-cell cellular constituent abundance dataset) in some contexts. In other contexts, the term “dataset” can refer to a plurality of high-dimensional sets of data collected from single cells (e.g., a plurality of single-cell cellular constituent abundance datasets), each set of data of the plurality collected from one cell of a plurality of cells.

As used herein, the term “differential abundance” or “differential expression” refers to differences in the quantity and/or the frequency of a cellular constituent present in a first entity (e.g., a first cell, plurality of cells, and/or sample) as compared to a second entity (e.g., a second cell, plurality of cells, and/or sample). In some embodiments, a first entity is a sample characterized by a first cell state (e.g., a diseased phenotype) and a second entity is a sample characterized by a second cell state (e.g., a normal or healthy phenotype). For example, a cellular constituent can be a polynucleotide (e.g., an mRNA transcript) which is present at an elevated level or at a decreased level in entities characterized by a first cell state compared to entities characterized by a second cell state. In some embodiments, a cellular constituent can be a polynucleotide which is detected at a higher frequency or at a lower frequency in entities characterized by a first cell state compared to entities characterized by a second cell state. A cellular constituent can be differentially abundant in terms of quantity, frequency or both. In some instances, a cellular constituent is differentially abundant between two entities if the amount of the cellular constituent in one entity is statistically significantly different from the amount of the cellular constituent in the other entity. For example, a cellular constituent is differentially abundant in two entities if it is present at least about 120%, at least about 130%, at least about 150%, at least about 180%, at least about 200%, at least about 300%, at least about 500%, at least about 700%, at least about 900%, or at least about 1000% greater in one entity than it is present in the other entity, or if it is detectable in one entity and not detectable in the other. In some instances, a cellular constituent is differentially expressed in two sets of entities if the frequency of detecting the cellular constituent in a first subset of entities (e.g., cells representing a first subset of annotated cell states) is statistically significantly higher or lower than in a second subset of entities (e.g., cells representing a second subset of annotated cell states). For example, a cellular constituent is differentially expressed in two sets of entities if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of entities than the other set of entities.

As used herein, the term “healthy” refers to a sample characterized by a healthy state (e.g., obtained from a subject possessing good health). A healthy subject can demonstrate an absence of any malignant or non-malignant disease. A “healthy” individual can have other diseases or conditions, unrelated to the condition being assayed, which can normally not be considered “healthy.”

As used herein, the term “perturbation” in reference to a cell (e.g., a perturbation of a cell or a cellular perturbation) refers to any exposure of the cell to one or more conditions, such as a treatment by one or more compounds. These compounds can be referred to as “perturbagens.” In some embodiments, the perturbagen can include, e.g., a small molecule, a biologic, a therapeutic, a protein, a protein combined with a small molecule, an ADC, a nucleic acid, such as an siRNA or interfering RNA, a cDNA over-expressing wild-type and/or mutant shRNA, a cDNA over-expressing wild-type and/or mutant guide RNA (e.g., Cas9 system or other gene editing system), or any combination of any of the foregoing. A perturbation can induce or be characterized by a change in the phenotype of the cell and/or a change in the expression or abundance level of one or more cellular constituents in the cell (e.g., a perturbation signature). For instance, a perturbation can be characterized by a change in the transcriptional profile of the cell.

As used herein, the term “sample,” “biological sample,” or “patient sample,” refers to any sample taken from a subject, which can reflect a biological state associated with the subject. Examples of samples include, but are not limited to, blood, whole blood, plasma, serum, urine, cerebrospinal fluid, fecal, saliva, sweat, tears, pleural fluid, pericardial fluid, or peritoneal fluid of the subject. A sample can include any tissue or material derived from a living or dead subject. A sample can be a cell-free sample. A sample can comprise one or more cellular constituents. For instance, a sample can comprise a nucleic acid (e.g., DNA or RNA) or a fragment thereof, or a protein. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A sample can be a bodily fluid. A sample can be a stool sample. A sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, an “effective amount” or “therapeutically effective amount” is an amount sufficient to affect a beneficial or desired clinical result upon treatment. An effective amount, in some embodiments, is administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the therapeutic agent being administered.

As used herein the term “fingerprint” as in a fingerprint of a compound is a digital digest of the compound. Nonlimiting examples of such a digital digest include Daylight fingerprints, a BCI fingerprint, an ECFC4 fingerprint, an ECFP4 fingerprint, an EcFC fingerprint, an MDL fingerprint, an atom pair fingerprint (APFP fingerprint), a topological torsion fingerprint (TTFP) fingerprint, a UNITY 2D fingerprint, an RNNS2S fingerprint, or a GraphConv fingerprint. See Franco, 2014, “The Use of 2D fingerprint methods to support the assessment of structural similarity in orphan drug legislation,” J. Cheminform 6, p. 5, and Rensi and Altman, 2017, “Flexible Analog Search with Kernel PCA Embedded Molecule Vectors,” Computational and Structural Biotechnology Journal, doi: 10.1016/j.csbj.2017.03.003, each of which is hereby incorporated by reference. See also Raymond and Willett, 2002, “Effectiveness of graph-based and fingerprint-based similarity measures for virtual screening of 2D chemical structure databases,” Journal of Computer-Aided Molecular Design 16, 59-71, and Franco et al., 2014, “The use of 2D fingerprint methods to support the assessment of structural similarity in orphan drug legislation” Journal of chemoinformatics 6(5), each of which is hereby incorporated by reference.

As used interchangeably herein, the term “classifier”, “model”, algorithm, “regressor”, and/“or classifier” refers to a machine learning model or algorithm. In some embodiments, a model is an unsupervised learning algorithm. In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model). In some embodiments, a classifier or model of the present disclosure has 25 or more, 100 or more, 1000 or more 10,000 or more, 100,000 or more or 1×10⁶ or more parameters and thus the calculations of the model cannot be mentally performed.

Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n>75; n>100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1 ×10⁷. As such, the algorithms, models, regressors, and/or classifiers of the present disclosure cannot be mentally performed. In some embodiments, n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Neural networks. In some embodiments, the model is a neural network (e.g., a convolutional neural network and/or a residual neural network). Neural network models, also known as artificial neural networks (ANNs), include convolutional and/or residual neural network models (deep learning models). Neural networks can be machine learning models that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning model (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing an image of a subject. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,”′ CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network models, including convolutional neural network models, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

As used interchangeably herein, the term “neuron,” “node,” “unit,” “hidden neuron,” “hidden unit,” or the like, refers to a unit of a neural network that accepts input and provides an output via an activation function and one or more parameters (e.g., coefficients and/or weights). For example, a hidden neuron can accept one or more inputs from a prior layer and provide an output that serves as an input for a subsequent layer. In some embodiments, a neural network comprises only one output neuron. In some embodiments, a neural network comprises a plurality of output neurons. Generally, the output is a prediction value, such as a probability or likelihood, a binary determination (e.g., a presence or absence, a positive or negative result), and/or a label (e.g., a classification and/or a correlation coefficient) of a condition of interest such as a covariate, a cell state annotation, or a cellular process of interest. For single-class classification models, the output can be a likelihood (e.g., a correlation coefficient and/or a weight) of an input feature (e.g., one or more cellular constituent modules) having a condition (e.g., a covariate, a cell state annotation, and/or a cellular process of interest). For multi-class classification models, multiple prediction values can be generated, with each prediction value indicating the likelihood of an input feature for each condition of interest.

As used herein, the term “parameter” refers to any coefficient or, similarly, any value of an internal or external element (e.g., weight and/or hyperparameter) in a model, classifier, or algorithm that can affect (e.g., modify, tailor, and/or adjust) one or more inputs, outputs, and/or functions in the model, classifier, or algorithm. In some embodiments, parameters are coefficients (e.g., weights) that modulate one or more inputs, outputs, or functions in a model. For instance, a value of a parameter can be used to upweight or down-weight the influence of an input (e.g., a feature) to a model. Features can be associated with parameters, such as in a logistic regression, SVM, or naïve Bayes model. A value of a parameter can, alternately or additionally, be used to upweight or down-weight the influence of a node in a neural network (e.g., where the node comprises one or more activation functions that define the transformation of an input to an output), a class, or an instance (e.g., of a cell in a plurality of cells). Assignment of parameters to specific inputs, outputs, functions, or features is not limited to any one paradigm for a given model but can be used in any suitable model architecture for optimal performance. In some instances, reference to the parameters (e.g., coefficients) associated with the inputs, outputs, functions, or features of a model can similarly be used as an indicator of the number, performance, or optimization of the same, such as in the context of the computational complexity of machine learning models. In some embodiments, a parameter has a fixed value. In some embodiments, a value of a parameter is manually and/or automatically adjustable (e.g., using a hyperparameter optimization method). In some embodiments, a value of a parameter is modified by a model validation and/or training process (e.g., by error minimization and/or backpropagation methods, as described elsewhere herein).

As used herein, the term “vector” is an enumerated list of elements, such as an array of elements, where each element has an assigned meaning. As such, the term “vector” as used in the present disclosure is interchangeable with the term “tensor.” As an example, if a vector comprises the abundance counts, in a plurality of cells, for a respective cellular constituent, there exists a predetermined element in the vector for each one of the plurality of cells. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents abundance count of cell 1 of a plurality of cells, etc.).

### I. EXEMPLARY SYSTEM EMBODIMENTS

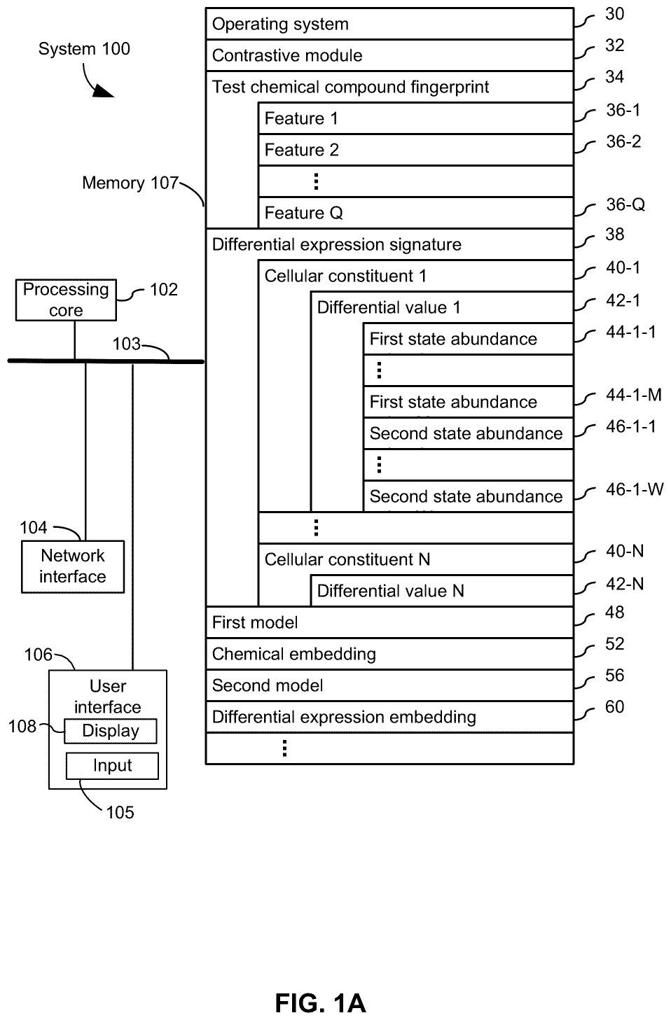

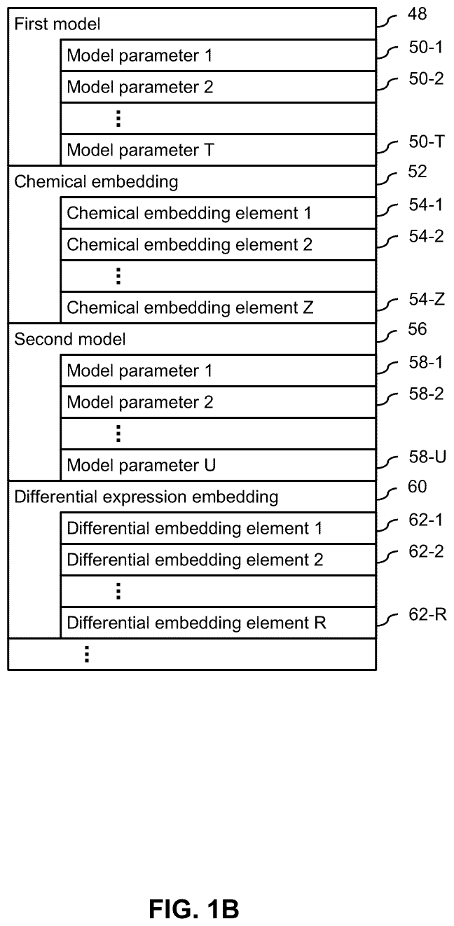

Now that an overview of some aspects of the present disclosure and some definitions used in the present disclosure have been provided, details of an exemplary system are described in conjunction with FIG. 1.

FIGS. 1A and 1B provide a block diagram illustrating a system 100 in accordance with some embodiments of the present disclosure. The system 100 determines a likelihood that a test chemical compound causes (is associated with, causal for) a differential expression signature between a first cell state and a second cell state. In FIG. 1, the system 100 is illustrated as a computing device. Other topologies of the computer system 100 are possible. For instance, in some embodiments, the system 100 can in fact constitute several computer systems that are linked together in a network, or be a virtual machine or a container in a cloud computing environment. As such, the exemplary topology shown in FIG. 1 merely serves to describe the features of an embodiment of the present disclosure in a manner that will be readily understood to one of skill in the art.

Referring to FIG. 1, in some embodiments the computer system 100 (e.g., a computing device) includes a network interface 104. In some embodiments, the network interface 104 interconnects the system 100 computing devices within the system with each other, as well as optional external systems and devices, through one or more communication networks. In some embodiments, the network interface 104 optionally provides communication via the Internet, one or more local area networks (LANs), one or more wide area networks (WANs), other types of networks, or a combination of such networks.

Examples of networks include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

The system 100 in some embodiments includes one or more processing units (CPU(s)) 102 (e.g., a processor, a processing core, etc.), one or more network interfaces 104, a user interface 106 including (optionally) a display 108 and an input system 105 (e.g., an input/output interface, a keyboard, a mouse, etc.) for use by a user, memory 107, and one or more communication buses 103 for interconnecting the aforementioned components. The one or more communication buses 103 optionally include circuitry (sometimes called a chipset) that interconnects and controls communications between system components. The memory 107 typically includes high-speed random access memory, such as DRAM, SRAM, DDR RAM, ROM, EEPROM, flash memory, CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The memory 107 optionally includes one or more storage devices remotely located from the CPU(s) 102. In some embodiments, the memory includes non-transitory computer readable storage medium. In some embodiments, the memory 107 stores the following programs, modules and data structures, or a subset thereof:
an optional operating system 30 (e.g., ANDROID, IOS, DARWIN, RTXC, LINUX, UNIX, OS X, WINDOWS, or an embedded operating system such as VxWorks), which includes procedures for handling various basic system services and for performing hardware dependent tasks;
a contrastive module 32 for performing any of the computational methods described in the present disclosure;
a test chemical compound fingerprint 34 of a test chemical compound comprising a plurality of feature 36;
a differential expression signature 38 comprising, for each cellular constituent 40 in a set of cellular constituents 130 comprising a set of cellular constituents, a plurality of differential values 42, where each respective differential value in the plurality of differential values corresponds to a respective cellular constituent in a set of cellular constituents, and where the respective differential value represents a difference between (i) one or more abundance values (first state abundance values 44) measured for the respective cellular constituent in a first cell-based assay of a first plurality of cells that represent the first cell state and (ii) one or more abundance values (second state abundance values 46) measured for the respective cellular constituent in a second cell-based assay of a second plurality of cells that represent the second cell state;
a first model 48, comprising a plurality of parameters 50, that responsive to inputting a fingerprint of the test chemical compound into the first model, provides a respective chemical embedding 52 comprising a plurality of chemical embedding elements 54; and
a second model 56, comprising a plurality of parameters 58, that responsive to inputting a differential expression signature into the second model, provides a differential expression embedding 60 comprising a plurality of differential embedding elements 62.

In various embodiments, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some embodiments, the memory stores additional modules and data structures not described above. In some embodiments, one or more of the above identified elements is stored in a computer system, other than that of the system 100, that is addressable by the system 100 so that the system 100 may retrieve all or a portion of such data when needed.

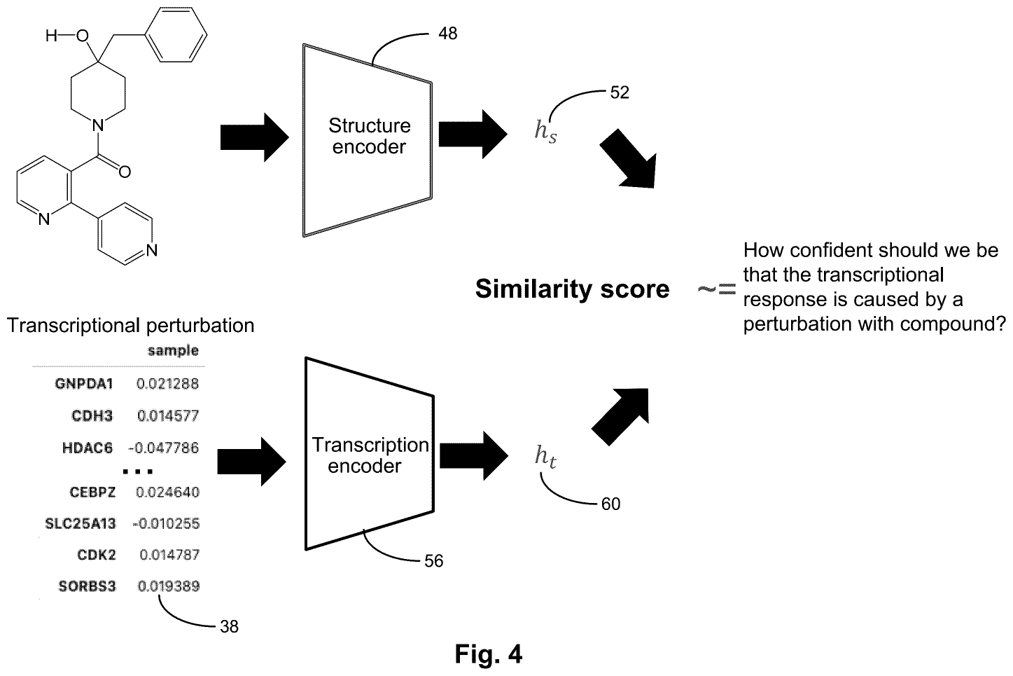

FIG. 1A and FIG. 4 illustrate major components used by the present disclosure, including a first model 48 that serves as a structure encoder. In some embodiments the first model 48 is a multilayer perceptron (fully connected neural network) containing dropout layers, batch normalization, and a ReLU non-linearity. In some embodiments the first model 48 produces an embedding E_{S} 52 of a structure S. Moreover, as illustrated in FIG. 1A, a second model 56 serves as a differential expression signature 38 encoder. In some embodiments the second model 56 is also a multilayer perceptron (fully connected neural network) with similar features as the structure encoder. The second model 56 produces a differential expression embedding 60 E_{T} of the differential expression signature 38 T. The contrastive module 32 uses the resulting chemical embedding 52 and differential expression embedding 60 to compute a similarity score between the two embeddings. In some embodiments the similarity score is the product of a function that maps the chemical embedding 52 and the differential expression embedding 60. In some embodiments this function F(E_{S}, E_{T}) is the normalized dot product:

$$F ( {E}_{S} , {E}_{T} ) = \frac{{E}_{T} \cdot {E}_{S}}{‖ {E}_{T} ‖ \vert \vert {E}_{S} ‖}$$

where E_{T} is the elements of the differential expression signature, and E_{S} is the corresponding elements of the chemical embedding.

Although FIG. 1 depicts a “system 100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in memory 107, some or all of these data and modules instead may be stored in more than one memory such as at a remote storage device that can be a part of a cloud-based infrastructure.

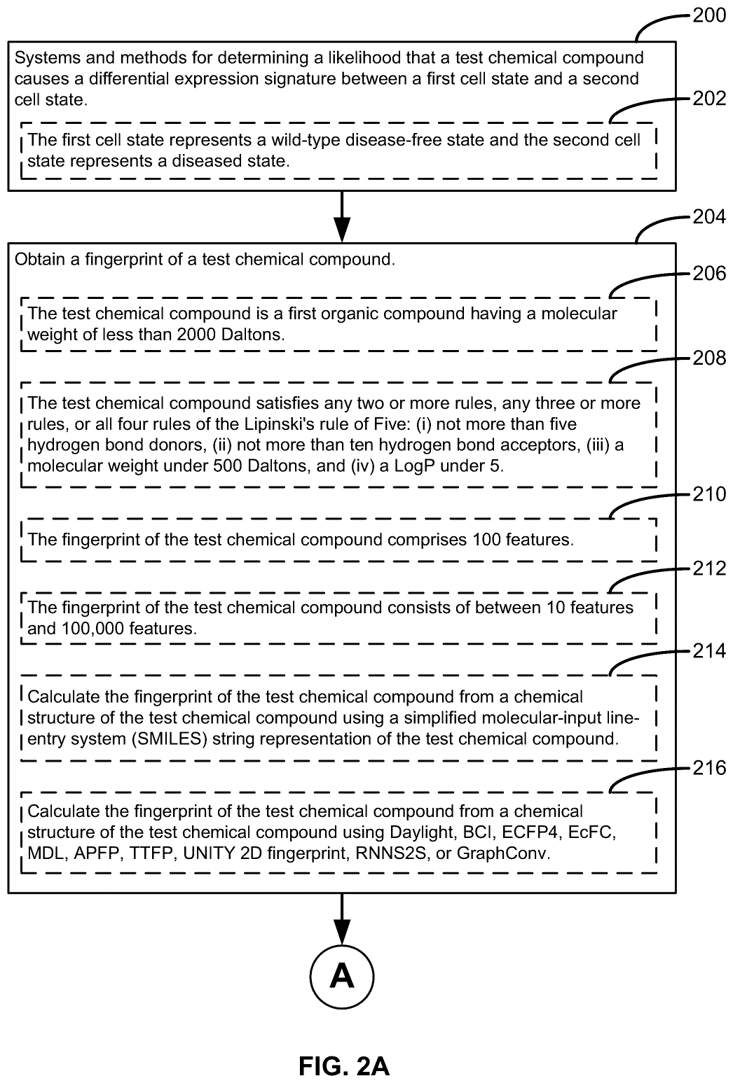

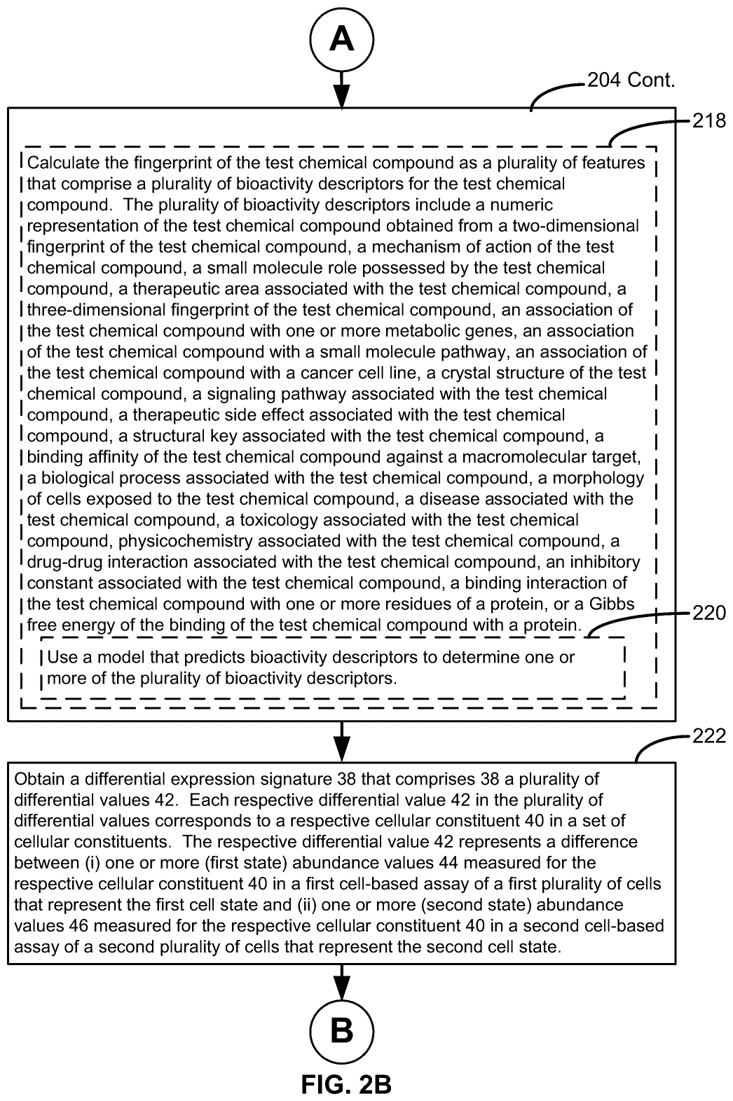

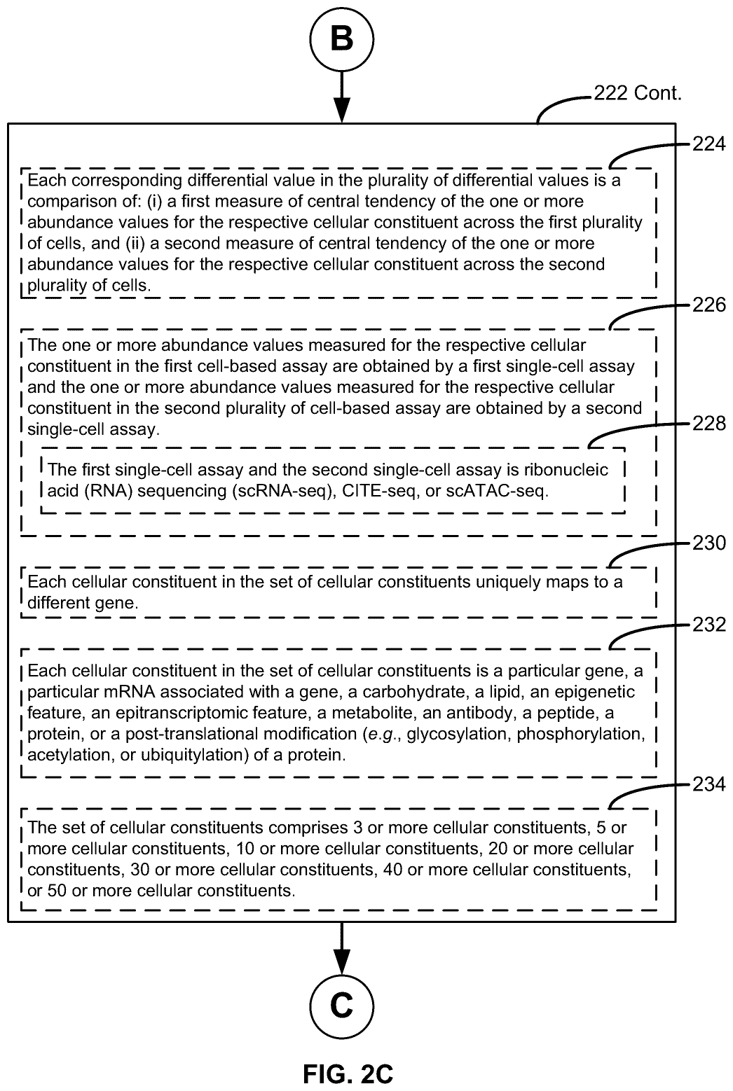

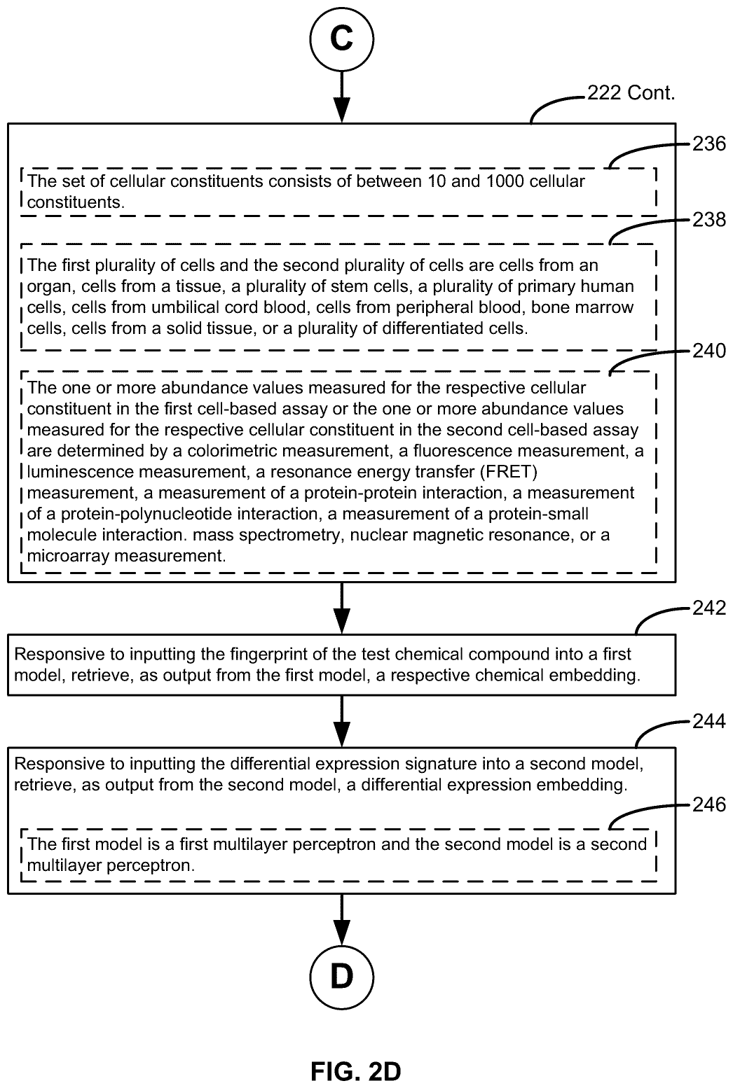

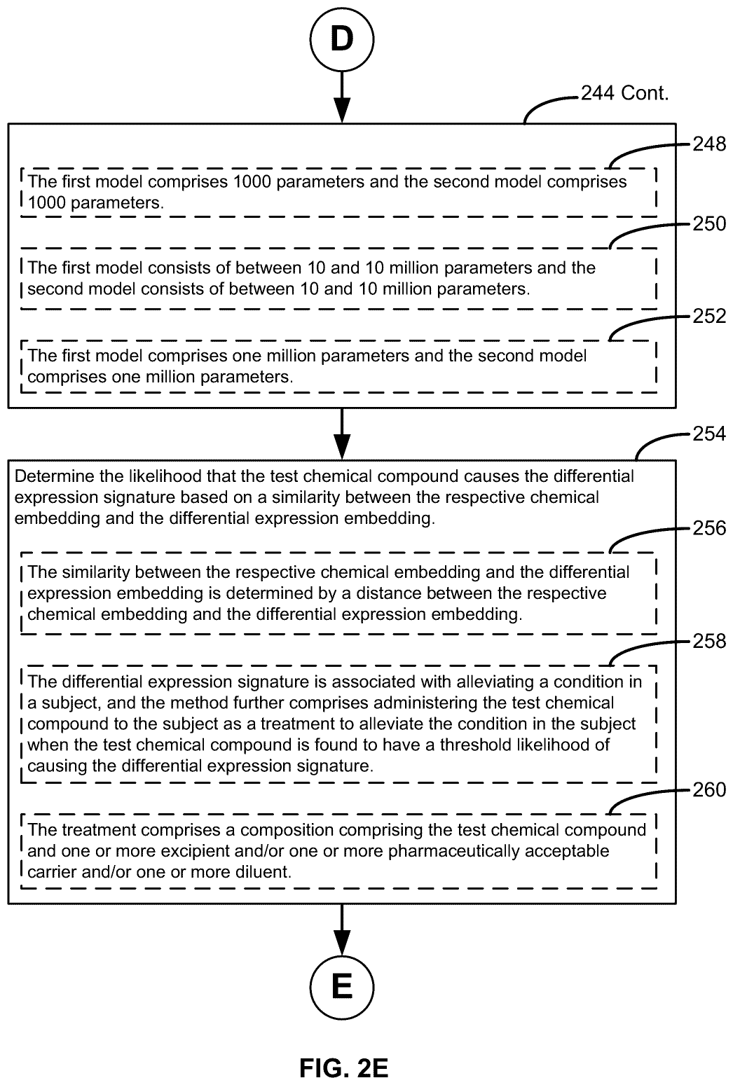

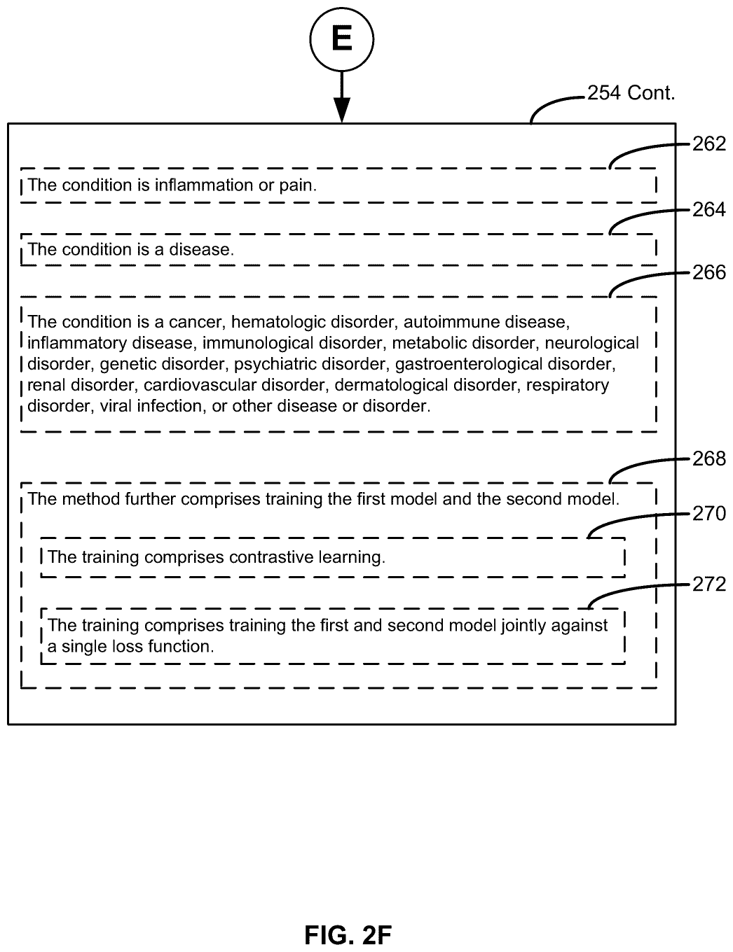

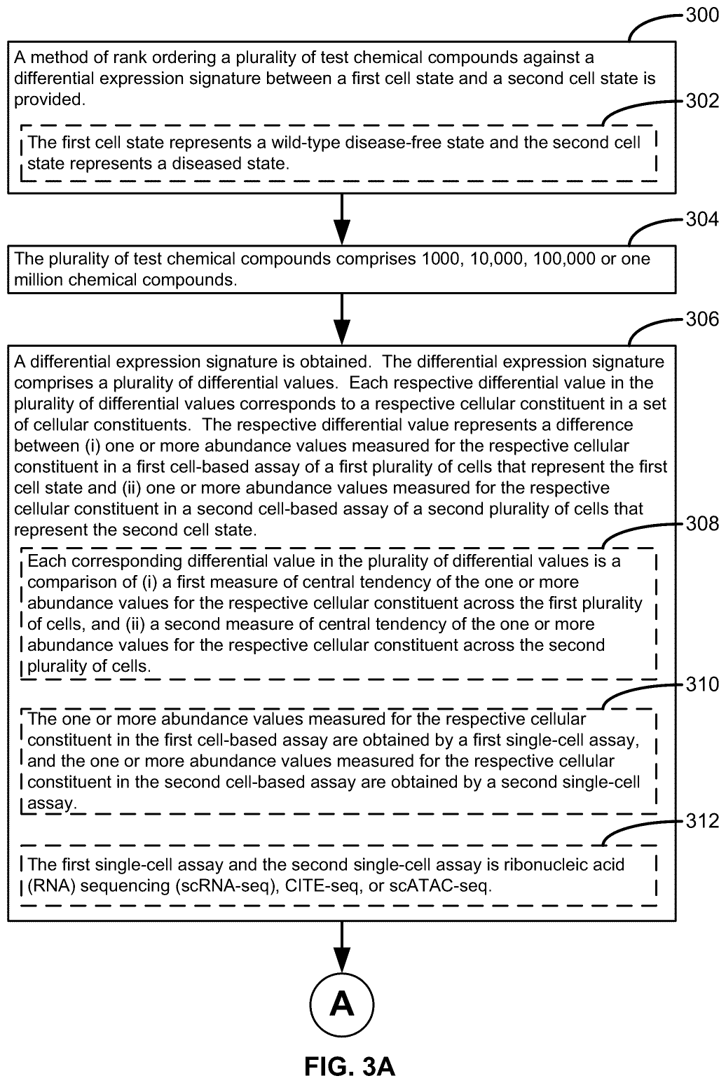

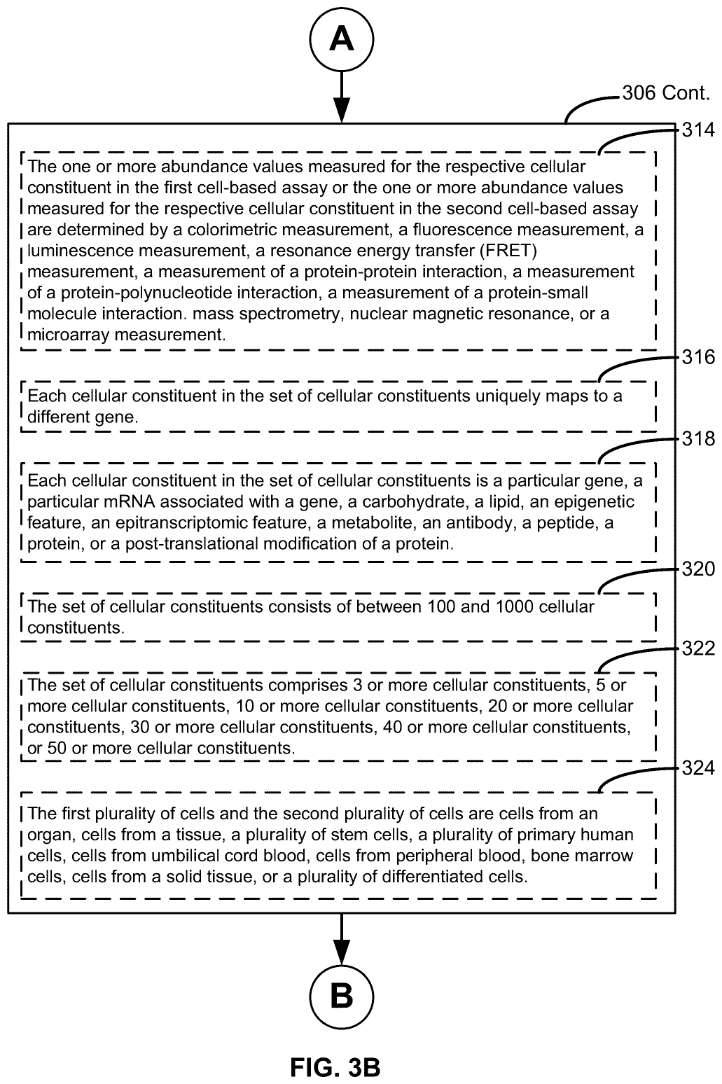

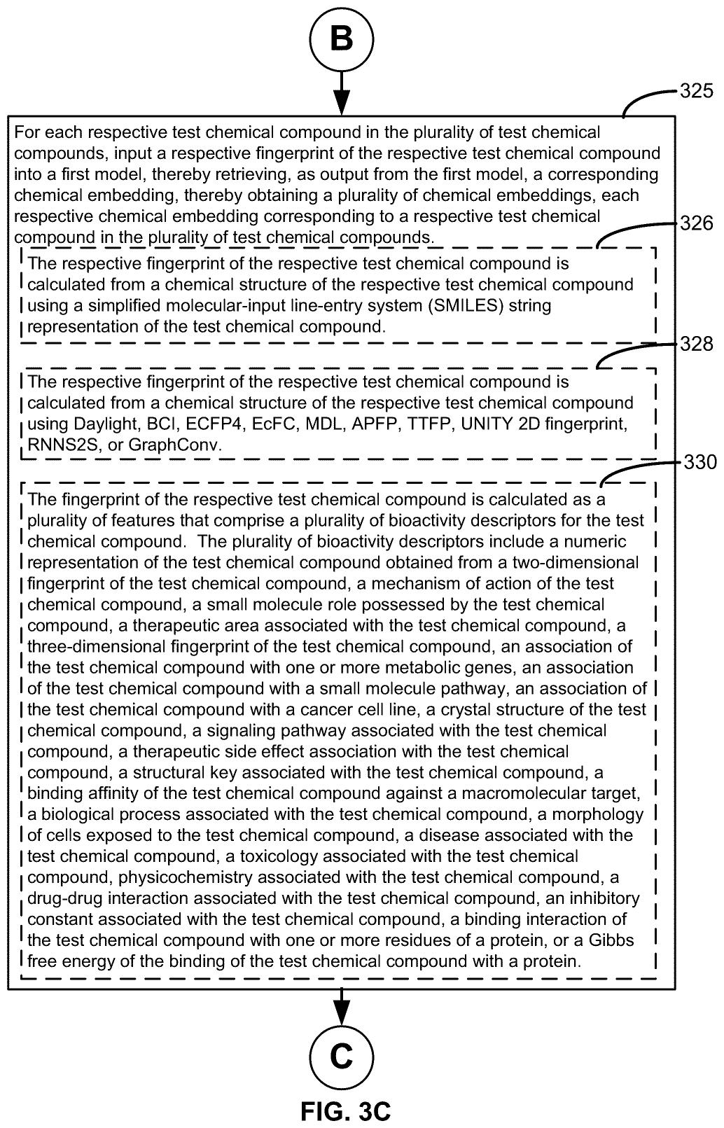

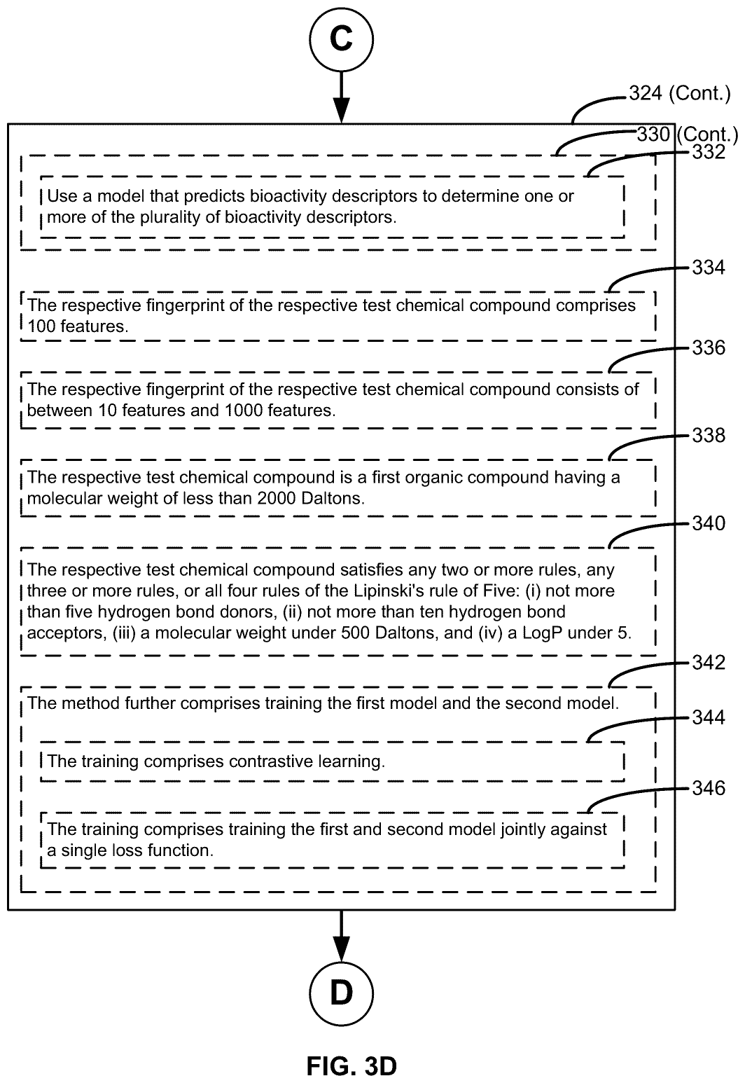

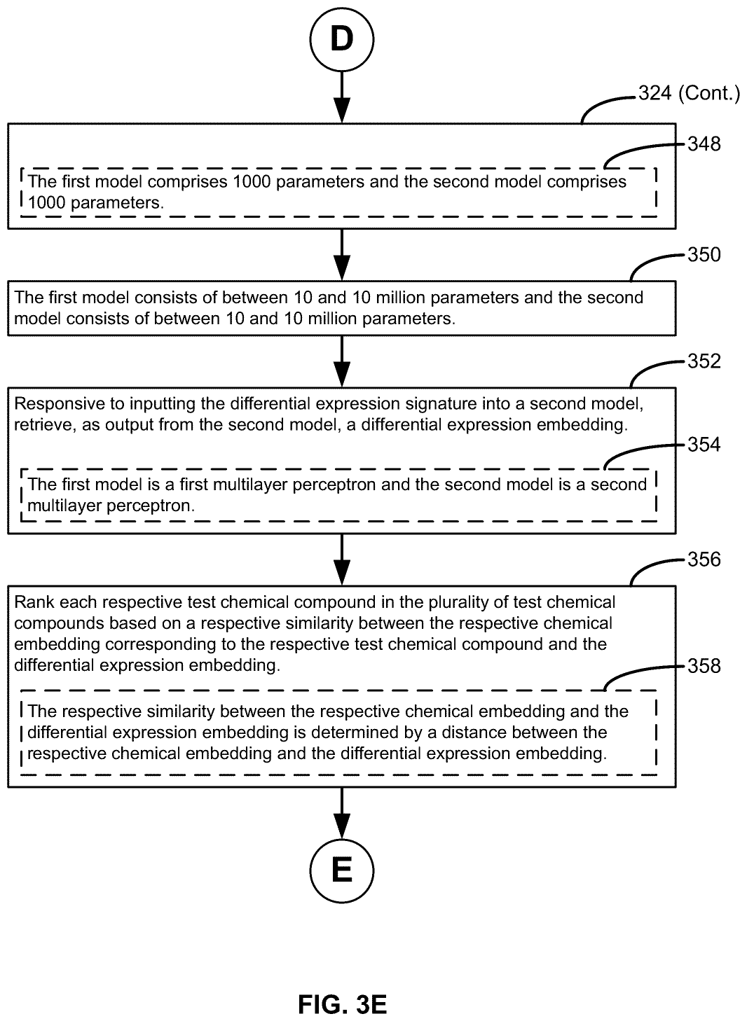

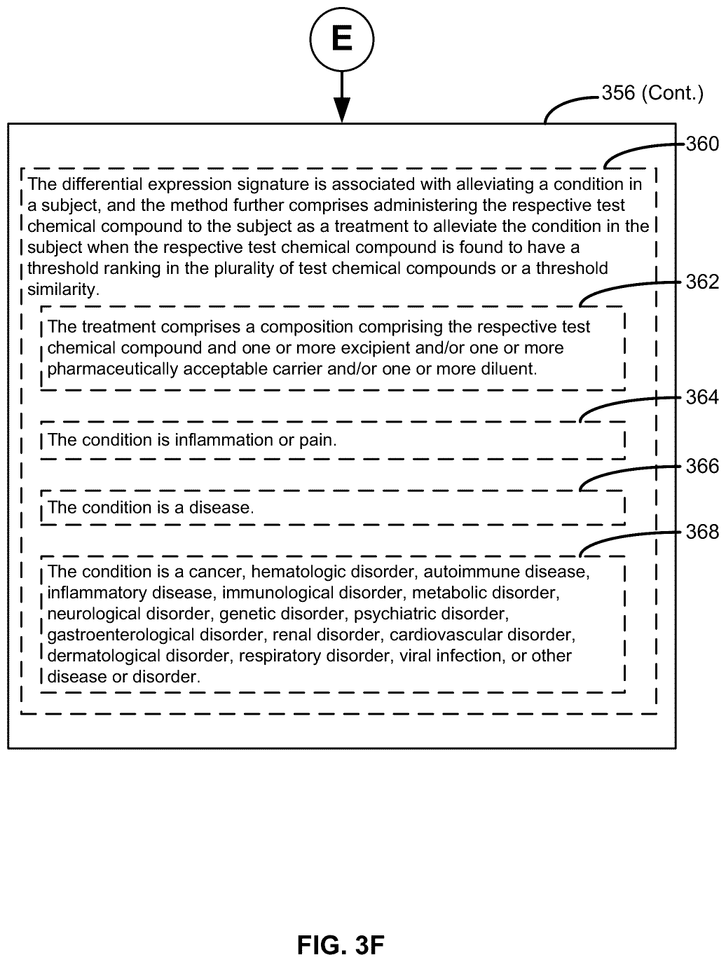

While a system in accordance with the present disclosure has been disclosed with reference to FIG. 1, methods 200 and 300 in accordance with the present disclosure are now detailed with reference to FIGS. 2 and 3.

### II. DETERMINING A LIKELIHOOD THAT A TEST CHEMICAL COMPOUND CAUSES A DIFFERENTIAL EXPRESSION SIGNATURE BETWEEN A FIRST CELL STATE AND A SECOND CELL STATE

In accordance with an aspect of the present disclosure the disclosed systems and methods take as input a pair “(S,T)” consisting of a fingerprint of a chemical structure S and a differential expression signature T. The disclosed systems and methods return a score/value predicting how likely the differential expression signature T is caused by the fingerprint of the chemical structure S. In some embodiments, the disclosed systems and methods are trained on one or more large perturbational data sets. In this way, the disclosed systems and methods learn general patterns of how chemical structures lead to differential expression signature (transcriptional perturbations) within a cell. By design, the disclosed systems and methods can make predictions on an molecule that model has not seen before because it only requires the knowledge of a molecule's structure. By replacing T with a desired transcriptional response associated with a disease, the disclosed systems and methods allow for the screening of large chemical spaces for the ability to reverse a disease-associated perturbation. Thereby, the disclosed systems and methods can help to speed up the search for a hit compound significantly and to find completely new hits—ultimately promising to find better or novel treatment for diseases faster and more successfully.

One embodiment of the present disclosure makes use of contrastive learning to train the models of the present disclosure. Contrastive learning is a self-supervised learning method, e.g. a machine learning paradigm that trains a model to extract features from data without the need for labels of that data. Contrastive learning is used in some embodiments of the present disclosure to learn the association between transcription and chemical structure. In such embodiments, the disclosed models are trained to create a joint embedding of a chemical structure and a transcriptional perturbation. “Closeness”, that is the distance between the resulting chemical structure embedding and the transcriptional perturbation embedding is then used to associate a perturbation with the right chemical structure. This space can also be visualized and bears potential for human interpretation—which is particularly useful in drug discovery to generate scientific hypothesis. The motivation to use contrastive learning for hit prediction is two-fold. First, regarding mapping structure to transcription, it is realized that chemical structure can have multiple cellular perturbations—depending on cell type, dose, laboratory, RNA measuring technology, and many other factors. Therefore, a regression model that maps chemical structure to transcriptional signature is likely not going to be useful as there would be no single correct output. Second, regarding mapping transcription to structure: it is realized that this approach alone severely restricts the number of compounds that can be considered for hit prediction. The disclosed contrastive learning circumvents both problems. It learns to map transcriptional perturbations from the same compound to the same embedding (it removes biological variability and batch effects). Moreover, it learns to map chemical structures to a useful embedding allowing to screen over compounds the model has been trained on and for which it has not seen transcriptional data.

Referring to block 200 of FIG. 2A, in some embodiments, systems and methods for determining a likelihood that a test chemical compound causes a differential expression signature between a first cell state and a second cell state.

Block 202. Referring to block 202, in some embodiments, the first cell state represents a wild-type disease-free state and the second cell state represents a diseased state.

In some embodiments, the second cell state is characterized by an aberrant cell process while the first cell state represents a wild-type disease-free state. In some embodiments, the aberrant cell process is associated with a disease. For example, in some embodiments, the aberrant cell process is indicative of or related to a mechanism underlying any of the characteristics of disease, including but not limited to onset, progression, symptoms, severity, and/or resolution of disease. In some embodiments, the aberrant cell process is a functional pathway. In some embodiments, the aberrant cell process is a signaling pathway. In some embodiments, the aberrant cell process is characterized and/or modulated by a transcriptional network (e.g., a gene regulatory network).

In some embodiments, the aberrant cell process is an annotation, such as a gene set enrichment assay (GSEA) annotation, a gene ontology annotation, a functional and/or signaling pathway annotation, and/or a cellular signature annotation associated with the second cell state. Annotations can be obtained from any public knowledge database, including but not limited to the NIH Gene Expression Omnibus (GEO), EBI ArrayExpress, NCBI, BLAST, EMBL-EBI, GenBank, Ensembl, the KEGG pathway database, the Library of Integrated Network-based Cellular Signatures (LINCS) L1000 dataset, the Reactome pathway database, the Gene Ontology project, and/or any disease-specific database.

Thus, in some embodiments, the second cell state is any respective disease, functional pathway, signaling pathway, mechanism of action, transcriptional network, discrepancy, and/or cellular or biological process as described herein.

In some embodiments, the second cell state is characterized by loss of a function of a cell, gain of a function of a cell, progression of a cell (e.g., transition of the cell into a differentiated state), stasis of a cell (e.g., inability of the cell to transition into a differentiated state), intrusion of a cell (e.g., emergence of the cell in an abnormal location), disappearance of a cell (e.g., absence of the cell in a location where the cell is normally present), disorder of a cell (e.g., a structural, morphological, and/or spatial change within and/or around the cell), loss of network of a cell (e.g., a change in the cell that eliminates normal effects in progeny cells or cells downstream of the cell), a gain of network of a cell (e.g., a change in the cell that triggers new downstream effects in progeny cells of cells downstream of the cell), a surplus of a cell (e.g., an overabundance of the cell), a deficit of a cell (e.g., a density of the cell being below a critical threshold), a difference in cellular constituent ratio and/or quantity in a cell, a difference in the rate of transitions in a cell, or any combination thereof.

Block 204. Referring to block 204, a fingerprint 34 of a test chemical compound is obtained. In some embodiments, the test chemical compound is a small molecule, a biologic, a protein, a protein combined with a small molecule, an ADC, a nucleic acid, such as an siRNA or interfering RNA, a cDNA over-expressing wild-type and/or mutant shRNA, a cDNA over-expressing wild-type and/or mutant guide RNA (e.g., Cas9 system or other cellular-component editing system), and/or any combination of any of the foregoing. In some embodiments, the test chemical compound is inorganic or organic.

Block 206. Referring to block 206, in some embodiments, the test chemical compound is a first organic compound having a molecular weight of less than 2000 Daltons (Da). In some embodiments, the test chemical compound has a molecular weight of at least 10 Da, at least 20 Da, at least 50 Da, at least 100 Da, at least 200 Da, at least 500 Da, at least 1 kDa, at least 2 kDa, at least 3 kDa, at least 5 kDa, at least 10 kDa, at least 20 kDa, at least 30 kDa, at least 50 kDa, at least 100 kDa, or at least 500 kDa. In some embodiments, the test chemical compound has a molecular weight of no more than 1000 kDa, no more than 500 kDa, no more than 100 kDa, no more than 50 kDa, no more than 10 kDa, no more than 5 kDa, no more than 2 kDa, no more than 1 kDa, no more than 500 Da, no more than 300 Da, no more than 100 Da, or no more than 50 Da. In some embodiments, the test chemical compound has a molecular weight of from 10 Da to 900 Da, from 50 Da to 1000 Da, from 100 Da to 2000 Da, from 1 kDa to 10 kDa, from 5 kDa to 500 kDa, or from 100 kDa to 1000 kDa. In some embodiments, the test chemical compound has a molecular weight that falls within another range starting no lower than 10 Daltons and ending no higher than 1000 kDa.

In some embodiments, a test chemical compound is a polymer that comprises at least 2, at least 5, at least 10, at least 20, at least 30, at least 40, or at least 50 amino acids. In some embodiments, a respective test chemical compound and/or a respective reference compound comprises no more than 60, no more than 50, no more than 40, no more than 30, no more than 20, or no more than 10 amino acids. In some embodiments, a respective test chemical compound and/or a respective reference compound consists of from 2 to 10, from 2 to 50, from 5 to 50, from 10 to 45, or from 35 to 60 amino acids. In some embodiments, a respective test chemical compound and/or a respective reference compound comprises a plurality of amino acids that falls within another range starting no lower than 2 amino acids and ending no higher than 60 amino acids.

In some embodiments, a test chemical compound is a small molecule. For instance, in some embodiments, a test chemical compound is an organic compound having a molecular weight of less than approximately 1000 Daltons (e.g., less than 900 Daltons).

In some embodiments, a test chemical compound is a peptide. For instance, in some embodiments, a test chemical compound is an organic compound having 41 amino acids or fewer. In some embodiments, a test chemical compound has a molecular weight of less than approximately 4500 Daltons (e.g., 41 amino acids*110 Daltons).

In some embodiments, a test chemical compound is a protein. For instance, in some embodiments, a test chemical compound is an organic polymer having at least 42 amino acids. In some embodiments, a test chemical compound has a molecular weight of at least approximately 4600 Daltons (e.g., 42 amino acids*110 Daltons).

Block 208. Referring to block 208, in some embodiments, the test chemical compound satisfies any two or more rules, any three or more rules, or all four rules of the Lipinski's rule of Five. The Lipinski rule of five (e.g., RO5) criteria are a set of guidelines used to evaluate druglikeness, such as to determine whether a respective compound with a respective pharmacological or biological activity has corresponding chemical or physical properties suitable for administration in humans. Lipinski's rule of five includes the following criteria for determining the druglikeness of a compound: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, a compound of the present disclosure satisfies one or more criteria in addition to Lipinski's Rule of Five. For example, in some embodiments, a compound of the present disclosure has five or fewer aromatic rings, four or fewer aromatic rings, three or fewer aromatic rings, or two or fewer aromatic rings. In some embodiments, the test chemical compound is an organic compound that satisfies at least two, three or four criteria of the Lipinski rule of five criteria. In some embodiments, the test chemical compound is an organic compound that satisfies zero, one, two, three, or all four criteria of the Lipinski rule of five criteria.

Blocks 210-212. Referring to block 210, in some embodiments, the fingerprint of the test chemical compound comprises 100 features. Referring to block 212, in some embodiments, the fingerprint of the test chemical compound consists of between 10 features and 100,000 features. In some embodiments, the fingerprint of the test chemical compound comprises consists of between 10 features and 1000 features. In some embodiments, the fingerprint of the test chemical compound comprises 10 or more, 50 or more 100 or more, 500 or more, 1000 or more, 2000 or more, 3000 or more, or 5000 or more features.

Block 214. Referring to block 214, in some embodiments, the fingerprint of the test chemical compound is calculated from a chemical structure of the test chemical compound using a simplified molecular-input line-entry system (SMILES) string representation of the test chemical compound. Thus, in some embodiments, the method comprises calculating the fingerprint as a simplified molecular-input line-entry system (SMILES) string representation of the test chemical compound. Molecular fingerprinting using SMILES strings is further described, for example, in Honda et al., 2019, “SMILES Transformer: Pre-trained Molecular Fingerprint for Low Data Drug Discovery,” arXiv: 1911.04738, which is hereby incorporated herein by reference in its entirety.

Block 216. Referring to block 216, in some embodiments, the fingerprint of the test chemical compound is calculated from a chemical structure of the test chemical compound using Daylight, BCI, ECFP4, EcFC, MDL, APFP, TTFP, UNITY 2D fingerprint, RNNS2S, or GraphConv.

In some embodiments the fingerprint for the test chemical compound is in the form of a graph-based molecular fingerprint. In graph-based molecular fingerprinting, the original molecular structure is represented by a graph, in which nodes represent individual atoms and edges represent bonds between atoms. Graph-based approaches provide several advantages, including the ability to efficiently encode multiple substructures with lower size requirements and thus lower computational burden, as well as the ability to encode indications of structural similarity between fingerprints. Graph-based fingerprinting is further described, for instance, in Duvenaud et al., 2015, “Convolutional networks on graphs for learning molecular fingerprints,” NeurIPS, 2224-2232, which is hereby incorporated herein by reference in its entirety. In some embodiments, the fingerprint is generated from a graph convolutional network. In some embodiments, the fingerprint is generated from a spatial graph convolutional network, such as a graph attention network (GAT), a graph isomorphism network (GIN), or a graph substructure index-based approximate graph (SAGA). In some embodiments, the fingerprint is generated from a spectral graph convolutional network, such as a spectral graph convolution using Chebyshev polynomial filtering.

Block 218. Referring to block 218, in some embodiments, the fingerprint of the test chemical compound is calculated as a plurality of features that comprise a plurality of bioactivity descriptors for the test chemical compound. In some embodiments, the plurality of bioactivity descriptors include a numeric representation of the test chemical compound obtained from a two-dimensional fingerprint of the test chemical compound, a mechanism of action of the test chemical compound, a small molecule role possessed by the test chemical compound, a therapeutic area associated with the test chemical compound, a three-dimensional fingerprint of the test chemical compound, an association of the test chemical compound with one or more metabolic genes, an association of the test chemical compound with a small molecule pathway, an association of the test chemical compound with a cancer cell line, a crystal structure of the test chemical compound, a signaling pathway associated with the test chemical compound, a therapeutic side effect associated with the test chemical compound, a structural key associated with the test chemical compound, a binding affinity of the test chemical compound against a macromolecular target, a biological process associated with the test chemical compound, a morphology of cells exposed the test chemical compound, a disease associated with the test chemical compound, a toxicology associated with the test chemical compound, a physicochemistry associated with the test chemical compound, a drug-drug interaction associated with the test chemical compound, an inhibitory constant associated with the test chemical compound, a binding interaction of the test chemical compound with one or more residues of a protein, a Gibbs free energy of the binding of the test chemical compound with a protein, or any combination thereof.

Block 220. Referring to block 220, in some embodiments, a model that predicts bioactivity descriptors is used to determine one or more of the plurality of bioactivity descriptors. For instance, in some embodiments a test chemical compound fingerprint 34 for a test chemical structure is obtained by inputting the test chemical structure into the python package “Signaturizer” (https://pypi.org/project/signaturizer/) to obtain a fingerprint that is relevant to the corresponding differential expression signature 38. Trained on the “Chemical Checker” database, Signaturizer predicts bioactivity for an arbitrary molecule. Mathematically, this is a high-dimensional vector (˜500 features). See Bertoni, 2021, “Bioactivity descriptors for uncharacterized chemical compounds,” Nature Communications 12, Article number: 3932, which is hereby incorporated by reference.

Block 222. Referring to block 222 of FIG. 2B and as further illustrated in FIG. 4, in some embodiments, a differential expression signature 38 is obtained. The differential expression signature comprises a plurality of differential values 42. Each respective differential value in the plurality of differential values corresponds to a respective cellular constituent 40 in a set of cellular constituents. The respective differential value 42 represents a difference between (i) one or more first state abundance values 44 measured for the respective cellular constituent 40 in a first cell-based assay of a first plurality of cells that represent the first cell state and (ii) one or more second state abundance values 46 measured for the respective cellular constituent 40 in a second cell-based assay of a second plurality of cells that represent the second cell state.

In some embodiments a differential expression signature 38 has the form T=(T₁, . . . , Tₙ) where each 7; in (T₁, . . . , Tₙ) is a differential value 42 that scores the differential expression of a particular cellular constituent 40 in a set of cellular constituents between (a) one or more perturbed cells and (b) one or more non-perturbed cells. In some such embodiments, each cellular constituent is a gene and thus, in such embodiments each dimension Tᵢ corresponds to the differential expression of a corresponding gene i. In some embodiments, the set of cellular constituents is drawn from the Library of Integrated Network-Based Cellular Constituents (LINCS) database (See Example 1). In some embodiments the set of genes comprises 1000 “landmark” genes in the LINCS database. As the scale of T is highly variable across experiments (strong batch effects), in some embodiments it is rescales and clipped T such that T_{b}=min(max(Δ·T, −2), 2), where A is chosen such that T_{b} has standard deviation of 1. It has been observed that the information sacrificed by this operation is negligible compared to the robustness to batch effects that is gained by it. Optionally, in some embodiments, T is converted into gene module expression (e.g., expression of certain genes are grouped into modules) thereby reducing the number of features of the model significantly. The output of this transcriptional preprocessing is one form of a differential expression signature T_{b} 38.

As illustrated in FIG. 1A, in some embodiments a differential expression signature 38 comprises an identification of a set of cellular constituents 40 and, for each respective cellular constituent 40 in the set plurality of cellular constituents, a corresponding differential value 42 that quantifies a change in abundance of the respective cellular constituent between a first cell state and a second cell state.

In some embodiments the first cell state represents a wild-type disease-free state and the second cell state represents a diseased state. In some embodiments, the first state represents a desirable disease-free state obtained by exposing cells that represent a particular second state (diseased state) to a compound that treats the cells. In some embodiments, the first state represents a desirable disease-free state that is obtained with cells that have not been exposed to any perturban.

In some embodiments, the second state represents a diseased state that is obtained by exposing cells that represent a particular first state (wild-type state) to a compound that causes the cells to transform to the diseased state. In some embodiments, the second state is represented by cells that have the second cell state that is possessed by the cells without been exposure to any perturban.

In some embodiments, data needed to compute a differential expression signature 38 is obtained from a publicly available database, such as the Genomics of Drug Sensitivity in Cancer, the Cancer Therapeutics Response Portal, the Connectivity Map, PharmacoDB, Base of Bioisosterically Exchangeable Replacements (BoBER), DrugBank, the Human Cell Atlas, the Molecular Signatures Database (MSigDB), and/or Enrichr. Such databases include cell-based cellular constituent abundance data from cells representing specific states. Other suitable databases from which cellular constituent abundance data can be obtained for cell based assays of cells representing various states include, but are not limited to, the NIH Gene Expression Omnibus (GEO), EBI ArrayExpress, NCBI, BLAST, EMBL-EBI, GenBank, Ensembl, the KEGG pathway database, the Library of Integrated Network-based Cellular Signatures (LINCS) L1000 dataset, the Reactome pathway database, and the Gene Ontology project.

Blocks 224-226. Referring to block 224, in some embodiments, each corresponding differential value in the plurality of differential values is a comparison of: (i) a first measure of central tendency of the one or more abundance values for the respective cellular constituent across the first plurality of cells, and (ii) a second measure of central tendency of the one or more abundance values for the respective cellular constituent across the second plurality of cells. In some embodiments, the measure of central tendency is a mean, median, mode, weighted mean, weighted median, and/or weighted mode.

In some embodiments, the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells. In some embodiments, the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells. In some embodiments the first cell-based assay is a bulk cell-based assay and a single abundance value 44 is measured for each cellular constituent 40 across the first plurality of cells. In some embodiments the second cell-based assay is a bulk cell-based assay and a single abundance value 44 is measured for each cellular constituent 40 across the second plurality of cells.

In some embodiments the first cell-based assay is a single cell-based assay and a plurality of abundance values 44 are measured for each cellular constituent 40 across the first plurality of cells. Referring to block 226, in some such embodiments, the plurality of abundance values (first stated abundance values 44) measured for the respective cellular constituent 40 in the first cell-based assay are obtained by the first single-cell assay. In some such embodiments the second cell-based assay is also a single-cell assay and a plurality of abundance values (second state abundance values) 46 measured for the respective cellular constituent in the second plurality of cell-based assay are obtained by a second single-cell assay.

In some embodiments, the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells.

In some embodiments, the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells.

In some embodiments, the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells, the first cell-based assay is a first single-cell based assay and the one or more first state abundance values 44 for the first single-cell based assay comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more first state abundance values 44 for each cellular constituent 40 in a plurality of cellular constituents.

In some embodiments, the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells, the second cell-based assay is a second single-cell based assay and the one or more second state abundance values 46 for the second single-cell based assay comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more second state abundance values 46 for each cellular constituent 40 in a plurality of cellular constituents.

In some embodiments, the first and second cell-based assay is the same single-cell based assay. In some embodiments, the first and second cell-based assay is the same bulk-cell based assay.

Any one of a number of abundance counting techniques (e.g., cellular constituent measurement techniques) may be used to obtain the first state abundance values 44 and the second states abundance values 46 for each cellular constituent in the first and second plurality of cells. For instance, Table 1 below lists non-limiting techniques for single-cell cellular constituent measurement, in accordance with some embodiments of the present disclosure. In other embodiments of the present disclosure bulk cell based assays are used to obtain the first state abundance values 44 and the second states abundance values 46 for each cellular constituent in the first and second plurality of cells.

In some embodiments, the abundance of a cellular constituent in the first or second cell-based assay is determined using one or more methods including microarray analysis via fluorescence, chemiluminescence, electric signal detection, polymerase chain reaction (PCR), reverse transcriptase polymerase chain reaction (RT-PCR), digital droplet PCR (ddPCR), solid-state nanopore detection, RNA switch activation, a Northern blot, and/or a serial analysis of gene expression (SAGE). In some embodiments, the corresponding abundance of the respective cellular constituent in the respective cell in the first and/or second plurality of cells is determined by a colorimetric measurement, a fluorescence measurement, a luminescence measurement, or a resonance energy transfer (FRET) measurement.

In some embodiments, gene expression in a respective cell in the first and/or the second plurality of cells is measured by sequencing RNA from the cell and then counting the quantity of each gene transcript identified during the sequencing. In some embodiments, the gene transcripts sequenced and quantified include RNA, such as mRNA. In some embodiments, the gene transcripts sequenced and quantified include a downstream product of mRNA, such as a protein (e.g., a transcription factor). In general, as used herein, the term “gene transcript” may be used to denote any downstream product of gene transcription or translation, including post-translational modification, and “gene expression” may be used to refer generally to any measure of gene transcripts.

In some embodiments, the abundance of a cellular constituent is RNA abundance (e.g., gene expression), and the abundance of the respective cellular constituent is determined by measuring polynucleotide levels of one or more nucleic acid molecules corresponding to the respective gene. The transcript levels of the respective gene can be determined from the amount of mRNA, or polynucleotides derived therefrom, present in the respective cell in the first and/or second plurality of cells. Polynucleotides can be detected and quantitated by a variety of methods including, but not limited to, microarray analysis, polymerase chain reaction (PCR), reverse transcriptase polymerase chain reaction (RT-PCR), Northern blot, serial analysis of gene expression (SAGE), RNA switches, RNA fingerprinting, ligase chain reaction, Qbeta replicase, isothermal amplification method, strand displacement amplification, transcription based amplification systems, nuclease protection assays (Si nuclease or RNAse protection assays), and/or solid-state nanopore detection. See, e.g., Draghici, Data Analysis Tools for DNA Microarrays, Chapman and Hall/CRC, 2003; Simon et al., Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al., (1995) Science 270: 484-487; Matsumura et al., (2005) Cell. Microbiol. 7: 11-18; Serial Analysis of Gene Expression (SAGE): Methods and Protocols (Methods in Molecular Biology), Humana Press, 2008; each of which is hereby incorporated herein by reference in its entirety.

In some embodiments, the abundance of a cellular constituent is obtained from expressed RNA or a nucleic acid derived therefrom (e.g., cDNA or amplified RNA derived from cDNA that incorporates an RNA polymerase promoter) from the plurality of cells in the first state and/or the plurality of cells in the second state, including naturally occurring nucleic acid molecules, as well as synthetic nucleic acid molecules. Thus, in some embodiments, the abundance of a cellular constituent is obtained from such non-limiting sources as total cellular RNA, poly(A)+ messenger RNA (mRNA) or a fraction thereof, cytoplasmic mRNA, or RNA transcribed from cDNA (e.g., cRNA). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (see, e.g., Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In some embodiments, the abundance of a cellular constituent in the cells in the first state and/or the plurality of cells in the second state is determined by sequencing. In some embodiments, the abundance of a cellular constituent in the cells in the first state and/or the plurality of cells in the second state is determined by single-cell ribonucleic acid (RNA) sequencing (scRNA-seq), scTag-seq, single-cell assay for transposase-accessible chromatin using sequencing (scATAC-seq), CyTOF/SCOP, E-MS/Abseq, miRNA-seq, CITE-seq, or any combination thereof.

The cellular constituent abundance measurement technique used for a given cellular constituent can be selected based on the desired cellular constituent to be measured. For instance, scRNA-seq, scTag-seq, and miRNA-seq can be used to measure RNA expression. Specifically, scRNA-seq measures expression of RNA transcripts, scTag-seq allows detection of rare mRNA species, and miRNA-seq measures expression of micro-RNAs. CyTOF/SCOP and E-MS/Abseq can be used to measure protein expression in the cell. CITE-seq simultaneously measures both gene expression and protein expression in the cell, and scATAC-seq measures chromatin conformation in the cell. Table 1 below provides example protocols for performing each of the cellular constituent abundance measurement techniques described above.

TABLE 1
Example Measurement Protocols

| Technique | Protocol |
|---|---|
| RNA-seq | Olsen et al., (2018), “Introduction to Single-Cell RNA |
| | Sequencing,” Current protocols in molecular biology |
| | 122(1), pg. 57. |
| Tag-seq | Rozenberg et al., (2016), “Digital gene expression |
| | analysis with sample multiplexing and PCR duplicate |
| | detection: A straightforward protocol,” BioTechniques, |
| | 61(1), pg. 26. |
| ATAC-seq | Buenrostro et al., (2015), “ATAC-seq: a method for |
| | assaying chromatic accessibility genome-wide,” Current |
| | protcols in molecular biology, 109(1), pg. 21. |
| miRNA-seq | Faridani et al., (2016), “Single-cell sequencing of the |
| | small-RNA transcriptome,” Nature biotechnology, |
| | 34(12), pg. 1264. |
| CyTOF/ | Bandura et al., (2009), “Mass cytometry: technique for |
| SCoPE-MS/ | real time single cell multitarget immunoassay based on |
| Abseq | inductively coupled plasma time-of-flight mass |
| | spectrometry,” Analytic chemistry, 81(16), pg. 6813. |
| | Budnik et al., (2018), “SCoPE-ME: mass spectrometry of |
| | single mammalian cells quantifies proteome |
| | heterogeneity during cell differentiation,” Genome |
| | biology, 19(1), pg. 161. |
| | Shahi et al., (2017), “Abseq: Ultrahigh-throughput single |
| | cell protein profiling with droplet microfluidic |
| | barcoding,” Scientific reports, 7, pg. 44447. |
| CITE-seq | Stoeckius et al., (2017), “Simultaneous epitope and |
| | transcriptome measurement in single cells,” Nature |
| | Methods, 14(9), pg. 856. |

In some embodiments, the plurality of cellular constituents in the first cell-based assay or the second cell-based assay is measured at a single time point. In some embodiments, the plurality of cellular constituents is measured at multiple time points. For instance, in some embodiments, the plurality of cellular constituents is measured at multiple time points throughout a cell state transition (e.g., a differentiation process, a response to an exposure to a compound, a developmental process, etc.).

It is to be understood that this is by way of illustration and not limitation, as the present disclosure encompasses analogous methods using measurements of other cellular constituents obtained from cells (e.g., single cells). It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

In some embodiments, the corresponding abundances for the plurality of cellular constituents in the first and/or the second plurality of cells (e.g., the one or more first datasets and/or the one or more second datasets) are preprocessed. In some embodiments, the preprocessing includes one or more of filtering, normalization, mapping (e.g., to a reference sequence), quantification, scaling, deconvolution, cleaning, dimension reduction, transformation, statistical analysis, and/or aggregation.

For example, in some embodiments, the plurality of cellular constituents is filtered based on a desired quality, e.g., size and/or quality of a nucleic acid sequence, or a minimum and/or maximum abundance value for a respective cellular constituent. In some embodiments, filtering is performed in part or in its entirety by various software tools, such as Skewer. See, Jiang, H. et al., BMC Bioinformatics 15(182): 1-12 (2014). In some embodiments, the plurality of cellular constituents is filtered for quality control, for example, using a sequencing data QC software such as AfterQC, Kraken, RNA-SeQC, FastQC, or another similar software program. In some embodiments, the plurality of cellular constituents is normalized, e.g., to account for pull-down, amplification, and/or sequencing bias (e.g., mappability, GC bias etc.). See, for example, Schwartz et al., PLOS ONE 6(1):e16685 (2011) and Benjamini and Speed, Nucleic Acids Research 40(10):e72 (2012), the contents of which are hereby incorporated by reference, in their entireties, for all purposes. In some embodiments, the preprocessing removes a subset of cellular constituents from the plurality of cellular constituents. In some embodiments, the preprocessing the corresponding abundances for the plurality of cellular constituents improves (e.g., lowers) a high signal-to-noise ratio.

In some embodiments, the preprocessing comprises performing a comparison of a corresponding abundance of a respective cellular constituent in a respective cell to a reference abundance. In some embodiments, the reference abundance is obtained from, e.g., a normal sample, a matched sample, a reference dataset comprising reference abundance values, a reference cellular constituent such as a housekeeping gene, and/or a reference standard. In some embodiments, this comparison of cellular constituent abundances is performed using any differential expression test including, but not limited to, a difference of means test, a Wilcoxon rank-sum test (Mann Whitney U test), a t-test, a logistic regression, and a generalized linear model. Those of skill in the art will appreciate that other metrics are also possible for comparison and/or normalization of cellular constituent abundances.

Thus, in some embodiments, the corresponding abundance of a respective cellular constituent in a respective cell in the one or more first datasets and/or in the one or more second datasets comprises any one of a variety of forms, including, without limitation, a raw abundance value, an absolute abundance value (e.g., transcript number), a relative abundance value (e.g., relative fluorescent units, transcriptome analysis, and/or gene set expression analysis (GSEA)), a compound or aggregated abundance value, a transformed abundance value (e.g., log₂ and/or log₁₀ transformed), a change (e.g., fold- or log-change) relative to a reference (e.g., a normal sample, matched sample, reference dataset, housekeeping gene, and/or reference standard), a standardized abundance value, a measure of central tendency (e.g., mean, median, mode, weighted mean, weighted median, and/or weighted mode), a measure of dispersion (e.g., variance, standard deviation, and/or standard error), an adjusted abundance value (e.g., normalized, scaled, and/or error-corrected), a dimension-reduced abundance value (e.g., principal component vectors and/or latent components), and/or a combination thereof. Methods for obtaining cellular constituent abundances using dimension reduction techniques are known in the art and further detailed below, including but not limited to principal component analysis, factor analysis, linear discriminant analysis, multi-dimensional scaling, isometric feature mapping, locally linear embedding, hessian eigenmapping, spectral embedding, t-distributed stochastic neighbor embedding, and/or any substitutions, additions, deletions, modification, and/or combinations thereof as will be apparent to one skilled in the art. See, for example, Sumithra et al., 2015, “A Review of Various Linear and Non Linear Dimensionality Reduction Techniques,” Int J Comp Sci and Inf Tech, 6(3), 2354-2360, which is hereby incorporated herein by reference in its entirety.

Block 228. Referring to block 228, in some embodiments, the first single-cell assay and/or the second single-cell assay is ribonucleic acid (RNA) sequencing (scRNA-seq), CyTOF/SCOP, E-MS/Abseq, CITE-seq, single-cell assay for transposase-accessible chromatin using sequencing (scATAC-seq), ATAC-seq, or any combination thereof. In some embodiments, the first single-cell assay and/or the second single-cell assay makes use of perturb-seq, CRISP-seq, CROP-seq, CRISPRi, TAP-seq, CRISPRa, perturb-CITE-seq, sci-Plex, multiplexed, MIX-seq, CyTOF, and/or scRNA-seq. In some embodiments, the first single-cell assay and/or the second single-cell assay is any method of obtaining omics data, including mass spectrometry (e.g., LCMS, GCMS), flow cytometry, quantitative polymerase chain reaction (qPCR), gel electrophoresis, gene-chip analysis, microarray, cytofluorimetric analysis, fluorescence microscopy, confocal laser scanning microscopy, laser scanning cytometry, affinity chromatography, manual batch mode separation, electric field suspension, sequencing, and/or any combination thereof. In some embodiments, any of the methods for obtaining cellular constituent abundance values disclosed herein are contemplated for use in obtaining first state abundance values and second state abundance values 46 for cellular constituent constituents 40 in order to derive the differential expression signature 38.

Block 230. Referring to block 230, in some embodiments, each cellular constituent in the set of cellular constituents uniquely maps to a different gene. In some such embodiments, the set of cellular constituents collectively maps to 3 or more, 4 or more, 5 or more, 6 or more, 7 or more, 8 or more, 9 or more, 10 or more, 100 or more, 200 or more, 300 or more, 400 or more, 500 or more, 600 or more, 700 or more, 800 or more, 900 or more, or 1000 or more different genes. In some embodiments the set of cellular constituents collectively maps to between 1000 and 20000 different genes.

Block 232. Referring to block 232, in some embodiments, each cellular constituent in the set of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification (e.g., glycosylation, phosphorylation, acetylation, or ubiquitylation) of a protein.

In some embodiments, a cellular constituent is a gene, a gene product (e.g., an mRNA and/or a protein), a carbohydrate, a lipid, an epigenetic feature, an epitranscriptomic feature, a metabolite, and/or a combination thereof. In some embodiments, each cellular constituent in the set of cellular constituents is a particular gene, a particular mRNA associated with a gene, a carbohydrate, a lipid, an epigenetic feature, an epitranscriptomic feature, a metabolite, a protein, or a combination thereof.

In some embodiments, the set of cellular constituents includes nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc.) proteins, lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular constituents such as oxidized and reduced forms of nicotinamide adenine dinucleotide (NADP/NADPH), and any combinations thereof.

Blocks 234-236. Referring to block 234, in some embodiments, the set of cellular constituents comprises 3 cellular constituents, 4 cellular constituents, 5 cellular constituents, 6 cellular constituents, 7 cellular constituents, 8 cellular constituents, 9 cellular constituents, 10 or more cellular constituents, 20 or more cellular constituents, 30 or more cellular constituents, 40 or more cellular constituents, or 50 or more cellular constituents. Referring to block 236, in some embodiments, the set of cellular constituents consists of between 10 and 1000 cellular constituents. In some embodiments, the set of cellular constituents comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 50,000, or more than 50,000 cellular constituents. In some embodiments, the set of cellular constituents comprises no more than 70,000, no more than 50,000, no more than 30,000, no more than 10,000, no more than 5000, no more than 1000, no more than 500, no more than 200, no more than 100, no more than 90, no more than 80, no more than 70, no more than 60, no more than 50, or no more than 40 cellular constituents. In some embodiments, the set of cellular constituents consists of between twenty and 10,000 cellular constituents. In some embodiments, the set of cellular constituents consists of between 100 and 8,000 cellular constituents. In some embodiments, the set of cellular constituents comprises from 5 to 20, from 20 to 50, from 50 to 100, from 100 to 200, from 200 to 500, from 500 to 1000, from 1000 to 5000, from 5000 to 10,000, or from 10,000 to 50,000 cellular constituents. In some embodiments, the set of cellular constituents falls within another range starting no lower than 5 cellular constituents and ending no higher than 70,000 cellular constituents.

As an example, in some embodiments, the set of cellular constituents comprises a plurality of genes, optionally measured at the RNA level. In some embodiments, the plurality of genes comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, or at least 1000 genes. In some embodiments, the plurality of genes comprises at least 1000, at least 2000, at least 3000, at least 4000, at least 5000, at least 10,000, at least 30,000, at least 50,000, or more than 50,000 genes. In some embodiments, the plurality of genes consists of from 5 to 20, from 20 to 50, from 50 to 100, from 100 to 200, from 200 to 500, from 500 to 1000, from 1000 to 5000, from 5000 to 10,000, or from 10,000 to 50,000 genes.

As another example, in some embodiments, the set of cellular constituents comprises a plurality of proteins. In some embodiments, the plurality of proteins comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, or at least 1000 proteins. In some embodiments, the plurality of proteins comprises at least 1000, at least 2000, at least 3000, at least 4000, at least 5000, at least 10,000, at least 30,000, at least 50,000, or more than 50,000 proteins. In some embodiments, the plurality of proteins comprises from 5 to 20, from 20 to 50, from 50 to 100, from 100 to 200, from 200 to 500, from 500 to 1000, from 1000 to 5000, from 5000 to 10,000, or from 10,000 to 50,000 proteins.

Block 238. Referring to block 238, in some embodiments, the first plurality of cells and the second plurality of cells are cells from an organ, cells from a tissue, a plurality of stem cells, a plurality of primary human cells, cells from umbilical cord blood, cells from peripheral blood, bone marrow cells, cells from a solid tissue, or a plurality of differentiated cells.

In some embodiments the first plurality of cells or the second plurality of cells comprises or consists of cells from an organ. In some such embodiments, the organ is heart, liver, lung, muscle, brain, pancreas, spleen, kidney, small intestine, uterus, or bladder.

In some embodiments the first plurality of cells or the second plurality of cells comprises or consists of cells from a tissue. In some such embodiments, the tissue is bone, cartilage, joint, tracheae, spinal cord, cornea, eye, skin, or blood vessel.

In some embodiments, the first plurality of cells or the second plurality of cells comprises or consists of a plurality of stem cells. In some such embodiments, the plurality of stem cells is a plurality of embryonic stem cells, a plurality of adult stem cells, or a plurality of induced pluripotent stem cells (iPSC).

In some embodiments, the first plurality of cells or the second plurality of cells comprises or consists of a plurality of primary human cells. In some such embodiments the plurality of primary human cells is a plurality of CD34+ cells, a plurality of CD34+ hematopoietic stems, a plurality of progenitor cells (HSPC), a plurality of T-cells, a plurality of mesenchymal stem cells (MSC), a plurality of airway basal stem cells, or a plurality of induced pluripotent stem cells.

In some embodiments, the first plurality of cells or the second plurality of cells comprises or consists of a plurality of human cell lines. In some such embodiments, the first plurality of cells or the second plurality of cells comprises or consists of cells from umbilical cord blood, from peripheral blood, or from bone marrow.

In some embodiments, the first plurality of cells or the second plurality of cells comprises or consists of cells in or from a solid tissue. In some such embodiments, the solid tissue is placenta, liver, heart, brain, kidney, or gastrointestinal tract.

In some embodiments, the first plurality of cells or the second plurality of cells comprises or consists of a plurality of differentiated cells. In some such embodiments, the plurality of differentiated cells is a plurality of megakaryocytes, a plurality of osteoblasts, a plurality of chondrocytes, a plurality of adipocytes, a plurality of hepatocytes, a plurality of hepatic mesothelial cells, a plurality of biliary epithelial cells, a plurality of hepatic stellate cells, a plurality of hepatic sinusoid endothelial cells, a plurality of Kupffer cells, a plurality of pit cells, a plurality of vascular endothelial cells, a plurality of pancreatic duct epithelial cells, a plurality of pancreatic duct cells, a plurality of centroacinous cells, a plurality of acinar cells, a plurality of islets of Langerhans, a plurality of cardiac muscle cells, a plurality of fibroblasts, a plurality of keratinocytes, a plurality of smooth muscle cells, a plurality of type I alveolar epithelial cells, a plurality of type II alveolar epithelial cells, a plurality of Clara cells, a plurality of ciliated epithelial cells, a plurality of basal cells, a plurality of goblet cells, a plurality of neuroendocrine cells, a plurality of kultschitzky cells, a plurality of renal tubular epithelial cells, a plurality of urothelial cells, a plurality of columnar epithelial cells, a plurality of glomerular epithelial cells, a plurality of glomerular endothelial cells, a plurality of podocytes, a plurality of mesangium cells, a plurality of nerve cells, a plurality of astrocytes, a plurality of microglia, or a plurality of oligodendrocytes.

Block 240. Referring to block 240, in some embodiments, the one or more abundance values measured for the respective cellular constituent in the first cell-based assay or the one or more abundance values measured for the respective cellular constituent in the second cell-based assay are determined by a colorimetric measurement, a fluorescence measurement, a luminescence measurement, a resonance energy transfer (FRET) measurement, a measurement of a protein-protein interaction, a measurement of a protein-polynucleotide interaction, a measurement of a protein-small molecule interaction. mass spectrometry, nuclear magnetic resonance, or a microarray measurement.

Block 242. Referring to block 242, in some embodiments, responsive to inputting the fingerprint 34 of the test chemical compound into a first model 48, retrieve, as output from the first model, a respective chemical embedding 52. In some embodiments the first model 48 is a multilayer perceptron (fully connected neural network) containing dropout layers, batch normalization, and a ReLU non-linearity. In some embodiments model 48 produces an chemical embedding E_{S} 52 of the structure S of the test chemical compound.

In some embodiments the first model 48 comprises a plurality (e.g., 100, 200, 300, 500, 1000, 10,000 or more) of model parameters 50.

Block 244. Referring to block 244 of FIG. 2D, in some embodiments, responsive to inputting the differential expression signature 38 into a second model 56, a differential expression embedding is retrieved as output from the second model. Thus, in some such embodiments, the second model 56 serves as a differential expression signature 38 encoder. In some embodiments the second model 56, like the first model 48, is a multilayer perceptron (fully connected neural network) with similar features as the first model 48. The second model 56 produces a differential expression embedding 60 E_{T} of the differential expression signature 38 T.

Block 246. Referring to block 246, in some embodiments, the first model is a first multilayer perceptron and the second model is a second multilayer perceptron. In some such embodiments the first model 48 and/or the second model 56 is a fully connected second neural network, also known as a multilayer perceptron (MLP). In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. More disclosure on suitable MLPs that serve as the first model 48 and/or the second model 56 in some embodiments of the present disclosure is found in Vang-mata ed., 2020, Multilayer Perceptrons: Theory and Applications, Nova Science Publishers, Hauppauge, New York, which is hereby incorporated by reference.

In some embodiments, the first model 48 and/or the second model 56 is a neural network that is a fully connected neural network with ReLU activation. For instance, in some embodiments, the first model 48 and/or the second model 50 is a neural network comprising a corresponding one or more inputs (for the features 36 of the respective test chemical compound fingerprint 34 in the case of the first model 48 and for the differential values 42 of the differential expression signature 38 in the case of the second model 56), a corresponding first hidden layer comprising a corresponding plurality of hidden neurons, where each hidden neuron in the corresponding plurality of hidden neurons (i) is fully connected to each input in the plurality of inputs, (ii) is associated with a first activation function type, and (iii) is associated with a corresponding parameter (e.g., weight) in a plurality of parameters for the neural network, and one or more corresponding neural network outputs, where each respective neural network output in the corresponding one or more neural network outputs (i) directly or indirectly receives, as input, an output of each hidden neuron in the corresponding plurality of hidden neurons, and (ii) is associated with a second activation function type. In some such embodiments, the neural network is a fully connected network.

In some embodiments, where the first model 48 and/or the second model 50 is a neural network, the neural network comprises a plurality of hidden layers. As described above, hidden layers are located between input and output layers (e.g., to capture additional complexity). In some embodiments, where there is a plurality of hidden layers, each hidden layer may have a same or a different respective number of neurons.

In some embodiments, each hidden neuron (e.g., in a respective hidden layer in a neural network) is associated with an activation function that performs a function on the input data (e.g., a linear or non-linear function). Generally, the purpose of the activation function is to introduce nonlinearity into the data such that the neural network is trained on representations of the original data and can subsequently “fit” or generate additional representations of new (e.g., previously unseen) data. Selection of activation functions (e.g., a first and/or a second activation function) is dependent on the use case of the neural network, as certain activation functions can lead to saturation at the extreme ends of a dataset (e.g., tanh and/or sigmoid functions). For instance, in some embodiments, an activation function (e.g., a first and/or a second activation function) is selected from any suitable activation functions known in the art, including but not limited to any activation function disclosed herein.

In some embodiments, each hidden neuron is further associated with a parameter (e.g., a weight and/or a bias value) that contributes to the output of the neural network, determined based on the activation function. In some embodiments, the hidden neuron is initialized with arbitrary parameters (e.g., randomized weights). In some alternative embodiments, the hidden neuron is initialized with a predetermined set of parameters.

In some embodiments, the plurality of hidden neurons in a neural network (e.g., across one or more hidden layers) is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 neurons. In some embodiments, the plurality of hidden neurons is at least 100, at least 500, at least 800, at least 1000, at least 2000, at least 3000, at least 4000, at least 5000, at least 6000, at least 7000, at least 8000, at least 9000, at least 10,000, at least 15,000, at least 20,000, or at least 30,000 neurons. In some embodiments, the plurality of hidden neurons is no more than 30,000, no more than 20,000, no more than 15,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, or no more than 50 neurons. In some embodiments, the plurality of hidden neurons is from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 neurons. In some embodiments, the plurality of hidden neurons falls within another range starting no lower than 2 neurons and ending no higher than 30,000 neurons.

In some embodiments, the neural network comprises from 1 to 50 hidden layers. In some embodiments, the neural network comprises from 1 to 20 hidden layers. In some embodiments, the neural network comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 hidden layers. In some embodiments, the neural network comprises no more than 100, no more than 90, no more than 80, no more than 70, no more than 60, no more than 50, no more than 40, no more than 30, no more than 20, no more than 10, no more than 9, no more than 8, no more than 7, no more than 6, or no more than 5 hidden layers. In some embodiments, the neural network comprises from 1 to 5, from 1 to 10, from 1 to 20, from 10 to 50, from 2 to 80, from 5 to 100, from 10 to 100, from 50 to 100, or from 3 to 30 hidden layers. In some embodiments, the neural network comprises a plurality of hidden layers that falls within another range starting no lower than 1 layer and ending no higher than 100 layers.

In some embodiments, the neural network comprises a shallow neural network. A shallow neural network refers to a neural network with a small number of hidden layers. In some embodiments, such neural network architectures improve the efficiency of neural network training and conserve computational power due to the reduced number of layers involved in the training. In some embodiments, the neural network comprises one hidden layer. In some embodiments, the neural network comprises two, three, four, or five hidden layers.

Blocks 248-250. Referring to block 248, in some embodiments, the first model comprises 1000 model parameters and the second model comprises 1000 model parameters. Referring to block 250, in some embodiments, the first model consists of between 10 and 10 million parameters and the second model consists of between 10 and 10 million parameters.

In some embodiments, the first model 48 comprises a plurality of model parameters (e.g., weights and/or hyperparameters) 50. In some embodiments, the plurality of model parameters for the first model 48 comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million model parameters 50. In some embodiments, the plurality of parameters for the first model 48 comprises no more than 8 million, no more than 5 million, no more than 4 million, no more than 1 million, no more than 500,000, no more than 100,000, no more than 50,000, no more than 10,000, no more than 5000, no more than 1000, or no more than 500 model parameters 50. In some embodiments, the plurality of parameters for the first model 48 comprises from 10 to 5000, from 500 to 10,000, from 10,000 to 500,000, from 20,000 to 1 million, or from 1 million to 5 million model parameters 50. In some embodiments, the plurality of parameters for the first model 48 falls within another range starting no lower than 10 parameters and ending no higher than 8 million model parameters 50.

In some embodiments, the second model 56 comprises a plurality of model parameters (e.g., weights and/or hyperparameters) 58. In some embodiments, the plurality of model parameters for the second model 56 comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million model parameters 58. In some embodiments, the plurality of parameters for the second model 56 comprises no more than 8 million, no more than 5 million, no more than 4 million, no more than 1 million, no more than 500,000, no more than 100,000, no more than 50,000, no more than 10,000, no more than 5000, no more than 1000, or no more than 500 model parameters 58. In some embodiments, the plurality of parameters for the second model 56 comprises from 10 to 5000, from 500 to 10,000, from 10,000 to 500,000, from 20,000 to 1 million, or from 1 million to 5 million model parameters 58. In some embodiments, the plurality of parameters for the second model 56 falls within another range starting no lower than 10 parameters and ending no higher than 8 million model parameters 58.

Block 252. Referring to block 252, in some embodiments, the first model comprises one million parameters and the second model comprises one million parameters.

Block 254. Referring to block 254, in some embodiments, the likelihood that the test chemical compound causes the differential expression signature is determined based on a similarity between the respective chemical embedding and the differential expression embedding. In some embodiments the similarity score is the product of a function that maps the chemical embedding 52 and the differential expression embedding 60. In some embodiments this function F (E_{S}, E_{T}) is the normalized dot product:

$$F ( {E}_{S} , {E}_{T} ) = \frac{{E}_{T} \cdot {E}_{S}}{‖ {E}_{T} ‖ \vert \vert {E}_{S} ‖}$$

where E_{T} is the elements of the differential expression signature, and E_{S} is the corresponding elements of the chemical embedding 52. In some embodiments, the similarity is some other measure of distance between the chemical embedding 52 and the differential expression embedding 60 such as any of those disclosed on the Internet at en.wikipedia.org/wiki/Metric_space#Definition. In some embodiments, the number of chemical embedding elements 54 in the chemical embedding 52 matches the number of differential embedding elements in the differential expression embedding 60. In some such embodiments the chemical embedding 52 has 10 or more element 54, 100 or more elements 54, 1000 or more elements 54, or 10,000 or more elements 54 while the differential expression embedding 60 has 10 or more element 62, 100 or more elements 62, 1000 or more elements 62, or 10,000 or more elements 62. In some such embodiments the chemical embedding 52 has between 10 and one million elements 54 while the differential expression embedding 60 has between 10 and one million elements 62. Thus, upon inputting the pair (S_{b}, T_{b}) consisting of the fingerprint S_{b} 34 of a chemical structure and a differential expression signature T_{b} 38, the systems and methods of the present disclosure converts this pair into a similarity score. In some embodiments, the similarity score is between −1 and 1. In some such embodiments, the higher the score, the stronger the belief that the differential expression signature T_{b} 38 was caused by (or can be caused by) a perturbation with the structure S that was used to generate S_{b}.

Block 256. Referring to block 256, in some embodiments, the similarity between the respective chemical embedding and the differential expression embedding is determined by a distance between the respective chemical embedding and the differential expression embedding. In some such embodiments the distance is a cosine distance, Euclidian distance, Manhattan distance, Jaccard distance, correlation distance, Chi-square distance, or Mahalanobis distance between the chemical embedding 52 and the differential expression embedding 60.

Block 258. Referring to block 258, in some embodiments, the differential expression signature is associated with alleviating a condition in a subject, and the method further comprises administering the test chemical compound to the subject as a treatment to alleviate the condition in the subject when the test chemical compound is found to have a threshold likelihood of causing the differential expression signature.

Block 260. Referring to block 260, in some embodiments, the treatment comprises a composition comprising the test chemical compound and one or more excipient and/or one or more pharmaceutically acceptable carrier and/or one or more diluent.

These include all conventional solvents, dispersion media, fillers, solid carriers, coatings, antifungal and antibacterial agents, dermal penetration agents, surfactants, isotonic and absorption agents and the like. It will be understood that the compositions of the present disclosure may also include other supplementary physiologically active agents.

An exemplary carrier is pharmaceutically “acceptable” in the sense of being compatible with the other ingredients of the composition (e.g., the composition comprising the test chemical compound) and not injurious to the patient. The compositions may conveniently be presented in unit dosage form and may be prepared by any methods well known in the art of pharmacy. Such methods include the step of bringing into association the active ingredient with the carrier that constitutes one or more accessory ingredients. In general, the compositions are prepared by uniformly and intimately bringing into association the active ingredient with liquid carriers or finely divided solid carriers or both, and then if necessary shaping the product.

Exemplary compounds, compositions or combinations of the present disclosure (e.g., the test chemical compound) formulated for intravenous, intramuscular or intraperitoneal administration, or a pharmaceutically acceptable salt, solvate or prodrug thereof may be administered by injection or infusion.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

The compound, composition or combinations of the present disclosure (e.g., the test chemical compound) may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient (e.g., the test chemical compound) in a free-flowing form such as a powder or granules, optionally mixed with a binder (e.g., inert diluent, preservative disintegrant (e.g. sodium starch glycolate, cross-linked polyvinyl pyrrolidone, cross-linked sodium carboxymethyl cellulose) surface-active or dispersing agent). Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound, composition or combinations of the present disclosure (e.g., the test chemical compound) may be suitable for topical administration in the mouth including lozenges comprising the active ingredient in a flavored base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound, composition or combinations of the present disclosure (e.g., the test chemical compound) may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The compound, composition or combination of the present disclosure (e.g., the test chemical compound) may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the active ingredients particularly mentioned above, the composition or combination of this present disclosure (e.g., the test chemical compound) may include other agents conventional in the art having regard to the type of composition or combination in question, for example, those suitable for oral administration may include such further agents as binders, sweeteners, thickeners, flavoring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Block 262-264. Referring to block 262, in some embodiments, the condition is inflammation or pain. Referring to block 264, in some embodiments, the condition is a disease. In some such embodiments, the disease is selected from the group consisting of infectious or parasitic diseases; neoplasms; diseases of the blood or blood-forming organs; diseases of the immune system; endocrine, nutritional or metabolic diseases; mental, behavioral or neurodevelopmental disorders; sleep-wake disorders; diseases of the nervous system; diseases of the visual system; diseases of the ear or mastoid process; diseases of the circulatory system; diseases of the respiratory system; diseases of the digestive system; diseases of the skin; diseases of the musculoskeletal system or connective tissue; diseases of the genitourinary system; conditions related to sexual health; diseases related to pregnancy, childbirth or the puerperium; certain conditions originating in the perinatal period; and developmental anomalies. In some embodiments, the disease is one or more entries of the ICD-11 MMS, or the International Classification of Disease. The ICD provides a method of classifying diseases, injuries, and causes of death. The World Health Organization (WHO) publishes the ICDs to standardize the methods of recording and tracking instances of diagnosed disease. In some embodiments, the condition is a disease stimulant such as a disease precondition or comorbidity.

In some embodiments, the condition occurs in, or is measured in the context of, a cell system. In some embodiments, the condition occurs in, or is measured in the context of, one or more cells, where the one or more cells includes single cells, cell lines, biopsy sample cells, and/or cultured primary cells. In some embodiments, the condition is a physiological condition occurring in human cells. In some embodiments, the condition is a physiological condition occurring in a sample, such as any of the samples described herein (see, for example, Definitions: Samples). In some embodiments, the condition is a physiological condition occurring in a subject, such as a human or an animal. In some embodiments, the condition of interest is, or is related to, a cellular process of interest.

Block 266. Referring to block 266, in some embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

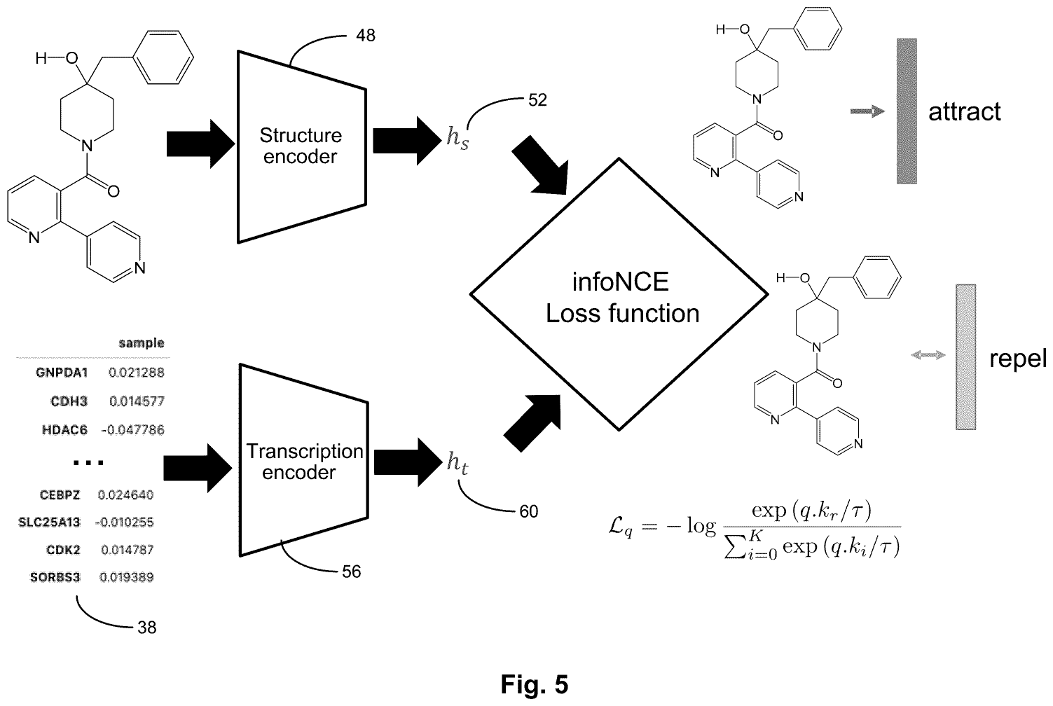

Blocks 268-272. Referring to block 268, in some embodiments, the method further comprises training the first model and the second model. Referring to block 270, in some embodiments, the training comprises contrastive learning. In some such embodiments, an Adam optimizer, a version of stochastic gradient descent used in deep learning, is used to minimize a loss score. In some embodiments the first model 48 and the second model 56 are jointly trained in batches of n≈1000 structure-differential expression pairs (S₁, T₁), . . . , (Sₙ, Tₙ). That is, each of these structure-differential expression pairs comprises the fingerprint of chemical structure of a respective chemical and a differential expression signature 38 measured for the respective chemical. For each i,j=1, . . . , n a similarity score is computed δᵢⱼ=F(E_{S}_{ᵢ},E_{T}_{ⱼ}). Referring to FIG. 5, in some such embodiments, a loss is used that maximizes the similarity scores corresponding to positive pairs (δ₁₁, . . . , δₙₙ) and minimize the negative pairs (δᵢⱼ for i≠j). More specifically, in some embodiments the InfoNCE loss function L:

$$L = - {\sum }_{i = 1}^{n} log ( \frac{exp ( τ \cdot {δ}_{ii} )}{{\sum }_{j = 1}^{n} exp ( τ \cdot {δ}_{i j} )} ) - {\sum }_{i = 1}^{n} log ( \frac{exp ( τ \cdot {δ}_{ii} )}{{\sum }_{j = 1}^{n} exp ( τ \cdot {δ}_{i j} )} )$$

is used. See Oord, 2019, “Representation Learning with Contrastive Predictive Coding,” arXiv:1807.03748v2, which is hereby incorporated by reference. In some embodiments to address any imbalance in the number of transcriptional samples per compounds in the training set (e.g., where some compounds have less than 5 transcriptional measurements while some compounds have more than 1000), every batch of size b is sampled in an Adam optimization algorithm by sampling b random compounds, for each compound, sample a single random transcriptional sample. Referring to block 272, in some embodiments, the training comprises training the first and second model jointly against a single loss function.

### III. RANK ORDERING A PLURALITY OF TEST CHEMICAL COMPOUNDS AGAINST A DIFFERENTIAL EXPRESSION SIGNATURE BETWEEN A FIRST CELL STATE AND A SECOND CELL STATE

Referring to FIG. 3, another aspect of the present disclosure provides for rank ordering a plurality of test chemical compounds against a differential expression signature between a first cell state and a second cell state.

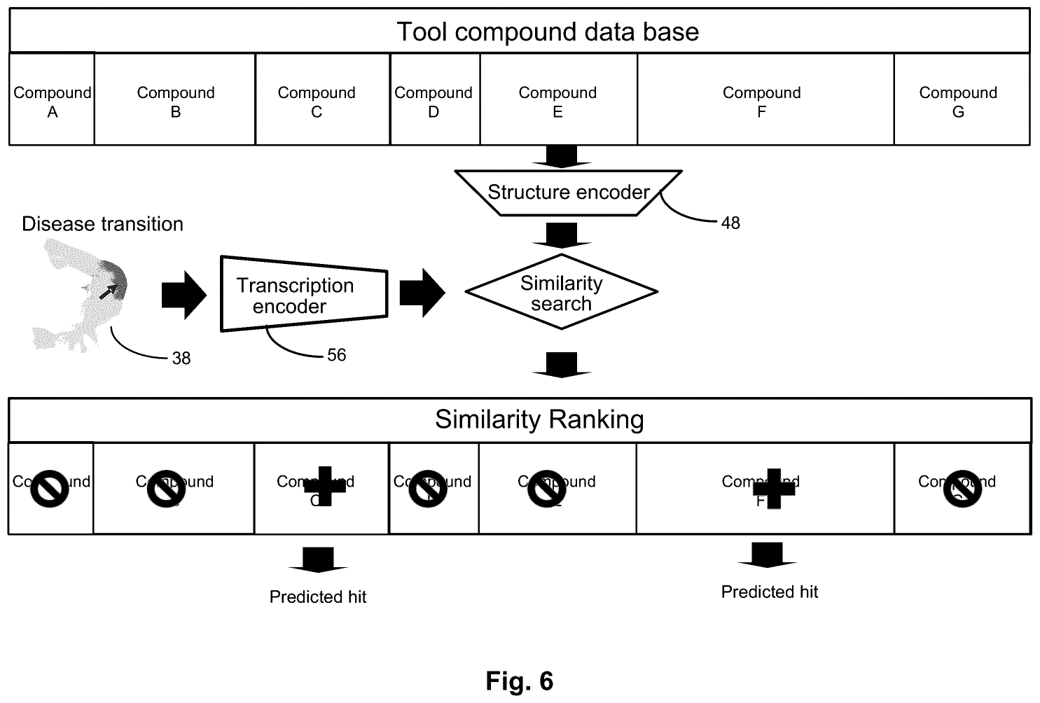

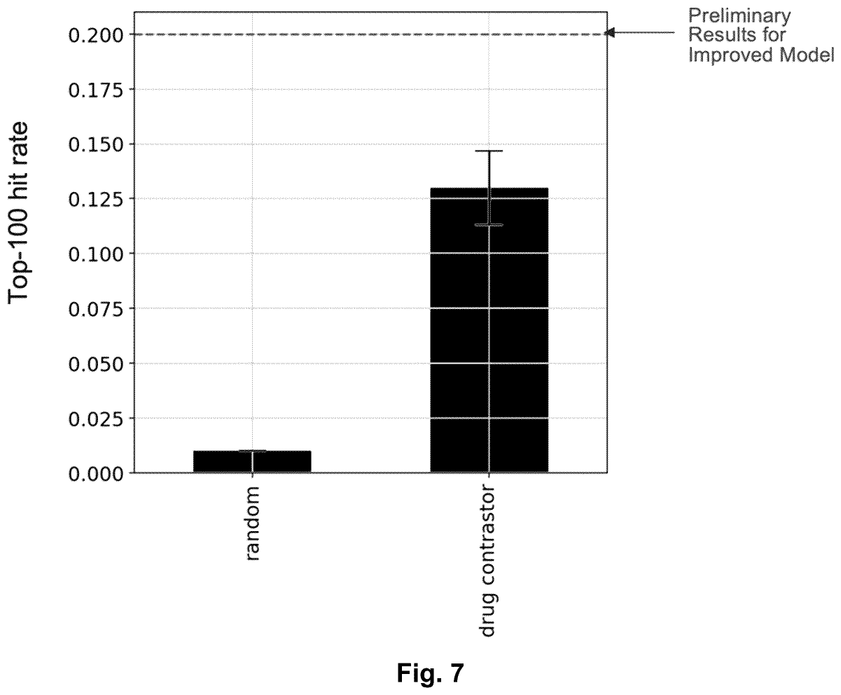

Block 300. Referring to block 300, and as further illustrated in FIG. 6, in some embodiments, a method of rank ordering a plurality of test chemical compounds against a differential expression signature between a first cell state and a second cell state is provided.

Block 302. Referring to block 302, in some embodiments, the first cell state represents a wild-type disease-free state and the second cell state represents a diseased state. More discussion on representative first and second cell states is provided above with reference to block 202.

Block 304. Referring to block 304, in some embodiments, the plurality of test chemical compounds comprises 1000, 10,000, 100,000 or one million chemical compounds. In some embodiments, the plurality of test chemical compounds includes at least 5, at least 10, at least 15, at least 20, at least 30, at least 40, at least 50, at least 100, at least 200, at least 300, at least 400, at least 500, at least 800, at least 1000, at least 2000, at least 3000, at least 4000, at least 5000, at least 8000, at least 10,000, at least 20,000, at least 30,000, at least 50,000, at least 80,000, at least 100,000, at least 200,000, at least 500,000, at least 800,000, at least 1 million, or at least 2 million test chemical compounds.

In some embodiments, the plurality of test chemical compounds includes no more than 10 million, no more than 5 million, no more than 1 million, no more than 500,000, no more than 100,000, no more than 50,000, no more than 10,000, no more than 8000, no more than 5000, no more than 2000, no more than 1000, no more than 800, no more than 500, no more than 200, or no more than 100 test chemical compounds.

Block 306. Referring to block 306, in some embodiments, a differential expression signature 38 is obtained. The differential expression signature 38 comprises a plurality of differential values 42. Each respective differential value 42 in the plurality of differential values corresponds to a respective cellular constituent in a set of cellular constituents. The respective differential value represents a difference between (i) one or more abundance values (first abundance values 44) measured for the respective cellular constituent in a first cell-based assay of a first plurality of cells that represent the first cell state and (ii) one or more abundance values (second abundance values 46) measured for the respective cellular constituent in a second cell-based assay of a second plurality of cells that represent the second cell state. More discussion on differential expression signatures 38 is provided in block 222 above.

Blocks 308-310. Referring to block 308, in some embodiments, each corresponding differential value in the plurality of differential values is a comparison of (i) a first measure of central tendency of the one or more abundance values for the respective cellular constituent across the first plurality of cells, and (ii) a second measure of central tendency of the one or more abundance values for the respective cellular constituent across the second plurality of cells. In some embodiments, the measure of central tendency is a mean, median, mode, weighted mean, weighted median, and/or weighted mode.

In some embodiments, the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells. In some embodiments, the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells. In some embodiments the first cell-based assay is a bulk cell-based assay and a single abundance value 44 is measured for each cellular constituent 40 across the first plurality of cells. In some embodiments the second cell-based assay is a bulk cell-based assay and a single abundance value 44 is measured for each cellular constituent 40 across the second plurality of cells.

In some embodiments the first cell-based assay is a single cell-based assay and a plurality of abundance values 44 are measured for each cellular constituent 40 across the first plurality of cells. Referring to block 226, in some such embodiments, the plurality of abundance values (first stated abundance values 44) measured for the respective cellular constituent 40 in the first cell-based assay are obtained by the first single-cell assay. In some such embodiments the second cell-based assay is also a single-cell assay and a plurality of abundance values (second state abundance values) 46 measured for the respective cellular constituent in the second plurality of cell-based assay are obtained by a second single-cell assay.

In some embodiments, the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells.

In some embodiments, the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells.

In some embodiments, the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells, the first cell-based assay is a first single-cell based assay and the one or more first state abundance values 44 for the first single-cell based assay comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more first state abundance values 44 for each cellular constituent 40 in a plurality of cellular constituents.

In some embodiments, the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells, the second cell-based assay is a second single-cell based assay and the one or more second state abundance values 46 for the second single-cell based assay comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more second state abundance values 46 for each cellular constituent 40 in a plurality of cellular constituents.

In some embodiments, the first and second cell-based assay is the same single-cell based assay. In some embodiments, the first and second cell-based assay is the same bulk-cell based assay.

Block 312. Referring to block 312, in some embodiments, the first single-cell assay and the second single-cell assay is ribonucleic acid (RNA) sequencing (scRNA-seq), CITE-seq, or scATAC-seq.

Block 314. Referring to block 314, in some embodiments, the one or more abundance values measured for the respective cellular constituent in the first cell-based assay or the one or more abundance values measured for the respective cellular constituent in the second cell-based assay are determined by a colorimetric measurement, a fluorescence measurement, a luminescence measurement, a resonance energy transfer (FRET) measurement, a measurement of a protein-protein interaction, a measurement of a protein-polynucleotide interaction, a measurement of a protein-small molecule interaction. mass spectrometry, nuclear magnetic resonance, or a microarray measurement.

More discussion on suitable first and second cell-based assays is provided above with reference to blocks 224-228.

Block 316. Referring to block 316, in some embodiments, each cellular constituent in the set of cellular constituents uniquely maps to a different gene. In some such embodiments, the set of cellular constituents collectively maps to 3 or more, 4 or more, 5 or more, 6, or more, 7 or more, 8 or more, 9 or more, 10 or more, 100 or more, 200 or more, 300 or more, 400 or more, 500 or more, 600 or more, 700 or more, 800 or more, 900 or more, or 1000 or more different genes. In some embodiments the set of cellular constituents collectively maps to between 1000 and 10000 different genes.

Block 318. Referring to block 318, in some embodiments, each cellular constituent in the set of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein. More discussion of suitable cellular constituents is provided above with respect to block 232.

Blocks 320-322. Referring to block 320, in some embodiments, the set of cellular constituents consists of between 100 and 1000 cellular constituents. Referring to block 322, in some embodiments, the set of cellular constituents comprises 3 cellular constituents, 4 cellular constituents, 5 cellular constituents, 6 cellular constituents, 7 cellular constituents, 8 cellular constituents, 9 cellular constituents, 10 or more cellular constituents, 20 or more cellular constituents, 30 or more cellular constituents, 40 or more cellular constituents, or 50 or more cellular constituents. More suitable ranges for the set of cellular constituents is provide above in blocks 234-236.

Block 324. Referring to block 324, in some embodiments, the first plurality of cells and the second plurality of cells are cells from an organ, cells from a tissue, a plurality of stem cells, a plurality of primary human cells, cells from umbilical cord blood, cells from peripheral blood, bone marrow cells, cells from a solid tissue, or a plurality of differentiated cells. More discussion of suitable cells for the first plurality of cells and the second plurality of cells is give in block 238 above.

Block 325. Referring to block 325 of FIG. 3C, in some embodiments, for each respective test chemical compound in the plurality of test chemical compounds, a respective fingerprint of the respective test chemical compound is inputted into a first model, thereby retrieving, as output from the first model, a corresponding chemical embedding, thereby obtaining a plurality of chemical embeddings, each respective chemical embedding corresponding to a respective test chemical compound in the plurality of test chemical compounds.

Block 326. Referring to block 326, in some embodiments, the respective fingerprint of the respective test chemical compound is calculated from a chemical structure of the respective test chemical compound using a simplified molecular-input line-entry system (SMILES) string representation of the test chemical compound. More discussion of SMILES of string representations of test chemical compounds is provide in block 214 above.

Block 328. Referring to block 328, in some embodiments, the respective fingerprint of the respective test chemical compound is calculated from a chemical structure of the respective test chemical compound using Daylight, BCI, ECFP4, EcFC, MDL, APFP, TTFP, UNITY 2D fingerprint, RNNS2S, or GraphConv. Move discussion of representative calculations of fingerprints is provided in block 216 above.

Block 330. Referring to block 330, in some embodiments, the fingerprint of the test chemical compound is calculated as a plurality of features that comprise a plurality of bioactivity descriptors for the test chemical compound. In some embodiments the plurality of bioactivity descriptors include a numeric representation of the test chemical compound obtained from a two-dimensional fingerprint of the test chemical compound, a mechanism of action of the test chemical compound, a small molecule role possessed by the test chemical compound, a therapeutic area associated with the test chemical compound, a three-dimensional fingerprint of the test chemical compound, an association of the test chemical compound with one or more metabolic genes, an association of the test chemical compound with a small molecule pathway, an association of the test chemical compound with a cancer cell line, a crystal structure of the test chemical compound, a signaling pathway associated with the test chemical compound, a therapeutic side effect associated with the test chemical compound, a structural key associated with the test chemical compound, a binding affinity of the test chemical compound against a macromolecular target, a biological process associated with the test chemical compound, a morphology of cells exposed to the test chemical compound, a disease associated with the test chemical compound, a toxicology associated with the test chemical compound, a physicochemistry associated with the test chemical compound, a drug-drug interaction associated with the test chemical compound, an inhibitory constant associated with the test chemical compound, a binding interaction of the test chemical compound with one or more residues of a protein, a Gibbs free energy of the binding of the test chemical compound with a protein, or any combination thereof.

Block 332. Referring to block 332, in some embodiments, use a model that predicts bioactivity descriptors to determine one or more of the plurality of bioactivity descriptors. More discuss of such bioactivity descriptors is provided in block 220 above.

Blocks 334-336. Referring to block 334, in some embodiments, the respective fingerprint of the respective test chemical compound comprises 100 features. Referring to block 336, in some embodiments, the respective fingerprint of the respective test chemical compound consists of between 10 features and 1000 features. In some embodiments, the respective fingerprint of the respective test chemical compound comprises 10 or more, 50 or more 100 or more, 500 or more, 1000 or more, 2000 or more, 3000 or more, or 5000 or more features.

Block 338. Referring to block 338, in some embodiments, the respective test chemical compound is a first organic compound having a molecular weight of less than 2000 Daltons. More disclosure on the mass of respective test chemicals is provided in block 206 above.

Block 340. Referring to block 340, in some embodiments, the respective test chemical compound satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, a compound of the present disclosure satisfies one or more criteria in addition to Lipinski's Rule of Five. For example, in some embodiments, a compound of the present disclosure has five or fewer aromatic rings, four or fewer aromatic rings, three or fewer aromatic rings, or two or fewer aromatic rings. More disclosure on the characteristics of respective test chemicals is provided in block 208 above.

Block 342-346. Referring to block 342, in some embodiments, the method further comprises training the first model and the second model. Referring to block 344, in some embodiments, the training comprises contrastive learning. Referring to block 346, in some embodiments, the training comprises training the first and second model jointly against a single loss function. More details on suitable model training in accordance with some embodiments of the present disclosure is provided above with respect to blocks 268-272.

Blocks 348-350. Referring to block 348, in some embodiments, the first model comprises 1000 parameters and the second model comprises 1000 parameters. Referring to block 350, in some embodiments, the first model consists of between 10 and 10 million parameters and the second model consists of between 10 and 10 million parameters. In some embodiments, the first model comprises 1000, 2000, 5000, 10,000, 100,000 or 1 million parameters. In some embodiments, the second model comprises 1000, 2000, 5000, 10,000, 100,000 or 1 million parameters. More suitable numbers of parameters for the first and second model are provided in blocks 248-250 above.

Block 352. Referring to block 352, in some embodiments, responsive to inputting the differential expression signature into a second model, retrieve, as output from the second model, a differential expression embedding. In some embodiments the first model 48 is a multilayer perceptron (fully connected neural network) containing dropout layers, batch normalization, and a ReLU non-linearity. In some embodiments model 48 produces an chemical embedding E_{S} 52 of the structure S of the test chemical compound.

Block 354. Referring to block 354, in some embodiments, the first model is a first multilayer perceptron and the second model is a second multilayer perceptron. In some such embodiments the first model 48 and/or the second model 56 is a fully connected second neural network, also known as a multilayer perceptron (MLP). In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. More disclosure on suitable MLPs that serve as the first model 48 and/or the second model 56 in some embodiments of the present disclosure is found in Vang-mata ed., 2020, Multilayer Perceptrons: Theory and Applications, Nova Science Publishers, Hauppauge, New York, which is hereby incorporated by reference.

Block 356. Referring to block 356, in some embodiments, rank each respective test chemical compound in the plurality of test chemical compounds based on a respective similarity between the respective chemical embedding corresponding to the respective test chemical compound and the differential expression embedding.

Block 358. Referring to block 358, in some embodiments, the respective similarity between the respective chemical embedding and the differential expression embedding is determined by a distance between the respective chemical embedding and the differential expression embedding. In some such embodiments the distance is a cosine distance, Euclidian distance, Manhattan distance, Jaccard distance, correlation distance, Chi-square distance, or Mahalanobis distance between the chemical embedding 52 and the differential expression embedding 60. More details on suitable metrics for determining similarity are provided in block 254 above.

Block 360. Referring to block 360, in some embodiments, the differential expression signature is associated with alleviating a condition in a subject, and the method further comprises administering the respective test chemical compound to the subject as a treatment to alleviate the condition in the subject when the respective test chemical compound is found to have a threshold ranking in the plurality of test chemical compounds or a threshold similarity.

Block 326. Referring to block 362, in some embodiments, the treatment comprises a composition comprising the respective test chemical compound and one or more excipient and/or one or more pharmaceutically acceptable carrier and/or one or more diluent. More disclosure on suitable excipients, pharmaceutically acceptable carrier, and diluent is provided in block 260 above.

Block 364-366. Referring to block 364, in some embodiments, the condition is inflammation or pain. Referring to block 366, in some embodiments, the condition is a disease. Non-limiting examples of suitable conditions and diseases is provided above in blocks 262-264.

Block 368. Referring to block 368, in some embodiments, the condition is a cancer, hematologic disorder, autoimmune disease, inflammatory disease, immunological disorder, metabolic disorder, neurological disorder, genetic disorder, psychiatric disorder, gastroenterological disorder, renal disorder, cardiovascular disorder, dermatological disorder, respiratory disorder, viral infection, or other disease or disorder.

### IV. ADDITIONAL COMPUTER SYSTEM EMBODIMENTS

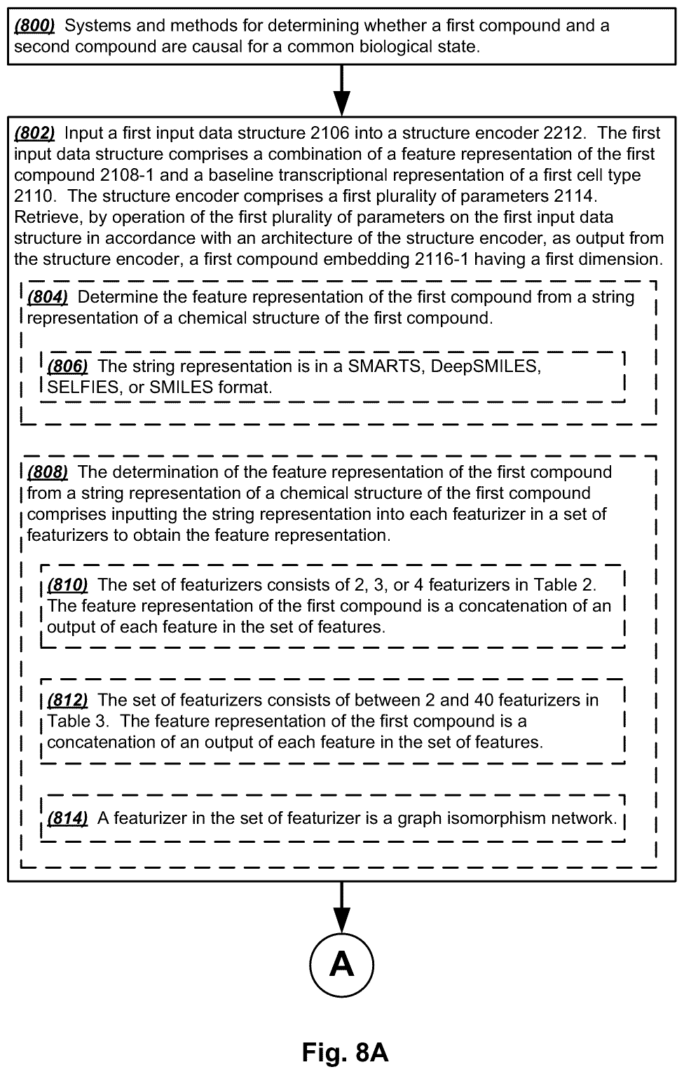

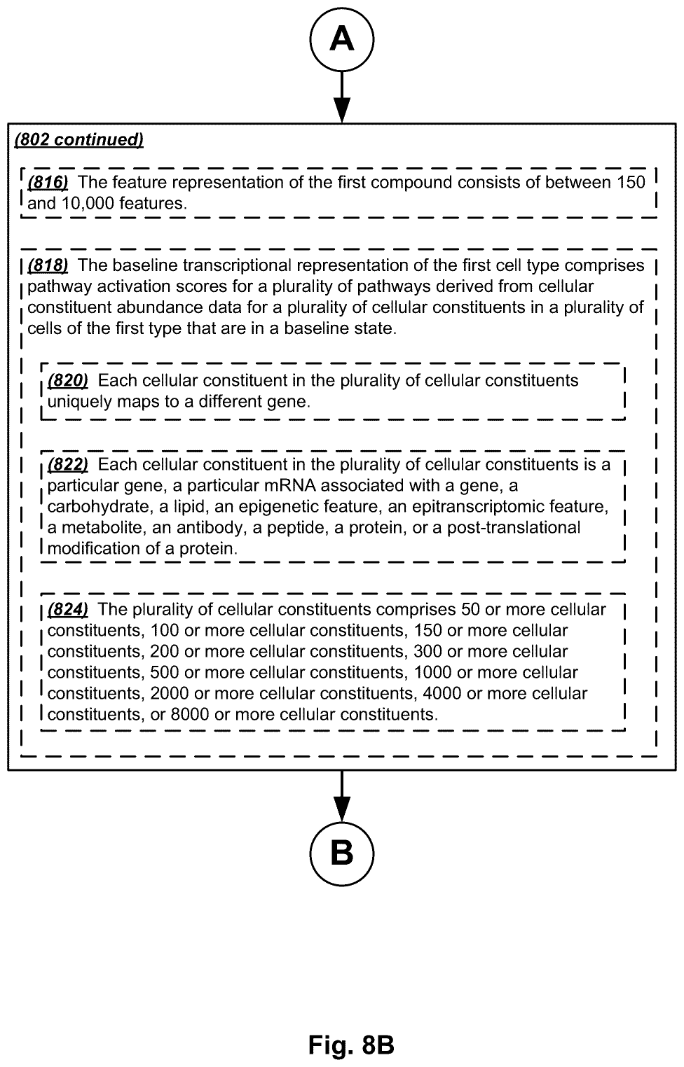

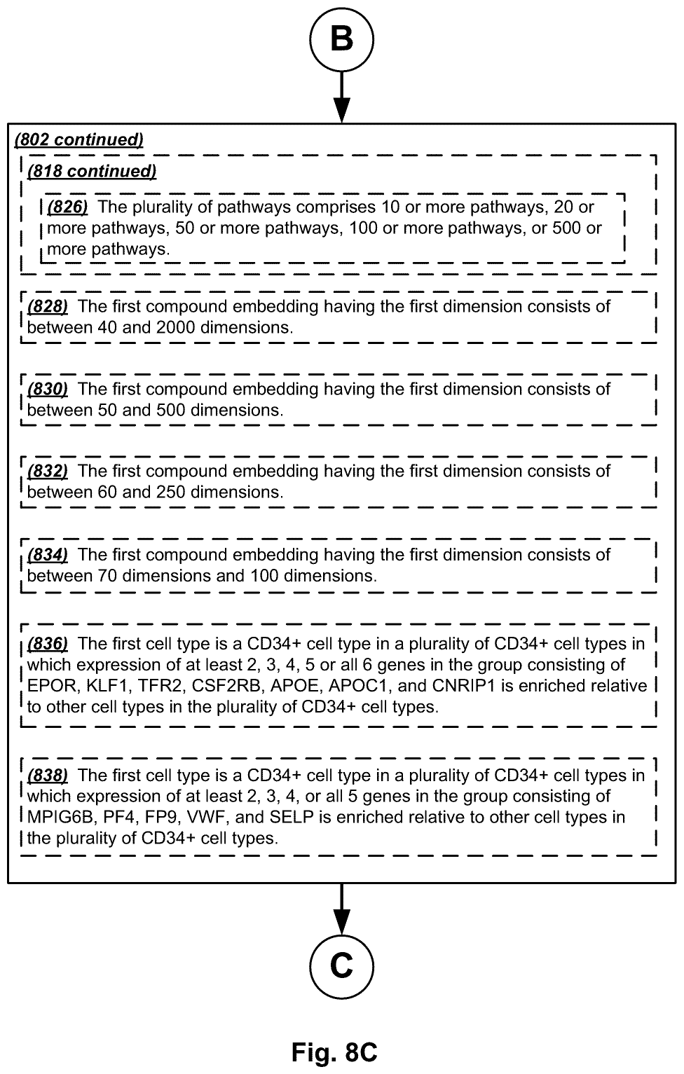

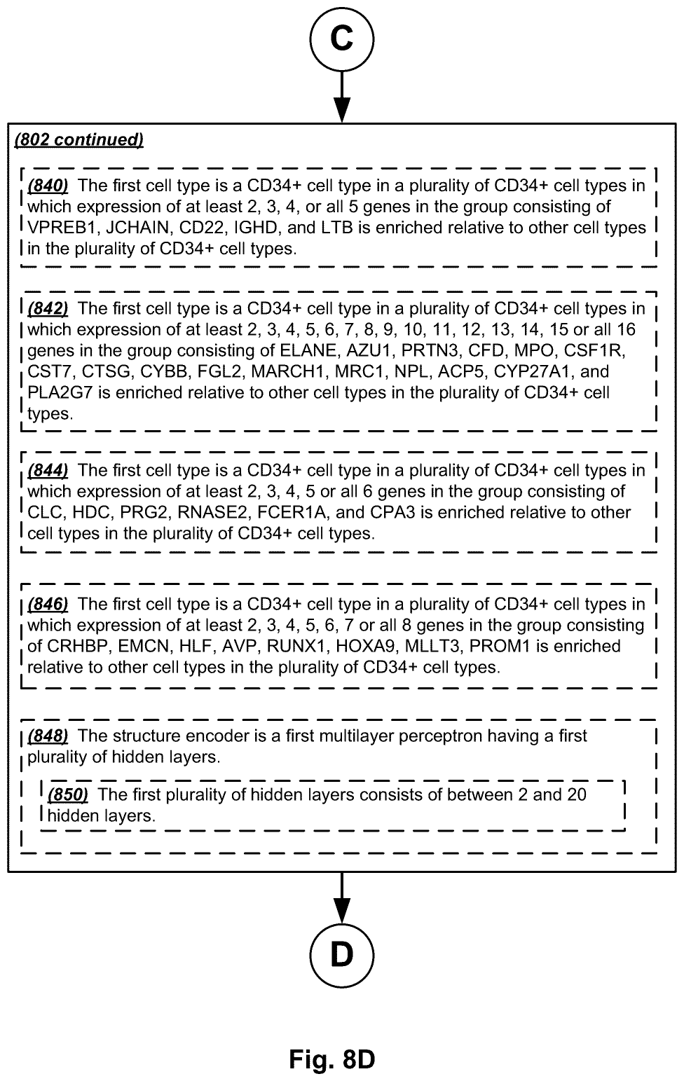

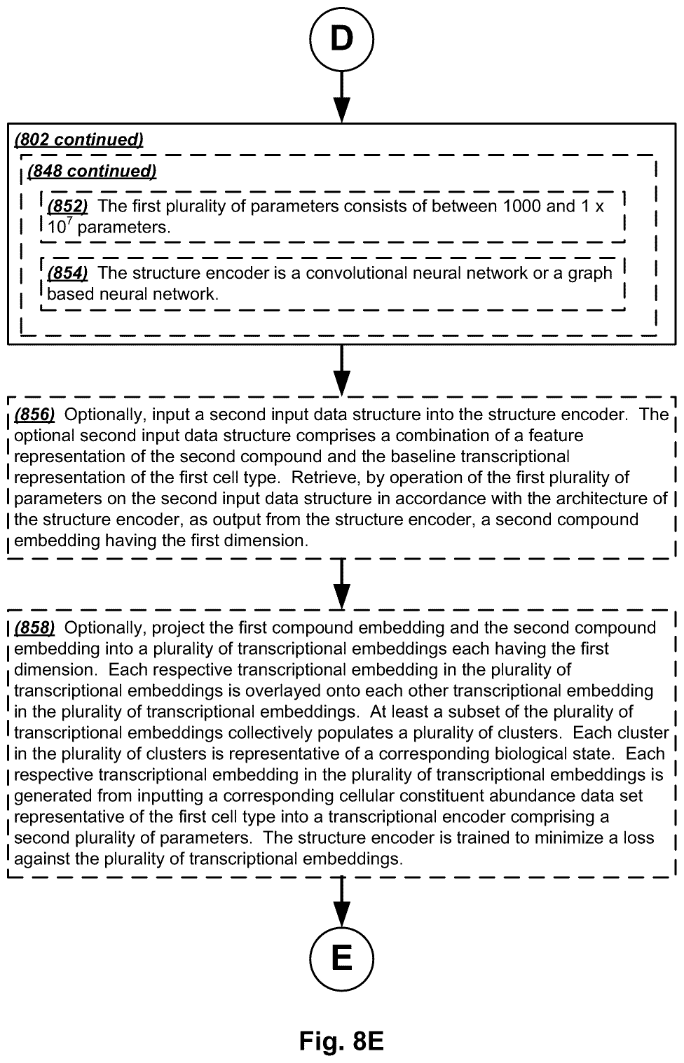

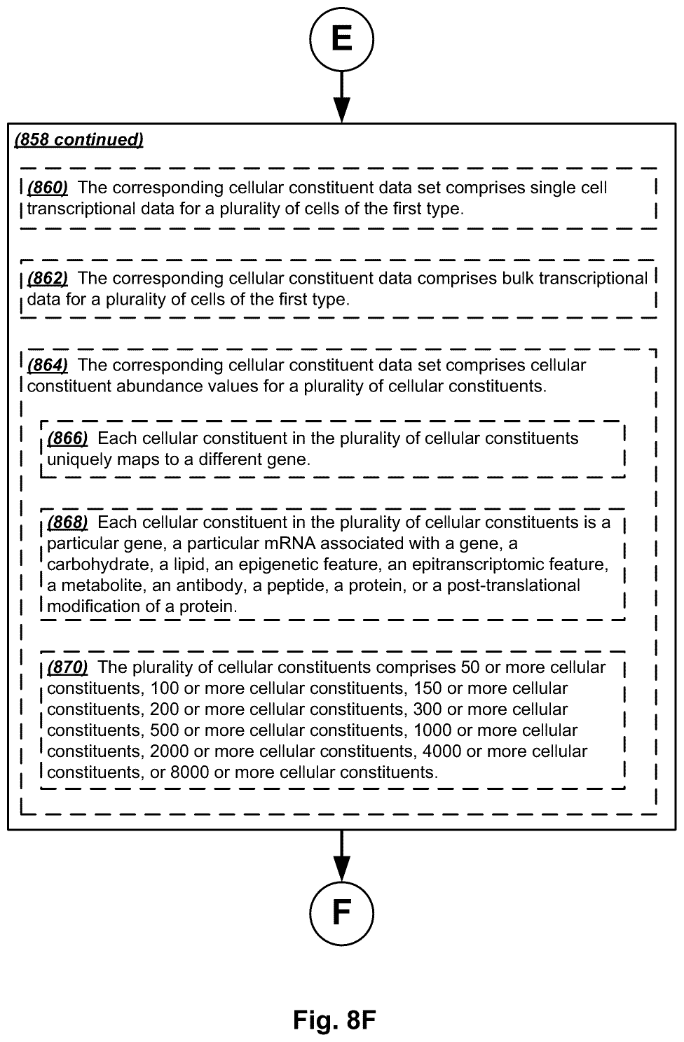

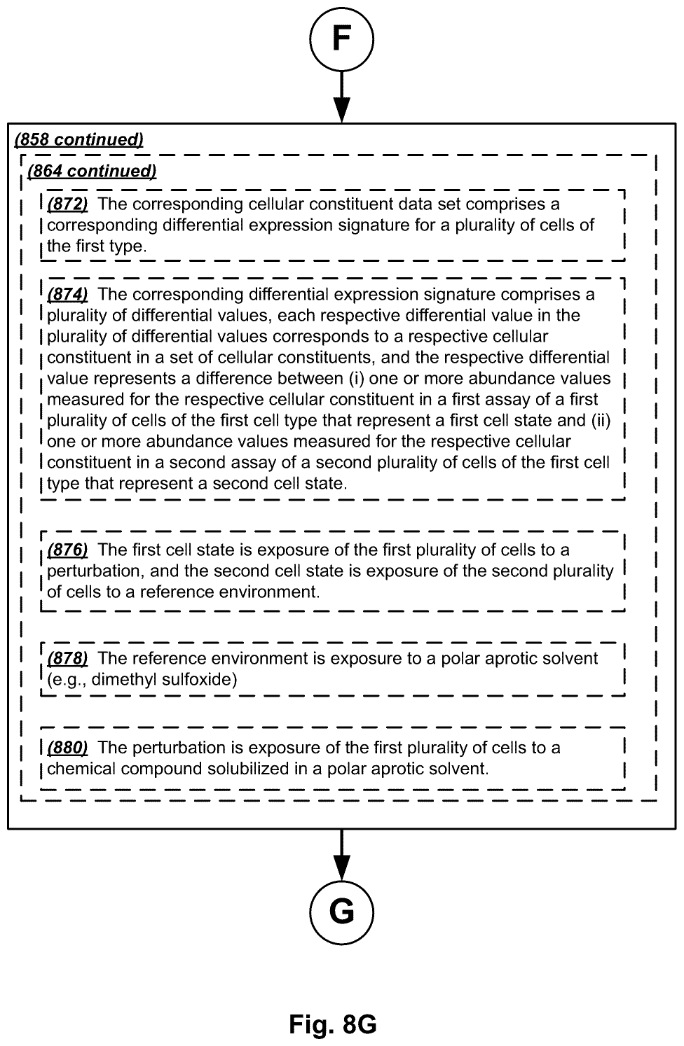

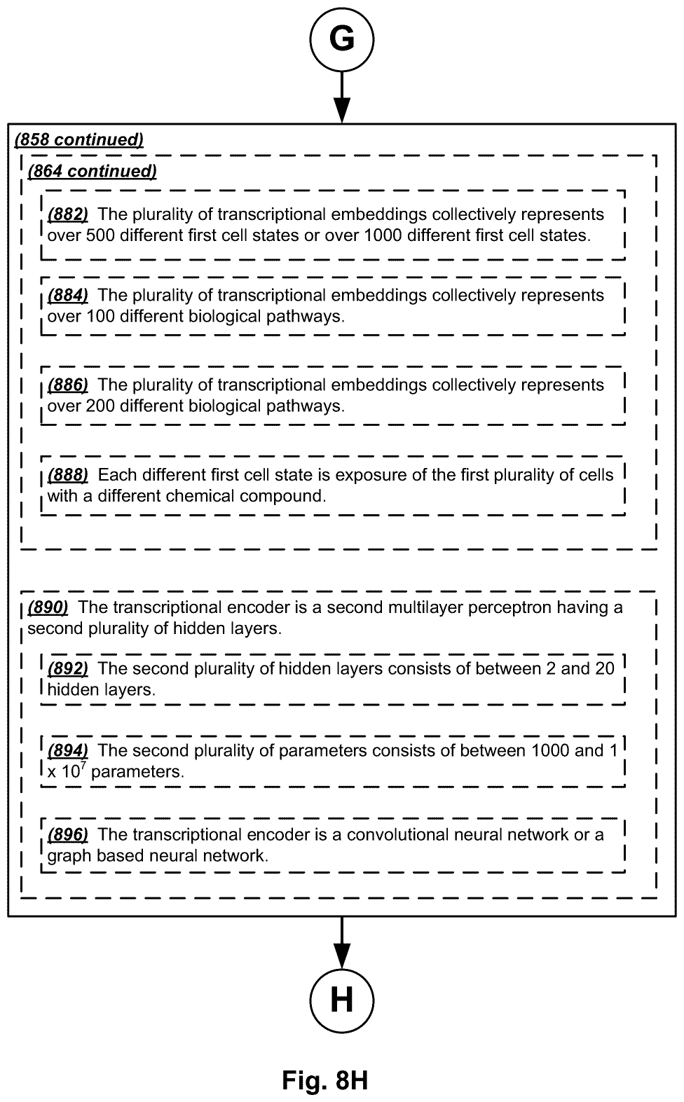

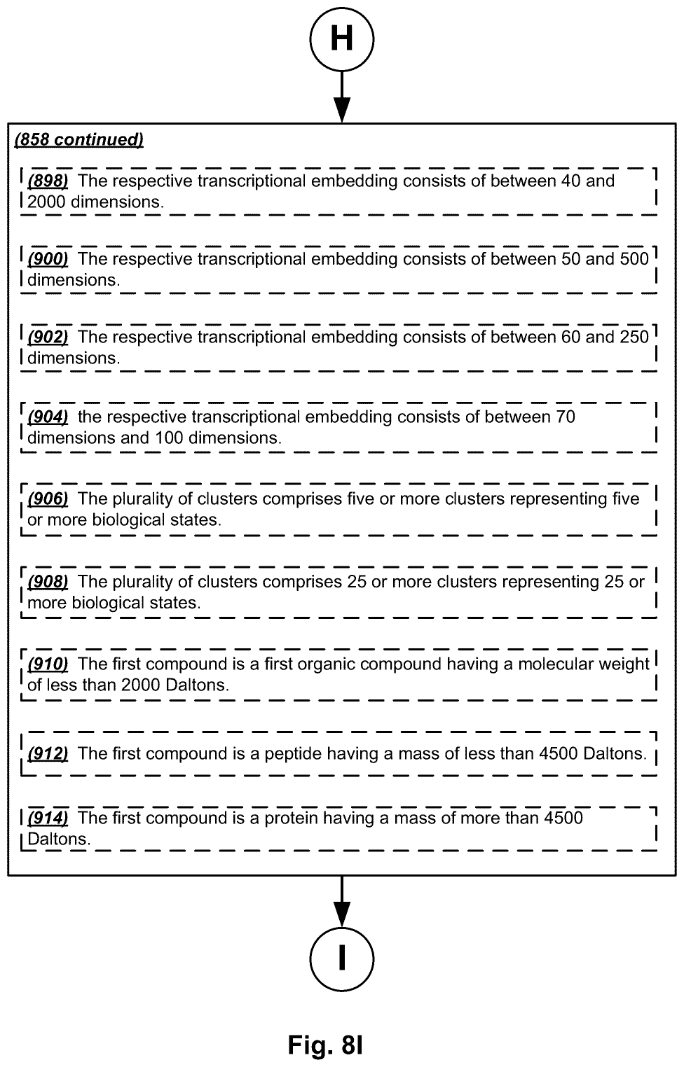

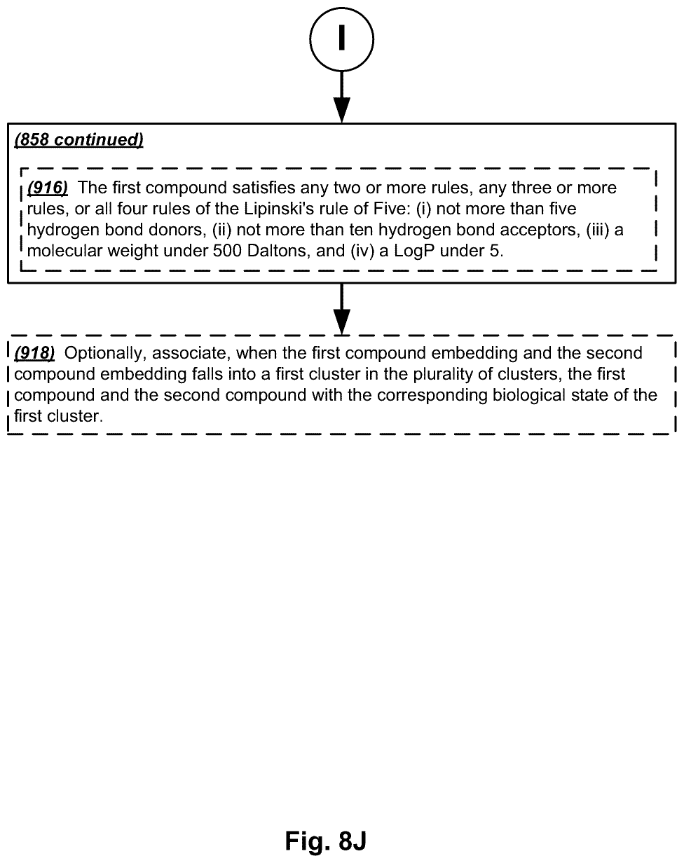

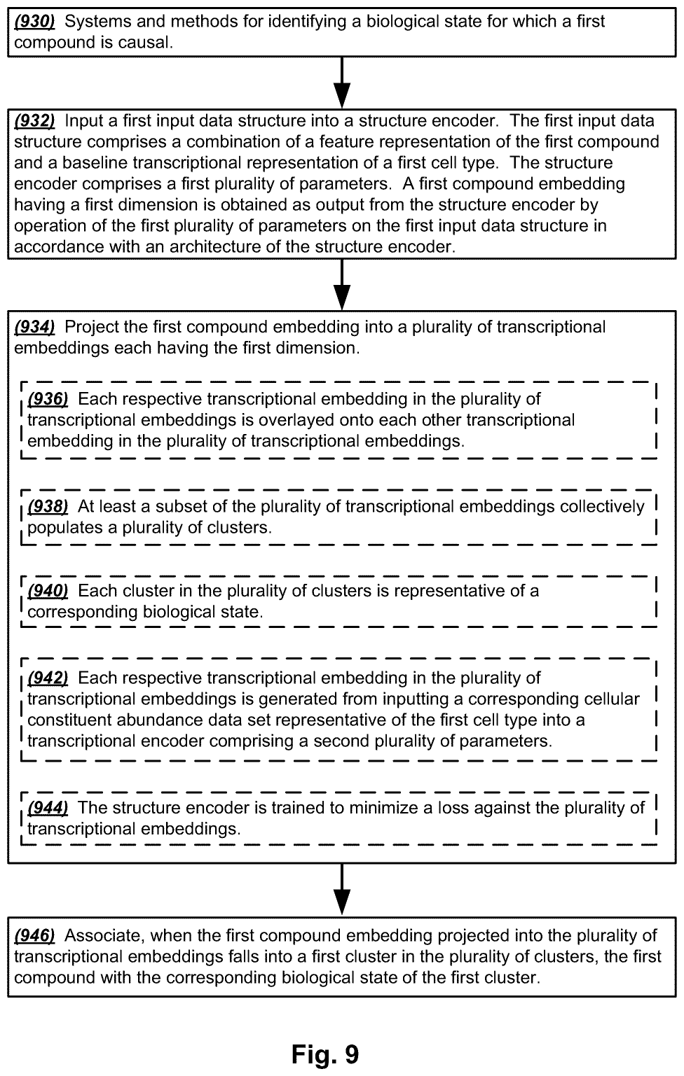

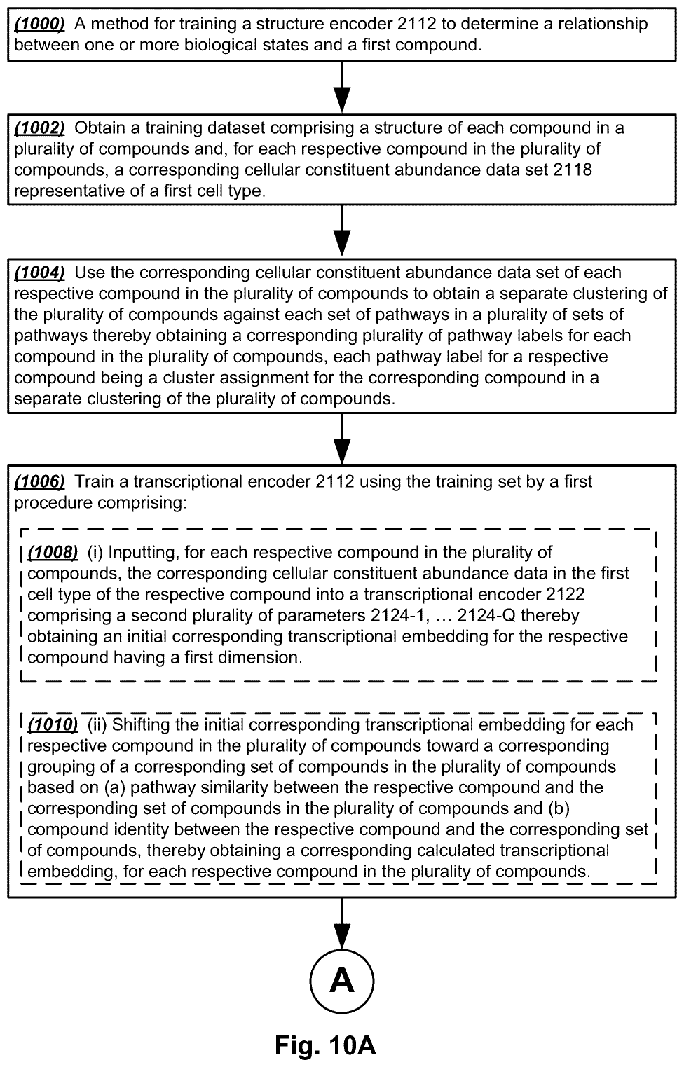

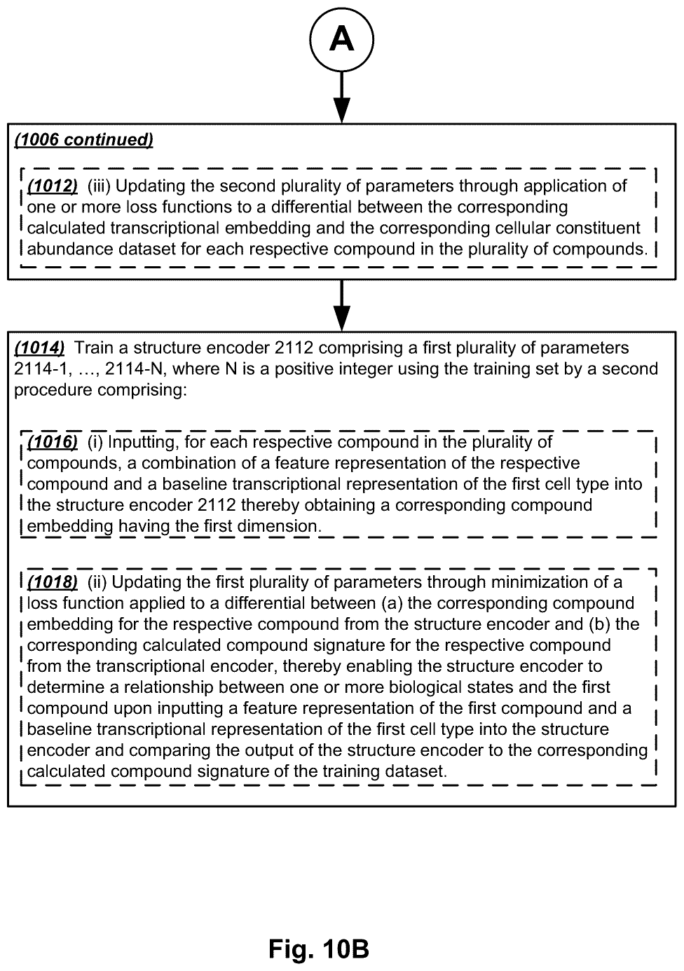

FIG. 10 provides a block diagram illustrating a system 2100 in accordance with some embodiments of the present disclosure. The system 2100 performs, for example, the methods disclosed in FIGS. 8A, 8B, 8C, 8D, 8E, 8F, 8G, 8H, 8I, and 8J (determining whether a first compound and a second compound are causal for a common biological state), FIG. 9 (identifying a biological state for which a first compound is causal.) and/or FIGS. 10A and 10B (training a structure encoder to determine a relationship between one or more biological states and a first compound).

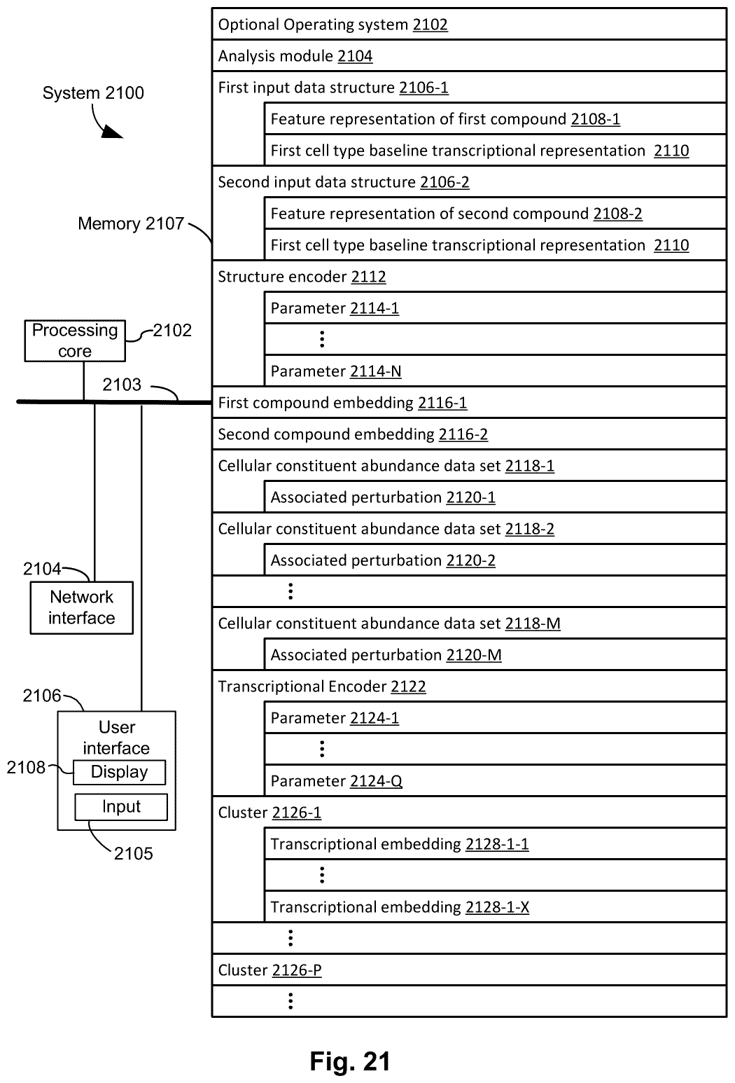

In FIG. 21, the system 2100 is illustrated as a computing device. Other topologies of the computer system 2100 are possible. For instance, in some embodiments, the system 2100 can in fact constitute several computer systems that are linked together in a network, or be a virtual machine or a container in a cloud computing environment. As such, the exemplary topology shown in FIG. 1 merely serves to describe the features of an embodiment of the present disclosure in a manner that will be readily understood to one of skill in the art.

Referring to FIG. 21, in some embodiments the computer system 2100 (e.g., a computing device) includes a network interface 2104. In some embodiments, the network interface 2104 interconnects the system 2100 computing devices within the system with each other, as well as optional external systems and devices, through one or more communication networks. In some embodiments, the network interface 2104 optionally provides communication via the Internet, one or more local area networks (LANs), one or more wide area networks (WANs), other types of networks, or a combination of such networks.

Examples of networks include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

The system 2100 in some embodiments includes one or more processing units (CPU(s)) 2102 (e.g., a processor, a processing core, etc.), one or more network interfaces 2104, a user interface 2106 including (optionally) a display 2108 and an input system 2105 (e.g., an input/output interface, a keyboard, a mouse, etc.) for use by a user, memory 2107, and one or more communication buses 2103 for interconnecting the aforementioned components. The one or more communication buses 2103 optionally include circuitry (sometimes called a chipset) that interconnects and controls communications between system components. The memory 2107 typically includes high-speed random access memory, such as DRAM, SRAM, DDR RAM, ROM, EEPROM, flash memory, CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The memory 2107 optionally includes one or more storage devices remotely located from the CPU(s) 2102. In some embodiments, the memory includes non-transitory computer readable storage medium. In some embodiments, the memory 2107 stores the following programs, modules and data structures, or a subset thereof:
an optional operating system 2102 (e.g., ANDROID, IOS, DARWIN, RTXC, LINUX, UNIX, OS X, WINDOWS, or an embedded operating system such as VxWorks), which includes procedures for handling various basic system services and for performing hardware dependent tasks;
an analysis module 2104 for performing any of the computational methods described in the present disclosure (e.g., those described in FIGS. 8, 9, and 10);
a first input data structure 2106-1 for a first compound comprising a feature representation 2108-1 of the first compound and a first call type baseline transcriptional representation 2110;
a second input data structure 2106-2 for a second compound comprising a feature representation 2108-2 of the second compound and the first call type baseline transcriptional representation 2110;
a structure encoder 2112, comprising a first plurality of parameters 2114-1, . . . , 2114-N, where N is a positive integer, for creating a compound embedding having a first dimension (e.g., first compound embedding 2116-1, second compound embedding 2116-2, etc.);
a plurality of cellular constituent abundance datasets 2118-1, 2118-2, . . . , 2118-M, each associated with a corresponding associated perturbation 2120-1, 2120-2, . . . , 21120-M, wherein M is a positive integer; and
a transcriptional encoder 2122, comprising a second plurality of parameters 2124-1, . . . , 2124-Q, where N is a positive integer, for creating transcriptional embeddings having the first dimension (e.g., transcriptional embedding 2128-1-1, . . . , 2128-1-X, etc.) from corresponding cellular constituent abundance data sets, where such transcriptional embedding is then clustered in clusters 2126-1, . . . , 2126-P, where P is a positive integer, based on similarity between the transcriptional embeddings.

In various embodiments, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some embodiments, the memory stores additional modules and data structures not described above. In some embodiments, one or more of the above identified elements is stored in a computer system, other than that of the system 2100, that is addressable by the system 2100 so that the system 2100 may retrieve all or a portion of such data when needed.

Although FIG. 21 depicts a “system 2100,” the figure is intended more as a functional description of the various features that may be present in computer systems than as a structural schematic of the implementations described herein. In practice, and as recognized by those of ordinary skill in the art, items shown separately could be combined and some items could be separated. Moreover, although FIG. 21 depicts certain data and modules in memory 2107, some or all of these data and modules instead may be stored in more than one memory such as at a remote storage device that can be a part of a cloud-based infrastructure.

While a system in accordance with the present disclosure has been disclosed with reference to FIG. 21, methods in accordance with the present disclosure are now detailed with reference to FIGS. 8, 9 and 10.

### V. DETERMINING WHETHER A FIRST COMPOUND AND A SECOND COMPOUND ARE CAUSAL FOR A COMMON BIOLOGICAL STATE

Referring to block 800 and FIG. 8, in some embodiments, systems and methods for determining whether a first compound and a second compound are causal for a common biological state in a first cell type are provided.

The elements of FIG. 8 can also be used to perform a method of determining whether a first compound and a second compound are causal for a common biological state that comprises inputting a first input data structure into a structure encoder, where the first input data structure comprises a combination of a feature representation of the first compound and a baseline transcriptional representation and the structure encoder comprises a first plurality of parameters. In this way, there is retrieved, by operation of the first plurality of parameters on the first input data structure in accordance with an architecture of the structure encoder, as output from the structure encoder, a first compound embedding having a first dimension. Then, a determination of respective similarity between the first compound embedding and each respective transcriptional embedding in a plurality of transcriptional embeddings is made, thereby determining a plurality of similarities. In embodiments, each transcriptional embedding in the plurality of transcriptional embeddings has the first dimension. In embodiments, each transcriptional embedding in the plurality of transcriptional embeddings is generated from inputting a corresponding cellular constituent abundance data set representative of the first cell type exposed to a different perturban (e.g., compounds) in a plurality of perturbans, into a transcriptional encoder comprising a second plurality of parameters. In embodiments, the plurality of perturbans includes the second compound. In embodiments, the plurality of transcriptional embeddings comprises at least 25 transcriptional embeddings. In embodiments the structure encoder is trained to minimize a loss against the plurality of transcriptional embeddings. In embodiments, the first compound is associated with a biological state that the second compound is known to be causal for when the embedding comparison determines that the similarity between the first compound embedding and the respective transcriptional embedding of the second compound satisfies a similarity criterion (e.g., being among the top embedding comparisons in terms of similarity).

Referring to block 802 of FIG. 8A, in some embodiments, a first input data structure 2106 is inputted into a structure encoder 2112. The first input data structure 2106 comprises a combination of a feature representation of the first compound 2108 and a baseline transcriptional representation 2110 of the first cell type. The structure encoder 2112 comprises a first plurality of parameters 2114-1, . . . , 2114-N, where N is a positive number. There is retrieved, by operation of the first plurality of parameters 2114 on the first input data structure 2106 in accordance with an architecture of the structure encoder 2112, as output from the structure encoder, a first compound embedding 2116-1 having a first dimension.

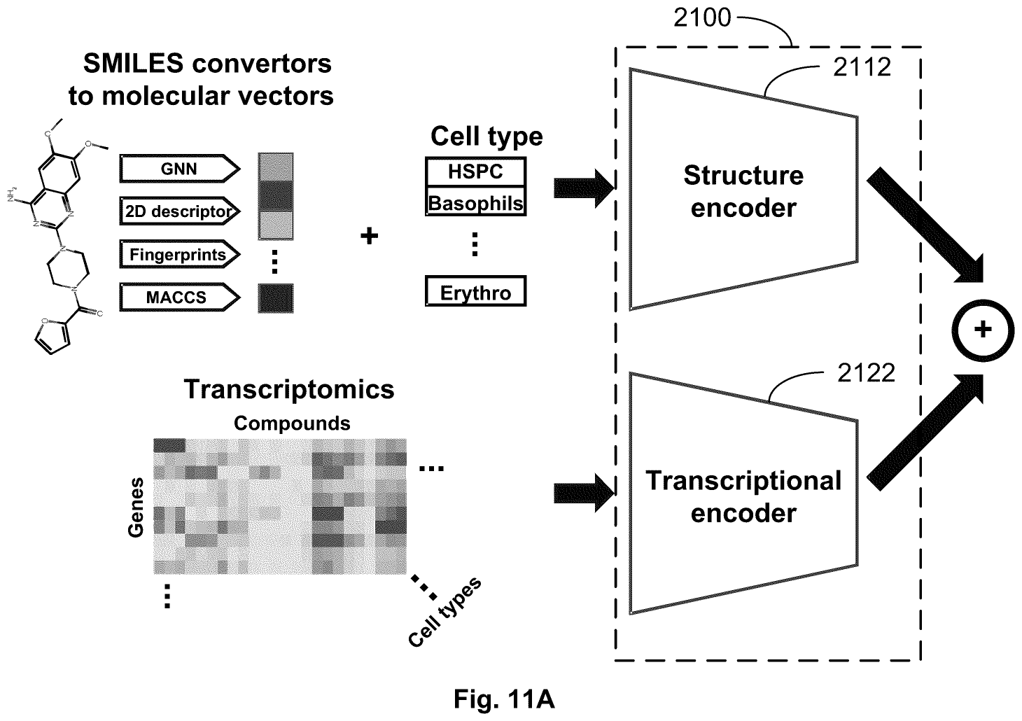

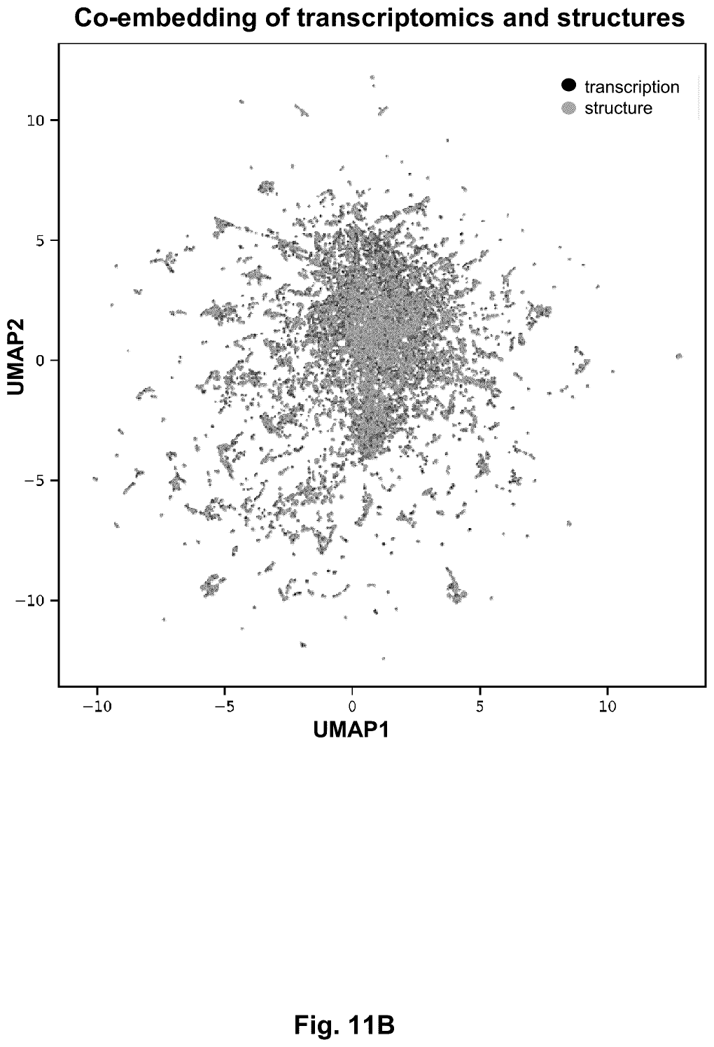

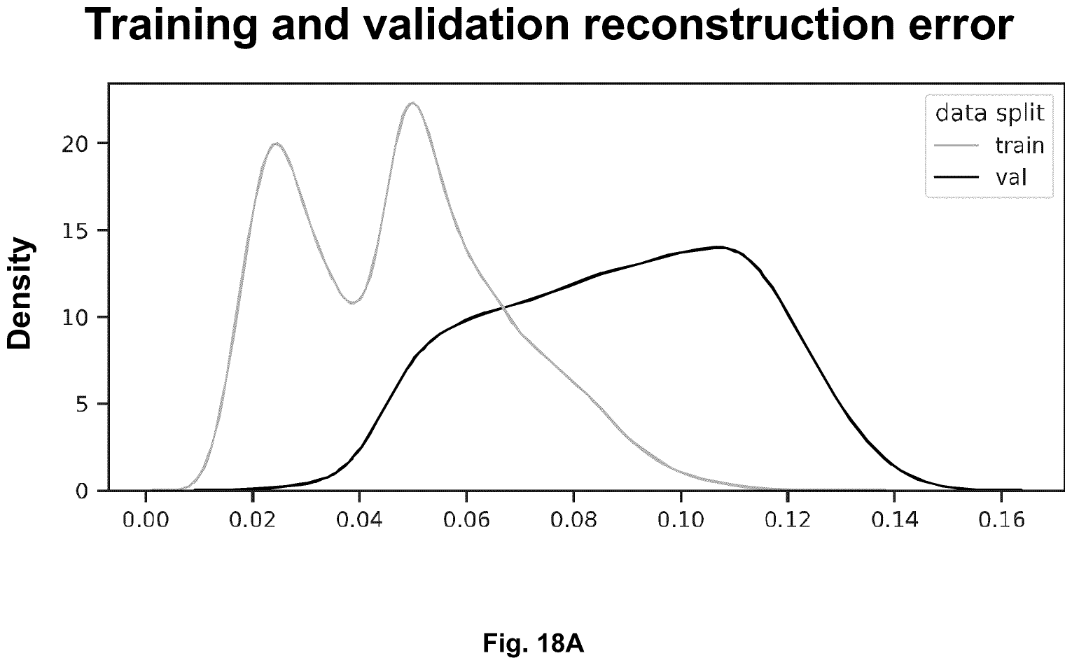

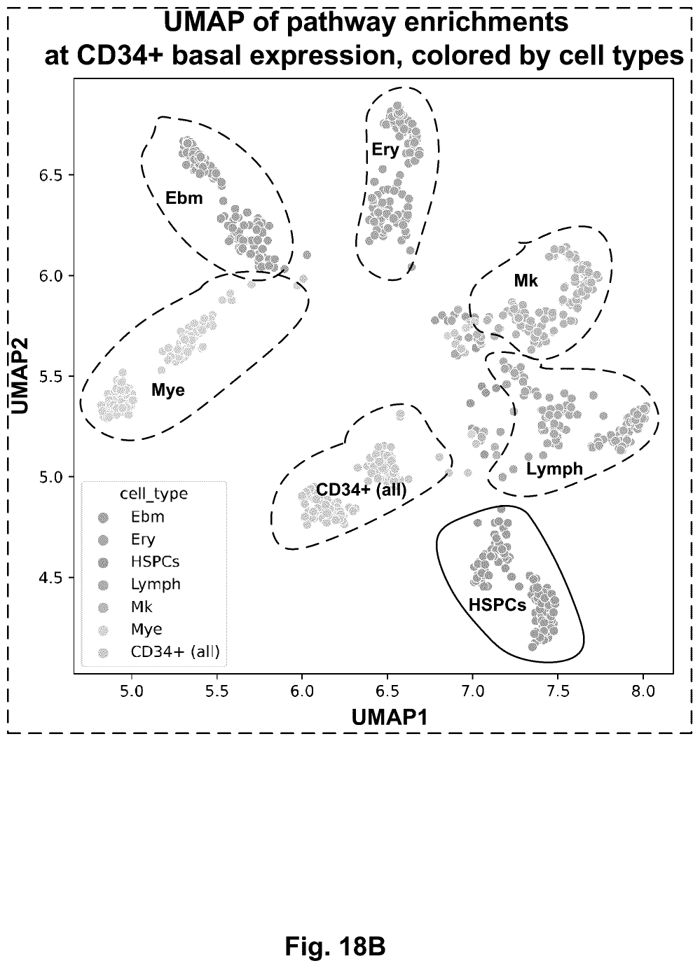

Referring to FIG. 11A, the structure encoder 2112 is part of a model that also includes a transcriptional encoder 2122. The structure encoder 2112 and the transcriptional encoder to generate a co-embedding. In some embodiments, the transcriptional encoder 2122 is first trained to project high-dimensional transcriptomics into a latent space of the first dimension (e.g., 96 dimensions in one example). The parameters of the transcriptional encoder 2122 are then frozen and the structure encoder 2122 is trained so that the projection of compound embeddings into the same latent space has the minimum L1 reconstruction loss compared to the fixed transcriptional embedding as illustrated in FIG. 18A. In some embodiments, the compound embeddings of 100 or more, 200 or more, or 300 or more different compounds are used to perform this training. Effectively this results in a multimodal co-embedding that can generate embeddings both from transcriptional and structural data.

In embodiments, the model of the present disclosure is a multi-modal model. In embodiments, the model of the present disclosure can receive input of new data modalities to augment models and predictions. In embodiments, data modalities comprise genomics, transcriptomics, epigenomics, proteomics, and multi-omics data modalities. In embodiments, the data modality comprises one or more of scDNA-seq, scRNA-seq, DNA methylation, histone modification, chromatic accessibility, protein expression, DNA methylation data and transcriptomic data, transcriptome and chromatin accessibility. In embodiments, the data modality comprises DOP-PCR, MDA, MALBAC, Full-length transcript: scNaUmi-seq, MATQ-seq, Smart-seq, Smart-seq2 3′ transcript: 10× Chromium, CEL-seq2, Drop-seq, InDrop, MARS-seq 5′ transcript: STRT-seq, scBS-seq, ChIP-seq, ATAC-seq, DNase-seq, Hi-C, CyTOF, FACS, scM&T-seq, scMT-seq, scTrio-seq, and snmCT-seq, Paired-seq and SNARE-seq.

In embodiments, data modalities comprise imaging/cell painting data, proteomic data, atacseq, and siteseq.

Referring to block 804, in some embodiments, the feature representation of the first compound 2108-1 is determined from a string representation of a chemical structure of the first compound. Referring to block 806, in some embodiments, the string representation is in a SMARTS (SMARTS—A Language for Describing Molecular Patterns,” 2022 on the Internet at daylight.com/dayhtml/doc/theory/theory.smarts.html (accessed December 2020), DeepSMILES (O'Boyle and Dalke, 2018, “DeepSMILES: an adaptation of SMILES for use in machine-learning of chemical structures,” Preprint at ChemRxiv. https://doi.org/10.26434/chemrxiv.7097960.v1.), self-referencing embedded string (SELFIES) (Krenn et al., 2022, “SELFIES and the future of molecular string representations,” Patterns 3(10), pp. 1-27), or simplified molecular-input line-entry system (SMILES) format. Molecular fingerprinting using SMILES strings is described, for example, in Honda et al., 2019, “SMILES Transformer: Pre-trained Molecular Fingerprint for Low Data Drug Discovery,” arXiv: 1911.04738, which is hereby incorporated herein by reference in its entirety.

Referring to block 808, in some embodiments, the determination of the feature representation of the first compound 2108-1 from a string representation of a chemical structure of the first compound comprises inputting the string representation into each featurizer in a set of featurizers to obtain the feature representation. Referring to block 810, in some embodiments, the set of featurizers consists of 2, 3, or 4 featurizers in Table 2.

TABLE 2
Example featurizers

| | No. of | |
|---|---|---|
| Featurizer name | features | Internet Reference, last accessed Dec. 6, 2023 |
| gin_supervised_edgepred | 300 | molfeat.datamol.io/featurizers/gin_supervised_edgepred |
| ECFP:4 | 2000 | molfeat.datamol.io/featurizers/ecfp |
| Desc2d | 211 | molfeat.datamol.io/featurizers/desc2D |
| MACCS | 167 | molfeat.datamol.io/featurizers/maccs |

In some embodiments, the feature representation of the first compound 2108-1 is a concatenation of an output of each feature in the set of features in such embodiments. For instance, in an embodiment in which all four featurizers of Table 1 are used, the feature representation of the first compound 2108-1 consists of 300+2000+211+167 or 2678 features.

Referring to block 812, in some embodiments, the set of featurizers consists of between 2 and 40 featurizers in Table 3. In some embodiments, the feature representation of the first compound 2108-1 is a concatenation of an output of each feature in the set of featurizers.

TABLE 3
Additional example featurizers

| Featurizer name | Internet Reference, last accessed Dec. 6, 2023 |
|---|---|
| Roberta-Zin480M-102M | molfeat.datamol.io/featurizers/Roberta-Zinc480M-102M |
| GPT2-Zinc480M-87M | molfeat.datamol.io/featurizers/GPT2-Zinc480M-87M |
| ChemGPT-1.2B | molfeat.datamol.io/featurizers/ChemGPT-1.2B |
| ChemGPT-19M | molfeat.datamol.io/featurizers/ChemGPT-19M |
| ChemGPT-4.7M | molfeat.datamol.io/featurizers/ChemGPT-4.7M |
| MolT5 | olfeat.datamol.io/featurizers/MolT5 |
| Desc3D | molfeat.datamol.io/featurizers/tags/physchem |
| Desc2d | molfeat.datamol.io/featurizers/desc2D |
| mordred | molfeat.datamol.io/featurizers/mordred |
| scaffoldkeys | molfeat.datamol.io/featurizers/scaffoldkeys |
| electroshape | molfeat.datamol.io/featurizers/electroshape |
| usrcat | molfeat.datamol.io/featurizers/usrcat |
| usr | molfeat.datamol.io/featurizers/usr |
| cats3d | molfeat.datamol.io/featurizers/cats3d |
| cats2d | molfeat.datamol.io/featurizers/cats2d |
| Pharm3D-cats | molfeat.datamol.io/featurizers/pharm3D-cats |
| Pharm2D-cats | molfeat.datamol.io/featurizers/pharm2D-cats |
| pharm2D-default | molfeat.datamol.io/featurizers/pharm2D-default |
| pharm3D-gobbi | molfeat.datamol.io/featurizers/pharm3D-gobbi |
| pharm3D-pmapper | molfeat.datamol.io/featurizers/pharm3D-pmapper |
| Pharm2D-pmapper | molfeat.datamol.io/featurizers/pharm2D-pmapper |
| ChemBERTa-77M-MTR | molfeat.datamol.io/featurizers/ChemBERTa-77M-MTR |
| ChemBERTa-77M-MLM | molfeat.datamol.io/featurizers/ChemBERTa-77M-MLM |
| atompair-count | molfeat.datamol.io/featurizers/atompair-count |
| topological-count | molfeat.datamol.io/featurizers/topological-count |
| fcfp-count | molfeat.datamol.io/featurizers/fcfp-count |
| ecfp-count | molfeat.datamol.io/featurizers/ecfp-count |
| estate | molfeat.datamol.io/featurizers/estate |
| Extended Reduced Graph | molfeat.datamol.io/featurizers/erg |
| approach (ErG) | |
| SMILES extended | molfeat.datamol.io/featurizers/secfp |
| connectivity fingerprint | |
| (SECFP) | |
| MinHashed atom-pair | molfeat.datamol.io/featurizers/map4 |
| fingerprint up to a diameter | |
| of four bonds (MAP4) | |
| pattern | molfeat.datamol.io/featurizers/pattern |
| rdkit | molfeat.datamol.io/featurizers/rdkit |
| topological | molfeat.datamol.io/featurizers/topological |
| Functional-class | molfeat.datamol.io/featurizers/fcfp |
| fingerprints (FCFPs) | |
| Extended-connectivity | molfeat.datamol.io/featurizers/ecfp |
| fingerprints (ECFPs) | |
| avalon | molfeat.datamol.io/featurizers/avalon |
| gin_supervised_masking | molfeat.datamol.io/featurizers/gin_supervised_masking |
| gin_supervised_infomax | molfeat.datamol.io/featurizers/gin_supervised_infomax |
| gin_supervised_edgepred | molfeat.datamol.io/featurizers/gin_supervised_edgepred |
| jtvae_zinc_no_kl | molfeat.datamol.io/featurizers/jtvae_zinc_no_kl |
| pcqm4mv2_graphormer_base | molfeat.datamol.io/featurizers/pcqm4mv2_graphormer_base |
| gin_supervised_contextpred | molfeat.datamol.io/featurizers/gin_supervised_contextpred |
| MACCS | molfeat.datamol.io/featurizers/maccs |

Referring to block 814, in some embodiments, a featurizer in the set of featurizers makes use of a deep graph convolutional neural network (e.g., Zhang et al, “An End-to-End Deep Learning Architecture for Graph Classification,” The Thirty-Second AAAI Conference on Artificial Intelligence), GraphSage (e.g., Hamilton et al., 2017, “Inductive Representation Learning on Large Graphs,” arXiv: 1706.02216 [cs.SI]), a graph isomorphism network (e.g., Hu et al., 2018, “How Powerful are Graph Neural Networks,” cs>arXiv:1810.00826, an edge-conditioned convolutional neural network (ECC) (e.g., Simonovsky and Komodakis, 2017, “Dynamic Edge-Conditioned Filters in Convolutional Neural Networks on Graphs,” arXiv:1704.02901 [cs.CV]), a differentiable graph encoder such as DiffPool (e.g., Ying et al., 2018, “Hierarchical Graph Representation Learning with Differentiable Pooling” arXiv: 1806.08804 [cs.LG]), a message-passing graph neural network such as MPNN (Gilmer et al., 2017, “Neural Message Passing for Quantum Chemistry,” arXiv: 1704.01212 [cs.LG]) or D-MPNN (Yang et al., 2019, “Analyzing Learned Molecular Representations for Property Prediction” J. Chem. Inf. Model. 59(8), pp. 3370-3388), or a graph neural network such as CMPNN (Song et al., “Communicative Representation Learning on Attributed Molecular Graphs,” Proceedings of the Twenty-Ninth International Joint Conference on Artificial Intelligence (IJCAI-20)). See also Rao et al., 2021, “MolRep: A Deep Representation Learning Library for Molecular Property Prediction,” doi.org/10.1101/2021.01.13.426489; posted Jan. 16, 2021. T; Rao et al., “Quantitative Evaluation of Explainable Graph Neural Networks for Molecular Property Prediction,” arXiv preprint arXiv:2107.04119; and github.com/biomed-AI/MolRep.

Referring to block 816, in some embodiments, the feature representation of the first compound consists of between 150 and 10,000 features. In some embodiments the feature representation consists of between 50 and 25,000 features, between 100 and 20,000 features, between 500 and 25,000 features, or between 1000 and 5000 features. In some embodiments the feature representation comprises at least 100, 200, 400, 800, 1000, 1500, 2000, 2500, 3000, 3500, 4000, or 5000 features.

Referring to block 818, in some embodiments, the baseline transcriptional representation of the first cell type 2110 comprises pathway activation scores for a plurality of pathways derived from cellular constituent abundance data for a plurality of cellular constituents in a plurality of cells of the first type that are in a baseline state. In some embodiments the pathways that are used to form pathway scores are KEGG pathways and so there is a pathway score for at least 100, at least 200, or each pathway in the KEGG database. In some embodiments the pathways that are used to form pathway scores are Reactome pathways and so there is a pathway score for at least 100, at least 200, or each pathway in the Reactome database. In some embodiments both KEGG and Reactome pathways are used. In some embodiments other or additional pathways are used to form pathway scores.

In some embodiments cellular constituent values are used instead of pathway activation scores. However, the use of pathway scores in some embodiments of the present disclosure rather than cellular constituent abundance values advantageously alleviates batch effects in some instances. Cellular constituent abundance values (e.g., gene expression abundance values) may reflect batch effects such a plate-specific variations (batch effects). Thus, in some embodiments the baseline expression in the form of pathway activation scores is used in accordance with block 818. This aggregation of cellular constituents into pathway activation scores removes the effect of individual cellular constituents (e.g., genes) and aids in overcoming batch effect in some embodiments.

In the following example the KEGG pathway library is used to calculate pathway activation scores in a novel advantageous manner. To calculate the activation score of each pathway from baseline expression, each sample of a format (cell type, plate) from DMSO (control) wells was used. The cellular constituents (e.g, genes) were arranged in descending order of expression and a random-walk-like algorithm was executed.

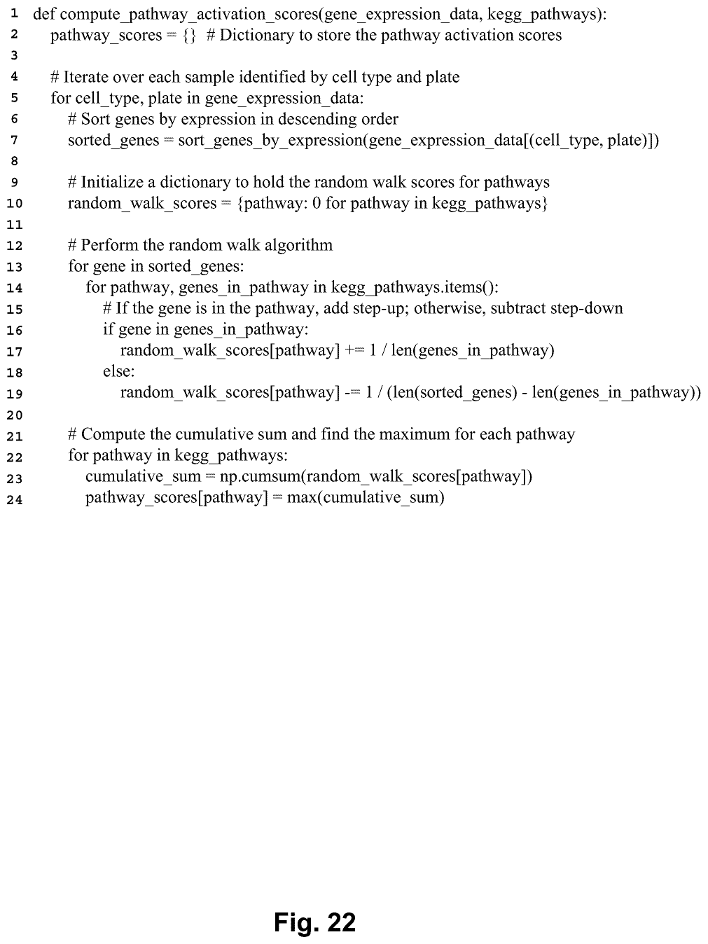

Referring to FIG. 22, in the random-walk-like algorithm, for each pathway, process control begins, as detailed in lines 1-13 of the pseudocode, with the most abundant cellular constituent (e.g., most expressed gene) in the pathway descending to the least abundant cellular constituent (e.g., least expressed gene) in the pathway. At each step (line 14 of the pseudocode), either a step-up p is added (equal to 1/(number of cellular constituents in a pathway)) if the current cellular constituent belongs to a pathway (lines 16-17 of the pseudocode) or a step-down q is subtracted (equal to 1/(number of all cellular constituents—number of cellular constituents in a pathway)) (lines 18-19 of the pseudocode) otherwise. Subsequently, the cumulative sum across the ranked cellular constituents is determined and the peak value is identified (lines 21-24), which represents the pathway activation score at the baseline level for a given cell type in a given sample (e.g., plate well). This allows for the representation of each cellular constituent baseline expression, not as raw expression values, but as pathway scores for a given cell type in a given sample. As FIG. 18B illustrates, computation of silhouette scores using the plate index as labels or cell type (subcluster of CD34+) as labels, as expected, produced strong clustering by cell type with the pathway activation scores rather than by clustering by plate. Clustering the pathway activation scores by plate would be expected if batch effects have not been removed.

Referring to block 820, in some embodiments, each cellular constituent in the plurality of cellular constituents uniquely maps to a different gene. In some such embodiments, the plurality of cellular constituents collectively maps to 3 or more, 4 or more, 5 or more, 6 or more, 7 or more, 8 or more, 9 or more, 10 or more, 100 or more, 200 or more, 300 or more, 400 or more, 500 or more, 600 or more, 700 or more, 800 or more, 900 or more, or 1000 or more different genes. In some embodiments the plurality of cellular constituents collectively maps to between 1000 and 20000 different genes.

Referring to block 822, in some embodiments, each cellular constituent in the plurality of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein.

In some embodiments, a cellular constituent is a gene, a gene product (e.g., an mRNA and/or a protein), a carbohydrate, a lipid, an epigenetic feature, an epitranscriptomic feature, a metabolite, and/or a combination thereof. In some embodiments, each cellular constituent in the plurality of cellular constituents is a particular gene, a particular mRNA associated with a gene, a carbohydrate, a lipid, an epigenetic feature, an epitranscriptomic feature, a metabolite, a protein, or a combination thereof.

In some embodiments, the plurality of cellular constituents includes nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc.) proteins, lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular constituents such as oxidized and reduced forms of nicotinamide adenine dinucleotide (NADP/NADPH), and any combinations thereof.

Referring to block 824, in some embodiments, the plurality of cellular constituents comprises 50 or more cellular constituents, 100 or more cellular constituents, 150 or more cellular constituents, 200 or more cellular constituents, 300 or more cellular constituents, 500 or more cellular constituents, 1000 or more cellular constituents, 2000 or more cellular constituents, 4000 or more cellular constituents, or 8000 or more cellular constituents.

In some embodiments, the plurality of cellular constituents consists of between 100 and 20000 cellular constituents. In some embodiments, the plurality of cellular constituents comprises at least 500, at least 1000, at least 1500, at least 2000, at least 2500, at least 3000, at least 4000, at least 5000, at least 6000, at least 7000, at least 8000, at least 9000, at least 10000, at least 12000, at least 13000, at least 14000, at least 15000, at least 16000, at least 17000, at least 18000, at least 19000, at least 20000, at least 21000, at least 22000, at least 23000, at least 25000, at least 26000, or at least 27000 cellular constituents. In some embodiments, the plurality of cellular constituents comprises no more than 70,000, no more than 50,000, no more than 30,000, no more than 10,000, no more than 5000, no more than 1000, no more than 500, no more than 200, no more than 100, no more than 90, no more than 80, no more than 70, no more than 60, no more than 50, or no more than 40 cellular constituents. In some embodiments, the plurality of cellular constituents consists of between twenty and 25,000 cellular constituents. In some embodiments, the plurality of cellular constituents consists of between 1000 and 20,000 cellular constituents. In some embodiments, the plurality of cellular constituents falls within another range starting no lower than 5 cellular constituents and ending no higher than 70,000 cellular constituents.

As an example, in some embodiments, the plurality of cellular constituents comprises a plurality of genes, optionally measured at the RNA level. In some embodiments, the plurality of genes comprises at least 500, at least 1000, at least 1500, at least 2000, at least 2500, at least 3000, at least 4000, at least 5000, at least 6000, at least 7000, at least 8000, at least 9000, at least 10000, at least 12000, at least 13000, at least 14000, at least 15000, at least 16000, at least 17000, at least 18000, at least 19000, or at least 20000 genes. In some embodiments, the plurality of genes consists of from 500 to 2000, from 2000 to 5000, from 5000 to 10000, from 10000 to 20000, from 20000 to 50000 genes.

As another example, in some embodiments, the plurality of cellular constituents comprises a plurality of proteins. In some embodiments, the plurality of proteins comprises at least 5, at least 10, at least 15, at least 20, at least 25, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 5000, or at least 10000 proteins. In some embodiments, the plurality of proteins comprises at least 1000, at least 2000, at least 3000, at least 4000, at least 5000, at least 10,000, at least 30,000, at least 50,000, or more than 50,000 proteins. In some embodiments, the plurality of proteins comprises from 5 to 20, from 20 to 50, from 50 to 100, from 100 to 200, from 200 to 500, from 500 to 1000, from 1000 to 5000, from 5000 to 10,000, or from 10,000 to 50,000 proteins.

Referring to block 826, in some embodiments, the plurality of pathways comprises 10 or more pathways, 20 or more pathways, 50 or more pathways, 100 or more pathways, or 500 or more pathways.

Referring to blocks 828-834 in some embodiments, the first compound embedding 2116-1 having the first dimension consists of between 40 and 2000 dimensions, between 50 and 500 dimensions, between 60 and 250 dimensions, or between 70 dimensions and 100 dimensions. In one particular example the first compound embedding 2116-1 has 96 dimensions.

Referring to blocks 836 through 846, in some embodiments the cells used are CD34+ cells and a determination is made for each cell what specific CD34+ cell type the cell is from among the possible CD34+ cell types listed in Table 4. In Table 4 each cell type is a different column and the genes that define the cell type are provided in the column. In some embodiments, to determine which of the cell types a given cell is, a separate score is determined for each of the possible cell types using the expression values measured for the genes of that cell type. So for instance, for a given cell, a score is determined for column 1 of Table 4 (Ery) using the expression values of EPRO, KLF1, TFR2, CSF2RB, APOE, APOC1, and CNRIP1. Then a score is determined for column 2 of Table 4 using MP1G6b, PF4, GP9, VWF and SELP. Likewise, scores ae determined for column 3 (Lymph), 4 (My3), 5 (Ebm), and 6 (HSPC) of Table 4. The cell is then assigned to the cell type among columns 1 through 6 for which it received the highest score.

TABLE 4
example CD34+ cell types

| Ery | Mk | Lymph | Mye | Ebm | HSPC |
|---|---|---|---|---|---|
| EPOR, | MPIG6B, | VPREB1, | ELANE, | CLC, | CRHBP, |
| KLF1, | PF4, | JCHAIN, | AZU1, | HDC, | EMCN, |
| TFR2, | GP9, | CD22, | PRTN3, | PRG2, | HLF, |
| CSF2RB, | VWF, | IGHD, | CFD, | RNASE2, | AVP, |
| APOE, | SELP | LTB | MPO, | FCER1A, | RUNX1, |
| APOC1, | | | CSF1R, | CPA3 | HOXA9, |
| CNRIP1 | | | CST7, | | MLLT3, |
| | | | CTSG, | | PROM1 |
| | | | CYBB, | | |
| | | | FGL2, | | |
| | | | MARCH1, | | |
| | | | MRC1, | | |
| | | | NPL, | | |
| | | | ACP5, | | |
| | | | CYP27A1, | | |
| | | | PLA2G7 | | |

In some embodiments the ‘score_genes’ function in scanpy is used to calculate the scores for each of the cell types described above. Thus, when computing the score for column 1 of Table 4 for a given cell, the scanpy “gene_list” is EPOR, KLF1, TFR2, CSF2RB, APOE, APOC1, CNRIP1 and the gene_pool is all genes measured. The ‘score_genes’ function in scanpy is an implementation of a scoring function described in Satija et al., 2015, “Spatial reconstruction of single-cell gene expression data,” Nature Biotechnology 33(5), pp. 495-502, which is hereby incorporated by reference.

Referring to block 836, in some embodiments, the first cell type is a CD34+ cell type in a plurality of CD34+ cell types in which expression of at least 2, 3, 4, 5 or all 6 genes in the group consisting of EPOR, KLF1, TFR2, CSF2RB, APOE, APOC1, and CNRIP1 is enriched relative to other cell types in the plurality of CD34+ cell types. In some such embodiments this is determined by using at least 2, 3, 4, 5 or all 6 genes in the group consisting of EPOR, KLF1, TFR2, CSF2RB, APOE, APOC1, and CNRIP1 to determine a first score and comparing this first score to scores used to calculate other candidate cell types in the plurality of CD34+ cell types and determining that the expression of these genes is enriched relative to other cell types in the plurality of CD34+ cell types when the first score is greater than the other scores (described in blocks 836 through 846). In some embodiments the score_genes' function in scanpy is used to calculate the first score, where the scanpy “gene_list” is at least 2, 3, 4, 5 or all 6 genes in the group consisting of EPOR, KLF1, TFR2, CSF2RB, APOE, APOC1, CNRIP1 and the gene_pool is all genes measured.

Referring to block 838, in some embodiments, the first cell type is a CD34+ cell type in a plurality of CD34+ cell types in which expression of at least 2, 3, 4, or all 5 genes in the group consisting of MPIG6B, PF4, FP9, VWF, and SELP is enriched relative to other cell types in the plurality of CD34+ cell types. In some such embodiments this is determined by using at least 2, 3, 4, or all 5 genes in the group consisting of MPIG6B, PF4, FP9, VWF, and SELP to determine a second score and comparing this second score to scores used to calculate other candidate cell types in the plurality of CD34+ cell types and determining that the expression of these genes is enriched relative to other cell types in the plurality of CD34+ cell types when the second score is greater than the other scores (described in blocks 836 through 846). In some embodiments the ‘score_genes’ function in scanpy is used to calculate the second score, where the scanpy “gene_list” is at least 2, 3, 4, or all 5 genes in the group consisting of MPIG6B, PF4, FP9, VWF, and SELP and the gene_pool is all genes measured.

Referring to block 840, in some embodiments, the first cell type is a CD34+ cell type in a plurality of CD34+ cell types in which expression of at least 2, 3, 4, or all 5 genes in the group consisting of VPREB1, JCHAIN, CD22, IGHD, and LTB is enriched relative to other cell types in the plurality of CD34+ cell types. In some such embodiments this is determined by using at least 2, 3, 4, or all 5 genes in the group consisting of VPREB1, JCHAIN, CD22, IGHD, and LTB to determine a third score and comparing this third score to scores used to calculate other candidate cell types in the plurality of CD34+ cell types and determining that the expression of these genes is enriched relative to other cell types in the plurality of CD34+ cell types when the third score is greater than the other scores (described in blocks 836 through 846). In some embodiments the ‘score_genes’ function in scanpy is used to calculate the second score, where the scanpy “gene_list” is at least 2, 3, 4, or all 5 genes in the group consisting of VPREB1, JCHAIN, CD22, IGHD, and LTB and the gene_pool is all genes measured.

Referring to block 842, in some embodiments, the first cell type is a CD34+ cell type in a plurality of CD34+ cell types in which expression of at least 2, 3, 4, 5, 6, 7, 8, 9, 10, 11, 12, 13, 14, 15 or all 16 genes in the group consisting of ELANE, AZUI, PRTN3, CFD, MPO, CSFIR, CST7, CTSG, CYBB, FGL2, MARCH1, MRC1, NPL, ACP5, CYP27A1, and PLA2G7 is enriched relative to other cell types in the plurality of CD34+ cell types. In some such embodiments this is determined by using at least 2, 3, 4, 5, 6, 7, 8, 9, 10, 11, 12, 13, 14, 15 or all 16 genes in the group consisting of ELANE, AZUI, PRTN3, CFD, MPO, CSFIR, CST7, CTSG, CYBB, FGL2, MARCH1, MRC1, NPL, ACP5, CYP27A1, and PLA2G7 to determine a fourth score and comparing this fourth score to scores used to calculate other candidate cell types in the plurality of CD34+ cell types and determining that the expression of these genes is enriched relative to other cell types in the plurality of CD34+ cell types when the fourth score is greater than the other scores (described in blocks 836 through 846). In some embodiments the ‘score_genes’ function in scanpy is used to calculate the second score, where the scanpy “gene_list” is at least 2, 3, 4, 5, 6, 7, 8, 9, 10, 11, 12, 13, 14, 15 or all 16 genes in the group consisting of ELANE, AZUI, PRTN3, CFD, MPO, CSFIR, CST7, CTSG, CYBB, FGL2, MARCH1, MRC1, NPL, ACP5, CYP27A1, and PLA2G7 and the gene_pool is all genes measured.

In some embodiments, the first cell type is a cell type other than a CD34+ cell type.

In some embodiments, the cell type is selected from a hematopoietic stem cell (HSC) (including but not limited to an induced pluripotent stem cell (IPSC)), hepatocyte, cholangiocyte, mesenchymal cell, stellate cell (including but not limited to a hepatic stellate cell), fibroblast, smooth muscle cell, pericyte, endothelial cell, liver sinusoidal endothelial cell (LSEC), periportal endothelial cell (PPEC), peritoneal exudate cell (PEC), myeloid cells, Kupffer cell, monocyte, optionally a non-classical monocyte. macrophage, optionally scar-associated macrophage (SAM), dendritic cell, optionally a conventional type 1 dendritic cell (cDC1), conventional type 2 dendritic cell (cDC2), or plasmacytoid dendritic cell; neutrophil, T-cell, optionally a proliferated T-cell, natural killer (NK) cell, optionally a proliferated conventional NK cell or cytotoxic NK cell, B-cell, plasma cell, erythrocyte, and mast cell.

In some embodiments, the cell type is selected from a B cell, T cell, basophil mast progenitor cell, common lymphoid progenitor, common myeloid progenitor, dendritic cell, erythroid lineage cell, erythroid progenitor cell, granulocyte monocyte progenitor cell, hematopoietic precursor cell, macrophage, mast cell, megakaryocyte-erythroid progenitor cell, mesenchymal cell, monocyte, natural killer cell, neutrophil, plasma cell, plasmacytoid dendritic cell, and pro-B cell.

In some embodiments, the cell type is selected from basophil, eosinophil, erythroid progenitor cell, hematopoietic precursor cell, erythroid cell, megakaryocyte-erythroid progenitor cell, granulocyte monocyte progenitor, erythroid-mast transitioning cell, megakaryocyte progenitor cell, monocyte, and mast cell.

In some embodiments at least 2, 3, 4, 5, 6, 7, 8, 9, 10, 11, 12, 13, 14, 15 or 15 or more gene markers of one or more cells selected from basophils/mast cells, CD14+, erythroid lineage cells, hematopoietic precursor cells, lymphoid lineage cells, megakaryocyte-erythroid progenitor cells, megakaryocytes, and myeloid lineage cells is enriched relative to other cell types.

In some embodiments at least 2, 3, 4, 5, 6, 7, 8, 9, 10, 11, 12, 13, 14, 15 or 15 or more gene markers of one or more cells selected from B cell, T cell, basophil mast progenitor cell, common lymphoid progenitor, common myeloid progenitor, dendritic cell, erythroid lineage cell, erythroid progenitor cell, granulocyte monocyte progenitor cell, hematopoietic precursor cell, macrophage, mast cell, megakaryocyte-erythroid progenitor cell, mesenchymal cell, monocyte, natural killer cell, neutrophil, plasma cell, plasmacytoid dendritic cell, and pro-B cell is enriched relative to other cell types.

In some embodiments at least 2, 3, 4, 5, 6, 7, 8, 9, 10, 11, 12, 13, 14, 15 or 15 or more gene markers of one or more cells selected from basophil, eosinophil, erythroid progenitor cell, hematopoietic precursor cell, erythroid cell, megakaryocyte-erythroid progenitor cell, granulocyte monocyte progenitor, erythroid-mast transitioning cell, megakaryocyte progenitor cell, monocyte, mast cell is enriched relative to other cell types.

Referring to block 844, in some embodiments, the first cell type is a CD34+ cell type in a plurality of CD34+ cell types in which expression of at least 2, 3, 4, 5 or all 6 genes in the group consisting of CLC, HDC, PRG2, RNASE2, FCER1A, and CPA3 is enriched relative to other cell types in the plurality of CD34+ cell types. In some such embodiments this is determined by using at least 2, 3, 4, 5 or all 6 genes in the group consisting of CLC, HDC, PRG2, RNASE2, FCER1A, and CPA3 to determine a fifth score and comparing this fifth score to scores used to calculate other candidate cell types in the plurality of CD34+ cell types and determining that the expression of these genes is enriched relative to other cell types in the plurality of CD34+ cell types when the fifth score is greater than the other scores (described in blocks 836 through 846). In some embodiments the ‘score_genes’ function in scanpy is used to calculate the first score, where the scanpy “gene_list” is at least 2, 3, 4, 5 or all 6 genes in the group consisting of CLC, HDC, PRG2, RNASE2, FCER1A, and CPA3 and the gene_pool is all genes measured.

Referring to block 846, in some embodiments, the first cell type is a CD34+ cell type in a plurality of CD34+ cell types in which expression of at least 2, 3, 4, 5, 6, 7 or all 8 genes in the group consisting of CRHBP, EMCN, HLF, AVP, RUNX1, HOXA9, MLLT3, PROM1 is enriched relative to other cell types in the plurality of CD34+ cell types. In some such embodiments this is determined by using at least 2, 3, 4, 5, 6, 7 or all 8 genes in the group consisting of CRHBP, EMCN, HLF, AVP, RUNX1, HOXA9, MLLT3, PROM1 to determine a sixth score and comparing this sixth score to scores used to calculate other candidate cell types in the plurality of CD34+ cell types and determining that the expression of these genes is enriched relative to other cell types in the plurality of CD34+ cell types when the sixth score is greater than the other scores (described in blocks 836 through 846). In some embodiments the ‘score_genes’ function in scanpy is used to calculate the sixth score, where the scanpy “gene_list” is at least 2, 3, 4, 5, 6, 7 or all 8 genes in the group consisting of CRHBP, EMCN, HLF, AVP, RUNX1, HOXA9, MLLT3, PROM1 and the gene_pool is all genes measured.

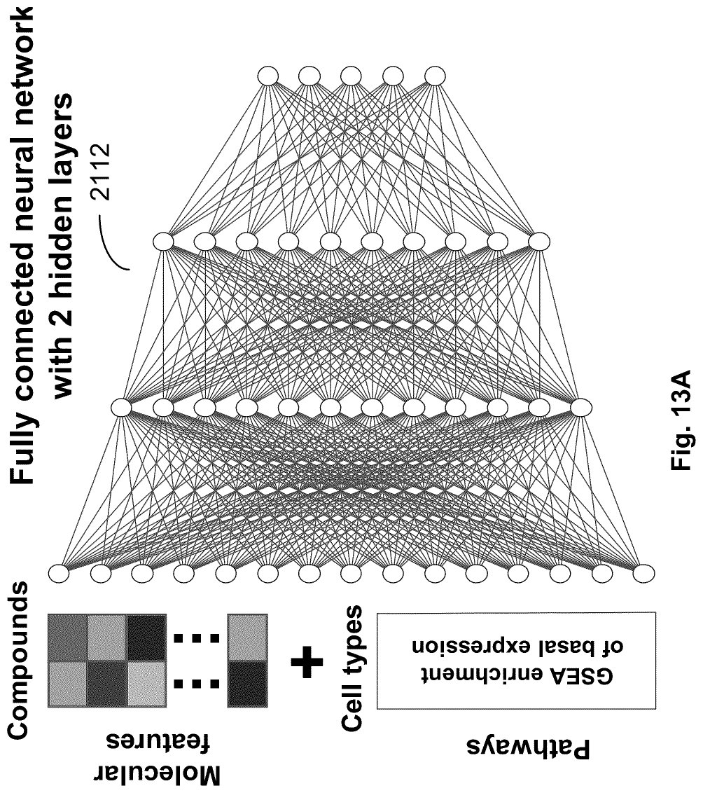

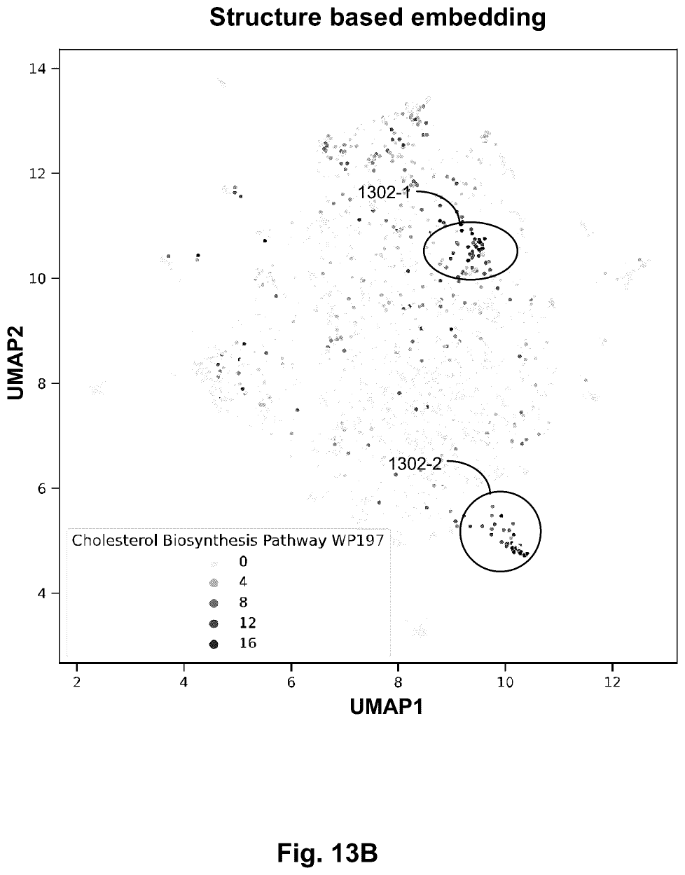

Referring to block 848, in some embodiments, as illustrated in FIG. 13A, the structure encoder 2112 is a first multilayer perceptron (MLP) having a first plurality of hidden layers. In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. In some embodiments the activation function is ReLU (Rectified Linear Unit), Sigmoid, or Tanh. Referring to block 850, in some embodiments, the first plurality of hidden layers consists of between 2 and 20 hidden layers. In some embodiments each hidden layer comprises between 100 and 50,000 nodes. More disclosure on suitable MLPs that can serve as the structure encoder 2112 in some embodiments of the present disclosure is found in Vang-mata ed., 2020, Multilayer Perceptrons: Theory and Applications, Nova Science Publishers, Hauppauge, New York, which is hereby incorporated by reference.

Referring to block 852, in some embodiments, the first plurality of parameters of the structure encoder 2112 consists of between 1000 and 1×10⁷ parameters. In the case of an MLP, the parameters include the weights connecting the nodes in the network. More disclosure on parameters is provided in the definitions section above.

Referring to block 854, in some embodiments, the structure encoder 2112 is a convolutional neural network or a graph based neural network. In some embodiments the structure encoder 2112 is a logistic regression model, neural network model, a support vector machine, a Naive Bayes model, a nearest neighbor model, a random forest model, a decision tree, a boosted trees model, a multinomial logistic regression model, a linear model, a linear regression model, a GradientBoosting model, a mixture model, a hidden Markov model, a Gaussian NB model, or a linear discriminant analysis model.

In some embodiments the structure encoder 2112 is a foundational model. Non-limiting examples of foundational models include, but are not limited to Geneformer (Theodoris et al., 2023, “Transfer learning enables predictions in network biology,” Nature, 618(7965):616-624, which is hereby incorporated by reference) and scGPT (Cui et al., 2023, “scGPT: Towards Building a Foundation Model for Single-Cell Multi-omics Using Generative AI” bioRxiv 2023.04.30.538439, which is hereby incorporated by reference). In some embodiments the structure encoder 2112 is a large language model. A non-limiting example of a large language model is Chemformer (Ross Irwin et al, 2022, “Chemformer: a pre-trained transformer for computational chemistry,” Mach. Learn.: Sci. Technol. 3 015022, which is hereby incorporated by reference).

Referring to block 856, as in the case of the first input data structure 2106-1 of block 802, a second input data structure 2106-2 is optionally inputted into the structure encoder 2112. The second input data structure 2106-2 comprises a combination of a feature representation of the second compound 2108-2 and the baseline transcriptional representation of the first cell type 2110. There is retrieved, by operation of the first plurality of parameters 2114 on the second input data structure 2106-2 in accordance with the architecture of the structure encoder 2112, as output from the structure encoder, a second compound embedding 2116-2 having the first dimension.

Referring to block 858, optionally the first compound embedding 2116-1 and the second compound embedding 2116-2 are projected into a plurality of transcriptional embeddings each having the first dimension. Each respective transcriptional embedding in the plurality of transcriptional embeddings is overlayed onto each other transcriptional embedding in the plurality of transcriptional embeddings 2128. FIG. 11B illustrates a UMAP visualization of the co-embedding of compound embeddings (black dots in FIG. 11B) and transcriptional embedding (grey dots in FIG. 11B). For FIG. 11B, each compound embedding and each transcriptional embedding has 96 dimensions and the UMAP algorithm (McInnes et al., 2018, “UMAP: Uniform Manifold Approximation and Projection for Dimension Reduction,” arXiv: 1802.03426 [stat. ML] is used to visualize this co-embedding in two dimensions.

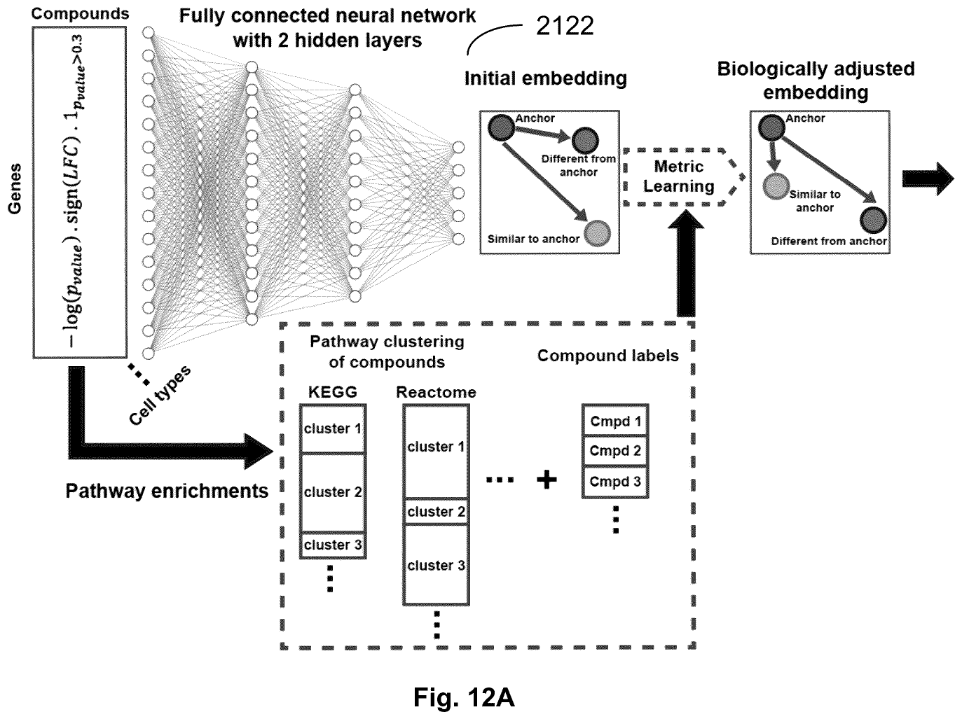

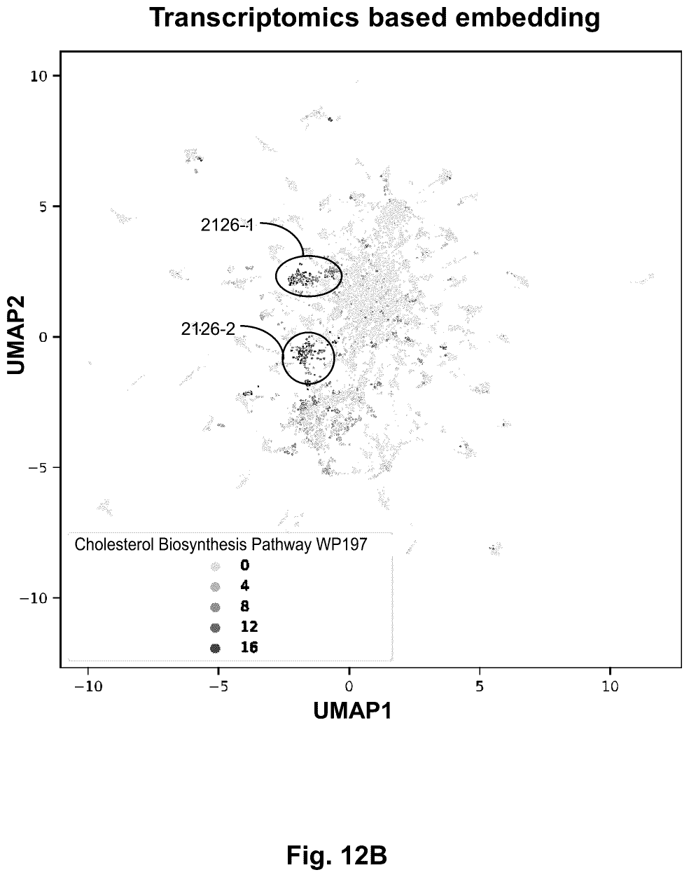

FIG. 12B illustrates a UMAP of the transcriptional embedding (96 dimensions), of a plurality of transcriptional embeddings. Each transcriptional embedding represented the transcriptome of cells exposed to a particular compound in a plurality of compounds. Transcriptome embeddings are colored by their cholesterol biosynthesis pathway score. FIG. 12B illustrates two clusters of transcriptional embeddings with similar pathway scores, clusters 2126-1 and 2126-2. The pathway scores of the samples used to construct the individual transcriptome embeddings within cluster 2126-1 of FIG. 12B can be used to ascertain a biological state for cluster 2126-1. For instance, the biological state could be a healthy state, a diseased state, a state of good prognosis for a disease condition, a state of bad prognosis for a disease condition, an ability to inhibit a particular drug target, etc. Likewise, the pathway scores of the samples used to construct the individual transcriptome embeddings within cluster 2126-2 of FIG. 12B can be used to ascertain a biological state for cluster 2126-2. Thus, as illustrated in FIG. 12B, at least a subset of the plurality of transcriptional embeddings collectively populate a plurality of clusters 2126-1, . . . , 2126-P, where P is a positive integer. Each cluster 2126 in the plurality of clusters is representative of a corresponding biological state. In some embodiments there are two different clusters 2126. In some embodiments, there are 3, 4, 5, 6, 7, 8, 9, 10, 11, 12, 13, 14, 15, 16, 17, 18, 19, 20, or more than 25 clusters. In some embodiments each such cluster occur in the dimensional space of the first dimension (e.g., 96 dimensions for FIG. 12B) but are visualized in two dimensions using an algorithm such as UMAP.

In the example of FIG. 12B, in accordance with block 858, the first compound embedding 2116-1 and the second compound embedding 2116-2 are projected into the plurality of transcriptional embeddings of FIG. 12B to see if they fall into the same or different clusters 2126.

Each respective transcriptional embedding 2128 in the plurality of transcriptional embeddings is generated from inputting a corresponding cellular constituent abundance data set 2118 representative of a first cell type into a transcriptional encoder 2122 comprising a second plurality of parameters 2124-1, . . . , 2124-Q, where Q is a positive integer.

The structure encoder 2112 is trained to minimize a loss against the plurality of transcriptional embeddings. For instance, in the case of FIG. 11B, the transcriptional encoder 2122 was first trained to project high-dimensional transcriptomics into an 96-dimensional latent space. The parameters of the transcriptional encoder 2122 were then frozen and the structure encoder is trained so that the projection of molecular features (compound embeddings 2116) into the same 96-dimensional latent space produced by the transcriptional encoder has the minimum L1 reconstruction loss compared to the fixed transcriptional embedding (FIG. 18A). Effectively this results in a multimodal co-embedding that can generate embeddings both from transcriptional and structural data.

Referring to block 860, in some embodiments, the corresponding cellular constituent data set 2118 comprises single cell transcriptional data for a plurality of cells of the first type. In some embodiments, the corresponding cellular constituent data set 2118 is determined by single-cell ribonucleic acid (RNA) sequencing (scRNA-seq), scTag-seq, single-cell assay for transposase-accessible chromatin using sequencing (scATAC-seq), CyTOF/SCOP, E-MS/Abseq, miRNA-seq, CITE-seq, or any combination thereof.

The cellular constituent abundance measurement technique used for a given cellular constituent can be selected based on the desired cellular constituent to be measured. For instance, scRNA-seq, scTag-seq, and miRNA-seq can be used to measure RNA expression. Specifically, scRNA-seq measures expression of RNA transcripts, scTag-seq allows detection of rare mRNA species, and miRNA-seq measures expression of micro-RNAs. CyTOF/SCOP and E-MS/Abseq can be used to measure protein expression in the cell. CITE-seq simultaneously measures both gene expression and protein expression in the cell, and scATAC-seq measures chromatin conformation in the cell. Table 1 above provides example protocols for performing each of the cellular constituent abundance measurement techniques described above. In some embodiments, any of the protocols described in Table 1 of Shen et al., 2022, “Recent advances in high-throughput single-cell transcriptomics and spatial transcriptomics,” Lab Chip 22, p. 4774, is used to measure the abundance of cellular constituents, such as genes, for the cellular constituent abundance data set.

In some embodiments, the corresponding cellular constituent data set 2118 contains measurements for a plurality of cellular constituents measured at a single time point. In some embodiments, the plurality of cellular constituents is measured at multiple time points. For instance, in some embodiments, the plurality of cellular constituents is measured at multiple time points throughout a cell state transition (e.g., a differentiation process, a response to an exposure to a compound, a developmental process, etc.).

It is to be understood that this is by way of illustration and not limitation, as the present disclosure encompasses analogous methods using measurements of other cellular constituents obtained from cells (e.g., single cells). It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

In some embodiments, the corresponding cellular constituent data set 2118 contains corresponding abundances for a plurality of cellular constituents that are preprocessed. In some embodiments, the preprocessing includes one or more of filtering, normalization, mapping (e.g., to a reference sequence), quantification, scaling, deconvolution, cleaning, dimension reduction, transformation, statistical analysis, and/or aggregation. For example, in some embodiments, the plurality of cellular constituents is filtered based on a desired quality, e.g., size and/or quality of a nucleic acid sequence, or a minimum and/or maximum abundance value for a respective cellular constituent. In some embodiments, filtering is performed in part or in its entirety by various software tools, such as Skewer. See, Jiang et al., 2014, BMC Bioinformatics 15(182): 1-12, which is hereby incorporated by reference. In some embodiments, the plurality of cellular constituents is filtered for quality control, for example, using a sequencing data QC software such as AfterQC, Kraken, RNA-SeQC, FastQC, or another similar software program. In some embodiments, the plurality of cellular constituents is normalized, e.g., to account for pull-down, amplification, and/or sequencing bias (e.g., mappability, GC bias etc.). See, for example, Schwartz et al., PLOS ONE 6(1):e16685 (2011) and Benjamini and Speed, Nucleic Acids Research 40(10): e72 (2012), the contents of which are hereby incorporated by reference, in their entireties, for all purposes. In some embodiments, the preprocessing removes a subset of cellular constituents from the plurality of cellular constituents. In some embodiments, the preprocessing the corresponding abundances for the plurality of cellular constituents improves (e.g., lowers) a high signal-to-noise ratio.

Thus, in some embodiments, the corresponding abundance of a respective cellular constituent in the corresponding cellular constituent data set 2118 comprises any one of a variety of forms, including, without limitation, a raw abundance value, an absolute abundance value (e.g., transcript number), a relative abundance value (e.g., relative fluorescent units, transcriptome analysis, and/or gene set expression analysis (GSEA)), a compound or aggregated abundance value, a transformed abundance value (e.g., log 2 and/or log 10 transformed), a change (e.g., fold- or log-change) relative to a reference (e.g., a normal sample, matched sample, reference dataset, housekeeping gene, and/or reference standard), a standardized abundance value, a measure of central tendency (e.g., mean, median, mode, weighted mean, weighted median, and/or weighted mode), a measure of dispersion (e.g., variance, standard deviation, and/or standard error), an adjusted abundance value (e.g., normalized, scaled, and/or error-corrected), a dimension-reduced abundance value (e.g., principal component vectors and/or latent components), and/or a combination thereof. Methods for obtaining cellular constituent abundances using dimension reduction techniques are known in the art and further detailed below, including but not limited to principal component analysis, factor analysis, linear discriminant analysis, multi-dimensional scaling, isometric feature mapping, locally linear embedding, hessian eigenmapping, spectral embedding, t-distributed stochastic neighbor embedding, and/or any substitutions, additions, deletions, modification, and/or combinations thereof as will be apparent to one skilled in the art. See, for example, Sumithra et al., 2015, “A Review of Various Linear and Non Linear Dimensionality Reduction Techniques,” Int J Comp Sci and Inf Tech, 6(3), 2354-2360, which is hereby incorporated herein by reference in its entirety.

In some embodiments scRNA-seq is used to obtain cellular constituent abundance data for each sample of cells in a plurality of samples. Each sample is exposed to a different compound in a plurality of compounds. The scRNA-seq is performed after the cells have been exposed to a compound for a period of time. In some embodiments this period of time is at least 4, 8, 12, 24, or 48 hours. In some embodiments pseudobulk differential expressed genes (DEGs) analysis was performed where cells perturbed by a compound at a cell type (at determined for instance by blocks 836 through 846 or by the techniques disclosed in block 836 through 846 were merged and Limma (limma-voom pipeline, which normalizes the expression counts and runs differential expression on the normalized values; Law et al., 2018, “RNA-seq analysis as easy as 1-2-3 with limma, Glimma, and edgeR,” F1000Research 5:1408, last updated 28 Dec. 2018, which is hereby incorporated by reference) was applied to compare the compound's expression profile to a DMSO control arm. The DMSO control arm consisted of samples of the same type of cells that are exposed to DMSO but no compounds. Differential expression scores (DESs) for a compound are then computed in some embodiment as the −log₁₀ (qᵥₐₗᵤₑ) sign(LFC) where LFC reflects the magnitude of the expression change compared to DMSO for a given cellular constituent and qᵥₐₗᵤₑ is the statistical significance of that change. Thus, as illustrated in FIG. 12A, a cellular constituent abundance data set 2118 in some embodiments comprises the DES value for each cellular constituent in the plurality of cellular constituents. In some such embodiments each cellular constituent abundance data set represents a particular compound that the cell sample was exposed to for the period of time.

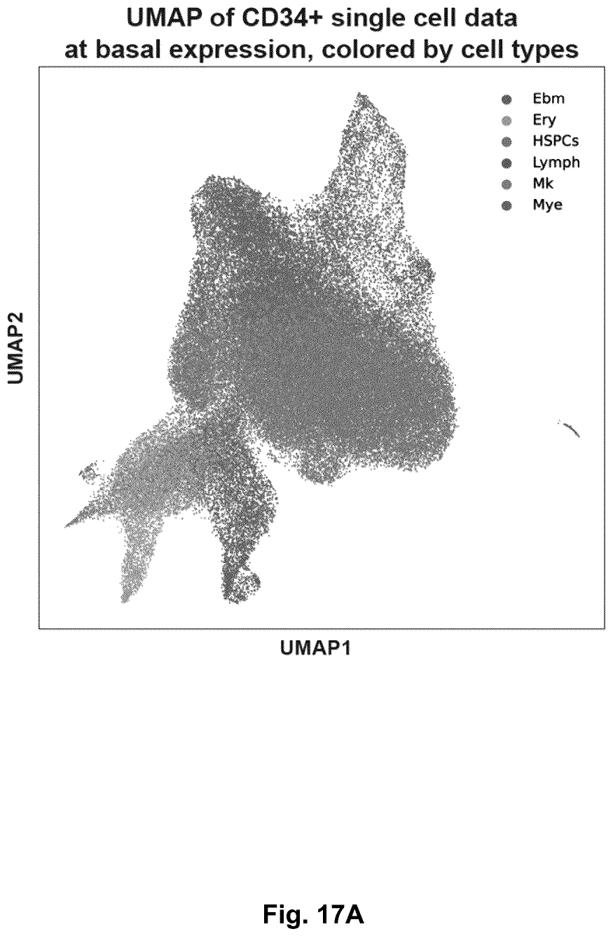

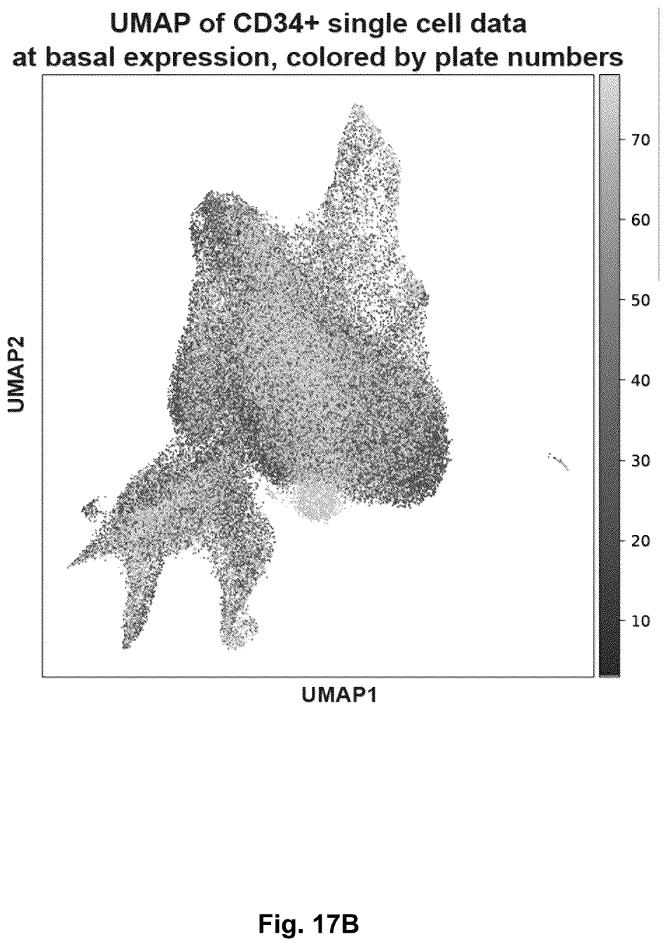

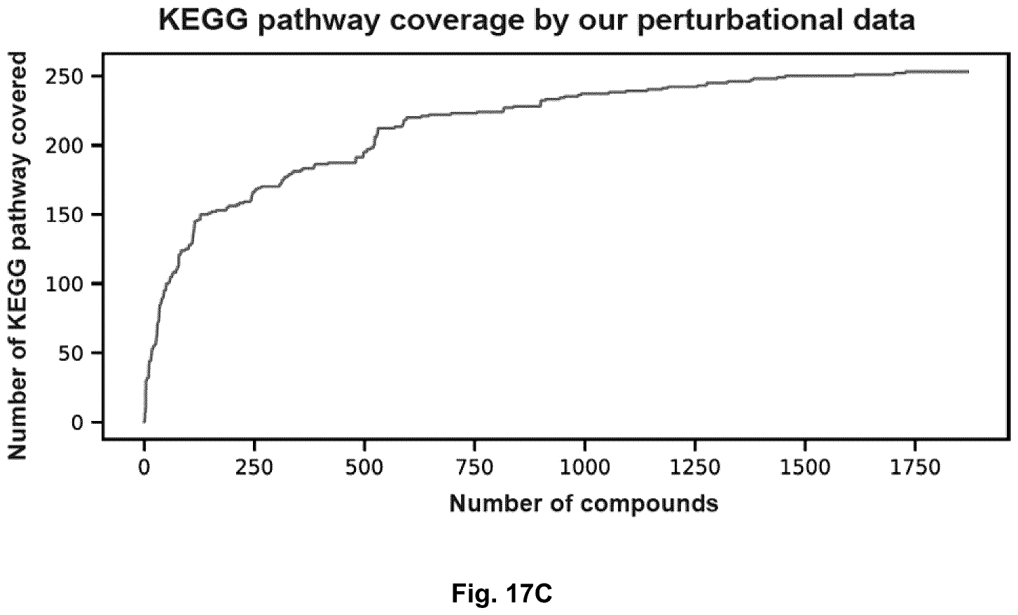

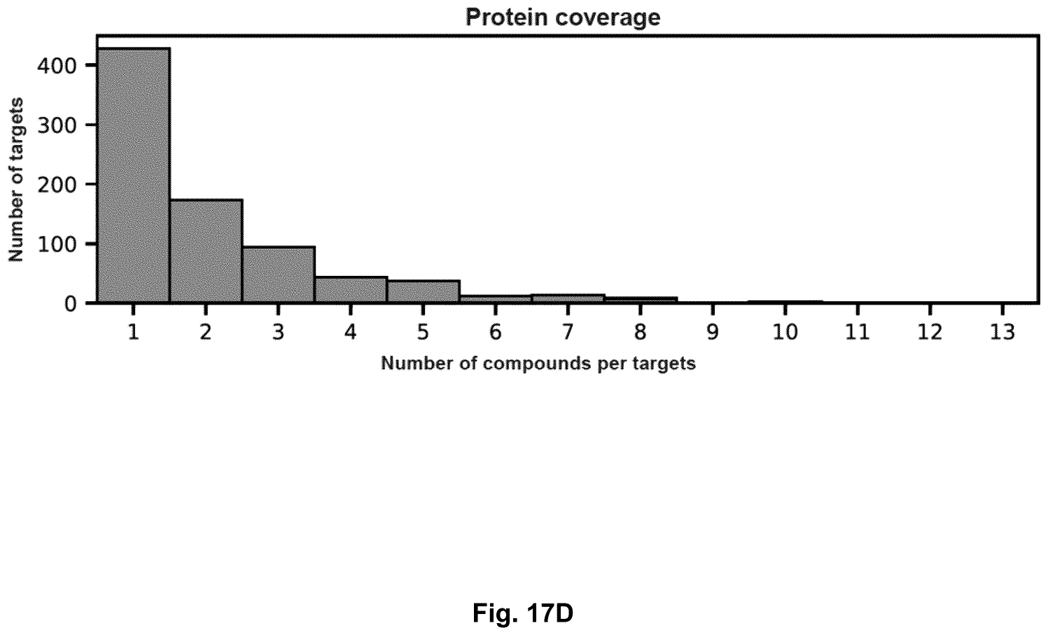

In the case of constructing the transcriptional embeddings illustrated in FIG. 12B, a set of known markers was used to annotate 6 well-characterized cell types within CD34+ population of each sample as described above in blocks 836 through 846. The pseudobulk differential expressed genes analysis was then performed where cells in each sample perturbed by a compound at a cell type were merged and Limma (limma-voom pipeline, which normalizes the counts and runs differential expression on the normalized values; Law et al., 2018, “RNA-seq analysis as easy as 1-2-3 with limma, Glimma, and edgeR,” F1000Research 5:1408, last updated 28 Dec. 2018, which is hereby incorporated by reference) was applied to compare the compound's expression profile to the DMSO control arm. The DES for each cellular constituent was calculated as described above. A compound that produced no differentially expressed gene at 5% FDR in all cells exposed to the compound for any cellular constituent were filtered out resulting in 1870 compounds that collectively represent 815 mechanism of actions (MOAs). A diverse coverage of pathways across active compounds' signatures was observed, with more than 80% of KEGG pathways enriched (FIGS. 17C and 17D). Together, a single-cell database was generated with access to full transcriptomics that covers most known pathways and diverse MOAs, serving as our training data for the transcriptional encoder 2122 in one example. In the example of FIG. 12B, the transcriptional encoder 2122 takes the DES of 14,649 genes as the input, which capture the significance of differential expression and the direction of gene regulation produced by compounds. The multilayer perceptron (MLP) with two hidden layers, as illustrated in FIG. 12A, for the initial dimensionality reduction into the 96 dimensions is applied.

Referring to block 862, in some embodiments, the corresponding cellular constituent data 2118 comprises bulk transcriptional data for a plurality of cells of the first type.

Referring to block 864, in some embodiments, the corresponding cellular constituent data set 2118 comprises cellular constituent abundance values for a plurality of cellular constituents.

Referring to block 866, in some embodiments, each cellular constituent in the plurality of cellular constituents uniquely maps to a different gene. In some such embodiments, the plurality of cellular constituents collectively maps to 30 or more, 40 or more, 50 or more, 60 or more, 70 or more, 80 or more, 90 or more, 100 or more, 1000 or more, 2000 or more, 3000 or more, 4000 or more, 5000 or more, 6000 or more, 7000 or more, 8000 or more, 9000 or more, or 10000 or more different genes. In some embodiments the set of cellular constituents collectively maps to between 1000 and 30000 different genes.

Referring to block 868, in some embodiments, each cellular constituent in the plurality of cellular constituents is a particular gene, a particular mRNA associated with a gene, a carbohydrate, a lipid, an epigenetic feature, an epitranscriptomic feature, a metabolite, an antibody, a peptide, a protein, a post-translational modification of a protein, or a combination thereof.

In some embodiments, the plurality of cellular constituents includes nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc.) proteins, lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular constituents such as oxidized and reduced forms of nicotinamide adenine dinucleotide (NADP/NADPH), and any combinations thereof.

Referring to block 870, in some embodiments, the plurality of cellular constituents comprises 50 or more cellular constituents, 100 or more cellular constituents, 150 or more cellular constituents, 200 or more cellular constituents, 300 or more cellular constituents, 500 or more cellular constituents, 1000 or more cellular constituents, 2000 or more cellular constituents, 4000 or more cellular constituents, or 8000 or more cellular constituents.

Referring to block 872, in some embodiments, the corresponding cellular constituent data set 2118 comprises a corresponding differential expression signature for a plurality of cells of the first type. Differential expression signatures have been described in block 860 above as the DES for each cellular constituent (e.g., gene) in a plurality of cellular constituents. In some embodiments the plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells of the first type.

Referring to block 874, in some embodiments, the corresponding differential expression signature comprises a plurality of differential values, each respective differential value in the plurality of differential values corresponds to a respective cellular constituent in a set of cellular constituents, and the respective differential value represents a difference between (i) one or more abundance values measured for the respective cellular constituent in a first assay of a first plurality of cells of the first cell type that represent a first cell state and (ii) one or more abundance values measured for the respective cellular constituent in a second assay of a second plurality of cells of the first cell type that represent a second cell state. For instance, such a differential expression signature has been described in block 860 above. In some embodiments the first plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells of the first type. In some embodiments the second plurality of cells comprises 2, 3, 4, 5, 6, 7, 8, 9, 10, 11, 12, 13, 14, 15, 16, 17, 18, 19, 20, 30, 40, 50, 60, 70, 80, 100, 200, or 1000 or more cells of the first type. In some embodiments the first plurality of cells is in one or more wells in a first plate and the second plurality of cells is in one or more control wells in the first plate.

Referring to block 876, in some embodiments, the first cell state is exposure of the first plurality of cells to a perturbation (e.g., for a period of time), and the second cell state is exposure of the second plurality of cells to a reference environment. In some embodiments the perturbation is exposure to a solubilized compound. In some embodiments the perturbation is exposure to a solubilized compound where the solubilized compound has a predetermined concentration. In some embodiments the perturbation is exposure to a solubilized compound where the solubilized compound has a concentration between 1×10−9 M and 1 M. In some embodiments the exposure is for a period of time. In some embodiments the period of time is between one minute and one week.

Referring to block 878, in some embodiments, the reference environment is exposure to a polar aprotic solvent (e.g., dimethyl sulfoxide).

Referring to block 880, in some embodiments, the associated perturbation 2120 is exposure of the first plurality of cells to a chemical compound solubilized in a polar aprotic solvent. In some such embodiments the solubilized compound has a concentration between 1×10⁻⁹ M and 1 M.

Referring to block 882, in some embodiments, the plurality of transcriptional embeddings collectively 2128 represents over 500 different first cell states or over 1000 different first cell states. For instance block 860 in conjunction with FIG. 17C describes a plurality of transcriptional embeddings that collectively enrich for 80% of the KEGG pathways and 815 different mechanisms of actions. In some embodiments a cell state is a state in which a particular pathway or predetermined combination of pathways is activated. In some embodiments each cell state represents a particular diseased state or a healthy state. In some embodiments a cell state is characterized by the activation (e.g. expression) of one or more particular cellular constituents.

Referring to block 884, in some embodiments, the plurality of transcriptional embeddings 2128 collectively represents over 100 different biological pathways. Referring to block 886, in some embodiments, the plurality of transcriptional embeddings 2128 collectively represents over 200 different biological pathways.

Referring to block 888, in some embodiments, each different first cell state is exposure of the first plurality of cells with a different chemical compound. For instance, in one example there are 50 different cell states each characterized by (represented by) a different transcriptional embedding 2128. In accordance with block 888, each respective transcriptional embedding is constructed by exposing a plurality of cells to a corresponding chemical compound for a period of time.

Referring to block 890, in some embodiments, the transcriptional encoder 2122 is a second multilayer perceptron (MLP) having a second plurality of hidden layers. In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. In some embodiments the activation function is ReLU (Rectified Linear Unit), Sigmoid, or Tanh. Referring to block 892, in some embodiments, the second plurality of hidden layers consists of between 2 and 20 hidden layers. In some embodiments each hidden layer comprises between 100 and 50,000 nodes. More disclosure on suitable MLPs that can serve as the transcriptional encoder 2122 in some embodiments of the present disclosure is found in Vang-mata ed., 2020, Multilayer Perceptrons: Theory and Applications, Nova Science Publishers, Hauppauge, New York, which is hereby incorporated by reference.

Referring to block 894, in some embodiments, the second plurality of parameters consists of between 1000 and 1×10⁷ parameters. In the case of an MLP, the parameters include the weights connecting the nodes in the network. More disclosure on parameters is provided in the definitions section above.

Referring to block 896, in some embodiments, the transcriptional encoder 2122 is a convolutional neural network or a graph based neural network. In some embodiments the transcriptional encoder 2122 is a logistic regression model, neural network model, a support vector machine, a Naive Bayes model, a nearest neighbor model, a random forest model, a decision tree, a boosted trees model, a multinomial logistic regression model, a linear model, a linear regression model, a GradientBoosting model, a mixture model, a hidden Markov model, a Gaussian NB model, or a linear discriminant analysis model.

In some embodiments the transcriptional encoder 2122 is a foundational model. Non-limiting examples of a foundational model include, but are not limited to Geneformer (Theodoris et al., 2023, “Transfer learning enables predictions in network biology,” Nature, 618(7965):616-624, which is hereby incorporated by reference) and scGPT (Cui et al., 2023, “scGPT: Towards Building a Foundation Model for Single-Cell Multi-omics Using Generative AI” bioRxiv 2023.04.30.538439, which is hereby incorporated by reference). In some embodiments the transcriptional encoder 2122 is a large language model. A non-limiting example of a large language model is Chemformer (Ross Irwin et al, 2022, “Chemformer: a pre-trained transformer for computational chemistry,” Mach. Learn.: Sci. Technol. 3 015022, which is hereby incorporated by reference).

Referring to block 898, in some embodiments, each respective transcriptional embedding 2128 consists of between 40 and 2000 dimensions. Referring to block 900, in some embodiments, the respective transcriptional embedding 2128 consists of between 50 and 500 dimensions. Referring to block 902, in some embodiments, the respective transcriptional embedding 2128 consists of between 60 and 250 dimensions. Referring to block 904, in some embodiments, the respective transcriptional embedding 2128 consists of between 70 dimensions and 100 dimensions. In a particular example, each respective transcriptional embedding 2128 consists of 96 dimensions.

Referring to block 906, in some embodiments, the plurality of clusters 2126-1, . . . , 2126-P discussed in block 858 comprises five or more clusters representing five or more biological states. That is, the first cluster represented the first biological state, the second cluster represents the second biological state, the third cluster represents the third biological state, the fourth cluster represents the fourth biological state, and the fifth cluster represents the fifth biological state. Referring to block 908, in some embodiments, the plurality of clusters 2126-1, . . . , 2126-P comprises 25 or more clusters representing 25 or more biological states.

Referring to block 910, in some embodiments, the first compound is a first organic compound having a molecular weight of less than 2000 Daltons. In some embodiments the first compound has any of the molecular weights described in block 206 for a test chemical compound.

Referring to block 912, in some embodiments the first compound is a peptide having a mass of less than 4500 Daltons. For instance, in some embodiments, the first compound is an organic compound having 41 amino acids or fewer. In some embodiments, the first compound has a molecular weight of less than approximately 4500 Daltons (e.g., 41 amino acids*110 Daltons).

Referring to block 914, in some embodiments, the first compound is a protein having a mass of more than 4600 Daltons. For instance, in some embodiments, the first compound is an organic polymer having at least 42 amino acids. In some embodiments, the first compound has a molecular weight of at least approximately 4600 Daltons (e.g., 42 amino acids*110 Daltons).

In some embodiments the first compound comprises at least 2, at least 5, at least 10, at least 20, at least 30, at least 40, or at least 50 amino acids. In some embodiments, the first compound comprises no more than 60, no more than 50, no more than 40, no more than 30, no more than 20, or no more than 10 amino acids. In some embodiments, the first compound consists of from 2 to 10, from 2 to 50, from 5 to 50, from 10 to 45, or from 35 to 60 amino acids. In some embodiments, the first compound comprises a plurality of amino acids that falls within another range starting no lower than 2 amino acids and ending no higher than 60 amino acids.

Referring to block 916, in some embodiments, the first compound satisfies any two or more rules, any three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, the first compound satisfies one or more criteria in addition to Lipinski's Rule of Five. For example, in some embodiments, the first compound has five or fewer aromatic rings, four or fewer aromatic rings, three or fewer aromatic rings, or two or fewer aromatic rings. In some embodiments, the first compound is an organic compound that satisfies at least two, three or four criteria of the Lipinski rule of five criteria. In some embodiments, the first compound is an organic compound that satisfies zero, one, two, three, or all four criteria of the Lipinski rule of five criteria.

Referring to block 918, optionally, when the first compound embedding 2116-1 and the second compound embedding 2116-2 falls into a first cluster 2126 in the plurality of clusters, the first compound is associated with the corresponding biological state of the first cluster or the corresponding biological state of the second compound. Such an embodiment is of particular interest where the activity of the second compound (and possibly the other compounds whose perturbational studies form the basis for the other transcriptional embeddings of the first cluster are known) and thus serves to validate that the first compound, for which activity is not known, has the desirable activity. As an example, if each of the transcriptional embeddings in the first cluster other than that associated with the first compound were from perturbation studies with a set of compounds that are known inhibitors of a particular drug target (e.g., an inhibitor of a particular protein), the discovery that the transcriptional embedding from the perturbation study with the first compound co-clusters with the transcriptional embeddings from the perturbation studies with the known compounds indicates that the first compound is likely also an inhibitor of the particular drug target.

As described in block 858 above, if the first and second compound fall into cluster 2161-1 of FIG. 12B, the first compound would be considered to have the corresponding biological state of the first cluster and/or the second compound. Advantageously, the discovery that the first compound falls into the first cluster and is therefore associated with the biological state of the first cluster or the second compound can be done in accordance with the disclosed methods without producing a cellular constituent abundance dataset using cells exposed to the first compound. This illustrates one of the capabilities of the disclosed models: projection of new compounds without transcriptional readouts into the co-embedding to assess if such compounds fall within the same cluster and thus show similar transcriptional activities (and therefore the same or similar biological state). For instance, in one embodiment the disclosed transcriptional encoder was trained on a perturbational data set comprising the cellular constituent abundance dataset for 3700 compounds across the 6 different cell types disclosed in blocks 836 through 856, to generate transcriptional embeddings for them, and these were combined with the embedding space from a pre-trained structure encoder that had been trained on millions of molecular structures. On benchmarks, this joint model achieved 25% hit rate in matching transcriptional readout of a compound with the corresponding structure, on a dataset of 255 unseen molecules. By creating a transcription-structure co-embedding, the search space for hits is advantageously expanded beyond the compounds tested in perturbation studies to roughly 370,000 compounds. Thus the disclosed model has great utility for transcriptomics-based drug discovery at lower cost, both in the space of approved compounds (for a drug repurposing task) and in the space of unexplored new chemical entities. Using the disclosed methods, perturbational cellular constituent data is only needed for a small fraction of the chemical space being explored.

In some embodiments, the first compound embedding 2116-1 and the second compound embedding 2116-2 are considered to fall into the same first cluster 2126 when the second compound embedding 2116-2 is an embedding of one of the training compounds in the training dataset described in Section VIII and the similarity between the compound embedding 2116 of the first compound and the compound embedding 2116 of the second compound is among the top N compounds in the training set based on a ranking of similarity between the compound embedding 2116 of the first compound and the respective compound embeddings of each compound in the training dataset calculated by the structure encoder 2112. While the choice of N is application dependent, for instance depending on the number of compounds in the training set, in some embodiments N is 5, 10, 50, 100, or 1000.

In some embodiments, the first compound embedding 2116-1 and the second compound embedding 2116-2 are considered to fall into the same first cluster 2126 when the transcriptional cosine similarity between the compound embedding 2116 of the first compound and the compound embedding 2116 of the second compound is greater than 0.50, 0.60, 0.70, 0.80, 0.85, 0.90, 0.95, or 0.98.

In alternatives to block 918, elements of FIG. 8 described above are used to perform a method of determining whether a first compound and a second compound are causal for a common biological state that comprises inputting a first input data structure into a structure encoder, where the first input data structure comprises a combination of a feature representation of the first compound and a baseline transcriptional representation and the structure encoder comprises a first plurality of parameters. In this way, there is retrieved, by operation of the first plurality of parameters on the first input data structure in accordance with an architecture of the structure encoder, as output from the structure encoder, a first compound embedding having a first dimension. Then, a determination of respective similarity between the first compound embedding and each respective transcriptional embedding in a plurality of transcriptional embeddings is made, thereby determining a plurality of similarities. In embodiments, each transcriptional embedding in the plurality of transcriptional embeddings has the first dimension. In embodiments, each transcriptional embedding in the plurality of transcriptional embeddings is generated from inputting a corresponding cellular constituent abundance data set representative of the first cell type exposed to a different perturban (e.g., compounds) in a plurality of perturbans, into a transcriptional encoder comprising a second plurality of parameters. In embodiments, the plurality of perturbans includes the second compound. In embodiments, the plurality of transcriptional embeddings comprises at least 25 transcriptional embeddings. In embodiments the structure encoder is trained to minimize a loss against the plurality of transcriptional embeddings. In embodiments, the first compound is associated with a biological state that the second compound is known to be causal for when the embedding comparison determines that the similarity between the first compound embedding and the respective transcriptional embedding of the second compound satisfies a similarity criterion (e.g., being among the top embedding comparisons in terms of similarity). In such embodiments, clustering and UMAP visualization can be performed, but are optional.

Example 2 below shows examples of the first compound embedding 2116-1 and the second compound embedding 2116-2 being considered to fall into the same first cluster 2126.

In some embodiments, the model disclosed in Section V (which includes the structure encoder 2112 and the transcriptional encoder 2122) is used to screen 10 or more, 100 or more, 1000 or more, 10,000 more, or 1×10⁶ or more first compounds to determine whether they are causal for a particular biological state (of the first cluster or the second compound) in accordance with the methods disclosed in Section V.

### VI. IDENTIFYING A BIOLOGICAL STATE FOR WHICH A FIRST COMPOUND IS CAUSAL

Section V detailed embodiments in which a first compound clustered into a transcriptional embedding cluster that included a second compound thus establishing that the first compound had similar properties as the other compounds, such as the second compound, whose perturbational studies formed the basis for the transcriptional embedding.

In this section, similar studies are performed to see which transcriptional embedding cluster a first compound clustered into. Upon co-clustering into a given transcriptional embedding cluster the first compound is consider to illicit the same biological state as the other transcriptional embeddings in the cluster. Such biological states can, for instance, arise because each of the transcriptional embeddings in the cluster enrich a common set of pathways associated with a biological condition, such as a disease state or the alleviation (treatment) of the disease state. To this end, referring to block 930 of FIG. 9A, systems and methods for identifying a biological state for which a first compound is causal are provided.

Referring to block 932, a first input data structure 2106 is inputted into a structure encoder 2112. The first input data structure 2106 comprises a combination of a feature representation of the first compound 2108-1 and a baseline transcriptional representation of a first cell type 2110 as described in Section V. The structure encoder 2112 comprises a first plurality of parameters 2114-1, . . . , 2114-N, where N is a positive integer as described in Section V. A first compound embedding 2116-1 having a first dimension is obtained as output from the structure encoder 2112 by operation of the first plurality of parameters 2114 on the first input data structure 2106-1 in accordance with an architecture of the structure encoder as described in Section V.

Referring to block 934, the first compound embedding 2116-1 is projected into a plurality of transcriptional embeddings 2128 each having the first dimension. This is illustrated for example in FIG. 11A where compound embeddings 2116 and transcriptional embeddings 2128 that are each 96-dimensional have been co-embedded. While each embedding is 96 dimensional in FIG. 11A, FIG. 11A is a UMAP of these 96 dimensions and thus is a two-dimensional representation of the multidimensional embeddings.

Referring to block 936, each respective transcriptional embedding in the plurality of transcriptional embeddings is overlayed onto each other transcriptional embedding in the plurality of transcriptional embeddings. This is illustrated in FIG. 11B as discussed in Section V.

Referring to block 938, at least a subset of the plurality of transcriptional embeddings collectively populates a plurality of clusters 2126-1, . . . , 2126-P. For instance, FIG. 12B illustrates two clusters of transcriptional embeddings, clusters 2126-1 and 2126-2. Cluster 2126-1 forms because the respective 96-dimensional vectors describing each respective transcriptional embedding in cluster 2126-1 are more similar to each other than they are to any other transcriptional embedding in the embedding space. Clusters 2126-2 forms because the respective 96-dimensional vectors describing each respective transcriptional embedding in cluster 2126-2 are more similar to each other than they are to any other transcriptional embedding in the embedding space.

Referring to block 940, each cluster in the plurality of clusters is representative of a corresponding biological state. For instance, the biological state could be a healthy state, a diseased state, a state of good prognosis for a disease condition, a state of bad prognosis for a disease condition, inhibition of a particular biological pathway, inhibition of a particular protein in a biological pathway, etc.

Referring to block 942, each respective transcriptional embedding 2128 in the plurality of transcriptional embeddings is generated from inputting a corresponding cellular constituent abundance data set 2118 representative of the first cell type into a transcriptional encoder 2122 comprising a second plurality of parameters 2124-1, . . . , 2124-Q, where Q is a positive integer. This has been described in Section V.

Referring to block 944, the structure encoder 2112 is trained to minimize a loss against the plurality of transcriptional embeddings. In such embodiments, the parameters of the transcriptional encoder 2122 are frozen once they have been trained and the structure encoder 2122 is trained so that the projection of compound embeddings into the same latent space as the transcriptional encoder 2122 has the minimum L1 reconstruction loss compared to the fixed trained transcriptional embedding as illustrated in FIG. 18A. In some embodiments, the compound embeddings of 100 or more, 200 or more, or 300 or more different compounds are used to perform this training.

Referring to block 946, when the first compound embedding 2116-1 projected into the plurality of transcriptional embeddings 2128 falls into a first cluster 2126 in the plurality of clusters, the first compound is associated with the corresponding biological state of the first cluster. For instance, as described in block 858 above, if the first compound embedding falls into cluster 2161-1 of FIG. 12B the first compound would be considered to have the corresponding biological state of the first cluster. Advantageously, the discovery that the first compound falls into the first cluster and is therefore associated with the biological state of the first cluster can be done in accordance with the disclosed methods without producing a cellular constituent abundance dataset using cells exposed to the first compound. This illustrates one of the capabilities of the disclosed models: projection of new compounds without transcriptional readouts into the co-embedding to assess if such compounds fall within the same cluster and thus show similar transcriptional activities (and therefore the same or similar biological state). For instance, in one embodiment the disclosed transcriptional encoder was trained on a perturbational data set comprising the cellular constituent abundance dataset for 3700 compounds across the 6 different cell types disclosed in blocks 836 through 856, to generate a transcriptional embedding, and combined with the embedding space from a pre-trained structure encoder that had been trained on millions of molecular structures. On benchmarks, this model achieved 25% hit rate in matching transcriptional readout of a molecule with the corresponding structure, on a dataset of 255 unseen molecules. By creating a transcription-structure co-embedding, the search space for hits is advantageously expanded beyond the compounds tested in perturbation studies to roughly 370,000 compounds. Thus the disclosed model has great utility for transcriptomics-based drug discovery at lower cost, both in the space of approved compounds (for a drug repurposing task) and in the space of unexplored new chemical entities. Using the disclosed methods perturbational cellular constituent data is only needed for a small fraction of the chemical space being explored. In some embodiments, perturbational cellular constituent data is only needed for 0.05 percent, 0.1 percent, or 1 percent of the chemical space being explored. In some embodiments the chemical space being explored comprises 1000 or more compounds, 10,000 or more compounds, 50,000 or more compounds, 100,000 or more compounds, 200,000 or more compounds, 300,000 or more compound, 500,000 or more compounds, 1×10⁶ or more compounds, 5×10⁶ or more compounds, 1×10⁷ compounds, or 1×10⁸ compounds.

In some embodiments, the model disclosed in Section VI (which includes the structure encoder 2112 and the transcriptional encoder 2122) is used to screen 10 or more, 100 or more, 1000 or more, 10,000 more, or 1×10⁶ or more first compounds to determine whether they are causal for a particular biological state in accordance with the methods disclosed in Section VI.

### VII. TRAINING A STRUCTURE ENCODER TO DETERMINE A RELATIONSHIP BETWEEN ONE OR MORE BIOLOGICAL STATES AND A FIRST COMPOUND

Referring to block 1000 of FIG. 10A, in some embodiments, a method for training a structure encoder 2112 to determine a relationship between one or more biological states and a first compound is provided.

Referring to block 1002, a training dataset comprising a structure of each compound in a plurality of compounds is obtained. For each respective compound in the plurality of compounds, a corresponding cellular constituent abundance data set 2118 representative of a first cell type is obtained. In some embodiments the training dataset comprises 100 or more compounds, 200 or more compounds, 400 or more compounds, 500 or more compounds, 1000 or more compounds, 2000 or more compounds, or 5000 or more compounds.

In one non-limiting example, a library of 3700 compounds with diverse mechanism of actions (MOAs), with more than 1200 main targets was prepared. Further, scRNA-seq technology was used to profile the single-cell perturbational response of these compounds 24 hours post intervention in CD34+. The mRNA counts were measured for all genes. A set of known markers was used to annotate 6 well-characterized cell types within CD34+ population as described in blocks 836 through 846. Pseudobulk differential expressed genes (DEGs) analysis was performed where cells of a given cell type perturbed by a compound were merged and Limma (the limma-voom pipeline, which normalizes the counts and runs differential expression on the normalized values; Law et al., 2018, “RNA-seq analysis as easy as 1-2-3 with limma, Glimma, and edgeR,” F1000Research 5:1408, last updated 28 Dec. 2018, which is hereby incorporated by reference) was applied to compare the compound's expression profile to DMSO control arm as described in block 860. Differential expression scores (DESs) for a compound were then computed in some embodiments as the −log₁₀(qᵥₐₗᵤₑ) sign(LFC) where LFC reflects the magnitude of the expression change compared to DMSO for a given cellular constituent and qᵥₐₗᵤₑ is the statistical significance of that change. Compounds that produce no DEG at 5% FDR in all cells for any gene were filtered out, resulting in 1870 compounds and 815 MOAs. A diverse coverage of pathways across active compounds' transcriptional profiles, with more than 80% of KEGG pathways enriched as illustrated in FIGS. 17C and 17D. Together, this generated a single-cell database with access to full transcriptomics that covers most known pathways and diverse MOAs, serving as training data.

Referring to block 1004, the corresponding cellular constituent abundance data set of each respective compound in the plurality of compounds is used to obtain a separate clustering of the plurality of compounds against each set of pathways in a plurality of sets of pathways thereby obtaining a corresponding plurality of pathway labels for each compound in the plurality of compounds, each pathway label for a respective compound being a cluster assignment for the respective compound in a separate clustering of the plurality of compounds. This provides a weak prior for the transcriptional encoder to infer compound grouping through metric learning (e.g., Gouk et al., 2019, “Learning distance metrics for multi-label classification,” Asian Conference on machine learning, PMLR; Kayar and Bilge, 2019, “Deep Metric Learing: A Survey” Symmetry 11(9), p. 1066; and Yang et al., 2006, “Distance metric learning: A comprehensive survey,” Michigan State University 2(2), p. 4, each of which is hereby incorporated by reference) as illustrated in FIG. 12A.

In some embodiments the plurality of sets of pathways comprises 1, 2, 3, 4, 5, 6, 7 or all 8 pathway libraries in the group consisting of ‘WikiPathways_2019_Human’, ‘KEGG_2021_Human’, ‘GO_Biological_Process_2023’, ‘GO_Cellular_Component_2023’, ‘Reactome_2022’, ‘Metabolomics_Workbench_Metabolites_2022’, ‘BioCarta_2016’ and ‘Panther_2016’.

In some embodiments the plurality of sets of pathways comprises 1, 2, 3, 4, 5, 6, 7 or all 8 pathway libraries in the group consisting of (i) any 10, 20, 30, or 40 or more pathways in ‘WikiPathways_2019_Human’, (ii) any 10, 20, 30, or 40 or more pathways in ‘KEGG_2021_Human’, (iii) any 10, 20, 30, or 40 or more pathways in ‘GO_Biological_Process_2023’, (iv) any 10, 20, 30, or 40 or more pathways in ‘GO_Cellular_Component_2023’, (v) any 10, 20, 30, or 40 or more pathways in ‘Reactome_2022’, (vi) any 10, 20, 30, or 40 or more pathways in ‘Metabolomics_Workbench_Metabolites_2022’, (vii) any 10, 20, 30, or 40 or more pathways in ‘BioCarta_2016’ and (viii) any 10, 20, 30, or 40 or more pathways in ‘Panther_2016.’

In some embodiments, a set of pathways is any library of sets of genes, regardless of whether each set of genes in the library is formally in a pathway or not. As such, in some embodiments, the plurality of sets of pathways comprises any 2, 3, 4, 5, 6, 7, 8, 9, 10, 11, 12, 13, 15, 16, 17, 18, 19, 20, 21, 22, 23, 24, 25, or 26 sets of genes set forth in Table 5. In some embodiments, all or a portion of such sets of genes in the selected sets of genes of Table 5 is used. More detail on the libraries of sets of genes listed in Table 5 is found in Fang et al., 2022, GSEApy: a comprehensive package for performing gene set enrichment analysis in Python, Bioinformatics, 2022, btac757, doi.org/10.1093/bioinformatics/btac757, which is hereby incorporated by reference.

TABLE 5
Example libraries of sets of genes
Name of Library of sets of genes

ARCHS4_Cell-lines
ARCHS4_IDG_Coexp
ARCHS4_Kinases_Coexp
ARCHS4_TFs_Coexp
ARCHS4_Tissues
Achilles_fitness_decrease
Achilles_fitness_increase
Aging_Perturbations_from_GEO_down
Aging_Perturbations_from_GEO_up
Allen_Brain_Atlas_10x_scRNA_2021
Allen_Brain_Atlas_down
Allen_Brain_Atlas_up
Azimuth_Cell_Types_2021
BioCarta_2013
BioCarta_2015
BioCarta_2016
BioPlanet_2019
BioPlex_2017
CCLE_Proteomics_2020
CORUM
COVID-19_Related_Gene_Sets
COVID-19_Related_Gene_Sets_2021
Cancer_Cell_Line_Encyclopedia
CellMarker_Augmented_2021
ChEA_2013
ChEA_2015
ChEA_2016
Chromosome_Location
Chromosome_Location_hg19
ClinVar_2019
DSigDB
Data_Acquisition_Method_Most_Popular_Genes
DepMap_WG_CRISPR_Screens_Broad_CellLines_2019
DepMap_WG_CRISPR_Screens_Sanger_CellLines_2019
Descartes_Cell_Types_and_Tissue_2021
DisGeNET
Disease_Perturbations_from_GEO_down
Disease_Perturbations_from_GEO_up
Disease_Signatures_from_GEO_down_2014
Disease_Signatures_from_GEO_up_2014
DrugMatrix
Drug_Perturbations_from_GEO_2014
Drug_Perturbations_from_GEO_down
Drug_Perturbations_from_GEO_up
ENCODE_Histone_Modifications_2013
ENCODE_Histone_Modifications_2015
ENCODE_TF_ChIP-seq_2014
ENCODE_TF_ChIP-seq_2015
ENCODE_and_ChEA_Consensus_TFs_from_ChIP-X
ESCAPE
Elsevier_Pathway_Collection
Enrichr_Libraries_Most_Popular_Genes
Enrichr_Submissions_TF-Gene_Coocurrence
Enrichr_Users_Contributed_Lists_2020
Epigenomics_Roadmap_HM_ChIP-seq
GO_Biological_Process_2013
GO_Biological_Process_2015
GO_Biological_Process_2017
GO_Biological_Process_2017b
GO_Biological_Process_2018
GO_Biological_Process_2021
GO_Cellular_Component_2013
GO_Cellular_Component_2015
GO_Cellular_Component_2017
GO_Cellular_Component_2017b
GO_Cellular_Component_2018
GO_Cellular_Component_2021
GO_Molecular_Function_2013
GO_Molecular_Function_2015
GO_Molecular_Function_2017
GO_Molecular_Function_2017b
GO_Molecular_Function_2018
GO_Molecular_Function_2021
GTEx_Aging_Signatures_2021
GTEx_Tissue_Expression_Down
GTEx_Tissue_Expression_Up
GWAS_Catalog_2019
GeneSigDB
Gene_Perturbations_from_GEO_down
Gene_Perturbations_from_GEO_up
Genes_Associated_with_NIH_Grants
Genome_Browser_PWMs
HDSigDB_Human_2021
HDSigDB_Mouse_2021
HMDB_Metabolites
HMS_LINCS_KinomeScan
HomoloGene
HuBMAP_ASCT_plus_B_augmented_w_RNAseq_Coexpression
HumanCyc_2015
HumanCyc_2016
Human_Gene_Atlas
Human_Phenotype_Ontology
InterPro_Domains_2019
Jensen_COMPARTMENTS
Jensen_DISEASES
Jensen_TISSUES
KEA_2013
KEA_2015
KEGG_2013
KEGG_2015
KEGG_2016
KEGG_2019_Human
KEGG_2019_Mouse
KEGG_2021_Human
Kinase_Perturbations_from_GEO_down
Kinase_Perturbations_from_GEO_up
L1000_Kinase_and_GPCR_Perturbations_down
L1000_Kinase_and_GPCR_Perturbations_up
LINCS_L1000_Chem_Pert_down
LINCS_L1000_Chem_Pert_up
LINCS_L1000_Ligand_Perturbations_down
LINCS_L1000_Ligand_Perturbations_up
Ligand_Perturbations_from_GEO_down
Ligand_Perturbations_from_GEO_up
MCF7_Perturbations_from_GEO_down
MCF7_Perturbations_from_GEO_up
MGI_Mammalian_Phenotype_2013
MGI_Mammalian_Phenotype_2017
MGI_Mammalian_Phenotype_Level_3
MGI_Mammalian_Phenotype_Level_4
MGI_Mammalian_Phenotype_Level_4_2019
MGI_Mammalian_Phenotype_Level_4_2021
MSigDB_Computational
MSigDB_Hallmark_2020
MSigDB_Oncogenic_Signatures
Microbe_Perturbations_from_GEO_down
Microbe_Perturbations_from_GEO_up
Mouse_Gene_Atlas
NCI-60_Cancer_Cell_Lines
NCI-Nature_2015
NCI-Nature_2016
NIH_Funded_PIs_2017_AutoRIF_ARCHS4_Predictions
NIH_Funded_PIs_2017_GeneRIF_ARCHS4_Predictions
NIH_Funded_PIs_2017_Human_AutoRIF
NIH_Funded_PIs_2017_Human_GeneRIF
NURSA_Human_Endogenous_Complexome
OMIM_Disease
OMIM_Expanded
Old_CMAP_down
Old_CMAP_up
Orphanet_Augmented_2021
PPI_Hub_Proteins
PanglaoDB_Augmented_2021
Panther_2015
Panther_2016
Pfam_Domains_2019
Pfam_InterPro_Domains
PheWeb_2019
PhenGenI_Association_2021
Phosphatase_Substrates_from_DEPOD
ProteomicsDB_2020
RNA-Seq_Disease_Gene_and_Drug_Signatures_from_GEO
RNAseq_Automatic_GEO_Signatures_Human_Down
RNAseq_Automatic_GEO_Signatures_Human_Up
RNAseq_Automatic_GEO_Signatures_Mouse_Down
RNAseq_Automatic_GEO_Signatures_Mouse_Up
Rare_Diseases_AutoRIF_ARCHS4_Predictions
Rare_Diseases_AutoRIF_Gene_Lists
Rare_Diseases_GeneRIF_ARCHS4_Predictions
Rare_Diseases_GeneRIF_Gene_Lists
Reactome_2013
Reactome_2015
Reactome_2016
SILAC_Phosphoproteomics
SubCell_BarCode
SysMyo_Muscle_Gene_Sets
TF-LOF_Expression_from_GEO
TF_Perturbations_Followed_by_Expression
TG_GATES_2020
TRANSFAC_and_JASPAR_PWMs
TRRUST_Transcription_Factors_2019
Table_Mining_of_CRISPR_Studies
TargetScan_microRNA
TargetScan_microRNA_2017
Tissue_Protein_Expression_from_Human_Proteome_Map
Tissue_Protein_Expression_from_ProteomicsDB
Transcription_Factor_PPIs
UK_Biobank_GWAS_v1
Virus-Host_PPI_P-HIPSTer_2020
VirusMINT
Virus_Perturbations_from_GEO_down
Virus_Perturbations_from_GEO_up
WikiPathway_2021_Human
WikiPathways_2013
WikiPathways_2015
WikiPathways_2016
WikiPathways_2019_Human
WikiPathways_2019_Mouse
dbGaP
huMAP
lncHUB_lncRNA_Co-Expression
miRTarBase_2017]

In one example of this, eight separate pathway labels were generated for each compound in the plurality of compounds. In some embodiments a pathway enrichment analysis based on gseapy.enrichr was performed with the following libraries of sets of genes from Table 5: WikiPathways_2019_Human, KEGG_2021_Human, GO_Biological_Process_2023, GO_Cellular_Component_2023, Reactome_2022, Metabolomics_Workbench_Metabolites_2022, BioCarta_2016 and Panther_2016. For each sample, identified by a (compound ID, plate ID) pair, the enrichr analysis was executed across all eight libraries of sets of genes. In this analysis, enrichr with the top 512 differentially expressed genes for every sample (perturbed with the compound versus DMSO control) to standardize the compound signatures to a constant length of 512 across all compounds. A table in the format of a (compound ID, plate ID, library, pathway, regulation score) was then generated for all samples and libraries. The table was subsetted to each library individually, that is (compound ID, plate ID, pathway, regulation score) and any compounds with biological replicates across plates was further aggregated to calculate the average regulation score. Next, a set of pathways (genes) and samples in each table were filtered per library. Only those pathways comprising 10 to 160 genes were retained in this example. Only those compounds that regulate (p-value <0.3) an adequate number of pathways were included (here: at least 5% of total number of pathways after filtering by size) in this example. Filtered compounds were labeled as missing, and thus did not contribute to the loss function. Pathways (sets of genes) that were regulated in less than 10% of the samples were removed. The objective was to construct a dense matrix of compound-by-pathway activations for clustering purposes. The filtered table was then reshaped to a matrix X with rows representing compound IDs and columns indicating pathway activation scores. Compounds were then clustered to group those with similar pathway (sets of genes) activities for each library of sets of genes. The clustering procedure involves standard scaling of X to ensure all features have a zero mean, running principal component analysis (PCA) to reduce X to a 32 dimensional dataset, and employing AffinityPropagation on the PCA-transformed data. This yields a cluster label for each compound, except for the ones previously labelled as missing that do not contribute to the model training. This procedure is repeated for every library of sets of genes, resulting in eight labels for every compound that are used in eight losses (in this example) in addition to the main model loss. In doing so, the goal is to bring compound signatures from the same cluster closer together in the latent space while spreading or repulsing samples from different clusters. For example, if there are two compounds (A and B), inducing similar biology (regulating a shared set of pathways), if we they were to be grouped in the latent space solely based on compound ID, this would have enforced the repulsion between A and B. However, by adding an additional objective function that utilizes pathway regulation labels, A and B are allowed to be brought together.

Referring to block 1006, a transcriptional encoder 2122 is trained using the training set by a first procedure.

Referring to block 1008, the first procedure comprises inputting, for each respective compound in the plurality of compounds, the corresponding cellular constituent abundance data set of the respective compound into a transcriptional encoder 2122 comprising a second plurality of parameters 2124-1, . . . , 2124-Q, where Q is a positive integer, thereby obtaining an initial corresponding transcriptional embedding having a first dimension as illustrated is FIG. 12A. More details on the possible architectures of the transcriptional encoder 2122 and parameters 2124 is found in blocks 890 through 896.

Referring to block 1010, the first procedure further comprises (ii) shifting the initial corresponding transcriptional embedding for each respective compound in the plurality of compounds toward a corresponding grouping of a corresponding set of compounds in the plurality of compounds based on (a) pathway similarity between the respective compound and the corresponding set of compounds in the plurality of compounds and (b) compound identity between the respective compound and the corresponding set of compounds, thereby obtaining a corresponding calculated transcriptional embedding, for each respective compound in the plurality of compounds. This is illustrated in FIG. 12A. In FIG. 12A metric learning (e.g., Gouk et al., 2019, “Learning distance metrics for multi-label classification,” Asian Conference on machine learning, PMLR; Kayar and Bilge, 2019, “Deep Metric Learing: A Survey” Symmetry 11(9), p. 1066; and Yang et al., 2006, “Distance metric learning: A comprehensive survey,” Michigan State University 2(2), p. 4, each of which is hereby incorporated by reference) is used to adjust the initial embedding to the corresponding calculated transcriptional embedding (labeled biologically adjusted embedding in FIG. 12A). In doing so, one goal is to bring initial embeddings that are in the same pathway clusters closer together in the latent space while spreading or repulsing initial embedding from different pathway clusters. For example, if there are two compounds (A and B), inducing similar biology (regulating a shared set of pathways) and thus in the same pathway clusters (and thus have the same pathway labels in the metric learning), if they were to be grouped in the latent space solely based on compound ID (that is brought together because they have the same compound ID in accordance with one aspect of the metric learning), this would have enforced the repulsion between A and B. However, by adding an additional objective function that utilizes pathway regulation labels, A and B are allowed to be brought together. Thus, through the metric learning, initial embeddings in accordance with FIG. 12A that have the same pathway clustering labels are brought closer together through the metric learning while initial embeddings in accordance with FIG. 12A that have different pathway clustering labels are repulsed (brought further apart) in forming the final corresponding calculated transcriptional embedding. Moreover, through the metric learning, initial embeddings in accordance with FIG. 12A that have the same compound ID are brought closer together through the metric learning while initial embeddings in accordance with FIG. 12A that have different compounds IDs are repulsed (brought further apart) in forming the final corresponding calculated transcriptional embedding.

Referring to block 1012, in some embodiments the first procedure further comprises: (iii) updating the second plurality of parameters of the transcriptional encoder through application of one or more loss functions to a differential between the corresponding calculated transcriptional embedding (labeled biological adjusted embedding in FIG. 12A) and the cellular constituent abundance dataset for each respective compound in the plurality of compounds.

In one example, the transcriptional encoder's 2122 architecture is a MLP that projects 14,649 genes into a 96-dimensional space. This process is accompanied by multiple optimization tasks such as predicting compound IDs and pathways cluster labels across the eight pathway libraries.

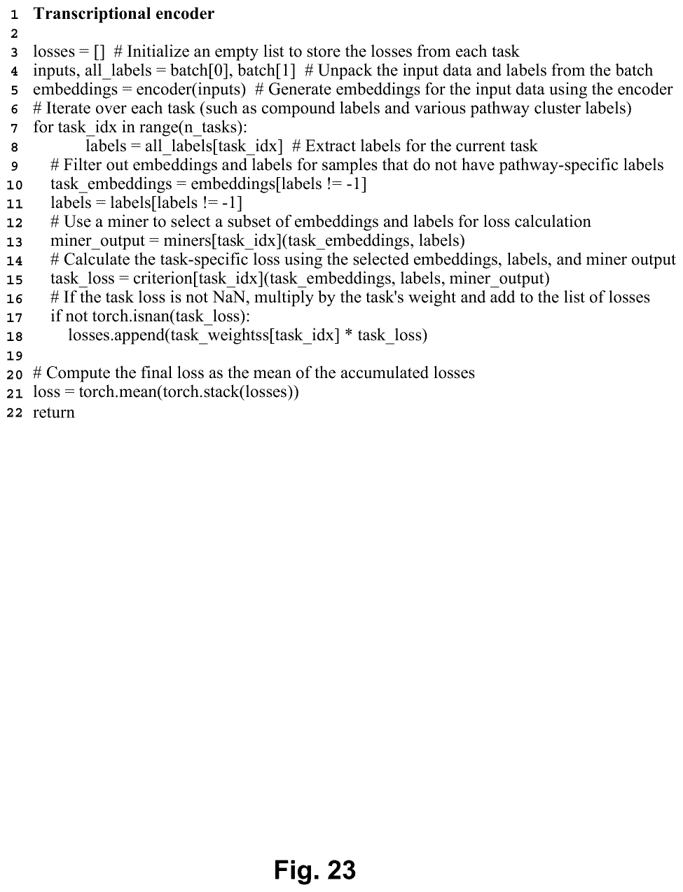

FIG. 23 provides one example of the first procedure described above. A selection of m samples per class is targeted to construct diverse sets of triplets. At each step, a sampler selects N classes (where N=batch_size//m), where classes are individual compounds labels. Next, the sampler selects m samples from each of the selected classes and forms a batch of X=[batch_size, input_size] and Y=[batch_size, n_tasks]. This constitutes a training step. FIG. 23 illustrates what occurs at each such training step. An empty container is prepared to hold the calculated losses from multiple optimization tasks (FIG. 23, line 4). The batch of data for the training step, which contains both the input features and the corresponding labels for each task (such as compound labels and various pathway cluster labels), is then processed. Subsequently, the input data is transformed into a latent space using the model's encoder (FIG. 23, line 5). The embedding is a lower-dimensional representation of the original input, capturing the needed essential features.

For each task (FIG. 23, line 7), e.g. identifying compound labels or clustering based on pathway activations, only those samples that have relevant labels are used. Samples that lack specific pathway labels are not used because they cannot contribute to the task's loss calculation (FIG. 23, lines 8-11).

Next, a mining procedure is used for each task (FIG. 23, lines 12-13). This involves selecting a strategic subset of samples and labels that will be most informative for learning.

With this refined set, the task-specific loss is calculated (FIG. 23, lines 14-18). The importance of each task's loss is adjusted by a predetermined weight, reflecting strategic emphasis on certain tasks over others. For instance, in some embodiments, compound label prediction is more critical, and therefore its loss is given more weight.

After evaluating all tasks, the weighted losses are compiled into a single measure. (FIG. 23, lines 20-21). This measure, the average loss, guides the transcriptional encoder's 2122 parameter adjustments to reduce errors across all tasks.

Finally, this average loss is returned (FIG. 23, line 22), which the transcriptional encoder 2122 will use to update its parameters (e.g., through gradient descent techniques, back-propagation techniques, etc.), gradually improving its predictions over successive training steps. Non-limiting examples of stochastic gradient descent techniques are disclosed in Zeiler, 2012 “ADADELTA: an adaptive learning rate method,” CoRR, vol. abs/1212.5701, which is hereby incorporated by reference. Non-limiting examples of back-propagation techniques are disclosed in Rumelhart et al., 1988, “Neurocomputing: Foundations of research,” ch. Learning Representations by Back-propagating Errors, pp. 696-699, Cambridge, MA, USA: MIT Press, which is hereby incorporated by reference. This iterative process aims to create a transcriptional encoder 2122 that not only identifies compounds accurately but also understands the biological pathways they influence, ultimately positioning similar compounds near each other in the latent space.

Referring to block 1014 of FIG. 10B, a structure encoder 2112 is then trained by a second procedure using the training set. The structure encoder 2112 comprises a first plurality of parameters 2114-1, . . . , 2114-N, where N is a positive integer. Non-limiting examples of structure encoder 2122 architectures and parameters 2114 in accordance with the present disclosure are detailed in blocks 848 through 854. In some embodiments, the second encoder is already pre-trained on the structure of hundreds, thousands, or millions of compounds before implementing the second procedure. The goal of the second procedure is to align the embedding space of this previously trained structure encoder with that of the transcriptional encoder.

Referring to block 1016, in some embodiments the second procedure comprises: inputting, for each respective compound in the plurality of compounds, a combination of a feature representation 2108 of the respective compound and a baseline transcriptional representation 2110 of a first cell type into the structure encoder 2112 thereby obtaining a corresponding compound embedding 2116 having the first dimension. Blocks 802 through 846 provide nonlimiting details of compound feature representations 2108 and baseline transcriptional representation 2110 and how they are constructed in accordance with some embodiments of the present disclosure.

Referring to block 1018, in some embodiments the second procedure further comprises updating the first plurality of parameters through minimization of a loss function applied to a differential between (a) the corresponding compound embedding for the respective compound from the structure encoder 2112 and (b) the corresponding calculated transcriptional embedding for the respective compound from the transcriptional encoder 2122.

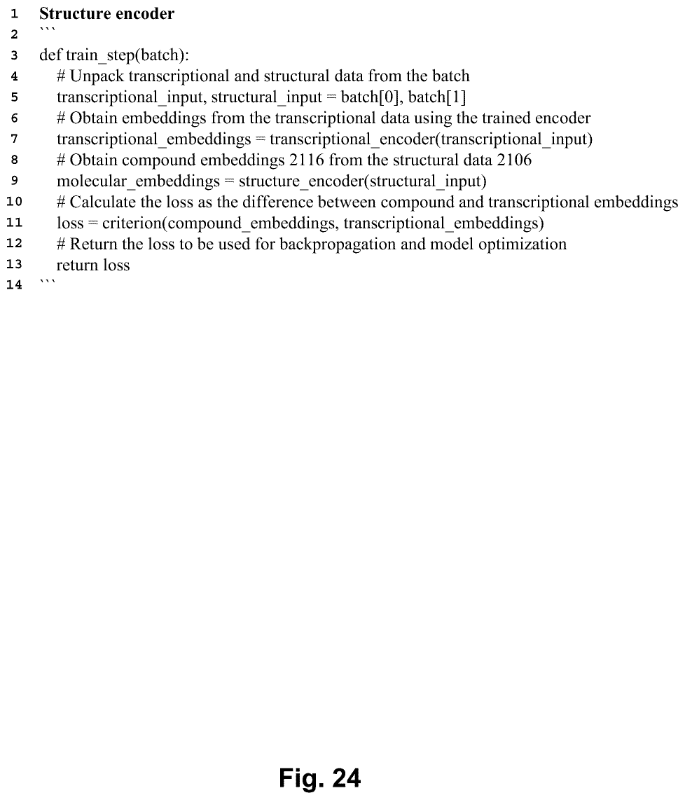

FIG. 24 provides one example of the second procedure described above. In some embodiments, the structure encoder's 2112 architecture is a multilayer perceptron. However, in other embodiments the architecture of the structure encoder 2112 takes a different form as described in blocks 848-854. The structure encoder 2112 addresses a multi-task regression challenge. Similar to the transcriptional encoder 2122, in some embodiments the structure encoder's architecture, optimizer, and loss function are fine-tuned through Optuna (Akiba et al., 2019, “Optuna: A Next-generation Hyperparameter Optimization Framework,” arXiv:1907.10902 [cs.LG], which is hereby incorporated by reference). However, for the structure encoder 2112 model training, the focus is on maximizing reconstruction fidelity rather than recall. Thus the goal of the structure encoder 2112 training in accordance with some embodiments of the present disclosure is to match the compound embedding 2116 of a compound to its matching (corresponding) transcriptional embedding 2128 as closely as possible.

The objective in accordance with the example of FIG. 24 is to reconstruct the transcriptional embedding 2128 (e.g., in the form of a latent space vector), derived from transcriptional data through the trained transcriptional encoder 2122, using only the molecular structure of the compound (and the baseline transcriptional representation 2110). In the example of FIG. 24, to represent the molecules, the molfeat package was employed to generate molecular features using the following methods: gin_supervised_edgepred, ECFP:4, desc2D, and MACCS. These methods yield molecular features of sizes 300, 2000, 211, and 167, respectively. A comprehensive molecular signature is then created by concatenating these features into a single vector with a length of 2678. Blocks 808 through 814 disclose additional methods for featurizing the molecule and block 816 discloses additional ranges of the number of features the feature representation of the molecule may have.

To incorporate cellular context, a cell type-specific representation (base line transcriptional representation 2110) is appended to this molecular signature, resulting in a final vector of 2935 elements (an example of an input data structure 210). Block 818 describes methods for determining this baseline transcriptional representation 2110.

In the training phase, a batch of samples is randomly selected, this time without the constraint of equal class representation. Each sample comprises both transcriptional data and structure (molecular features augmented with cell type features) data (FIG. 24, lines 1-5). The transcriptional data is processed through the transcriptional encoder 2122 to produce transcriptional embeddings 218 (FIG. 24, lines 6-7). Concurrently, the molecular (compound) input (input data structure 2106) is passed through the structure encoder 2112 to generate compound embeddings 2116 (FIG. 24, lines 8-9). In the example of FIG. 24, a training L1 loss is then computed as the discrepancy between the compound embeddings 2116 and the corresponding transcriptional embeddings 2128 (FIG. 24, line 10-11). However, other loss functions may be used. This loss quantifies how well the structure encoder 2112 can predict the corresponding transcriptional embedding 218 derived from the transcriptional encoder 2122, and the aim is to minimize this during training.

Once trained, for example as discussed above and illustrated in the example of FIG. 24, the structure encoder 2112 is able to determine a relationship between one or more biological states and the first compound upon inputting a feature representation of the first compound and a baseline transcriptional representation of a first cell type into the structure encoder 2112 and comparing the output of the structure encoder to the corresponding calculated transcriptional embeddings of the training dataset.

The co-embedding encourages chemical structures with a similar transcriptional response to group together even if they are different scaffolds. Thus, the disclosed joint structure encoder 2112 and transcriptional encoder 2122 model allows for two important capabilities: i) projection of new compounds without transcriptional readouts into the co-embedding to assess if they fall within the same cluster and thus show similar transcriptional activities and ii) utilization of training data within the cluster to estimate differentially express genes and enriched pathways for the new compounds.

### VIII. ADDITIONAL EMBODIMENTS

Another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors.

Another aspect of the present disclosure provides a non-transitory computer readable storage medium storing one or more programs configured for execution by a computer, the one or more programs comprising instructions for carrying out any of the methods disclosed herein.

### IX. EXAMPLES

### Example 1—Library of Integrated Network-Based Cellular Signatures (LINCS) Data

The Library of Integrated Network-Based Cellular Signatures (LINCS) consortium archives datasets consisting of assay results from cultured and primary human cells treated with perturbagens, e.g., bioactive small molecules, ligands such as growth factors and cytokines, or genetic perturbations. The LINCS consortium archives include data sets from many different types of assays used to monitor cell responses, providing data on transcriptional responses, protein expression responses, cell phenotypic responses measured, e.g., by biochemical and/or cellular imaging assays. In many cases, assays are performed across multiple cell lines, under multiple environmental conditions, and/or using multiple perturbagen concentrations. Accordingly, the LINCS consortium includes large-scale data on perturbation-induced molecular and cellular signatures. More information on the LINCS consortium can be found online at the URL lincsproject.org.

### Example 2—Construction and Performance of an Example Model Comprising a Structure Encoder 2112 and a Transcriptional Encoder 2122

Abstract. Prediction of drug candidates through transcriptomics relies on extensive data sets of transcriptional perturbations. Ideally, these data sets are comprised of varied small molecules with high structural and transcriptional diversity, across a variety of cell systems, to ensure comprehensive coverage of response of biological systems to interventions. As the generation of these data is very costly and hard to achieve given the almost infinite number of possible outcomes for a given intervention, machine learning methods that take advantage of both the structural and transcriptional spaces can be used to identify potential novel clinical candidates without the need to transcriptionally measure their impact. In this example a novel machine learning model, based on metric learning (e.g., Gouk et al., 2019, “Learning distance metrics for multi-label classification,” Asian Conference on machine learning, PMLR; Kayar and Bilge, 2019, “Deep Metric Learing: A Survey” Symmetry 11(9), p. 1066; and Yang et al., 2006, “Distance metric learning: A comprehensive survey,” Michigan State University 2(2), p. 4, each of which is hereby incorporated by reference), is described that learns a joint representation of both data types to predict interventions that impact disease states of interest. The model is trained on a perturbational data set, consisting of 3700 compounds with transcriptional profiles across six different cell types, to generate a transcriptional embedding. This is combined with the embedding space from a pre-trained chemical model that has been trained on millions of molecular structures. On benchmarks, the model achieved 25% hit rate in matching transcriptional readout of a molecule with the corresponding structure, on a dataset of 255 unseen molecules. By creating a transcription-structure co-embedding, the search space can confidently be expanded for hits beyond the compounds for which transcriptional data been obtained, roughly 370,000 compounds. The disclosed model holds great promise for transcriptomics-based drug discovery, both in the space of approved compounds (for a drug repurposing task) and in the space of unexplored new chemical entities.

Main. The discovery of novel therapeutics through transcriptional profile analysis can be framed as a drug repurposing exercise. Under this paradigm, known compound behaviors (as measured by transcriptional responses of a cellular system can be used to make predictions against disease states of interest. Using existing data sets and an ever-increasing number of machine learning approaches, these approaches can find novel uses for drugs in the market that can address therapeutic areas and patient populations in need of treatments. Moreover, through prediction of transcriptional profiles for unseen perturbations, small molecule selection can be achieved for compounds for which such transcriptional data is not available or two costly to obtain due to the number of compounds being screened.

In this example a novel model architecture that uses deep metric learning to predict which chemical structures will elicit a transcriptional response of interest is described. Working with biological data often presents challenges due to its inherent noise and sparsity, making it difficult to predict transcriptional responses of novel compounds or combinations accurately. This limitation hinders the potential for drug repurposing and discovery based on transcriptional data. To address this, metric learning is employed, a suitable approach for the present “few instances” learning scenario, where instances of the same class are limited. This model was trained on a library of 11220 transcriptional profiles from drug perturbations and a set of 1870 small molecule chemical structures. Both these data sets were used to generate a joint embedding that allows for the association of a transcriptional profile with the matching structures and vice versa.

Model overview. To build training data for the model, a library of 3700 compounds was generated with diverse mechanism of actions (MOAs), that is more than 1200 main targets. scRNA-seq technology was used to profile the single-cell perturbational response of these compounds 24 hours post intervention in CD34+ cells. The mRNA counts for all genes was measured. A set of known markers was used to annotate 6 well-characterized cell types within CD34+ population as described in blocks 836 through 846. Pseudobulk differential expressed genes (DEGs) analysis was then performed where cells perturbed by a compound at a cell type were merged and Limma (limma-voom pipeline, which normalizes the expression counts and runs differential expression on the normalized values; Law et al., 2018, “RNA-seq analysis as easy as 1-2-3 with limma, Glimma, and edgeR,” F1000Research 5:1408, last updated 28 Dec. 2018, which is hereby incorporated by reference) was applied to compare the compound's expression profile to DMSO control arm as described in block 860. Differential Expression Score (DES) is defined for a compound as the −log 10(qᵥₐₗᵤₑ) sign(LFC) where LFC reflects the magnitude of the expression change compared to DMSO and qᵥₐₗᵤₑ is the statistical significance of that change. Compounds that produce no DEG at 5% FDR in all cells were filtered out, resulting in 1870 compounds and 815 MOAs. A diverse coverage of pathways across active compounds' signatures, with more than 80% of KEGG pathways enriched. See FIGS. 17C and 17D and block 860. Together, a single-cell database was generated with access to full transcriptomics that covers most known pathways and diverse MOAs, serving as the training data.

As illustrated in FIG. 11A, the disclosed model in accordance with this example comprises a transcriptional encoder 2122 and a structure encoder 2112 to generate a co-embedding. The transcriptional encoder 2122 was first trained to project high-dimensional transcriptomics into an 96-dimensional latent space. The transcriptional encoder was then frozen and the structure encoder 2112 was trained so that the projection of molecular features into the same 96-dimensional latent space has the minimum L1 reconstruction loss compared to the fixed transcriptional embedding as illustrated in FIG. 18A. Effectively this results in a multimodal co-embedding that can generate embeddings both from transcriptional and structural data.

The co-embedding encourages chemical structures with a similar transcriptional response to group together even if they are different scaffolds. Thus, the disclosed model 2100 (FIG. 11A) allows for at least two important capabilities: i) projection of new compounds without transcriptional readouts into the co-embedding to assess if they fall within a same cluster and thus show similar transcriptional activities and ii) utilization of training data within the cluster to estimate DEGs and enriched pathways for the new compounds.

In this example, the transcriptional encoder 2122 took the DES of 14,649 genes as the input which captures the significance of differential expression and the direction of gene regulation produced by compounds as illustrated in FIG. 12A. A multilayer perceptron (MLP) with two hidden layers is then applied for the initial dimensionality reduction into the 96 dimensions. Next, matric learning (e.g., Gouk et al., 2019, “Learning distance metrics for multi-label classification,” Asian Conference on machine learning, PMLR; Kayar and Bilge, 2019, “Deep Metric Learing: A Survey” Symmetry 11(9), p. 1066; and Yang et al., 2006, “Distance metric learning: A comprehensive survey,” Michigan State University 2(2), p. 4, each of which is hereby incorporated by reference) is employed to reshape the initial embedding according to biological knowledge as illustrated in FIG. 12A. The pathway enrichment scores of compounds across several pathway databases (e.g. KEGG and Reactome) are calculated and compounds are clustered per each database as describe in Section VII (e.g., block 1004). Each cluster is treated as a label for the compounds in that cluster. Compound IDs are also used as another label. These labels are utilized in a weakly supervised learning to encourage i) compounds with similar pathway enrichments to group together and ii) replicates of a compound across all six cell types to be closer together compared to a different compound. Further details of this are described in Section VII. FIG. 12B shows the UMAP of the final transcriptional embedding (96 dimensions), where compounds are shaded by the cholesterol biosynthesis pathway score. Clearly, clusters of compounds with similar pathway scores are observed, indicating that the transcriptional embedding is indeed informed by prior biological information.

The structure encoder 2112 takes various molecular features of a compound as the input (FIGS. 11A and 13A). Particularly, a pre-trained GNN, 2D descriptors, Morgan Fingerprints, and MACCS were used to covert SMILES into different representations of a small molecule and combine them into a single molecular vector that goes into the encoder as described in more detail in block 808 through 816. In addition, because a compound can produce a different transcriptional signature at each cell type, stock cell type information was included in the molecular vector. Hence, the GSEA enrichment on the basal expression of cell types (e.g. DMSO) was performed and the enrichment scores was used as a representation of cell type features as described in more detail in block 860. Notably, the enrichment analysis of DMSO controls improved the separation of the cell types as illustrated in FIGS. 17A and 18B. Essentially, the molecular vector was combined with the enrichment scores and used as the input to a MLP with two hidden layers for the projection into the same 96-dimensional latent space as transcriptomics where the latent vectors of transcriptomics and structures corresponding to the same compounds were optimized to have the minimal L1 loss. In doing so, the latent space that group molecules based on biological activities was created. For instance, FIG. 13C shows that two clusters 1302 of compounds are enriched for the cholesterol biosynthesis pathway.

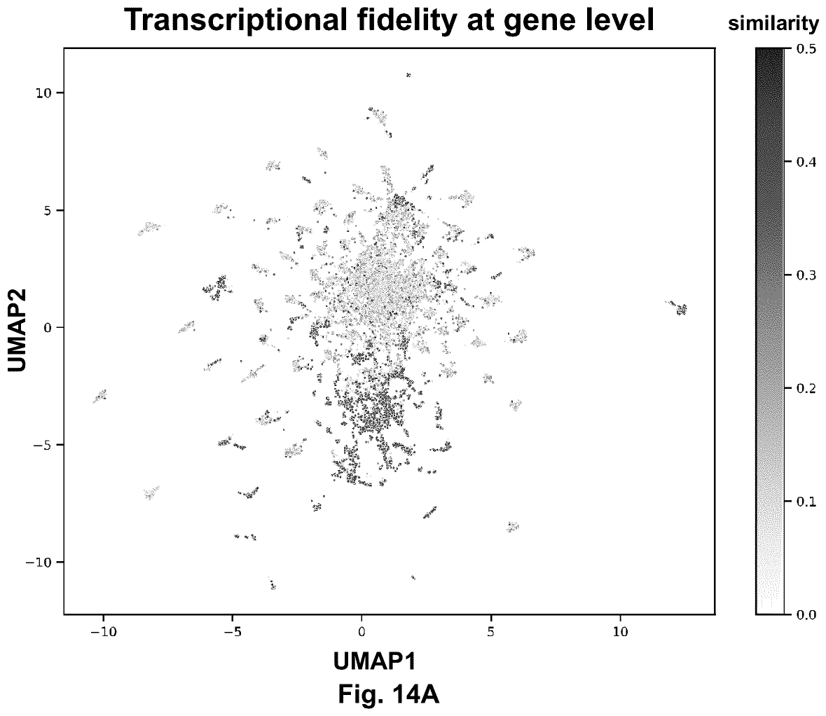

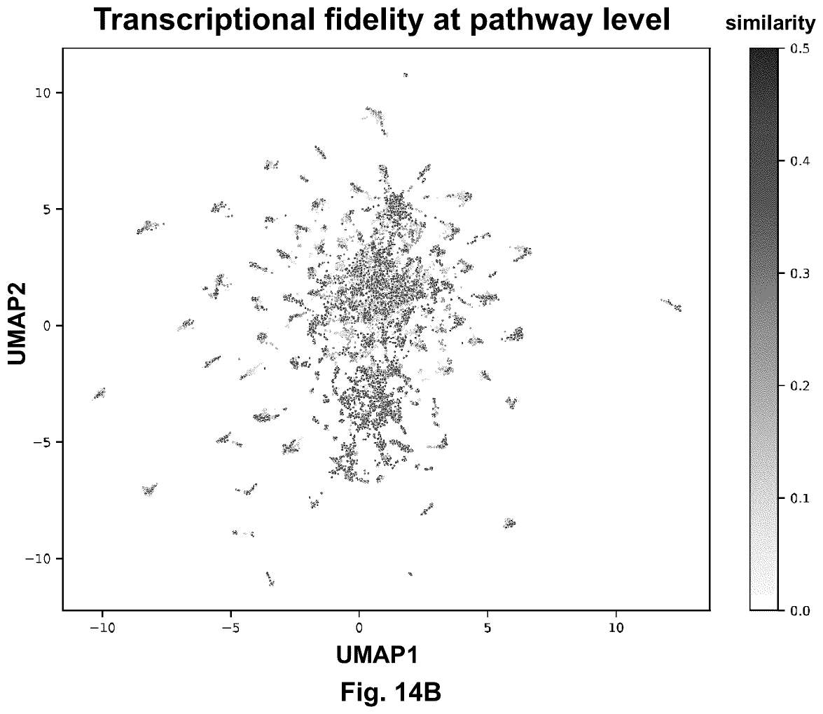

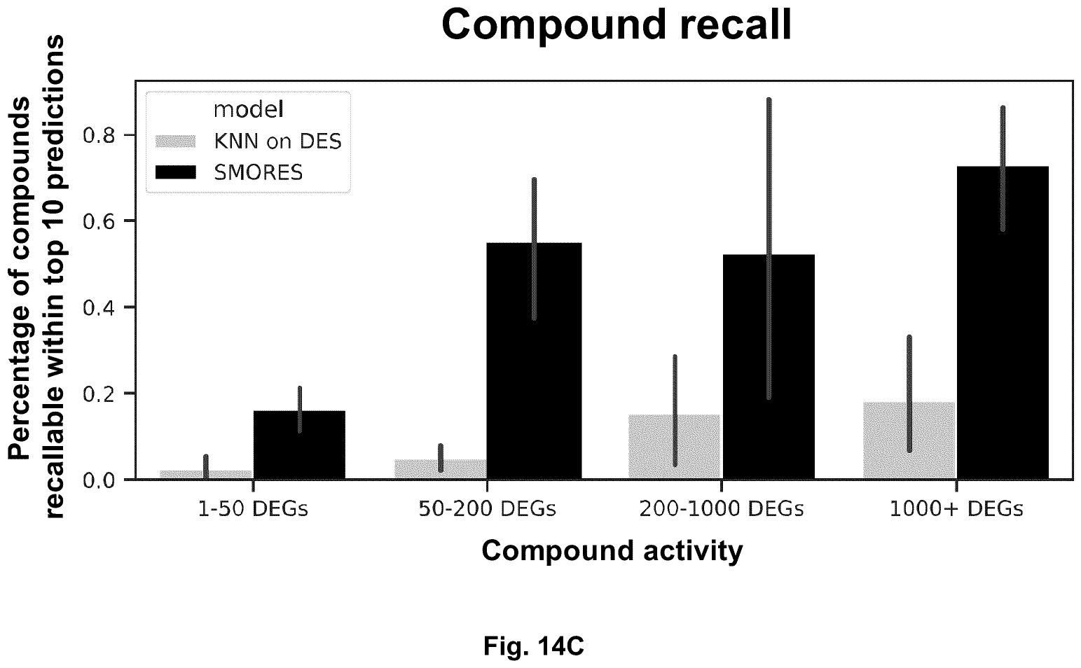

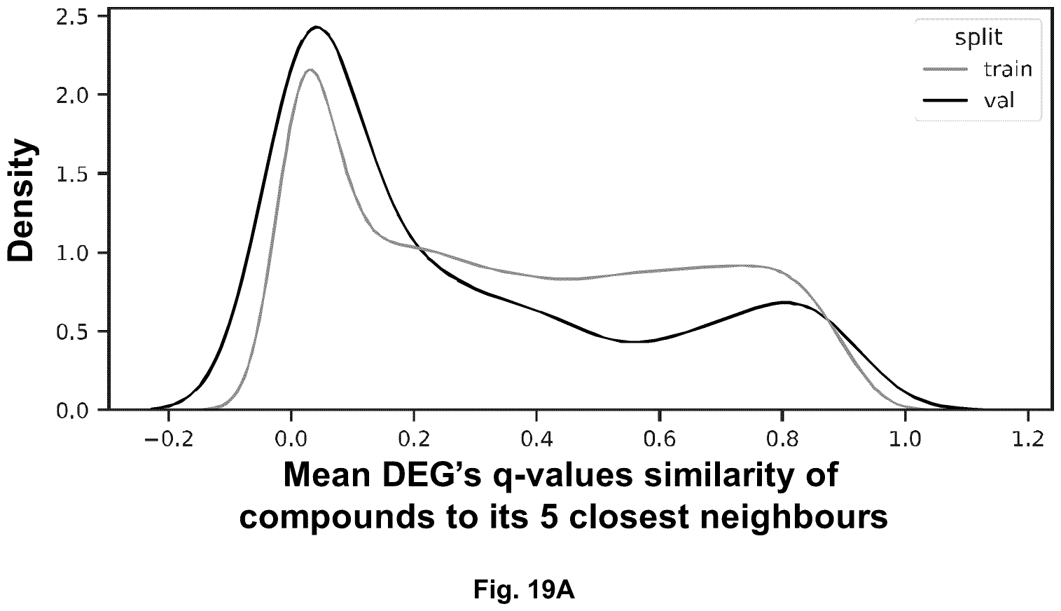

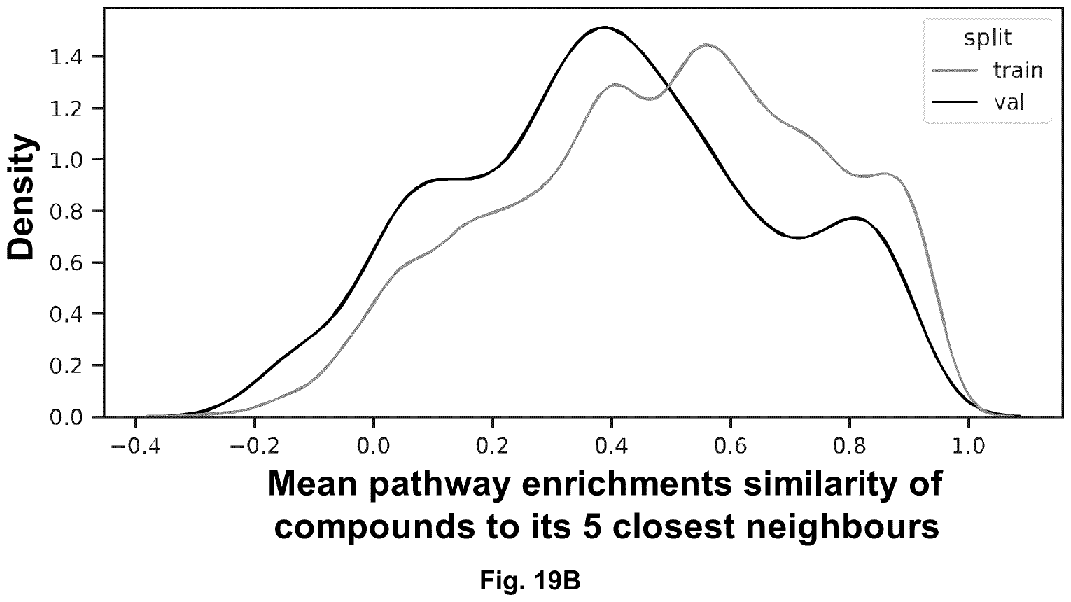

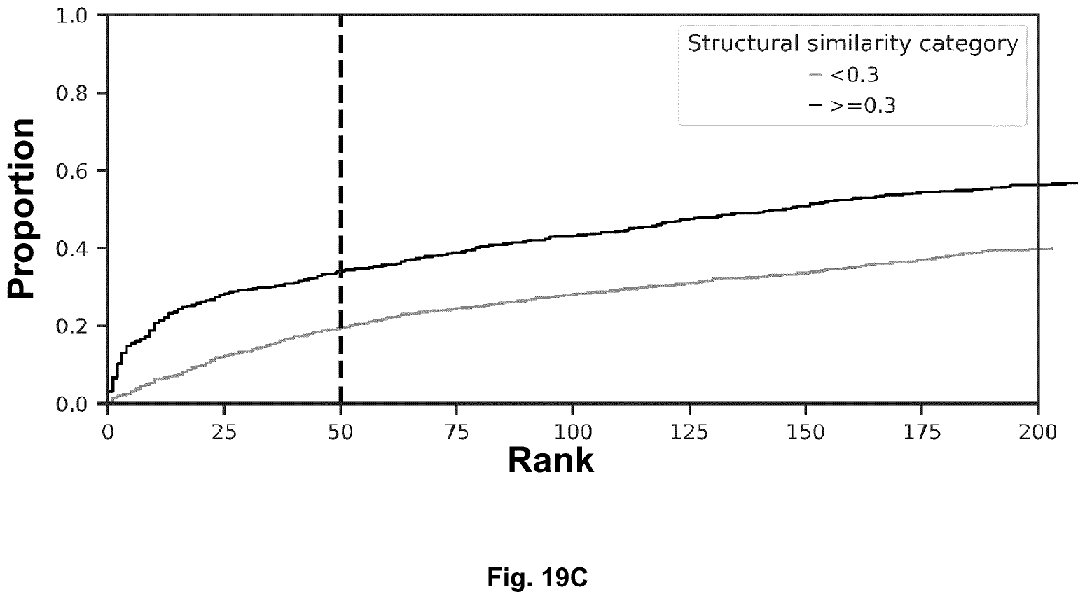

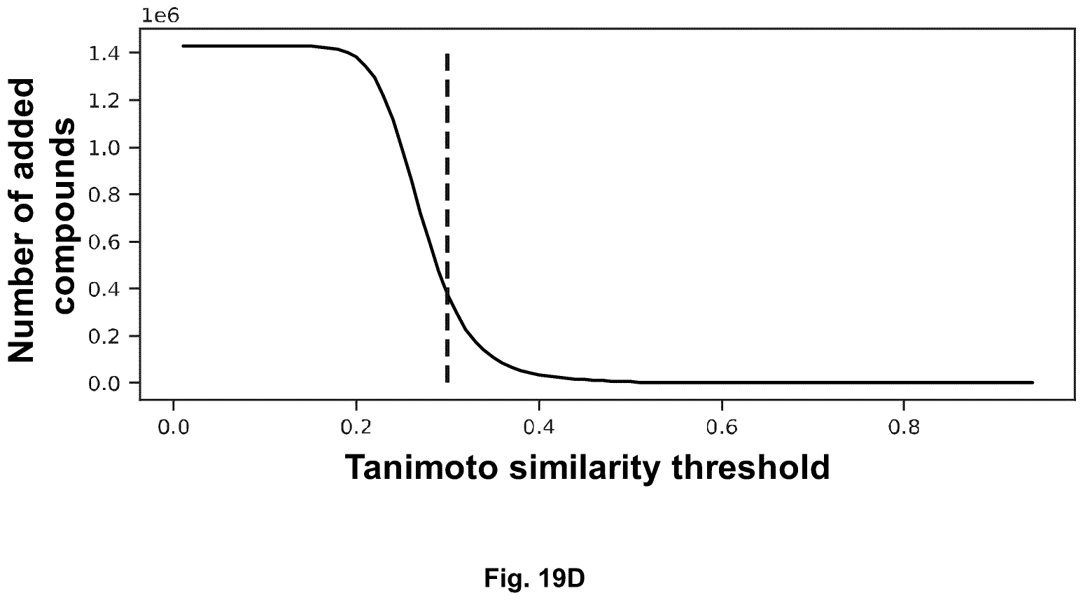

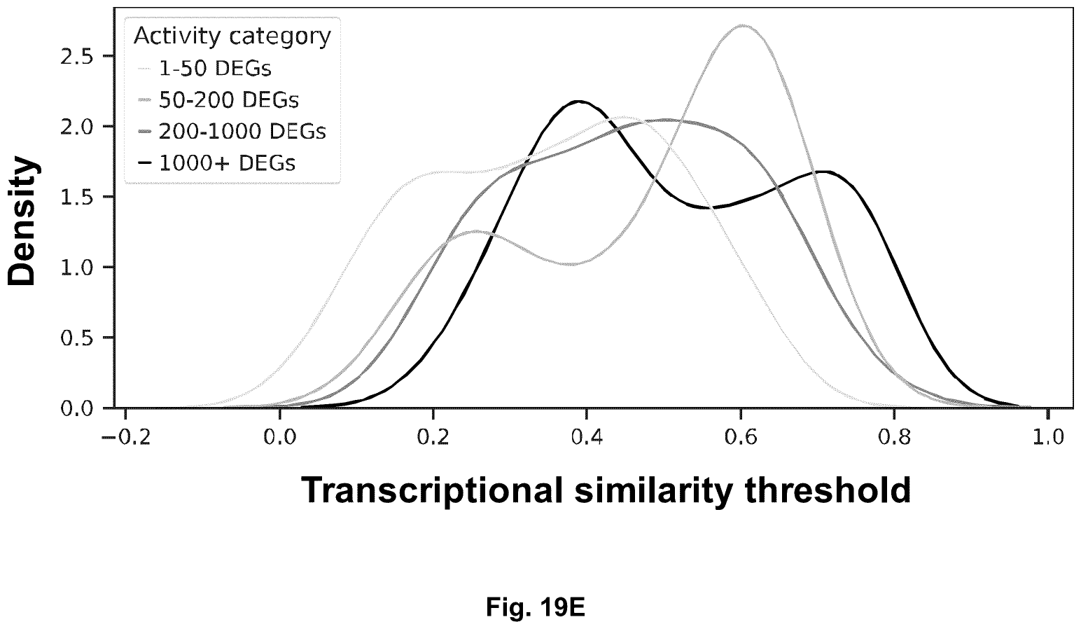

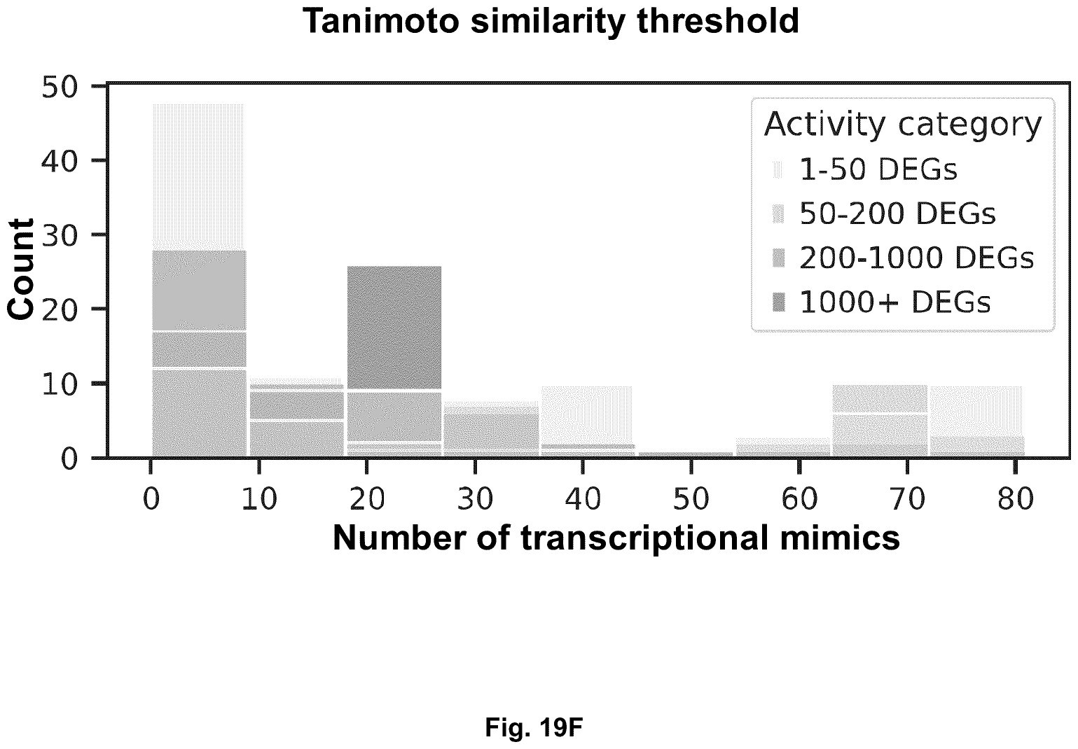

Transcriptional embedding leads to 5 fold stronger detection of compound signatures while capturing the biological relevance of perturbations. The transcriptional encoder 2122, important for creating transcriptional embeddings 2128 that are key to training the structure encoder 2112 and determining the model's overall quality, were evaluated. To achieve this, the average cosine similarity for each compound's DES with its five nearest neighbors within the latent space was computed, as shown in FIG. 14A. Significantly, over 56% of test compound signatures exhibited a cosine similarity exceeding 0.1, as detailed in Supplementary FIG. 19A. This similarity was significantly improved when pathway activation scores were utilized for the computation (up to 83%) (refer to FIG. 14B and FIG. 19B), indicating that the transcriptional encoder 2122 preserves the biological relevance of compounds. The embedding was further assessed to ensure that compound signatures can be recalled. Two KNN classifiers were trained, one using inputs from the models' embeddings, and the other using the original compound signatures (DES), where 1870 compound IDs were used as labels in both cases. Next, 136 compounds were randomly selected from the training data and perturbational responses of these compounds were generated using scRNA-seq in CD34+, that is a set of experiments independent from the training, named the validation samples. The two KNN classifiers were then run on the validation samples and a recordation was made of whether the correct class (compound ID) was predicted within the top 10 predictions out of 1870 compounds. FIG. 14C shows that the KNN trained on the embedding recalls compounds ˜5 fold higher as opposed to the original DES, a superior performance in recalling compounds across various transcriptomic activities annotated by number of DEGs at 5% FDR. Consequently, the transcriptional encoder 2122 generates a biologically meaningful latent space, which more effectively captures the compounds' biological signals compared to the original DES.

The disclosed co-embedding outperforms existing methods and accurately predicts transcriptional responses of compounds that have never seen by the model.

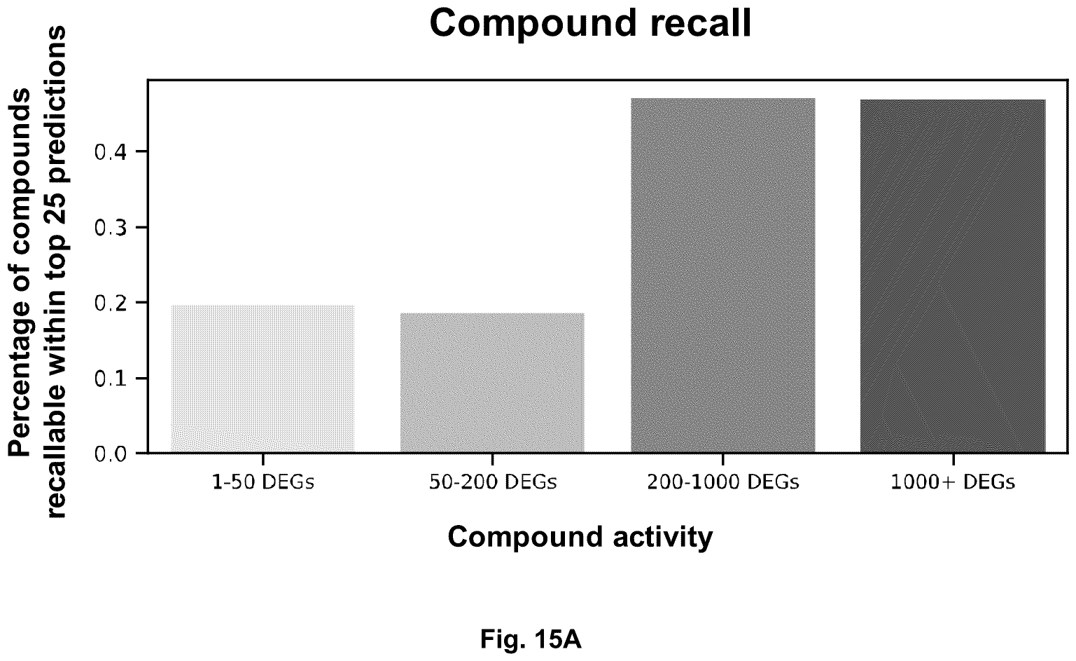

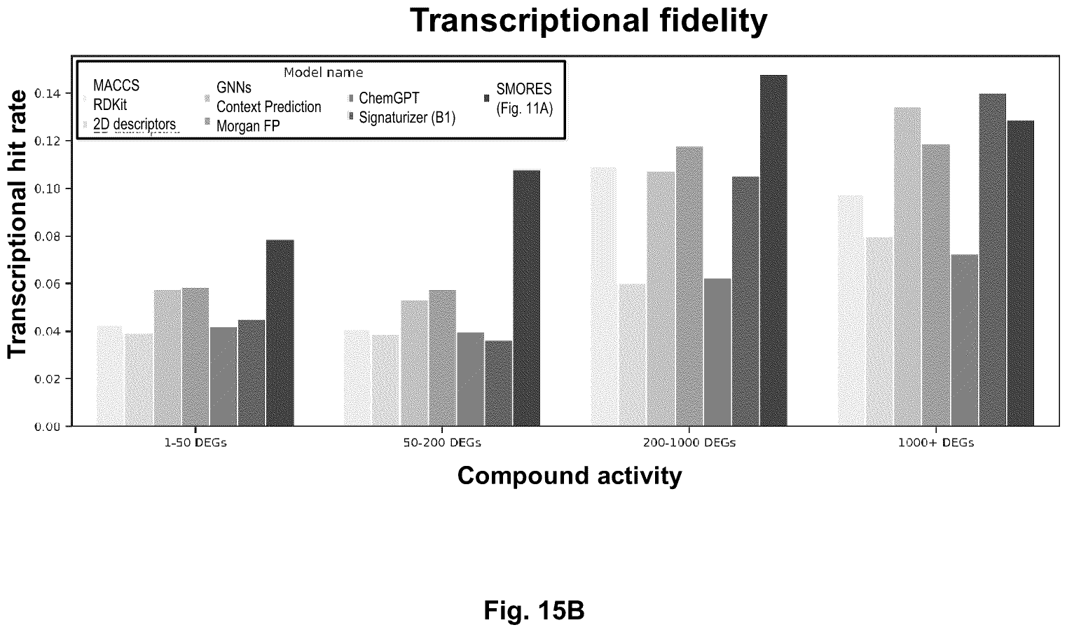

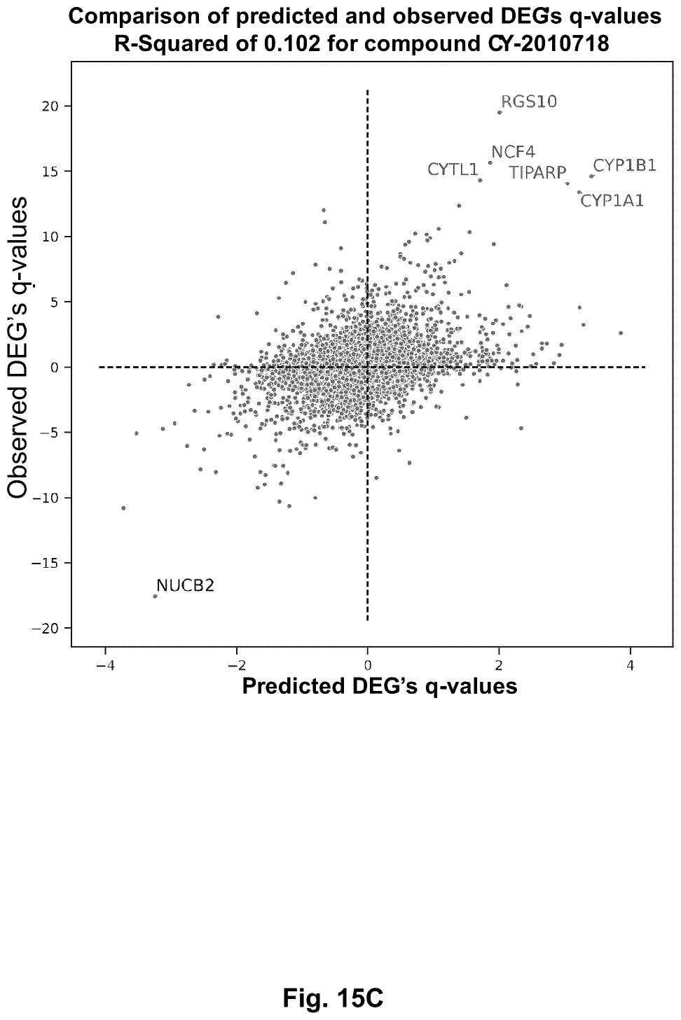

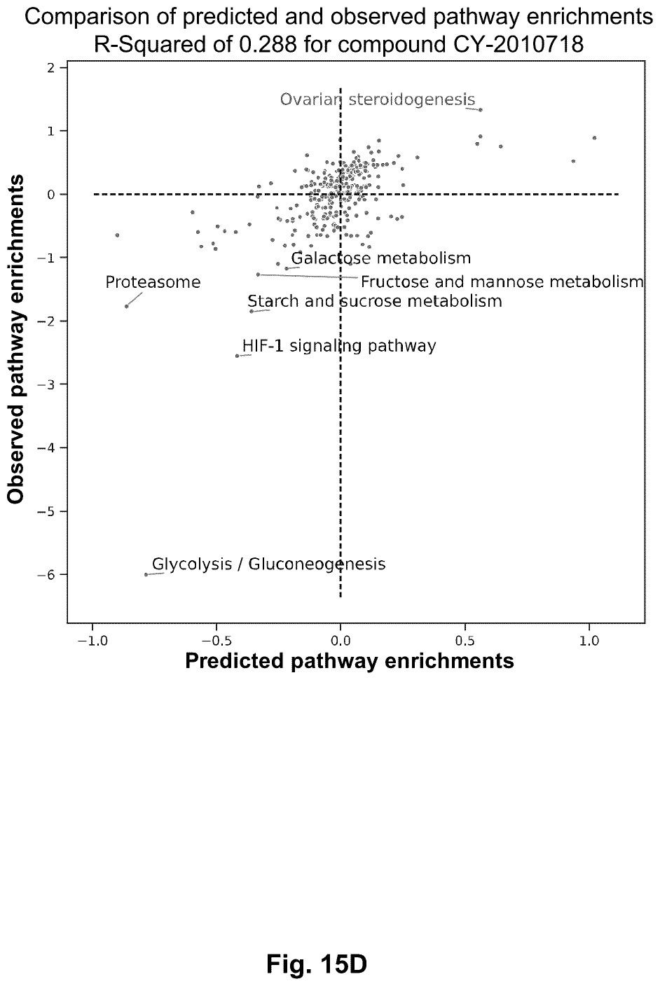

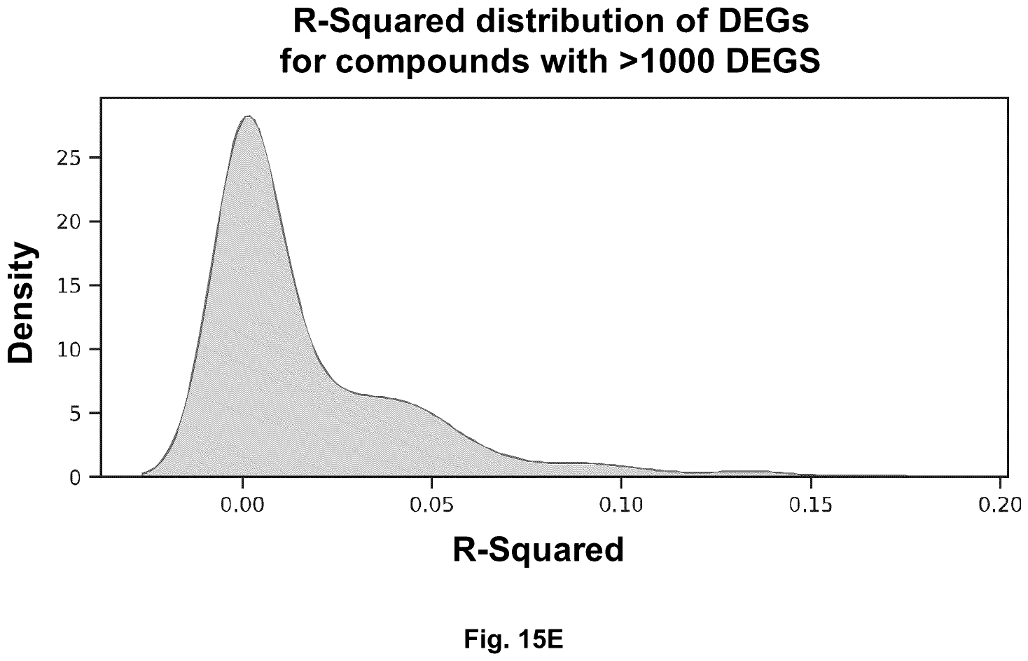

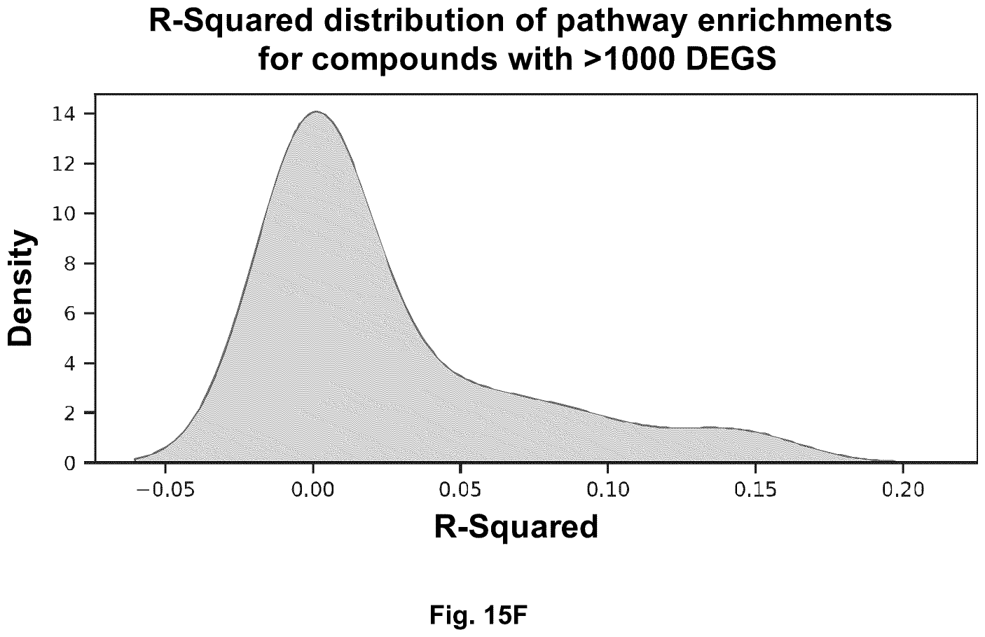

An important metric for the model's performance is its capacity to accurately map a compound's transcriptional embedding to its originating compound structure. To evaluate this, 1785 transcriptional responses were embedded (as transcriptional embedding 2128) alongside the molecular structures for all test compounds (as compound embeddings 2116) into the latent space, encompassing 255 test compounds across six distinct cell types. Next, for each test compound's transcriptional embedding, the cosine similarity with all compound embeddings in the latent space was computed, including both test and training compounds, totaling 1870 compounds. A test compound is defined as ‘recallable’ if its correct structure is identified within the top-50 nearest structures based on the similarity rankings in the co-embedded space (FIG. 15A). Notably, a variable recallability across compounds with different activity levels was detected, varying from 17% of compounds with 1-50 DEGs (106 compounds) to 37% for those with 200-1000 DEGs (32 compounds). These findings show that the disclosed model can effectively leverage transcriptional information to infer structural data, affirming the model's capability to integrate and interpret complex biological signals, although with variable rate depending on compound activity level. As expected, the quality of this matching also depended on whether the model has observed compounds similar to the test compound during training (Supplementary FIG. 19C), and the rate of correct mapping was significantly increased for the test compounds with Tanimoto similarity >=0.3 to train compounds (0.34 vs 0.19; Fisher's exact test p-value <1e-5). This allowed for the estimate that the size of the chemical space, which can be used to pair a compound with the target transcriptional readout, to be around 370,000 molecules (using bioactive molecules from ChEMBL 30 database, Supplementary FIG. 3D).

The ability of the model to project the structures of test compounds in proximity of transcriptionally similar compounds (albeit structurally diverse) was tested. First, for each of 255 test compounds, a set of transcriptional mimics among training compounds was defined, where a transcriptional mimic for a query compound (test) was defined as compounds that induce similar transcriptional responses. The ability of the disclosed model and 6 public models were tested to prioritize transcriptional mimics. Specifically, each method ranked training compounds against a query compound by embedding the query compound and all training compounds, using only structure information, and by computing a similarity in the resulting latent space. A correct pairing between the query and transcriptional mimic was recorded when the transcriptional mimic was ranked among top 50 closest compounds of its query. A hit rate was then computed as the proportion of correct pairs across all possible query-mimic pairs and the hit rate was reported across different DEG activities of query (FIG. 15B). As expected, many models produced non-zero hit rate, for example, using a simple Tanimoto similarity yields a hit rate of around 5%, which is expected, because structural analogs might induce similar transcription, especially if they inhibit the same target. Notably, model 2100 outperformed all methods in low and medium activity buckets, and for compounds with high activity our model performed similar to Signaturizer and a pretrained GNN model.

The ability of the disclosed model 2100 to predict DES of an unseen compound, given only its structure and cellular context was explored. As the disclosed architecture of the model 2100 doesn't inherently reconstruct input signatures from the latent space, a KNN regressor was trained to function as a decoder. This regressor uses the structure-based embeddings of training compounds to predict the DES for each gene. Additionally, models were created to predict pathway regulation scores. These models were then tested with the test compounds, using only their structure-based embeddings for prediction, and the results were promising. Although predicting the direction of gene regulation was challenging for many samples, as shown in FIG. 15E, the approach was generally effective FIG. 15C. Notably, predictions for pathway regulation resulted in a significant improvement in R squared values (FIG. 15D and FIG. 15F). These findings indicate that while the model 2100 accurately predicts biological processes, it also underscores the complexity of gene regulation prediction tasks.

The disclosed model identifies different scaffolds with similar transcriptional activities. To illustrate the applicability of the disclosed model 2100 in identifying different scaffolds with similar transcriptional responses, 255 of the test compounds were screened against all training compounds to explore what different structures from the training compounds can induce similar transcriptional response as the query, treating each test compound as a target for which we search the training database to identify new scaffolds. A few examples of this are provided as follows.

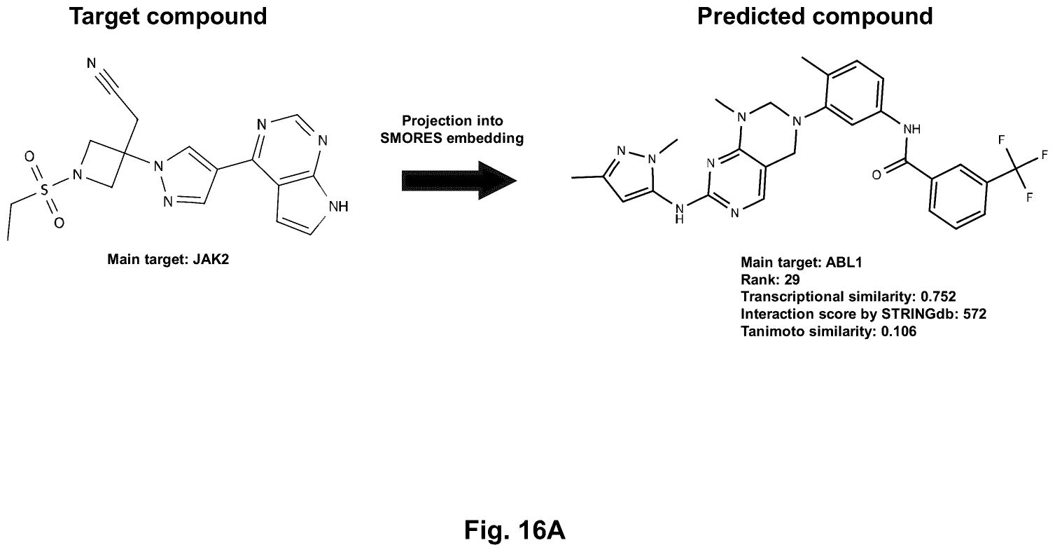

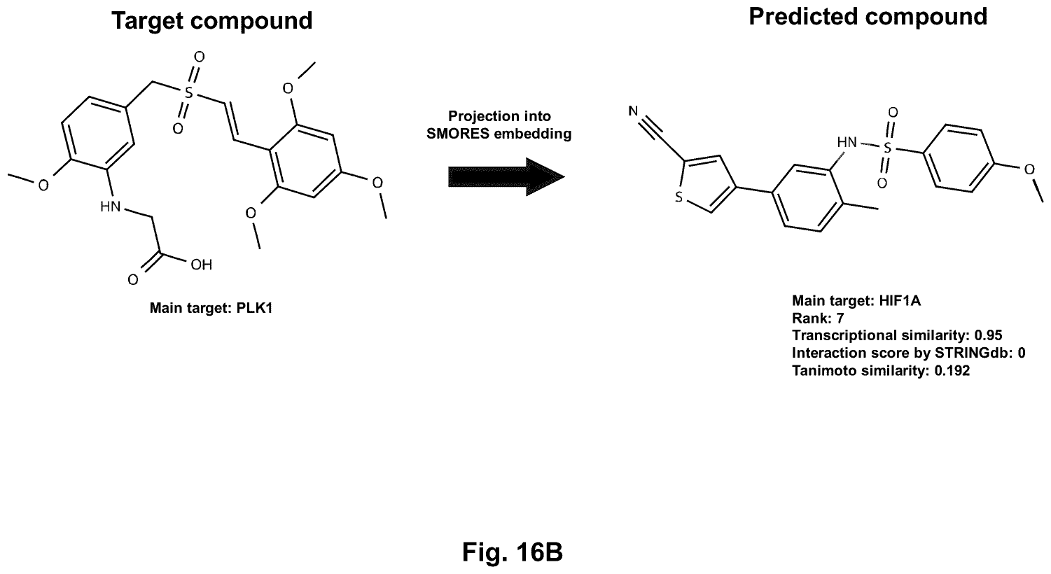

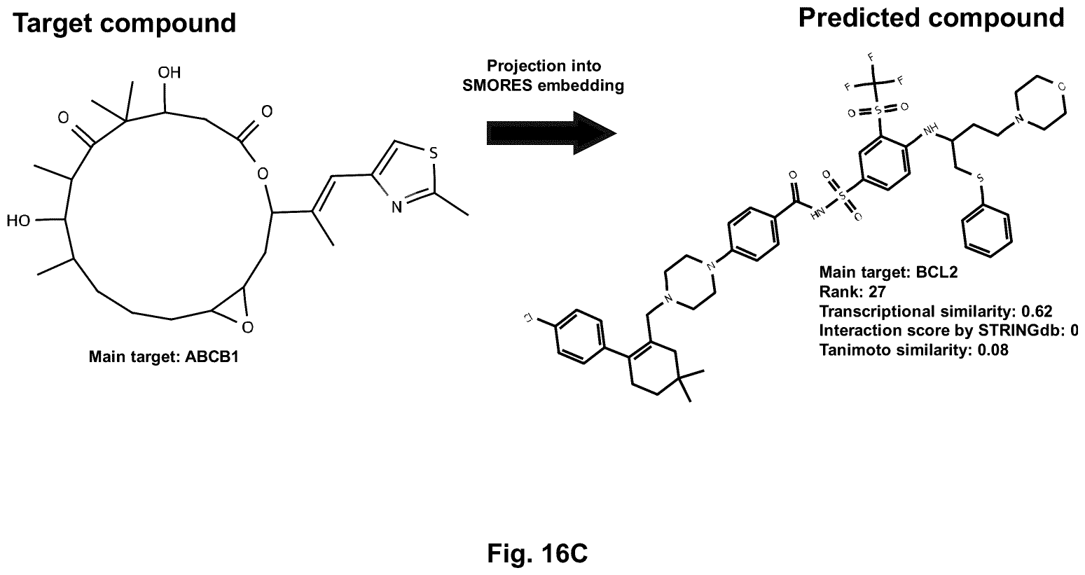

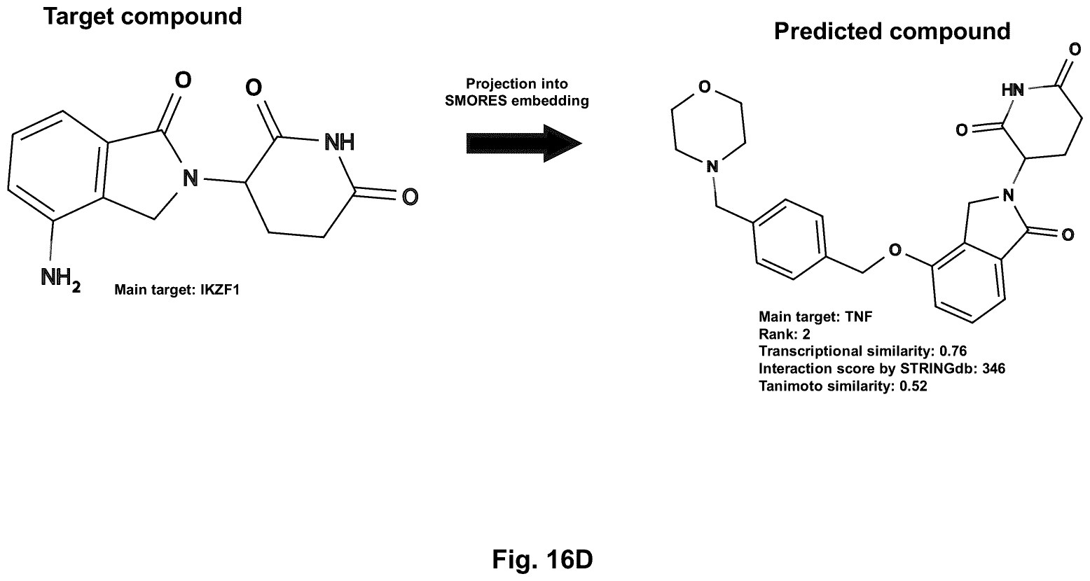

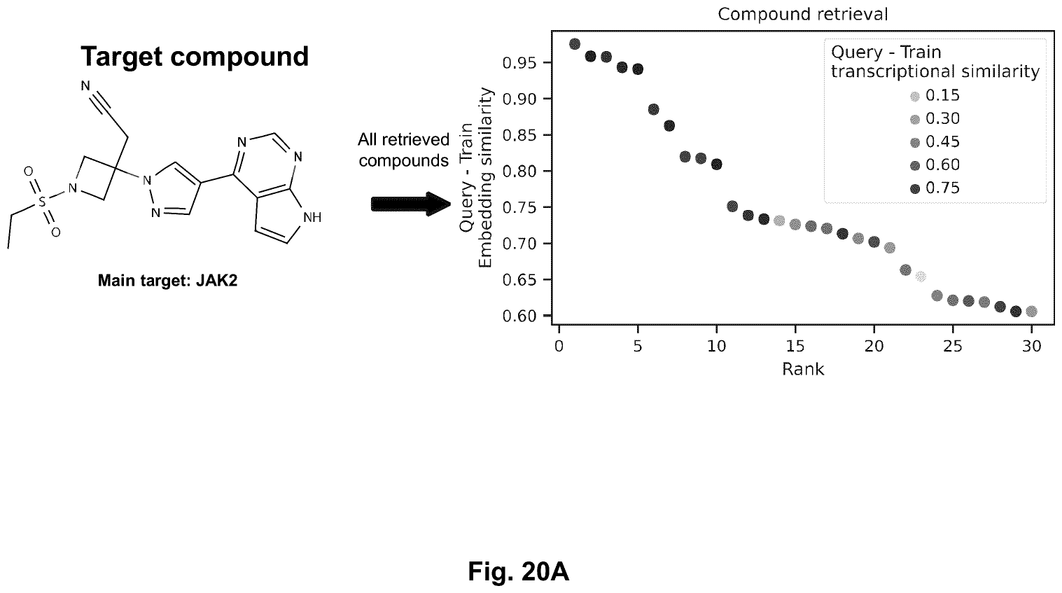

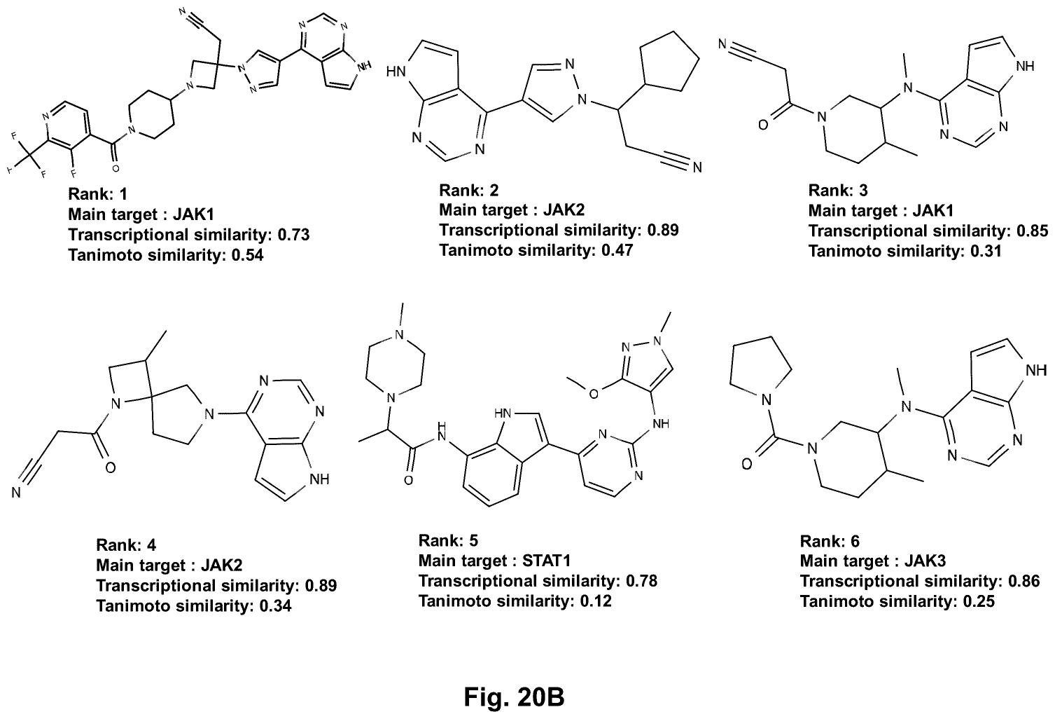

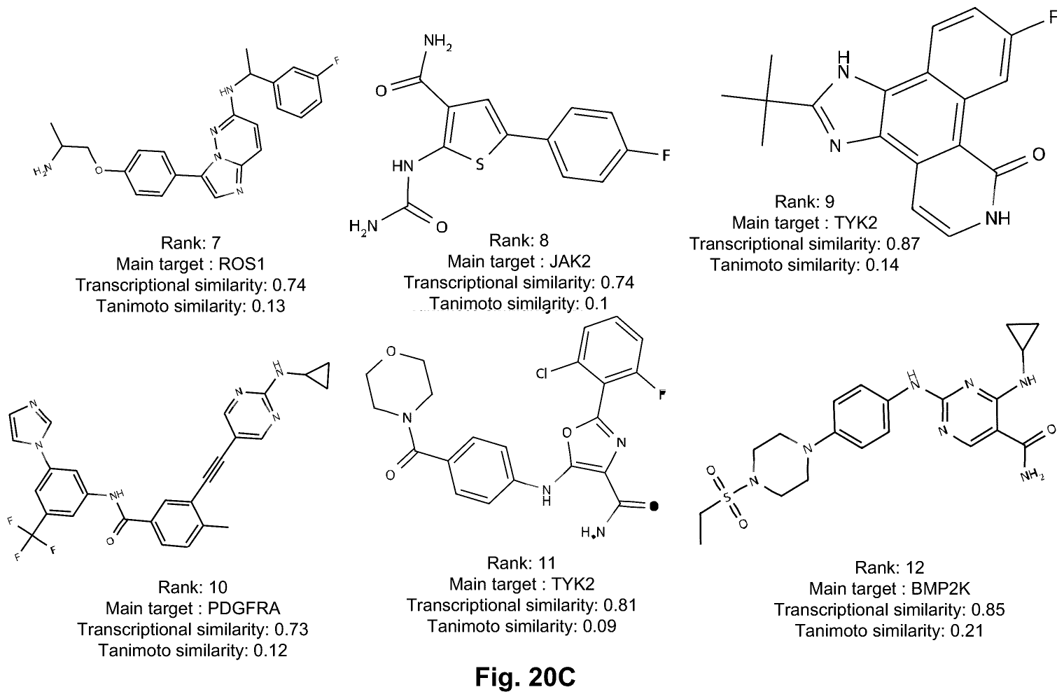

First, a JAK2 inhibitor was projected into the co-embedding and the top-50 most similar compounds were retrieved. Among expected JAK-STAT pathway inhibitors (FIG. 20A), we retrieved an ABL1 inhibitor, ranked 29, with a completely different molecular structure (FIG. 16A). Interestingly, several studies reported the coexistence of BCR-ABL1 and JAK2-V617F in patients with chronic myeloid leukemia. See Lorenzo et al., 2020, “Emergence of BCR-ABL1 Chronic Myeloid Leukemia in a JAK2-V617F Polycythemia Vera” J Hematol 9(1-2), pp. 23-29; and Cappetta et al., 2013, “Concomitant detection of BCR-ABL translocation and JAK2 V617F mutation in five patients with myeloproliferative neoplasm at diagnosis,” Int. J. Lab Hematol. 35(1):e-4-5, each of which is hereby incorporated by reference. Moreover, there is a clear link between this co-occurrence and myeloproliferative neoplasm pathogenesis (Soderquist et al., 2018, “,” Modern Pathology 31, pp. 690-704, which is hereby incorporated by reference) despite the lack of clarity on the order of acquisition of JAK2-V617F mutation and BCR-ABL1 translocation. In the present case, the JAK2 inhibitor and ABL1 inhibitor showed a high degree of transcriptional similarity (0.75+) and common regulation of 215 Rectome pathways, supporting the previous findings.

Second, a PLK1 inhibitor was projected, and among the retrieved compounds an HIF1A inhibitor was detected (FIG. 16B). Both these compounds induce extremely similar transcriptional response in CD34+(transcriptional cosine similarity of 0.95). According to StringDB (on the Internet at string-db.org; Szklarczyk et al., 2023, “The STRING database in 2023: protein-protein association networks and functional enrichment analyses for any sequenced genome of interest,” Nucleic Acids Res. 2023 Jan. 6; 51(D1):D638-646), no interaction between PLK1 and HIFIA is detected, however, PLK1 and HIF1A have correlated expression in cancer, such as melanoma (Dufies et al., 2021, “Plk1, upregulated by HIF-2, mediates metastasis and drug resistance of clear cell renal cell carcinoma,” Commun Biol. 4: 166, which is hereby incorporated by reference). It is also shown that HIFIA can induce PLK1 through HIF1α-NF-κB signaling cascade in hypoxia (Chen et al. 2023, “,” Respiratory Research 24, 204, which is hereby incorporated by reference). In this case, the disclosed model 2100 successfully placed these two compounds next to each other despite having drastically different structure (Tanimoto similarity of 0.192).

Third, an ABCB1 inhibitor was projected. Among recommended compounds retrieved was a BCL2 inhibitor (FIG. 16C). A high degree of transcription similarity was detected between the two (transcriptional cosine similarity 0.62) and no structural similarity (Tanimoto similarity of 0.08). Both ABCB1 and BCL2 are associated with multi drug resistance (MDR) in Acute myeloid leukemia (AML) pump-resistance (Svirnovski et al., 2009, “ABCB1 and ABCG2 proteins, their functional activity and gene expression in concert with drug sensitivity of leukemia cells,” Hematology 14(4): 204-12; and Robey et al., “Revisiting the role of ABC transporters in multidrug-resistant cancer,” Nat. Rev. Cancer 18(7), pp. 452-464, each of which is hereby incorporated by reference) and non-pump resistance mechanisms, (Kulsoom, et al., 2018, “Bax, Bcl-2, and Bax/Bcl-2 as prognostic markers in acute myeloid leukemia: are we ready for Bcl-2-directed therapy?,” Cancer Manag Res 2:10:403-416, which is hereby incorporated by reference), respectively. However, levels of BCL2 and ABCB1 are considered to be independent factors of MDR in AML (Pravdic et al., 2023, “The influence of BCL2, BAX, and ABCB1 gene expression on prognosis of adult de novo acute myeloid leukemia with normal karyotype patients,” Radiol Oncol. 57(2), pp. 239-248, which is hereby incorporated by reference) and in this case the disclosed model 2100 might have picked up a signal that results in similar drug-resistance outcomes in AML, but via two independent mechanisms.

Lastly, an IKZF1 inhibitor was projected and among identified compounds a TNF inhibitor was identified (FIG. 16D). The matched compound showed a high degree of transcriptional similarity with the query (0.76) and a moderate structural similarity (0.52). IKZF1 deficiency is associated with reduced production of TNF (along with IL-12 and IFN-α) in CD14+ monocytes (mono). CD14− monocytes and dendritic cell (DC) subtypes of PBMCs (Cytlak et al., 2018 “Ikaros family zinc finger 1 regulates dendritic cell development and function in humans,” Nat Commun. 9: 1239, which is hereby incorporated by reference), so inhibiting IKZF1 could lead to the same transcriptional changes as TNF inhibition.

These examples here illustrate the applicability of the disclosed model 2100 for recommending compounds with diverse structures but similar transcription, which can be directly used in transcription-based drug discovery programs.

In this work, we designed and tested SMORES, a multi-modal metric learning model that simultaneously projects the transcriptional signature of a compound and its structure into a shared latent space. Our model contributes to the expanding list of models in perturbational biology (GEARS, CHemCPA, PerturbNet) that are solving a task of finding compact representation of noisy biological data, by adding a weak biological priors and explicit way to model the effect of unseen compounds.

In this example a transcriptional encoder 2122 was pretrained, using multitask metric learning loss to increase the similarity between biological replicates of compounds and to incorporate grouping of compounds based on shared pathway regulation patterns as a weak prior to generate a biology-driven compounds clustering in the latent space. The example shows that this allows for the generation of meaningful embedding of transcriptional effect of compounds, and neighbors in the latent space exhibit high degree of similarity of pathway regulation.

In turn, the structure encoder 2112 was designed to align the embedding obtained through transcriptional encoder 2122 with the structure-based embedding, which allows for the prediction of a latent space vector even for compounds for which transcriptional perturbational data was not generated. Of note, this example demonstrated that by adding cellular context to the compound SMILES string, structure-based projections can effectively be generated that are conditioned on cell type and that this procedure yields biology-driven molecular representations.

By defining a concept of transcriptional mimics, the ability of the disclosed model to recommend molecules transcriptionally akin to a query, whilst structurally distinct, was tested and it showed 2-3 times enrichment over traditional structure-based models for compounds with mild activity. This example also demonstrated that despite being encoder-only model and not having an explicit reconstruction head, the latent representation of SMORES can be used to predict pathway regulation and differential gene expression from structure-based projections of unseen compounds.

This example further expanded upon that and illustrated how SMORES can be used in a task of hit augmentation, which is finding the compounds that are transcriptionally similar to the target compound and demonstrated that SMORES finds molecules from downstream targets or seemingly unrelated, which would be missed if traditional structure-only searches are used. This capability is important for a successful drug discovery, as it allows for predictions outside of the limited set of compounds for which expensive perturbational data have been obtained.

Since the disclosed model 2100 is a multi-modal model, new modalities can be easily integrated, for example, imaging in many cases can add orthogonal information to transcriptomics. Given the abundance of public datasets as well as the ability to generate imaging at scale, it is an attractive next modality to add. As another modality, genetic perturbations can be integrated into SMORES at the stage of the transcriptional encoder pre-training, as they can be added as pseudo compounds. Additionally, the disclosed metric learning procedure allows for the integration of different biological priors, e.g. protein-protein interactions. The disclosed model 2100 is a modular model and changes can be easily applied. For instance, SMILES conversion can be swapped with other methods or the MLP used in the transcriptional and structure encoder can be replaced with other AI architectures such as foundational models such as Geneformer (Theodoris et al., 2023, “Transfer learning enables predictions in network biology,” Nature, 618(7965):616-624, which is hereby incorporated by reference), scGPT (Cui et al., 2023, “scGPT: Towards Building a Foundation Model for Single-Cell Multi-omics Using Generative AI” bioRxiv 2023.04.30.538439, which is hereby incorporated by reference) and larger language models such as Chemformer (Ross Irwin et al, 2022, “Chemformer: a pre-trained transformer for computational chemistry,” Mach. Learn.: Sci. Technol. 3 015022), respectively. Finally, in some embodiments the disclosed model 2100 is a multi-modal model that has flexibility to receive input of new data modalities that are capable of augmenting and/or enhancing models and predictions.

Non-limiting examples of new modalities include genomics, transcriptomics, epigenomics, proteomics, and multi-omics data modalities, such as imaging/cell painting data, proteomic data, atacseq, and siteseq.

### Example 3 Assorted Methods

Dataset construction—The CD34+ intervention library (IL) used in Example 2 consisted of 3700 unique compounds across 130 96-well plates where 136 compounds were purposefully measured two times or more for the validation of the model's transcriptional encoder 2122. After selecting only compounds with at least one DEG at 5% FDR, 1870 active compounds with measurable and robust transcriptional response remained.

The IL compounds were divided into three groups. The first group was a test set for the transcriptional embedding by the transcriptional encode 2122. This first group consisted of 136 compounds that were used in the training but have been measured in different plates to test the ability of transcriptional encoder to correctly project unseen biological replicates of known compounds. The second group was a test set for the structure encoder 2112. This second group consisted of 255 compounds that have never been used and seen in the training. This second group was designed to test the ability of structure encoder 2112 to generalize across unseen compounds and pair these new structures with their transcriptional profiles. The third group was training compounds for the overall model 2100 and consisted of the remaining 1615 compounds.

Differential expression score. Differential expression (DE) analysis was conducted using two distinct methodologies: global DE and cell-type specific DE. For global DE, the raw expression counts was aggregated from all cells within each well, creating a comprehensive pseudobulk expression profile per well. For cell-type specific DE, the expression counts were compiled exclusively from cells identified as belonging to the same cell type, resulting in pseudobulk expression profiles that are cell-type specific within each well.

Subsequently, these pseudobulk profiles were processed through the limma-voom pipeline (limma-voom pipeline, which normalizes the expression counts and runs differential expression on the normalized values; Law et al., 2018, “RNA-seq analysis as easy as 1-2-3 with limma, Glimma, and edgeR,” F1000Research 5:1408, last updated 28 Dec. 2018, which is hereby incorporated by reference), which normalizes the counts and runs differential expression on the normalized values. The plate layout allowed for the inclusion of the sequencing lane, which corresponds to a row of the plate, as a covariate. Using the limma's outputs, Differential Expression Score (DES) were defined as −log₁₀(qᵥₐₗᵤₑ) sign(LFC).

Hyperparameter optimization. The construction of the disclosed model 2100 was met with an abundance of choices, each leading to a distinct model configuration. This diversity in potential models necessitated a systematic approach to discern the optimal combination of hyperparameters that yields the most accurate and biologically relevant results. To identify the ideal hyperparameters, the hyperparameter optimization framework Optuna was employed (Akiba et al., 2019, “Optuna: A Next-generation Hyperparameter Optimization Framework,” arXiv: 1907.10902 [cs.LG], which is hereby incorporated by reference), an open-source tool designed to automate the optimization process by efficiently searching through high-dimensional spaces. The objective function within Optuna was formulated to maximize compound recall, ensuring that the model was comprehensive in compound classification task. In each iteration of the optimization process, Optuna sampled a new training configuration. This encompassed variations in model architecture, the optimizer used, learning rate scheduling, and data handling parameters. The predefined parameter spaces for each component allowed for a broad yet directed search across the hyperparameter landscape. Utilizing these predefined parameter spaces, Optuna orchestrated a series of training cycles, each time recording the resulting compound recall. This metric served as a performance indicator, reflecting the model's ability to correctly project biological replicates of compounds in proximity of each other.

Performing extensive hyperparameter optimization was of significance for several reason. See example 4 for the exact hyperparameter search space used. First, it ensured that the model 2100 was well-tuned to the nuances of the transcriptional data, which is inherently complex and high-dimensional. Second, the optimization process aids in mitigating overfitting, as the recall-focused objective encourages the model 2100 to generalize well across different biological scenarios. Finally, by exploring a vast parameter space, the limits of the model's capabilities could be confidently approached, potentially uncovering novel insights into the transcriptional landscape that simpler models might overlook.

Compound recall at transcriptional encoder. To evaluate the quality of the embedding produced by the transcriptional encoder 2122, the proximity of biological replicates from the validation dataset to their corresponding training samples was examined, specifically, whether validation samples of a given compound were situated near the train samples of the same compound in the latent space. To streamline this process and conserve computational resources, a prototype-based approach was implemented. At the end of each epoch, a class prototype for each compound was derived by averaging the latent representation of all its training samples. Next, all validation samples were projected into the latent space. The quality of these embeddings was then quantified using the recall@10 metric: for each validation sample, the distance to every class prototype was calculated, the classes were ranked by proximity (using cosine similarity), and it was noted whether the true class was among the ten nearest. A “hit” was registered when the correct class prototype ranked within this top-10 threshold. The final recall@10, the aggregate measure of quality, was obtained by averaging these hit rates across all validation compounds.

Compound recall at co-embedding. A metric for quantifying the accuracy with which transcriptional data can be matched to their corresponding molecular structures was sought. Specifically, how reliably a transcriptional embedding of a sample can be assigned to its structural counterpart from the structure encoder was assessed. To enhance efficiency, during the training process, structure prototypes at epoch's end were calculated by averaging the structure-based embeddings for each compound, considering all replicates and cell types. Subsequently, each sample's transcription was projected into the latent space using the transcriptional encoder 2122, compared against all structure prototypes, and a determination was made if the correct structure was among the top-nearest, essentially computing recall@50. A “hit” was called when the correct structure was within a top-50 closest neighbor.

Of note, when calculating recall, a conditional projection method was adopted. For a given sample, its corresponding baseline expression was taken and used to project the structures of all compounds, thereby creating embeddings conditioned on that specific sample's baseline. The sample's transcription was then projected using the transcriptional encoder 2122, the similarity to these conditioned structure embeddings was calculated, and a hit recorded if the correct structure fell within the top-50.

Transcriptional mimics definition. A primary objective of the disclosed model 2100 was to identify molecules that elicit transcriptional responses akin to a given target compound. To facilitate this, the notion of a “transcriptional mimic” was introduced. Compound X was designated as a transcriptional mimic of compound Y if they generate sufficiently similar transcriptional responses, quantified by surpassing a predefined threshold of cosine similarity within the original DES space. The goal was to establish a reference set of mimics for each test compound, effectively compiling a roster of potential biochemical analogs.

Employing the DES, the similarity between every test compound and all training compounds was assessed. From this similarity distribution, the standard deviation (sd) and mean per each test compound was calculated, subsequently setting a compound-specific threshold for mimicry at mean+3 standard deviations. Therefore, any training compound exhibiting a similarity score that exceeds this threshold was classified as a transcriptional mimic for a corresponding test compound, and this threshold was dependent on overall compound activity level (FIG. 19E). It is noteworthy that for 35 of the 255 test compounds, a single mimic could not be identified, indicating that these compounds' transcriptional responses were distinct and unparalleled in the context of the training data. On average, each test compound shared transcriptional mimicry with 25 counterparts from the training set (FIG. 19F).

Transcriptional hit rate at co-embedding. To assess transcriptional fidelity, the proximity of test compounds to their training-set mimics within the co-embedding was evaluated. Here, only the structural information of test compounds was utilized to project them into our co-embedding, and an assessment of how close the transcriptional profiles of test compounds were to the nearest training compounds in the co-embedding was made. This measure reflects the ability of the encoder to learn a transcription-centric representation of molecular structures, which is particularly insightful when the structural similarity, indicated by Tanimoto coefficients, is low. Various molecular featurization models, including ECFP, ChemGPT, Signaturizer, and others capable of translating SMILES into a representative molecular embedding, were utilized for comparison. These models span a range from physics-informed descriptors to proximity-based fingerprints and learned embeddings.

For each model, embeddings for all molecules in the train and test datasets were generated. To benchmark the fidelity of these embeddings, a methodical approach was employed: for each test compound (query), relying only on structural information, the pairwise distances to all training compounds was calculated, ranked by similarity, cosine similarity for continuous embeddings and Jaccard similarity for binary vector representations like ECFP and MACCS. The top-50 most similar compounds for each query was then identified. The transcriptional hit rate was then calculated as the fraction of compounds within the top-50 sets of queries that satisfied the definition of transcriptional mimics (see previous subsection). Note that the metric was computed for each embedding model, stratified by compound activity as inferred from the number of DEGs, providing a comparative analysis of their performance.

Transcription & Pathway regulation reconstruction. The encoder model was architecturally designed without a reconstruction capability; it lacks an inbuilt mechanism to decode from the latent space back to the original input dimensions (a reconstruction head). Nevertheless, the latent space's predictive potential was explored for unseen compounds' DES or pathway regulation based solely on the co-embedding. To achieve this, sklearn.neighbors.KNeighborsRegressor (Scikit-learn: Machine Learning in Python, Pedregosa et al., JMLR 12, pp. 2825-2830, 2011, which is hereby incorporated by reference) was utilized by fitting it on the structure-based embedding of the training compounds as predictors (X) and using the DES of individual gene or individual pathway regulation score as Y. This generated a model that takes a structure-based embedding of a compound and predicts a regulation of a gene or a pathway. To handle 14,649 genes (in the task of predicting DE profile of an unseen compound) sklearn.multioutput.MultiOutputRegressor was used to wrap individual model into a single one. For each test compound, all 14,649 genes (in the task of DES prediction) were predicted and the predicted vector was compared against the observed DES, calculating the R square value to quantify the model's quality.

### Example 4—Hyperparameter Spaces for Optuna Optimization

Model architectures

| Parameter | Options/Range |
|---|---|
| Model Name | MLP |
| Dropout Probability | 0.05-0.75, step = 0.05 |
| Activation Function | ReLU, Mish, LeakyReLU, SELU |
| Batch Normalization | False, True |
| Number of Layers | 1-3 |
| Hidden Layer Sizes | 128-2048, step = 64 |
| Layer Order | act-bn-drop, bn-act-drop, drop-bn-act |
| Input Dimension | 14,649 |
| Output Dimension | 64-128, step = 8 |

Optimizer

| Parameter | Options/Range |
|---|---|
| Optimizer Name | AdamW, AdaBelief, NovoGrad, Ranger |
| Learning Rate | Log-uniform between 1e−4 and 5e−1 |
| Weight Decay | Log-uniform between 1e−4 and 1e−3 |

LR scheduler

| Parameter | Options/Range |
|---|---|
| LR Scheduler Name | ReduceLROnPlateau, |
| | CosineAnnealingWarmRestarts |
| factor (ReduceLROnPlateau) | 0.01-0.1, step = 0.01 |
| patience (ReduceLROnPlateau) | 5-10, step = 1 |
| mode (ReduceLROnPlateau) | max |
| T_0 (CosineAnnealingWarmRestarts) | 10-20, step = 1 |
| T_mult | 1-4, step = 1 |
| (CosineAnnealingWarmRestarts) | |
| eta_min | Log-uniform between |
| (CosineAnnealingWarmRestarts) | 1e−8 and 1e−6 |

Transcriptional encoder samplers

| Task | | | |
|---|---|---|---|
| Index | Miner Name | Parameter | Options/Range |
| Task 1 | AngularMiner | angle | 10-30, step = 2 |
| Task 1 | TripletMarginMiner | margin | 0.05-0.55, step = |
| | | | 0.05 |
| Task 1 | TripletMarginMiner | type_of_triplets | hard, semihard |
| Task 1 | MultiSimilarityMiner | epsilon | 0.05-0.55, step = |
| | | | 0.05 |
| . . . | . . . | . . . | . . . |
| Task N | AngularMiner | angle | 10-30, step = 2 |
| Task N | TripletMarginMiner | margin | 0.05-0.55, step = |
| | | | 0.05 |
| Task N | TripletMarginMiner | type_of_triplets | hard, semihard |
| Task N | MultiSimilarityMiner | epsilon | 0.05-0.55, step = |
| | | | 0.05 |

Note that each task has an individual sampler.

Transcriptional encoder regularizer

| Parameter | Options |
|---|---|
| Regularizer Name | CenterInvariantRegularizer, LpRegularizer, |
| | ZeroMeanRegularizer |

Transcriptional encoder losses

| Parameter | Options | Details |
|---|---|---|
| Criterion Name | List of possible criteria | One of [AngularLoss, |
| | | TripletMarginLoss, |
| | | ArcFaceLoss, CosFaceLoss, |
| | | CircleLoss, |
| | | ProxyAnchorLoss, |
| | | SphereFaceLoss, |
| | | NTXentLoss, |
| | | SupConLoss, |
| | | GeneralizedLiftedStructureLoss] |
| AngularLoss alpha | Int range(36, 55) | |
| TripletMarginLoss margin | Float range(0.02, 0.1) | |
| TripletMarginLoss | [True, False] | |
| smooth_loss | | |
| ArcFaceLoss num_classes | Derived from labels dict | |
| ArcFaceLoss embedding_size | Int | |
| ArcFaceLoss margin | Float range(16, 32) | |
| ArcFaceLoss scale | Float range(32, 76) | |
| CosFaceLoss num_classes | Derived from labels dict | |
| CosFaceLoss embedding_size | Int | |
| CosFaceLoss margin | Float range(0.20, 0.55) | |
| CosFaceLoss scale | Float range(32, 76) | |
| CircleLoss m | Float range(0.20, 0.55) | |
| CircleLoss gamma | Float range(80, 256) | |
| ProxyAnchorLoss | Derived from labels dict | |
| num_classes | | |
| ProxyAnchorLoss | Int | |
| embedding_size | | |
| ProxyAnchorLoss margin | Float range(0.05, 0.5) | |
| ProxyAnchorLoss alpha | Float range(16, 48) | |
| SphereFaceLoss num_classes | Derived from labels dict | |
| SphereFaceLoss | Int | |
| embedding_size | | |
| SphereFaceLoss margin | Float range(2, 10) | |
| SphereFaceLoss scale | Float range(1, 16) | |
| NTXentLoss temperature | Float range(0.05, 0.5) | |
| SupConLoss temperature | Float log range(1e−2, 5) | |
| GeneralizedLiftedStructureLoss | Float range(0.1, 1.5) | |
| neg_margin | | |
| GeneralizedLiftedStructureLoss | Float range(0.0, 0.1) | |
| pos_margin | | |

Note that Each task has an individual loss, but all tasks are using the same class.

Transcriptional encoder data parameters

| | | | Suggested | |
|---|---|---|---|---|
| Parameter | Description | Default Value | Range | Step/Method |
| batch_size | The number of | Not Applicable | 8 to 64 | 8 |
| | samples per | | | |
| | batch. | | | |
| m | Number of | Not Applicable | 2 to 3 | 1 |
| | samples per | | | |
| | class | | | |

Structural encoder loss

| Parameter | Type | Default | Description |
|---|---|---|---|
| criterion_name | Categorical | [“LogCoshLoss”, | The name of the loss |
| | | “L1Loss”, | criterion to be used. |
| | | “MSELoss”] | |

Structural encoder data parameters

| | | | Suggested | |
|---|---|---|---|---|
| Parameter | Description | Default Value | Range | Step/Method |
| batch_size | The number | Not Applicable | 128 to 4096 | 128 |
| | of samples | | | |
| | per batch. | | | |

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer readable storage medium. For instance, the computer program product could contain the program modules shown in any combination of FIGS. 1-3, 8-10, and 21-23. These program modules can be stored on a CD-ROM, DVD, magnetic disk storage product, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1-138. (canceled)

139. A method of determining whether a first compound and a second compound are causal for a common biological state, the method comprising:
A) inputting a first input data structure into a structure encoder, wherein
the first input data structure comprises a combination of a feature representation of the first compound and a baseline transcriptional representation, and
the structure encoder comprises a first plurality of parameters,
thereby retrieving, by operation of the first plurality of parameters on the first input data structure in accordance with an architecture of the structure encoder, as output from the structure encoder, a first compound embedding having a first dimension;
B) determining a respective similarity between the first compound embedding and each respective transcriptional embedding in a plurality of transcriptional embeddings thereby determining a plurality of similarities, wherein
each transcriptional embedding in the plurality of transcriptional embeddings has the first dimension,
each transcriptional embedding in the plurality of transcriptional embeddings is generated from inputting a corresponding cellular constituent abundance data set representative of the first cell type exposed to a different perturban in a plurality of perturbans, into a transcriptional encoder comprising a second plurality of parameters,
the plurality of perturbans includes the second compound,
the plurality of transcriptional embeddings comprises at least 25 transcriptional embeddings, and
the structure encoder is trained to minimize a loss against the plurality of transcriptional embeddings; and
C) associating the first compound with a biological state that the second compound is known to be causal for when the comparing B) determines that the similarity between the first compound embedding and the respective transcriptional embedding of the second compound satisfies a similarity criterion.

140. The method of claim 139, wherein the similarity criterion is satisfied when the similarity assigned to the respective transcriptional embedding by the determining B) is in a top N^{th} percentile of the plurality of similarities.

141. The method of claim 140, wherein the N^{th} percentile is between fifty percent and ninety-five percent.

142-143. (canceled)

144. The method of claim 139, wherein the similarity criterion is satisfied when the similarity assigned to the respective transcriptional embedding by the determining B) is in the top N similarities in the plurality of similarities.

145. The method of claim 144, wherein N is between 5 and 100 and the plurality of transcriptional embeddings comprises at least 1000 transcriptional embeddings.

146. (canceled)

147. The method of claim 139, the method further comprising determining the feature representation of the first compound from a string representation of a chemical structure of the first compound.

148. The method of claim 147, wherein the string representation is in a SMARTS, DeepSMILES, or SELFIES format.

149. The method of claim 147, wherein the string representation is in a simplified molecular-input line-entry system (SMILES) format.

150. The method of claim 147, wherein the determining the feature representation of the first compound from a string representation of a chemical structure of the first compound comprises inputting the string representation into each featurizer in a set of featurizers to obtain the feature representation.

151-152. (canceled)

153. The method of claim 139, wherein the feature representation of the first compound consists of between 150 and 10,000 features.

154. (canceled)

155. The method of claim 139, wherein the baseline transcriptional representation is that of a first cell type.

156. The method of claim 155, wherein the baseline transcriptional representation comprises pathway activation scores for a plurality of pathways derived from cellular constituent abundance data for a plurality of cellular constituents in a plurality of cells of the first type that are in a baseline state.

157. The method of claim 156, wherein each cellular constituent in the plurality of cellular constituents uniquely maps to a different gene.

158. The method of claim 156, wherein each cellular constituent in the plurality of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein.

159. The method of claim 156, wherein the plurality of cellular constituents comprises 50 or more cellular constituents, 100 or more cellular constituents, 150 or more cellular constituents, 200 or more cellular constituents, 300 or more cellular constituents, 500 or more cellular constituents, 1000 or more cellular constituents, 2000 or more cellular constituents, 4000 or more cellular constituents, or 8000 or more cellular constituents.

160. The method of claim 156, wherein the plurality of pathways comprises 10 or more pathways, 20 or more pathways, 50 or more pathways, 100 or more pathways, or 500 or more pathways.

161-166. (canceled)

167. The method of claim 139, wherein the structure encoder is a first multilayer perceptron having a first plurality of hidden layers.

168-170. (canceled)

171. The method of claim 139, wherein the first compound embedding having the first dimension consists of between 40 and 2000 dimensions.

172-174. (canceled)

175. The method claim 139, wherein the corresponding cellular constituent data set comprises single cell transcriptional data for a plurality of cells of a first type.

176. The method claim 139, wherein the corresponding cellular constituent data comprises bulk transcriptional data for a plurality of cells of the first type.

177. The method of claim 139, wherein the corresponding cellular constituent data set comprises cellular constituent abundance values for a plurality of cellular constituents.

178. (canceled)

179. The method of claim 177, wherein each cellular constituent in the plurality of cellular constituents is a particular gene, a particular mRNA associated with a gene, a carbohydrate, a lipid, an epigenetic feature, an epitranscriptomic feature, a metabolite, an antibody, a peptide, a protein, or a post-translational modification of a protein.

180. The method of claim 177, wherein the plurality of cellular constituents comprises 50 or more cellular constituents, 100 or more cellular constituents, 150 or more cellular constituents, 200 or more cellular constituents, 300 or more cellular constituents, 500 or more cellular constituents, 1000 or more cellular constituents, 2000 or more cellular constituents, 4000 or more cellular constituents, or 8000 or more cellular constituents.

181. The method of claim 139, wherein the corresponding cellular constituent abundance data set comprises a corresponding differential expression signature for a plurality of cells of a first type.

182. The method of claim 181, wherein
the corresponding differential expression signature comprises a plurality of differential values,
each respective differential value in the plurality of differential values corresponds to a respective cellular constituent in a set of cellular constituents, and
the respective differential value represents a difference between (i) one or more abundance values measured for the respective cellular constituent in a first assay of a first plurality of cells of the first cell type that represent a first cell state and (ii) one or more abundance values measured for the respective cellular constituent in a second assay of a second plurality of cells of the first cell type that represent a second cell state.

183. The method of claim 182, wherein
the first cell state is exposure of the first plurality of cells to a respective perturban in the plurality of perturbans, and
the second cell state is exposure of the second plurality of cells to a reference environment.

184-186. (canceled)

187. The method of claim 139, wherein the plurality of transcriptional embeddings collectively represents over 500 different first cell states.

188-195. (canceled)

196. The method of claim 139, wherein the respective transcriptional embedding consists of between 40 and 2000 dimensions.

197-203. (canceled)

204. A computer system, comprising one or more processors and memory, the memory storing instructions for performing a method of determining whether a first compound and a second compound are causal for a common biological state, the method comprising:
A) inputting a first input data structure into a structure encoder, wherein
the first input data structure comprises a combination of a feature representation of the first compound and a baseline transcriptional representation, and
the structure encoder comprises a first plurality of parameters,
thereby retrieving, by operation of the first plurality of parameters on the first input data structure in accordance with an architecture of the structure encoder, as output from the structure encoder, a first compound embedding having a first dimension;
B) determining a respective similarity between the first compound embedding and each respective transcriptional embedding in a plurality of transcriptional embeddings thereby determining a plurality of similarities, wherein
each transcriptional embedding in the plurality of transcriptional embeddings has the first dimension,
each transcriptional embedding in the plurality of transcriptional embeddings is generated from inputting a corresponding cellular constituent abundance data set representative of the first cell type exposed to a different perturban in a plurality of perturbans, into a transcriptional encoder comprising a second plurality of parameters,
the plurality of perturbans includes the second compound,
the plurality of transcriptional embeddings comprises at least 25 transcriptional embeddings, and
the structure encoder is trained to minimize a loss against the plurality of transcriptional embeddings; and
C) associating the first compound with a biological state that the second compound is known to be causal for when the comparing B) determines that the similarity between the first compound embedding and the respective transcriptional embedding of the second compound satisfies a similarity criterion.

205. A non-transitory computer-readable medium storing one or more computer programs, executable by a computer, for determining whether a first compound and a second compound are causal for a common biological state, the computer comprising one or more processors and a memory, the one or more computer programs collectively encoding computer executable instructions that perform a method comprising:
A) inputting a first input data structure into a structure encoder, wherein
the first input data structure comprises a combination of a feature representation of the first compound and a baseline transcriptional representation, and
the structure encoder comprises a first plurality of parameters,
thereby retrieving, by operation of the first plurality of parameters on the first input data structure in accordance with an architecture of the structure encoder, as output from the structure encoder, a first compound embedding having a first dimension;
B) determining a respective similarity between the first compound embedding and each respective transcriptional embedding in a plurality of transcriptional embeddings thereby determining a plurality of similarities, wherein
each transcriptional embedding in the plurality of transcriptional embeddings has the first dimension,
each transcriptional embedding in the plurality of transcriptional embeddings is generated from inputting a corresponding cellular constituent abundance data set representative of the first cell type exposed to a different perturban in a plurality of perturbans, into a transcriptional encoder comprising a second plurality of parameters,
the plurality of perturbans includes the second compound,
the plurality of transcriptional embeddings comprises at least 25 transcriptional embeddings, and
the structure encoder is trained to minimize a loss against the plurality of transcriptional embeddings; and
C) associating the first compound with a biological state that the second compound is known to be causal for when the comparing B) determines that the similarity between the first compound embedding and the respective transcriptional embedding of the second compound satisfies a similarity criterion.
